(19) United States
(12) Patent Application Publication
(10) Pub. No.: US 2026/0071117 A1
(43) Pub. Date: Mar. 12, 2026

(54) FUNABLE FLUORESCENT COMPOUNDS AND USES THEREOF

(71) Applicant: Arizona Board of Regents on Behalf of the University of Arizona, Tucson, AZ (US)

(72) Inventors: Christopher Hulme, Tucson, AZ (US); Nathan Bedard, Tucson, AZ (US); Andrew Sennett, Tucson, AZ (US)

(21) Appl. No.: 18/705,765

(22) PCT Filed: Nov. 3, 2022
(86) PCT No.: PCT/US2022/048782

§ 371 Date: Apr. 29, 2024

Related U.S. Application Data
(60) Provisional application No. 63/275,022, filed on Nov. 3, 2021.

(51) Int. Cl. C09K 11/06; C07D 471/04; C07D 471/14; C07D 487/04; C07F 5/02; G01N 33/58

(52) U.S. Cl. CPC C09K 11/06; C07D 471/04; C07D 471/14; C07D 487/04; C07F 5/025; G01N 33/582; C09K 2211/1007; C09K 2211/1011; C09K 2211/1018

(57) ABSTRACT

Provided herein are compositions and methods for detecting analytes. In particular, provided herein are tunable fluorescent indolizine and imidazo-triazines compounds and uses thereof (e.g., as probes or biosensors).

### STATEMENT REGARDING RELATED APPLICATIONS

This application claims priority to U.S. Provisional Application No. 63/275,022, filed Nov. 3, 2021, the entire contents of which are incorporated herein by reference for all purposes.

### STATEMENT OF GOVERNMENT SUPPORT

This invention was made with government support under Grant No. MH090878 awarded by National Institutes of Health. The government has certain rights in the invention

### FIELD OF THE INVENTION

Provided herein are compositions and methods for detecting analytes. In particular, provided herein are tunable fluorescent indolizine and imidazo-triazines compounds and uses thereof (e.g., as probes or biosensors).

### INTRODUCTION

Indolizines are condensed [6,5]-heterocycles containing an inner nitrogen atom.1 Importantly, this aromatic chemotype in its reduced form (indolizidines) is prevalent in natural products, which include (−)-swainsonine 1, and tashirome 2, both possessing anti-cancer activity1-3. Indeed indolizines exhibit a wide range of pharmaco-logical effects, such as the toxicity associated with pu-miliotoxins extracted from poison-dart frogs, inhibitory activity for both the phosphodiesterase PDE5A 3 and vascular endothelial growth factor (VEGF) 4,1,4,5 Moreover, indolizines are found in Seoul-Fluor 5 and PITE 6, which have utility as biomedical fluorescent probes for ratiometric pH sensing, reactive oxygen species sensing, and lipid droplet characterization.4,6

Tunable fluorescent compounds are particularly useful in biomedical applications as they allow for multiplexing.

Tunable indolizines and methods for their synthesis are needed.

### SUMMARY

The present disclosure provides tunable fluorescent tetracyclic indolizines and imidazo-triazines generated by an improved method that simplifies their synthesis. The method is a two-step multicomponent reaction oxidation protocol.

For example, in some embodiments, provided herein is a composition, comprising a compound selected from, for example,

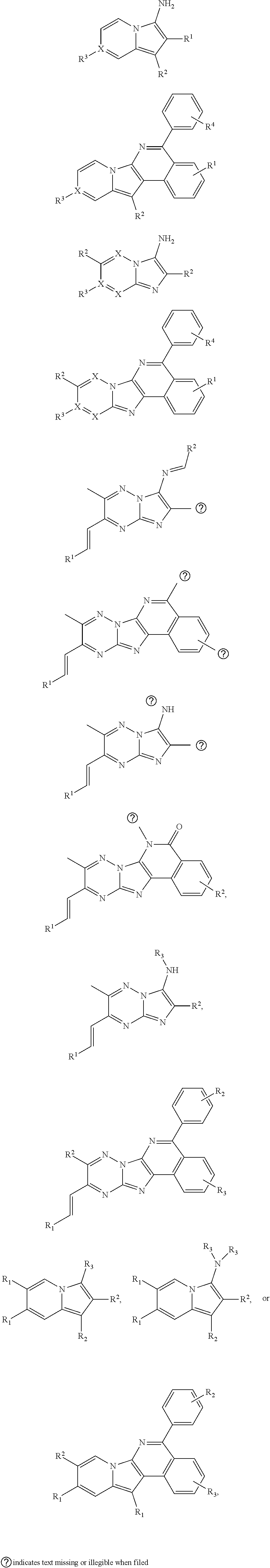

wherein X is CR, CH, or N, and R₁, R₂, R₃, and R₄ are independently selected from, for example, null, H, (CH₂)ₙ—O—(CH₂)ₘ, substituted or unsubstituted aryl, substituted or unsubstituted C₁-C₆ alkyl, where n and m are independently integers between 0 and 6, CN, halide, N-alkyl, O-alkyl, or N-aryl. In some embodiments, R₁ and R₄ are —OCH₃. In some embodiments, R₂ is CH₂CN. In some embodiments, R₁, R₂, R₃, and R₄ are independently selected from, for example isobutyl,

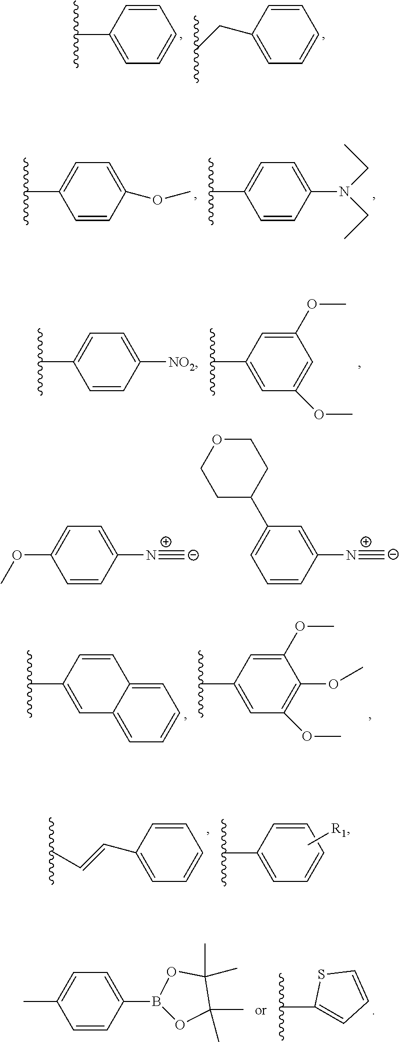

In some embodiments, R₁ and R₄ are independently ortho, meta, para, or a combination thereof.

Exemplary compounds of the present disclosure include but are not limited to,

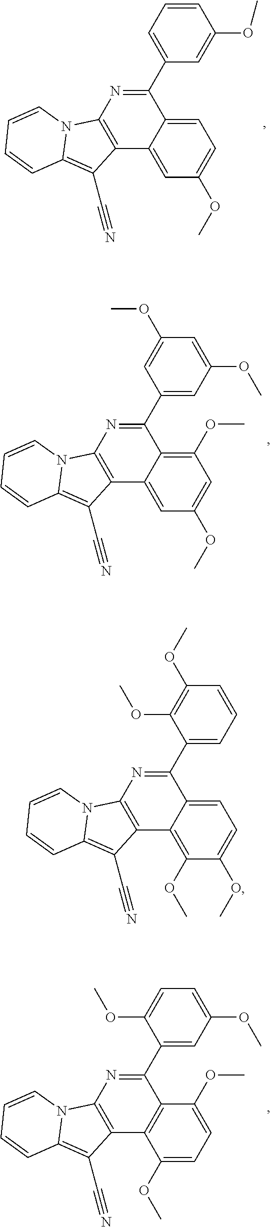 [SMILES: COc1cccc(-c2nc3c(c(C#N)c4ccccn43)c3cc(OC)ccc23)c1 ; COc1cc(OC)cc(-c2nc3c(c(C#N)c4ccccn43)c3cc(OC)cc(OC)c23)c1 ; COc1cccc(-c2nc3c(c(C#N)c4ccccn43)c3c(OC)c(OC)ccc23)c1OC ; COc1ccc(OC)c(-c2nc3c(c(C#N)c4ccccn43)c3c(OC)ccc(OC)c23)c1]

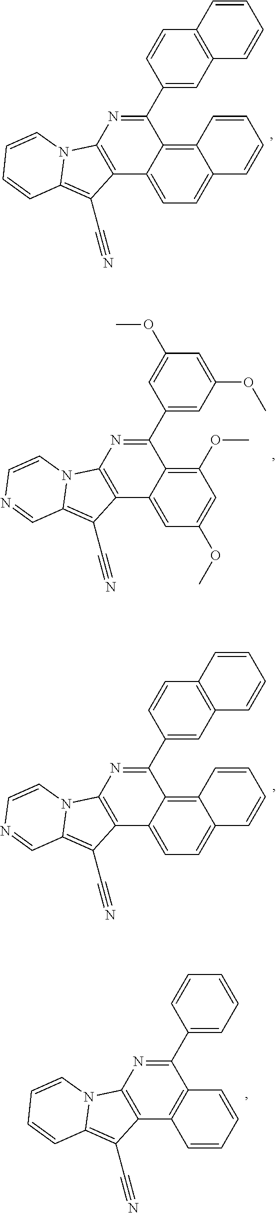 [SMILES: N#Cc1c2c3ccc4ccccc4c3c(-c3ccc4ccccc4c3)nc2n2ccccc12 ; COc1cc(OC)cc(-c2nc3c(c(C#N)c4cnccn43)c3cc(OC)cc(OC)c23)c1 ; N#Cc1c2c3ccc4ccccc4c3c(-c3ccc4ccccc4c3)nc2n2ccncc12 ; N#Cc1c2c3ccccc3c(-c3ccccc3)nc2n2ccccc12]

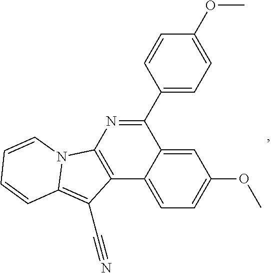 [SMILES: COc1ccc(-c2nc3c(c(C#N)c4ccccn43)c3ccc(OC)cc23)cc1]

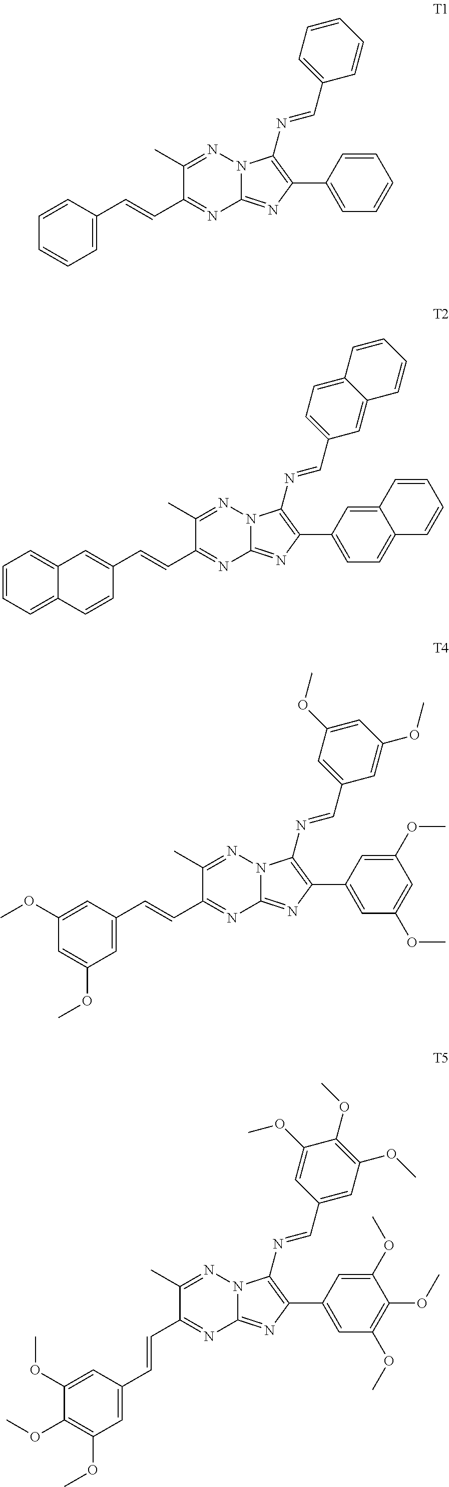

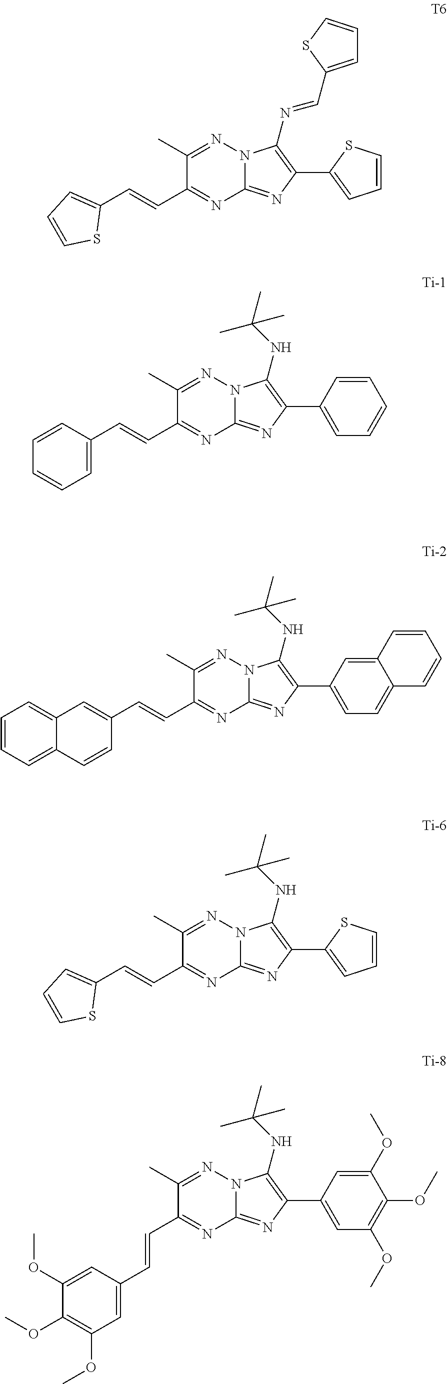

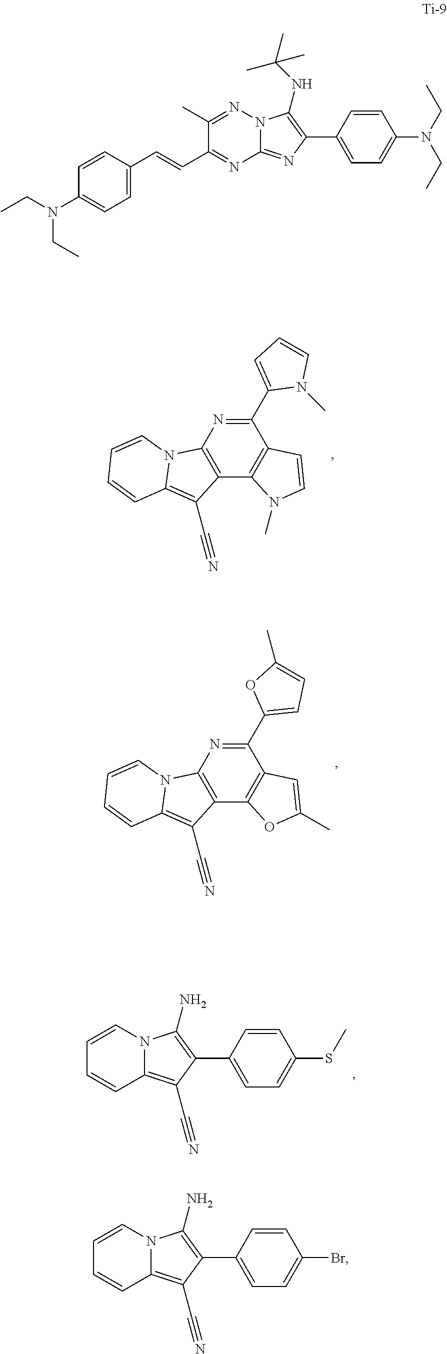 [SMILES: CCN(CC)c1ccc(/C=C/c2nc3nc(-c4ccc(N(CC)CC)cc4)c(NC(C)(C)C)n3nc2C)cc1 ; Cn1cccc1-c1nc2c(c(C#N)c3ccccn32)c2c1ccn2C ; Cc1ccc(-c2nc3c(c(C#N)c4ccccn43)c3oc(C)cc23)o1 ; CSc1ccc(-c2c(C#N)c3ccccn3c2N)cc1 ; N#Cc1c(-c2ccc(Br)cc2)c(N)n2ccccc12]

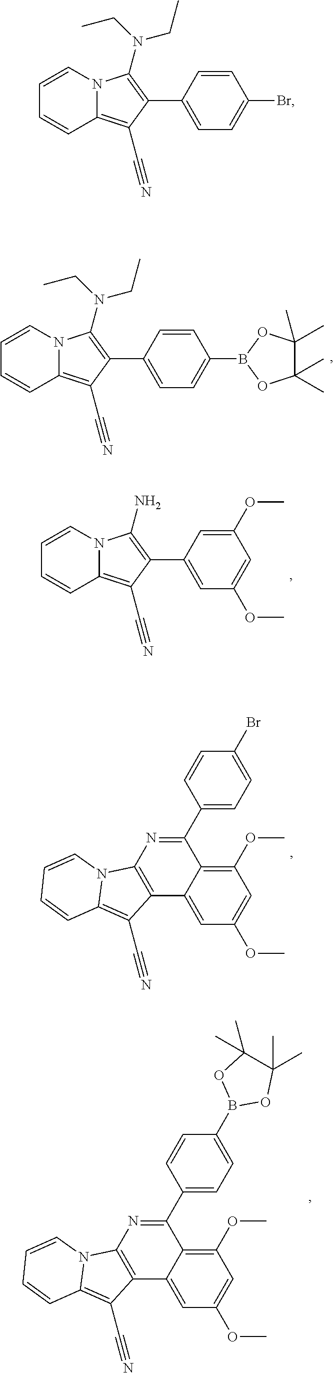 [SMILES: CCN(CC)c1c(-c2ccc(Br)cc2)c(C#N)c2ccccn12 ; CCN(CC)c1c(-c2ccc(B3OC(C)(C)C(C)(C)O3)cc2)c(C#N)c2ccccn12 ; COc1cc(OC)cc(-c2c(C#N)c3ccccn3c2N)c1 ; COc1cc(OC)c2c(-c3ccc(Br)cc3)nc3c(c(C#N)c4ccccn43)c2c1 ; COc1cc(OC)c2c(-c3ccc(B4OC(C)(C)C(C)(C)O4)cc3)nc3c(c(C#N)c4ccccn43)c2c1]

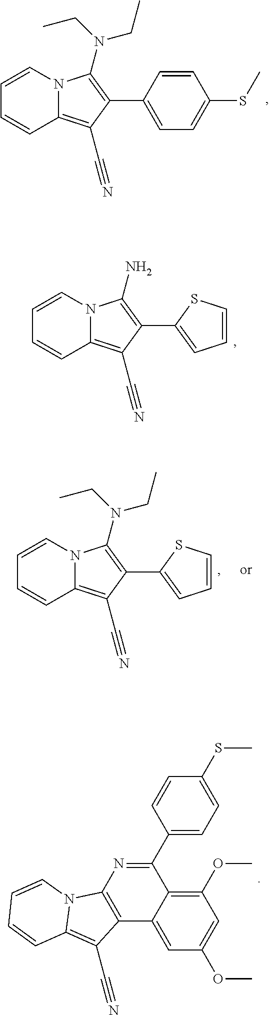 [SMILES: CCN(CC)c1c(-c2ccc(SC)cc2)c(C#N)c2ccccn12 ; N#Cc1c(-c2cccs2)c(N)n2ccccc12 ; CCN(CC)c1c(-c2cccs2)c(C#N)c2ccccn12 ; COc1cc(OC)c2c(-c3ccc(SC)cc3)nc3c(c(C#N)c4ccccn43)c2c1]

In some embodiments, the compound is fluorescent. In some embodiments, the compound is a probe. In certain embodiments, the compound is conjugated to one or more second molecules (e.g., via a linker, for example, at R₃) In some embodiments, the second molecule is a solubilizing group (e.g., any group that enhances aqueous solubility). In some embodiments, the compound comprises a solubilizing group (e.g., as a substituent).

Further embodiments provide a kit or system comprising one or more compounds described herein. For example, in some embodiments, the one or more compounds comprises two or more compounds, wherein each of the compounds emits fluorescence of a different wavelength. In some embodiments, the system further comprises one or more additional components necessary, useful, or sufficient for using the compounds. Examples include but are not limited to one or more buffers, one or more detection reagents, a fluorescent spectrometer, one or more control agents, or software for analysis or detection of an analyte of interest.

Other embodiments provide a method of detecting an analyte of interest, comprising: a) contacting a sample comprising the analyte of interest with a compound, kit or system described herein, and b) detecting fluorescence from the compound. The present disclosure is not limited to particular analytes. Examples include but are not limited to, a protein, a small molecule, or an acid. In some embodiments, the method detects the pH of the sample.

Also provided herein is the use of a compound, kit or system described herein to detect any analyte.

Additional embodiments are described herein.

### BRIEF DESCRIPTION OF THE DRAWINGS

FIG. 1 shows reaction scheme 1.

FIG. 2 shows reaction scheme 2.

FIG. 3 shows reaction scheme 3.

FIG. 4 shows reaction scheme 4.

FIG. 5 shows images of compounds 18a-15f (0.1 mg in 3 mL DMSO) using 365 nm TLC UV lamp for fluorescence visualization.

FIG. 6 shows a crystal structure of an exemplary compound of the present disclosure.

FIG. 7 shows hydrogen bonding in a crystal structure of an exemplary compound of the present disclosure.

FIG. 8 shows unit cell and packing in a crystal structure of an exemplary compound of the present disclosure.

FIG. 9 shows fluorescent comparisons of exemplary compounds of the present disclosure.

FIG. 10 shows aldehyde substituent variation of exemplary compounds of the present disclosure.

FIG. 11 shows aminopyridine variation of exemplary compounds of the present disclosure.

FIG. 12 shows pyridazine and para-methoxy ester variation of exemplary compounds of the present disclosure.

FIG. 13 shows pyridine and para-methoxy ester variation of exemplary compounds of the present disclosure.

FIG. 14 shows an exploration of the EDG-EWG relationship of exemplary compounds of the present disclosure.

FIG. 15 shows cyclized fluorescence of exemplary compounds of the present disclosure.

FIG. 16 shows electronic effects of aldehyde variations of exemplary compounds of the present disclosure.

FIG. 17 shows solvent effects and quantum yield of exemplary compounds of the present disclosure.

FIG. 18 shows the low-energy onset absorption spectra of 10f, 10q, 10k, and 10ba and the TD-DFT calculated wavelengths of the first excitations.

FIG. 19 shows experimental and computed (CAMY-B3LYP) properties of 10f, 10q, 10k and 10ba.

FIG. 20 show exemplary products of the GBB reaction.

FIG. 21 shows explorations of the GBB reaction's viability.

FIG. 22 shows solvent effects on fluorescence of exemplary compounds of the present disclosure.

FIG. 23A shows ¹H NMR of exemplary compounds of the present disclosure.

FIG. 23B shows HRMS spectra of exemplary compounds of the present disclosure.

FIG. 23C shows TD-DFT calculations of exemplary compounds of the present disclosure.

FIG. 23D shows CAMY-B3LYP Computed HOMO-LUMO Electron Density of

Molecules 10f, 10q, 10k, and 10ba.

### Definitions

To facilitate an understanding of the present disclosure, a number of terms and phrases are defined below: As used herein, the term “aliphatic” represents the groups including, but not limited to, alkyl, alkenyl, alkynyl, alicyclic.

The term “halo” or “halogen” refers to any radical of fluorine, chlorine, bromine or iodine.

As used herein, the term “alkyl” refers to an unsaturated carbon chain substituent group.

In general, alkyls have the general formula CₙH₂ₙ₊₁. Exemplary alkyls include, but are not limited to, methyl (CH₃), ethyl (C₂H₅), propyl (C₃H₇), butyl (C₄H₉), pentyl (C₅H₁₁), etc.

The term “alkenyl” refers to a monovalent straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. In certain aspects, the term “alkenyl” refers to a monovalent straight or branched hydrocarbon chain containing 2-6 carbon atoms and having one or more double bonds. In other aspects, the term “alkenyl” refers to a monovalent straight or branched hydrocarbon chain containing 2-4 carbon atoms and having one or more double bonds.

The term “alkynyl” refers to a monovalent straight or branched hydrocarbon chain containing 2-12 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

As used herein, the term “aryl” represents a single aromatic ring such as a phenyl ring, or two or more aromatic rings (e.g., bisphenyl, naphthalene, anthracene), or an aromatic ring and one or more non-aromatic rings. The aryl group can be optionally substituted with a lower aliphatic group (e.g., alkyl, alkenyl, alkynyl, or alicyclic). Additionally, the aliphatic and aryl groups can be further substituted by one or more functional groups including, but not limited to, chemical moieties comprising N, S, O, —NH₂, —NHCOCH₃, —OH, lower alkoxy (C₁-C₄), and halo (—F, —Cl, —Br, or —I).

As used herein, the term “substituted aliphatic” refers to an alkane, alkene, alkyne, or alicyclic moiety where at least one of the aliphatic hydrogen atoms has been replaced by, for example, a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such include, but are not limited to, 1-chloroethyl and the like.

As used herein, the term “substituted aryl” refers to an aromatic ring or fused aromatic ring system consisting of at least one aromatic ring, and where at least one of the hydrogen atoms on a ring carbon has been replaced by, for example, a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, hydroxyphenyl and the like.

As used herein, the term “cycloaliphatic” refers to an aliphatic structure containing a fused ring system. Examples of such include, but are not limited to, decalin and the like.

As used herein, the term “substituted cycloaliphatic” refers to a cycloaliphatic structure where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic).

Examples of such include, but are not limited to, 1-chlorodecalyl, bicyclo-heptanes, octanes, and nonanes (e.g., nonrbornyl) and the like.

As used herein, the term “heterocyclic” represents, for example, an aromatic or nonaromatic ring containing one or more heteroatoms. The heteroatoms can be the same or different from each other. Examples of heteroatoms include, but are not limited to nitrogen, oxygen and sulfur. Aromatic and nonaromatic heterocyclic rings are well-known in the art. Some nonlimiting examples of aromatic heterocyclic rings include pyridine, pyrimidine, indole, purine, quinoline and isoquinoline. Nonlimiting examples of nonaromatic heterocyclic compounds include piperidine, piperazine, morpholine, pyrrolidine and pyrazolidine. Examples of oxygen containing heterocyclic rings include, but not limited to furan, oxirane, 2H-pyran, 4H-pyran, 2H-chromene, and benzofuran. Examples of sulfur-containing heterocyclic rings include, but are not limited to, thiophene, benzothiophene, and parathiazine. Examples of nitrogen containing rings include, but not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted with one or more groups selected from aliphatic, nitro, acetyl (i.e., —C(═O)—CH₃), or aryl groups.

As used herein, the term “substituted heterocyclic” refers to a heterocylic structure where at least one of the ring carbon atoms is replaced by oxygen, nitrogen or sulfur, and where at least one of the aliphatic hydrogen atoms has been replaced by a halogen, hydroxy, a thio, nitro, an amino, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to 2-chloropyranyl.

As used herein, the term “linker” refers to an organic or inorganic molecule that links multiple functional units of a molecule. In some embodiments, the linker is a single moiety or chain containing up to and including eight contiguous atoms connecting two different structural moieties where such atoms are, for example, carbon, nitrogen, oxygen, or sulfur.

As used herein, the term “lower-alkyl-substituted-amino” refers to any alkyl unit containing up to and including eight carbon atoms where one of the aliphatic hydrogen atoms is replaced by an amino group. Examples of such include, but are not limited to, ethylamino and the like.

The term “derivative” of a compound, as used herein, refers to a chemically modified compound wherein the chemical modification takes place either at a functional group of the compound or backbone.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the terms “purified” or “to purify” refer, to the removal of undesired components from a sample. As used herein, the term “substantially purified” refers to molecules that are at least 60% free, preferably 75% free, and most preferably 90%, or more, free from other components with which they usually associated.

### DETAILED DESCRIPTION OF THE INVENTION

Indolizines are condensed [6,5]-heterocycles containing an inner nitrogen atom (Singh, G. S.; Mmatli, E. E. Recent Progress in Synthesis and Bioactivity Studies of Indolizines. Eur. J. Med. Chem. 2011, 46 (11), 5237-5257). Importantly, this aromatic chemotype in its reduced form (indolizidines) is prevalent in natural products, which include (−)-swainsonine 1, and tashirome 2, both possessing anti-cancer activity (Singh et al., supra; Pyne, S. Recent Developments on the Synthesis of (−)-Swainsonine and Analogues. Curr. Org. Synth. 2005, 2 (1), 39-57; Marsden, S. P.; McElhinney, A. D. Total Synthesis of the Indolizidine Alkaloid Tashiromine. Beilstein J. Org. Chem. 2008, 4, 1-5). Indeed indolizines exhibit a wide range of pharmaco-logical effects, such as the toxicity associated with pu-miliotoxins extracted from poison-dart frogs, inhibitory activity for both the phosphodiesterase PDE5A 3 and vascular endothelial growth factor (VEGF) 4 (singh et al., supra; Sandeep, C.; Venugopala, K. N.; Khedr, M. A.; Attimarad, M.; Padmashali, B.; Kulkarni, R. S.; Venugopala, R.; Odhav, B. Review on Chemistry of Natural and Synthetic Indolizines with Their Chemical and Pharmacological Properties. J. Basic Clin. Pharm. 2017, 8 (2), 49-61; Sadowski, B.; Klajn, J.; Gryko, D. T. Recent Advances in the Synthesis of Indolizines and Their 7r-Expanded Analogues. Org. Biomol. Chem. 2016, 14 (33), 7804-7828). Moreover, indolizines are found in Seoul-Fluor 5 and PITE 6, which have utility as biomedical fluorescent probes for ratiometric pH sensing, reactive oxygen species sensing, and lipid droplet characterization (Sandeep et al., supra; Kim, E.; Lee, Y.; Lee, S.; Park, S. B. Discovery, Understanding, and Bioapplication of Organic Fluorophore: A Case Study with an Indolizine-Based Novel Fluorophore, Seoul-Fluor. Acc. Chem. Res. 2015, 48 (3), 538-547).

Several synthetic routes to indolizines have been developed with one of the earliest proceding via the Chichi-babin reaction. The route begins with the condensation of 2-methyl pyridine 7 and bromoketone 8. The penultimate step then affords 9, which is primed for congener preparation of 10 via Suzuki coupling (Scheme 2) (R Bragg, B. D.; Wibberley, D. G. Borrows and Holland, J., 1947, 672. 2 Stepanow and Grineva; 1962; Vol. 42; Singh, D. K.; Kim, S.; Lee, J. H.; Lee, N. K.; Kim, J.; Lee, J.; Kim, I. 6-(Hetero)Arylindolizino[1,2-c]Quinolines as Highly Fluorescent Chemical Space: Synthesis and Photophysical Properties. J. Heterocycl. Chem. 2020, 57 (8), 3018-3028; Kostik, E. I.; Abiko, A.; Oku, A. Chichibabin Indolizine Synthesis Revisited: Synthesis of Indolizinones by Solvolysis of 4-Alkoxycarbonyl-3-Oxotetrahydroquinolizinium Ylides. J. Org. Chem. 2001, 66 (8), 2618-2623.7-9 Final tetracyclic products 10 displayed photophysical properties and were used for fluorescent imaging in HeLa cells (Singh et al, 2020; supra). Alternate methods toward indolizines involve cycloadditions of 1-cyanocyclopropane 1-esters and pyridine10 and others deploy multicomponent reactions (MCRs) (Bora, U.; Saikia, A.; Boruah, R. C. A Novel Microwave-Mediated One-Pot Synthesis of Indolizines via a Three-Component Reaction. Org. Lett. 2003, 5 (4), 435-438). One such MCR affords access to indolizines through a base-catalyzed 3-component reaction with isocyanides, aldehydes, and 2-cyanomethylpyridine via Knoevenagel condensation (Bedjeguelal, K.; Bienayme, H.; Poigny, S.; Schmitt, P.; Tam, E. Expeditious Access to Diversely Substituted Indolizines Using aNew Multi-Component Condensation. QSAR Comb. Sci. 2006, 25 (5-6), 504-508). Previous studies modified this reaction utilizing cyanides (acetyl cyanide, trimethylsilyl cyanide) to generate products with a functionalizable amine handle that exhibited fluorescence in one operation (Martinez-Ariza, G.; Mehari, B. T.; Pinho, L. A. G.; Foley, C.; Day, K.; Jewett, J. C.; Hulme, C. Organic & Biomolecular Chemistry Synthesis of Fluorescent Heterocycles via a Knoevenagel/[4+1]-Cycloaddition Cascade Using Acetyl Cyanide t. Org. Biomol. Chem. 2017, 15, 6076).

Experiments described herein developed a synthesis method comprised of an initial unique 4CR-3C MCR primed to rapidly undergo cyclization and oxidation in one pot. Indeed, the chemotypes presented offer high diversity by utilization of an MCR, higher quantum yields, and thus find use in a variety of applications.

For example, in some embodiments, provided herein is a composition, comprising a compound selected from, for example,

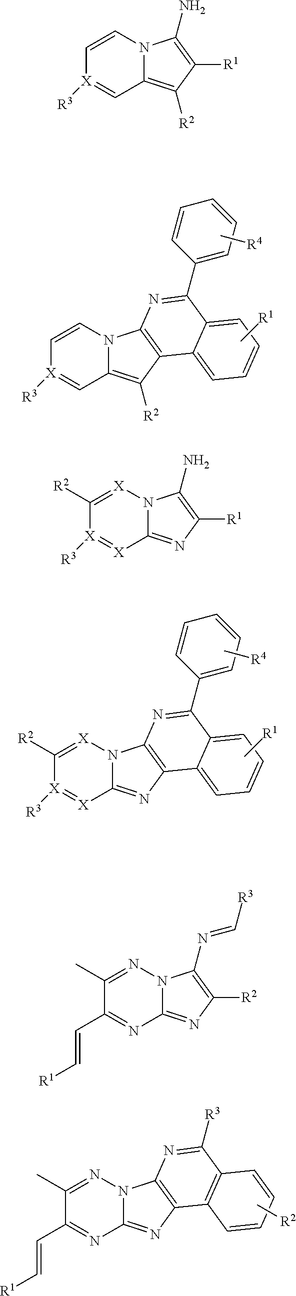

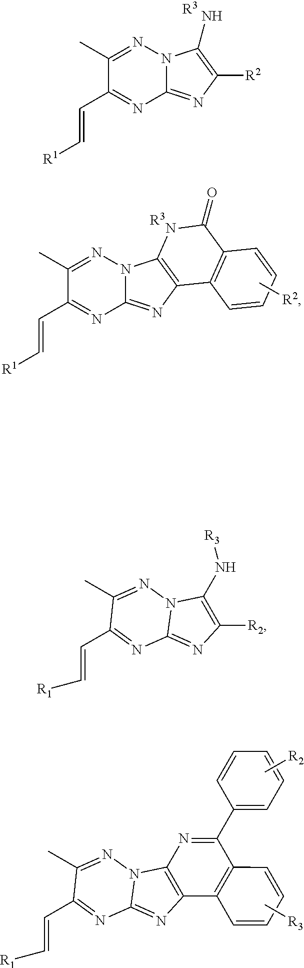

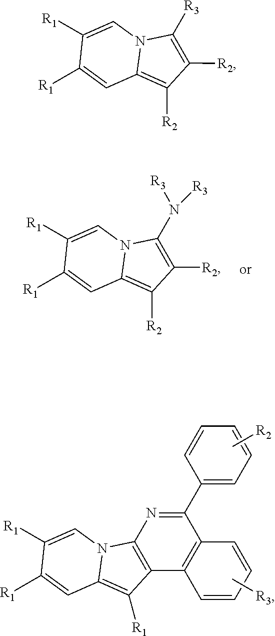

wherein X is CR, CH, or N, and R₁, R₂, R₃, and R₄ are independently selected from, for example, null, H, (CH₂)ₙ—O—(CH₂)ₘ, Substituted or unsubstituted aryl, substituted or unsubstituted C₁-C₆ alkyl, where n and m are independently integers between 0 and 6, CN, halide, N-alkyl, O-alkyl, or N-aryl. In some embodiments, R₁ and R₄ are —OCH₃. In some embodiments, R₂ is CH₂CN. In some embodiments, R₁ and R₄ are independently ortho, meta, para, or a combination thereof. In some embodiments, R₁, R₂, R₃, and R₄ are independently selected from, for example isobutyl,

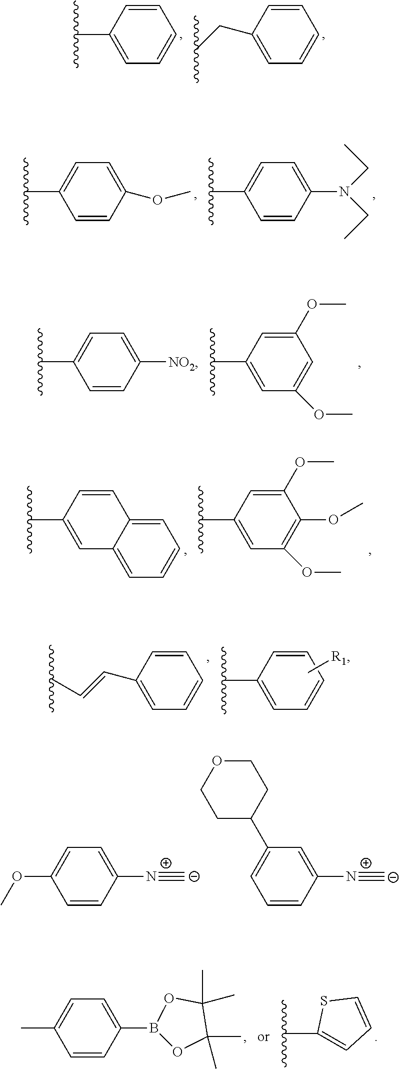

Exemplary compounds of the present disclosure include but are not limited to,

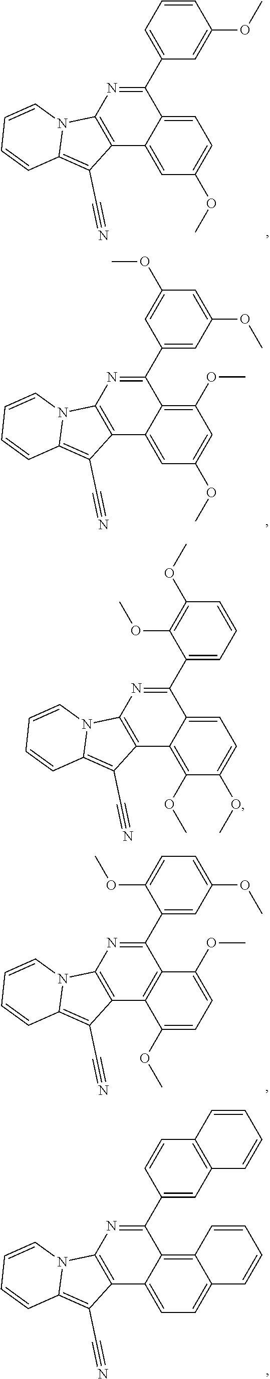

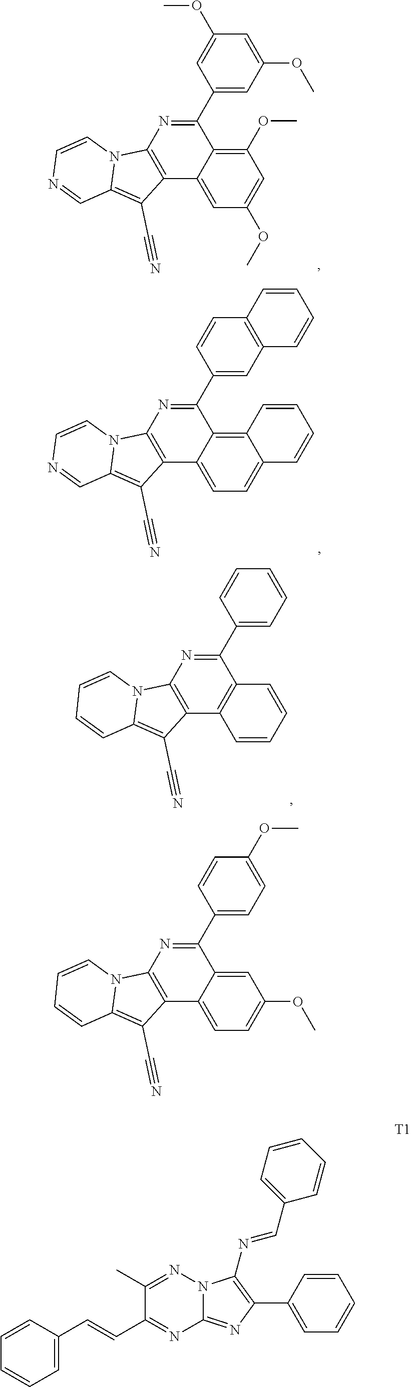

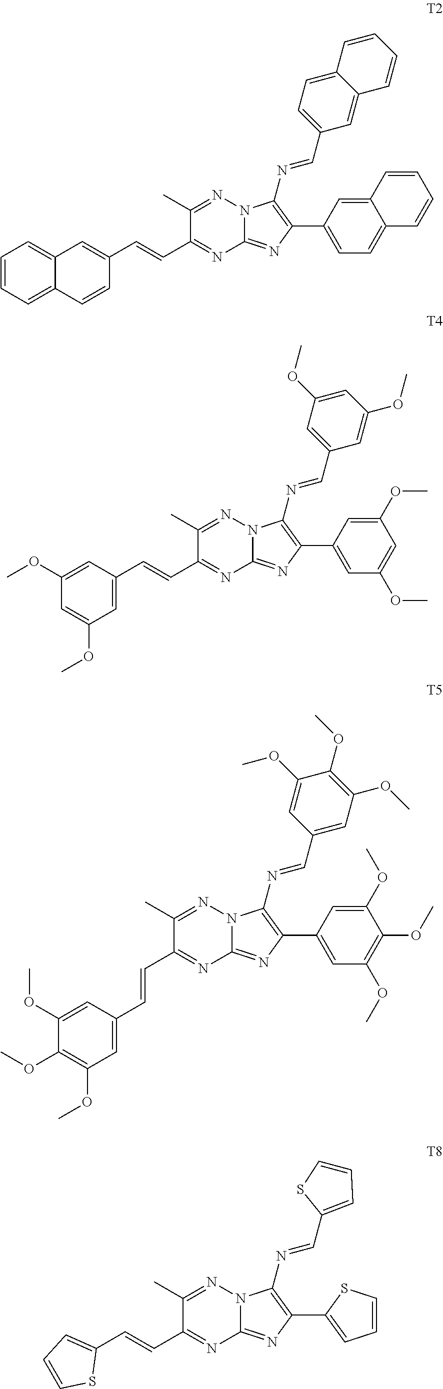

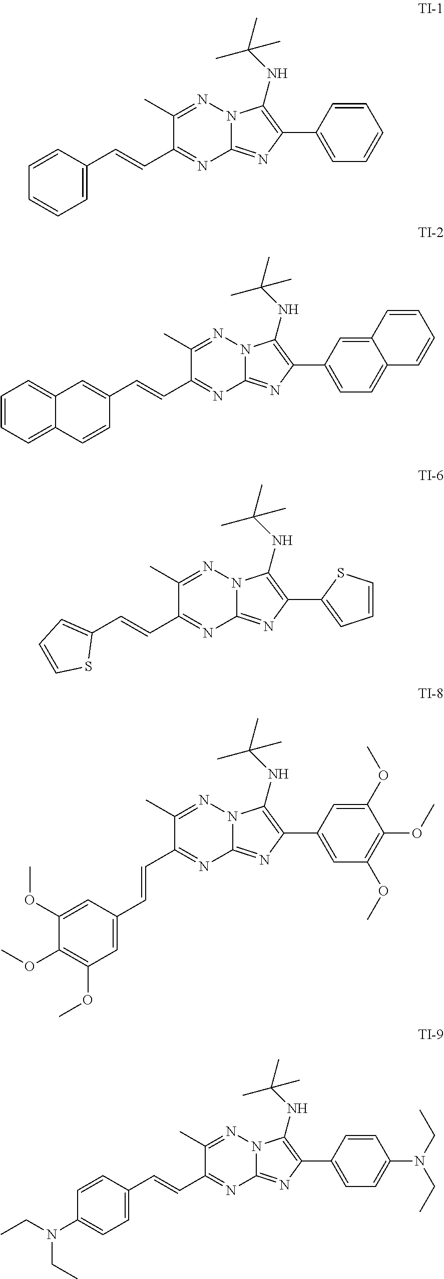

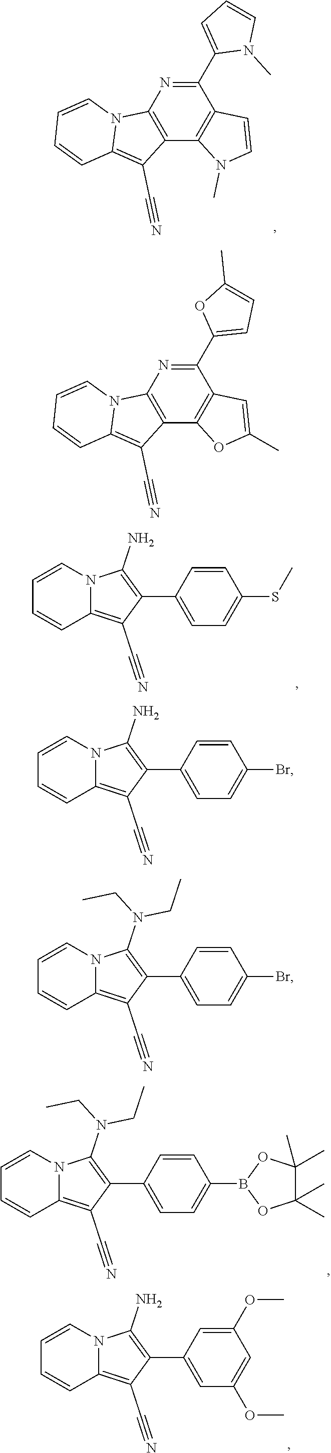

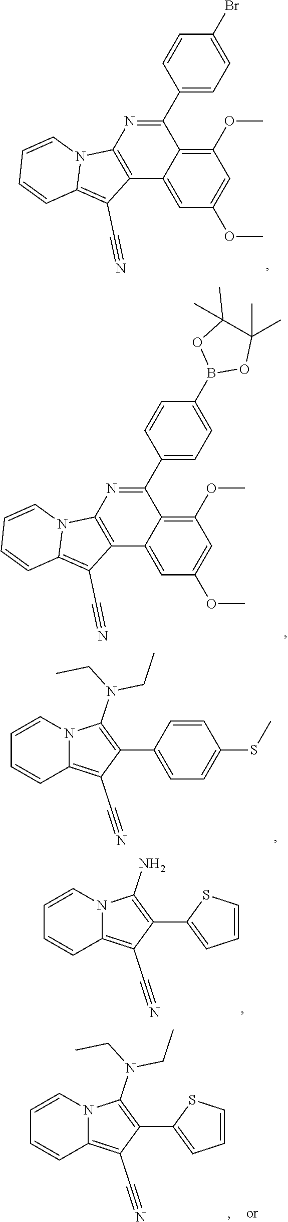

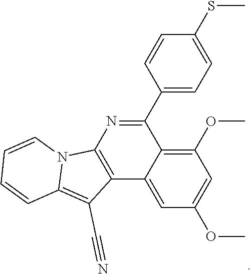 [SMILES: COc1cc(OC)c2c(-c3ccc(SC)cc3)nc3c(c(C#N)c4ccccn43)c2c1]

In some embodiments, the compound is fluorescent. In some embodiments, the compound is a probe. In certain embodiments, the compound is conjugated to a second molecule (e.g., via a linker).

In some embodiments, compounds of the present disclosure are provided in the form of a kit or system (e.g., for use in detecting an analyte). For example, in some embodiments, the kit comprises two or more compounds, wherein each of the compounds emits fluorescence of a different wavelength (e.g., for multiplex applications). In some embodiments, the kit further comprises one or more additional components necessary, useful, or sufficient for using the compounds. Examples include but are not limited to one or more buffers, one or more detection reagents, a fluorescent spectrometer, one or more control agents, or software for analysis or detection of an analyte of interest.

The compounds of the present disclosure find use in a variety of applications in medicine (e.g., bioimaging, clinical chemistry, etc.), biotechnology (e.g., detection of analytes, reactive oxygen species sensing, pH determination, etc.), and research. For example, in some embodiments, the present disclosure provides a method of detecting an analyte of interest, comprising: a) contacting a sample comprising the analyte of interest with a compound, kit or system described herein, and b) detecting fluorescence from the compound. The present disclosure is not limited to particular analytes. Examples include but are not limited to, a protein, a nucleic acid, a small molecule, or an acid.

Having now fully described the invention, it will be understood by those of skill in the art that the same can be performed within a wide and equivalent range of conditions, formulations, and other parameters without affecting the scope of the invention or any embodiment thereof.

All patents, patent applications and publications cited herein are fully incorporated by reference herein in their entirety.

### EXPERIMENTAL

### Example I

### Methods

### Synthetic Experimental Procedures

All reagents and solvents were acquired from commercially available suppliers and used without further purification. Methanol(dried on molecular sieves-Acroseal) was obtained from Acros. The products were purified using a Teledyne CombiFlash Rf automated flash chromatography apparatus with a cartridge utilizing the compounds dry loaded onto approximately an inch of Silicycle Siliaflash P60 Gel C60 (particle size 40-63 μm) and the main column, typically a Teledyne Isco silica column (4 g, normal or gold) or through recrystallization described in the procedure. 1H and 13C NMR spectra were obtained on a Bruker NMR spectrometer at 500/400 and 125/100 MHz respectively. The data is reported as follows: chemical shift in ppm (δ), multiplicity (s=singlet, d=doublet, dd=doublet of doublets, t=triplet, dt=doublet of triplets, td=triplet of doublets, q=quartet, p=pentet, m=multiplet). Coupling constants are reported in Hertz (Hz) and were automatically generated using known NMR analyzer software (MestReNova), these were then subsequently curated. High-resolution mass spectra were obtained using the positive ESI method for all the compounds, obtained in an Ion Cyclotron Resonance (ICR) spectrometer. Absorbance spectra were obtained using an Agilent 8453 UV-visible spectroscopy system. Fluorescence was measured on a Varian (Agilent) Cary Eclipse Fluorimeter. Quantum yield measurements were calculated and reported using coumarin 151 in ethanol as a standard.¹ Fluorescence scans were obtained using a 2.5 mm slit width unless otherwise noted.

### General Procedures

General Cyclization Reaction Procedure 1: One-pot GBB condensation H2SO4-Catalyzed Aza-Friedel-Crafts Intramolecular Cyclization-Oxidation.

A mixture of aldehyde (2.2 equiv., 2.2 mmol) and aminopyridine(1.0 eq., 1.0 mmol) in dry MeOH (3.3 mL, 0.3 M) was added to a microwave-vial (MWV) with a stir bar. The MWV was capped and Trimethylsilylnitrile (102 mg, 129 μL, 97% Wt, 1.0 Eq, 1.00 mmol) was injected. The resulting mixture was stirred and heated at 140° C. in microwave for 20 min. H2SO4 (2.66 mL, 50 eq., 50.0 mmol) was injected with venting and the reaction was heated to 140° C. in a microwave for another 20 min. The crude mixture was treated with saturated sodium bicarbonate(10 mL) and extracted with DCM (3×10 mL). The solvent was evaporated in vacuo and the solid product was washed with cold EtOAc (25 mL) to leave the title compound.

### General Multicomponent Reaction (MCR) Procedure 2: GBB to Afford 10 Analogs

A mixture of aldehyde (1.0 equiv., 1.0 mmol), aminopyridine(1.0 eq., 1.0 mmol), and Scandiumtrifluoromethanesulfonate (98.4 mg, 0.200 Eq, 0.200 mmol) in dry MeOH (5 mL, 0.2 M) was added to a microwave-vial (MWV) with stirbar. The MWV was capped and (Trimethylsilyl nitrile) (102 mg, 129 μL, 97% Wt, 1.0 Eq, 1.00 mmol) was injected. The resulting mixture was stirred and heated at 140° C. in microwave for 20 min. The solvent was evaporated in vacuo and the crude product was dissolved in DCM, dry loaded onto silica, and purified via automated flash chromatography using a Teledyne ISCOTM (0-80% EtOAc/Hexane, usually) to afford the title compound. Note

### General Procedure 3

A mixture of aminopyridine (1 eq., 0.63 mmol), aldehyde (2.2 eq., 1.4 mmol), and scandium (III) trifluoromethanesulfonate (0.23 eq, 0.15 mmol) were added to a microwave-vial (MWV) with a stirbar. The MWV was capped and dry MeOH (2.0 mL, 0.3 M) injected followed by trimethylsilylnitrile (90 μL, 97% Wt., 1.1 eq, 0.70 mmol). The reaction was stirred and heated to 140° C. in microwave for 20 min. The solvent was distilled off in vacuo and the crude product dissolved in DCM, and dry loaded onto silica. The dry loaded crude product was purified via automated flash chromatography via a Teledyne ISCOTM (usually 0-3% MeOH/DCM) to afford the title compound.

### General Procedure 4

A mixture of the GBB imine product (1 eq., 0.22 mmol) and copper (II) trifluoromethanesulfonate (1 eq., 0.22 mmol) to a MWV with a stir bar. The MWV was capped and dry 1,2-dichloroethane (2 mL, 0.1 M) injected. The reaction was stirred and heated to 120° C. in microwave for 20 min. The solvent was distilled off in vacuo and the crude product dissolved in DCM then washed with saturated sodium bicarbonate (aq). The organic layer was collected, dried over either sodium sulfate (anhyd.) or magnesium sulfate (anhyd.). The crude product in DCM was then dry loaded onto silica and purified via automated flash chromatography via a Teledyne ISCOTM (usually 0-3% MeOH/DCM) to afford the title compound.

### General Procedure 5

A mixture of aldehyde (1.2 equiv., 0.60 mmol), 2-(3,5-dimethoxyphenyl)imidazo[1,2-a]pyridin-3-amine (135 mg, 1 Eq, 0.500 mmol), and Copper(II)trifluoromethanesulfonate (181 mg, 1.0 Eq, 500 μmol) in DCM (2.5 mL, 0.2 M) was added to a MWV with stirbar. The MWV was capped and stirred at 100° C. in the microwave for 20 min. The solvent was evaporated in vacuo and the crude product was recrystallized with EtOAc to afford the title compound.

### Compound Characterization Data

### 2-(3,4,5-trimethoxyphenyl)imidazo[1,2-a]pyridin-3-amine

Compound 10a was prepared by General procedure 2. (200 mg, 0.83 mmol, 83% yield).

Orange solid. 1H NMR (400 MHz, Chloroform-d) δ 7.99 (dd, J=6.8, 1.5 Hz, 1H), 7.60-7.52 (m, 3H), 7.38 (t, J=7.9 Hz, 1H), 7.11 (ddd, J=9.4, 6.6, 1.3 Hz, 1H), 6.93-6.86 (m, 1H), 6.81 (td, J=6.7, 1.1 Hz, 1H), 3.91 (s, 3H), 3.45 (s, 2H). 13C{1H} NMR (101 MHz, CDCl3) δ 160.0, 140.9, 135.9, 132.8, 129.6, 123.1, 122.8, 121.8, 119.4, 117.4, 113.2, 112.3, 111.7, 77.3, 77.0, 76.72, 55.3. HRMS (ESI) Calculated for C14H13N3O [M+H]+ 240.1131, found 240.1130. λex=343 nm; λem=474 nm, Quantum Yield: 0.36.

### 2-(2,3-dimethoxyphenyl)imidazo[1,2-a]pyridin-3-amine

Compound 10b was prepared by General procedure 2. (194 mg, 0.720 mmol, 72% yield).

Orange-black solid. 1H NMR (400 MHz, Chloroform-d) δ 7.99 (dt, J=6.9, 1.2 Hz, 1H), 7.52 (td, J=8.1, 1.4 Hz, 2H), 7.19 (t, J=8.0 Hz, 1H), 7.11-7.03 (m, 1H), 6.93 (dd, J=8.1, 1.5 Hz, 1H), 6.77 (td, J=6.7, 1.1 Hz, 1H), 4.08 (s, 2H), 3.94 (s, 3H), 3.65 (s, 3H). 13C{1H} NMR (101 MHz, CDCl3) δ 152.7, 145.3, 140.8, 128.9, 128.1, 125.4, 124.9, 123.11, 122.5, 121.8, 117.2, 111.4, 111.0, 77.4, 77.0, 76.7, 61.1, 55.8. HRMS (ESI) Calculated for C15H15N3O2 [M+H]+ 270.1237, found 270.1235. λex=341 nm; λem=471 nm, Quantum Yield: 0.40.

### 2-(3,4,5-trimethoxyphenyl)imidazo[1,2-a]pyridin-3-amine

Compound 10c was prepared by General procedure 2. (165 mg, 0.55 mmol, 55% yield).

Yellow solid. 1H NMR (400 MHz, Chloroform-d) δ 7.99 (dt, J=6.9, 1.2 Hz, 1H), 7.56 (dt, J=9.1, 1.1 Hz, 1H), 7.26 (s, 2H), 7.13 (ddd, J=9.1, 6.7, 1.3 Hz, 1H), 6.83 (td, J=6.8, 1.1 Hz, 1H), 3.94 (d, J=17.4 Hz, 10H), 3.44 (s, 2H). 13C{1H} NMR (101 MHz, CDCl3) δ 153.5, 140.8, 137.5, 133.0, 130.1, 123.2, 122.3, 121.6, 117.2, 111.8, 104.5, 77.3, 77.0, 76.7, 60.9, 56.3. HRMS (ESI) Calculated for C16H17N3O3 [M+H]+ 300.1343, found 1340. λex=343 nm; λem=472 nm, Quantum Yield: 0.38.

### 2-(3,5-dimethoxyphenyl)imidazo[1,2-a]pyridin-3-amine

Compound 10d was prepared by General procedure 2. (210 mg, 0.780 mmol, 78% yield).

Yellow solid. 1H NMR (400 MHz, Chloroform-d) δ 8.01 (dt, J=6.9, 1.2 Hz, 1H), 7.57 (dt, J=9.1, 1.1 Hz, 1H), 7.19-7.08 (m, 3H), 6.83 (td, J=6.7, 1.1 Hz, 1H), 6.47 (t, J=2.3 Hz, 1H), 3.90 (s, 6H), 3.47 (s, 2H). 13C{1H} NMR (101 MHz, CDCl3) δ 161.0, 148.0, 140.7, 137.7, 136.4, 132.4, 123.1, 121.7, 117.3, 113.9, 111.7, 108.6, 105.9, 105.8, 105.0, 99.5, 77.3, 77.0, 76.7, 55.5, 55.4. HRMS (ESI) Calculated for C15H15N3O2 [M+H]+ 270.1237, found 270.1237. λex=344 nm; λem=473 nm, Quantum Yield: 0.35.

### 2-(naphthalen-2-yl)imidazo[1,2-a]pyridin-3-amine

Compound 10e was prepared by General procedure 2. (228 mg, 0.88 mmol, 88% yield).

Yellow solid. 1H NMR (400 MHz, Chloroform-d) δ 8.44 (d, J=1.7 Hz, 1H), 8.19 (dd, J=8.5, 1.7 Hz, 1H), 8.01 (dt, J=6.8, 1.3 Hz, 1H), 7.93 (dd, J=8.8, 6.5 Hz, 2H), 7.89-7.82 (m, 1H), 7.60 (dt, J=9.1, 1.1 Hz, 1H), 7.56-7.45 (m, 2H), 7.14 (ddd, J=9.1, 6.7, 1.3 Hz, 1H), 6.82 (td, J=6.8, 1.2 Hz, 1H), 3.50 (s, 2H). 13C{1H} NMR (101 MHz, CDCl3) δ 141.1, 133.6, 133.1, 132.5, 131.9, 128.2, 128.1, 127.7, 126.2, 125.8, 125.7, 125.2, 123.3, 121.7, 117.3, 111.7, 77.3, 77.0, 76.7. HRMS (ESI) Calculated for C17H13N3 [M+H]+ 260.1182, found 260.1183. λex=345 nm; λem=475 nm, Quantum Yield: 0.28.

### 2-phenylimidazo[1,2-a]pyridin-3-amine

Compound 10f was prepared by General procedure 2. (193 mg, 0.92 mmol, 92% yield).

Yellow-Orange solid. 1H NMR (400 MHz, Chloroform-d) δ 8.06 (d, J=6.9 Hz, 1H), 8.01 (dd, J=7.9, 1.4 Hz, 2H), 7.64-7.58 (m, 1H), 7.49 (t, J=7.7 Hz, 2H), 7.39-7.32 (m, 1H), 7.20-7.11 (m, 1H), 6.86 (t, J=6.8 Hz, 1H), 3.49 (d, J=6.4 Hz, 2H). 13C{1H} NMR (126 MHz, DMSO) δ 139.2, 135.6, 128.7, 127.8, 126.9, 126.6, 126.3, 122.9, 122.3, 117.0, 111.3, 40.5, 40.3, 40.2, 40.1, 40.0, 40.0, 39.8, 39.6, 39.5. HRMS (ESI) Calculated for C13H11N3 [M+H]+ 210.1026, found 210.1026. λex=339 nm; λem=473 nm, Quantum Yield: 0.30.

### 2-(4-methoxyphenyl)imidazo[1,2-a]pyridin-3-amine

Compound 10g was prepared by General procedure 2. (151 mg, 0.63 mmol, 63% yield).

Yellow solid. 1H NMR (400 MHz, Chloroform-d) δ 8.00 (dq, J=6.9, 1.1 Hz, 1H), 7.97-7.90 (m, 2H), 7.54 (dq, J=9.1, 1.0 Hz, 1H), 7.11 (ddt, J=8.8, 6.6, 1.0 Hz, 1H), 7.07-6.97 (m, 2H), 6.80 (tt, J=6.8, 1.0 Hz, 1H), 3.88 (d, J=0.8 Hz, 3H), 3.34 (s, 2H). 13C{1H} NMR (101 MHz, CDCl3) δ 158.8, 140.9, 133.57, 128.3, 127.0, 123.0, 121.7, 121.6, 117.1, 114.14, 111.5, 77.3, 77.0, 76.7, 55.3. HRMS (ESI) Calculated for C14H13N3O [M+H]+ 240.1131, found 240.1131. λex=340 nm; λem=472 nm, Quantum Yield: 0.39.

### 2-(pyridin-2-yl)imidazo[1,2-a]pyridin-3-amine

Compound 10h was prepared by General procedure 2. (88 mg, 0.42 mmol, 42% yield).

Black solid. 1H NMR (400 MHz, Chloroform-d) δ 8.57 (ddd, J=4.9, 1.9, 0.9 Hz, 1H), 8.21 (dd, J=8.1, 1.4 Hz, 1H), 7.84 (d, J=7.0 Hz, 1H), 7.77 (td, J=7.8, 1.8 Hz, 1H), 7.57 (d, J=9.1 Hz, 1H), 7.17-7.05 (m, 2H), 6.82 (td, J=6.8, 1.1 Hz, 1H), 5.48 (s, 2H). 13C{1H} NMR (101 MHz, CDCl3) δ 148.4, 138.9, 136.7, 130.1, 121.4, 120.7, 119.8, 116.9, 112.4, 77.3, 77.02, 76.7. HRMS (ESI) Calculated for C12H10N4 [M+H]+ 211.0978, found 211.0978. λex=370 nm; λem=N/A nm, Quantum Yield: N/A.

### 2-(2-nitrophenyl)imidazo[1,2-a]pyridin-3-amine

Compound 10i was prepared by General procedure 2. (100 mg, 0.47 mmol, 47% yield).

Brown solid. 1H NMR (400 MHz, Chloroform-d) δ 8.09-8.02 (m, 1H), 7.95 (dd, J=8.2, 1.3 Hz, 1H), 7.84 (dd, J=7.7, 1.4 Hz, 1H), 7.68 (td, J=7.6, 1.3 Hz, 1H), 7.60-7.48 (m, 2H), 7.23-7.14 (m, 1H), 6.88 (td, J=6.8, 1.0 Hz, 1H), 3.21 (s, 2H). 13C{1H} NMR (101 MHz, CDCl3) δ 141.2, 132.7, 132.5, 128.5, 124.4, 124.0, 122.1, 117.6, 112.3, 77.3, 77.0, 76.7. HRMS (ESI) Calculated for C13H10N4O2 [M+H]+ 255.0876, found 255.0877. λex=330 nm; λem=N/A nm, Quantum Yield: N/A.

### 2-(3-nitrophenyl)imidazo[1,2-a]pyridin-3-amine

Compound 10j was prepared by General procedure 2. (131 mg, 0.53 mmol, 53% yield).

Red solid. 1H NMR (400 MHz, Chloroform-d) δ 9.00 (s, 1H), 8.49 (d, J=7.8 Hz, 1H), 8.18 (d, J=8.4 Hz, OH), 8.08 (d, J=6.6 Hz, 1H), 7.70-7.57 (m, 2H), 7.29 (s, 1H), 7.26-7.18 (m, 1H), 6.91 (t, J=6.8 Hz, 1H), 3.43 (s, 2H). 13C{1H} NMR (126 MHz, DMSO) δ 148.6, 139.3, 136.9, 132.2, 130.3, 128.4, 123.9, 123.4, 120.7, 120.6, 117.0, 112.1, 40.5, 40.4, 40.3, 40.2, 40.1, 40.0, 40.0, 39.9, 39.8, 39.6, 39.5. HRMS (ESI) Calculated for C13H10N4O2 [M+H]+ 255.0877, found 255.0873. λex=350 nm; λem=N/A, Quantum Yield: N/A.

### 2-(4-nitrophenyl)imidazo[1,2-a]pyridin-3-amine

Compound 10k was prepared by General procedure 2. (61 mg, 0.24 mmol, 24% yield).

Red solid. 1H NMR (500 MHz, DMSO) δ 8.09-8.06 (m, 1H), 8.05 (d, J=3.5 Hz, 4H), 7.21 (dt, J=9.2, 1.1 Hz, 1H), 6.88 (ddd, J=9.1, 6.6, 1.2 Hz, 1H), 6.64 (td, J=6.7, 1.1 Hz, 1H), 5.56 (s, 2H). 13C NMR (126 MHz, DMSO) δ 142.6, 139.7, 130.4, 126.4, 124.2, 123.5, 123.3, 117.5, 111.8, 40.3, 40.2, 40.0, 39.8, 39.7, 39.5. HRMS (ESI) Calculated for C13H10N4O2 [M+H]+ 255.0877, found 255.0878. λex=304 nm; λem=N/A, Quantum Yield: N/A.

### 2-(3,5-dimethylphenyl)imidazo[1,2-a]pyridin-3-amine

Compound 101 was prepared by General procedure 2. (191 mg, 0.82 mmol, 82% yield).

Yellow-Brown solid. 1H NMR (400 MHz, Chloroform-d) δ 8.02 (dt, J=6.9, 1.2 Hz, 1H), 7.62-7.53 (m, 3H), 7.12 (ddd, J=9.1, 6.7, 1.3 Hz, 1H), 6.99 (s, 1H), 6.82 (td, J=6.8, 1.1 Hz, 1H), 3.48 (s, 2H), 2.41 (s, 6H). 13C{1H} NMR (101 MHz, CDCl3) δ 140.7, 138.2, 134.0, 128.9, 124.9, 123.2, 121.8, 117.2, 111.7, 77.3, 77.0, 76.7, 21.4. HRMS (ESI) Calculated for C15H15N3 [M+H]+ 238.1339, found 238.1336. λex=343 nm; λem=474 nm, Quantum Yield: 0.28.

### 2-(4-phenoxyphenyl)imidazo[1,2-a]pyridin-3-amine

Compound 10m was prepared by General procedure 2. (184 mg, 0.61 mmol, 61% yield).

Yellow solid. 1H NMR (400 MHz, Chloroform-d) δ 8.05 (dt, J=6.8, 1.1 Hz, 1H), 8.02-7.90 (m, 2H), 7.57 (dt, J=9.0, 1.0 Hz, 1H), 7.44-7.33 (m, 2H), 7.26-7.05 (m, 6H), 6.85 (td, J=6.8, 1.1 Hz, 1H), 3.45 (s, 2H). 13C{1H} NMR (101 MHz, CDCl3) δ 157.1, 156.6, 140.6, 129.8, 128.6, 123.7, 123.3, 122.1, 121.9, 119.0, 118.9, 116.9, 112.0, 77.3, 77.0, 76.7. HRMS (ESI) Calculated for C19H15N3O [M+H]+ 302.1288, found 302.1285. λex=343 nm; λem=474 nm, Quantum Yield: 0.40.

### 2-(3-phenoxyphenyl)imidazo[1,2-a]pyridin-3-amine

Compound 10n was prepared by General procedure 2. (221 mg, 0.73 mmol, 73% yield).

Orange solid. 1H NMR (400 MHz, Chloroform-d) δ 8.01 (dt, J=6.8, 1.1 Hz, 1H), 7.75 (dt, J=7.7, 1.3 Hz, 1H), 7.69 (t, J=2.1 Hz, 1H), 7.54 (dt, J=9.1, 1.0 Hz, 1H), 7.43 (t, J=7.9 Hz, 1H), 7.39-7.30 (m, 2H), 7.18-7.03 (m, 4H), 7.03-6.93 (m, 1H), 6.82 (td, J=6.8, 1.1 Hz, 1H), 3.54-3.35 (m, 2H). 13C{1H} NMR (101 MHz, CDCl3) δ 157.5, 157.3, 140.8, 136.0, 132.1, 130.0, 129.7, 123.5, 123.1, 122.9, 122.1, 121.9, 118.8, 117.7, 117.7, 117.2, 111.9, 77.3, 77.0, 76.7. HRMS (ESI) Calculated for C19H15N3O [M+H]+ 302.1288, found 302.1283. λex=339 nm; λem=474 nm, Quantum Yield: 0.40.

### 2-(1-methyl-1H-pyrrol-2-yl)imidazo[1,2-a]pyridin-3-amine

Compound 10o was prepared by General procedure 2. (141 mg, 0.66 mmol, 66% yield).

Black solid. 1H NMR (400 MHz, Chloroform-d) δ 7.96 (dt, J=6.8, 1.2 Hz, 1H), 7.52 (dt, J=9.1, 1.1 Hz, 1H), 7.09 (ddd, J=9.1, 6.7, 1.3 Hz, 1H), 6.86-6.75 (m, 2H), 6.38 (dd, J=3.6, 1.8 Hz, 1H), 6.26 (dd, J=3.6, 2.7 Hz, 1H), 3.93 (s, 3H), 3.50 (d, J=3.5 Hz, 2H). 13C{1H} NMR (101 MHz, CDCl3) δ 140.2, 137.9, 126.1, 126.0, 123.8, 122.3, 121.6, 117.1, 111.5, 108.8, 108.6, 107.6, 77.3, 77.0, 76.7, 35.5. HRMS (ESI) Calculated for C12H12N4 [M+H]+ 213.1135, found 213.1137. λex=349 nm; λem=478 nm, Quantum Yield: 0.20.

### 2-(4-(diphenylamino)phenyl)imidazo[1,2-a]pyridin-3-amine

Compound 10p was prepared by General procedure 2. (267 mg, 0.71 mmol, 71% yield). Red-Orange solid. 1H NMR (400 MHz, Chloroform-d) δ 8.04 (dd, J=6.7, 1.3 Hz, 1H), 7.92-7.84 (m, 2H), 7.57 (dd, J=9.0, 1.3 Hz, 1H), 7.33-7.24 (m, 3H), 7.21-7.14 (m, 7H), 7.14-7.10 (m, 1H), 7.08-7.02 (m, 2H), 6.83 (td, J=6.7, 1.1 Hz, 1H), 3.42 (s, 2H). 13C{1H} NMR (101 MHz, CDCl3) δ 147.6, 147.0, 129.2, 128.0, 124.4, 123.8, 123.3, 122.9, 121.9, 121.8, 117.0, 111.8, 77.3, 77.0, 76.7. HRMS (ESI) Calculated for C25H20N4 [M+H]+ 377.1761, found 377.1757. λex=353 nm; λem=471 nm, Quantum Yield: 0.34.

### 2-(4-(diethylamino)phenyl)imidazo[1,2-a]pyridin-3-amine

Compound 10q was prepared by General procedure 2. (162 mg, 0.57 mmol, 57% yield).

Black solid. 1H NMR (400 MHz, Chloroform-d) δ 7.99 (dt, J=6.8, 1.1 Hz, 1H), 7.88-7.80 (m, 2H), 7.52 (dt, J=9.1, 1.1 Hz, 1H), 7.07 (ddd, J=9.2, 6.7, 1.3 Hz, 1H), 6.84-6.74 (m, 3H), 3.42 (q, J=7.1 Hz, 6H), 1.22 (t, J=7.0 Hz, 6H). 13C{1H} NMR (101 MHz, CDCl3) δ 147.0, 140.6, 133.9, 128.2, 122.7, 121.6, 121.1, 121.0, 116.6, 111.8, 111.4, 77.3, 77.0, 76.7, 44.3, 12.6, 12.4. HRMS (ESI) Calculated for C17H20N4 [M+H]+ 281.1761, found 281.1760. λex=341 nm; λem=486 nm, Quantum Yield: 0.12.

### 2-(3,5-dimethoxyphenyl)imidazo[1,2-a]pyrimidin-3-amine

Compound 10r was prepared by General procedure 2. (95 mg, 0.35 mmol, 35% yield).

Yellow solid. 1H NMR (400 MHz, Chloroform-d) δ 8.47 (dd, J=4.0, 2.0 Hz, 1H), 8.35 (dd, J=6.8, 2.0 Hz, 1H), 7.24 (d, J=2.3 Hz, 2H), 6.87 (dd, J=6.8, 4.0 Hz, 1H), 6.48 (t, J=2.3 Hz, 1H), 3.89 (s, 6H), 3.50 (d, J=12.4 Hz, 2H). 13C{1H} NMR (101 MHz, CDCl3) δ ¹³C NMR (101 MHz, CDCl3) δ 161.0, 148.4, 129.4, 108.0, 105.4, 100.0, 77.3, 77.0, 76.7, 55.5. HRMS (ESI) Calculated for C14H14N4O2 [M+H]+ 271.1190, found 271.1189. λex=371 nm; λem=553 nm, Quantum Yield: 0.02.

### 2-(3,5-dimethoxyphenyl)imidazo[1,2-a]pyrazin-3-amine

Compound 10s was prepared by General procedure 2. (197 mg, 0.72 mmol, 72% yield).

Tan solid. 1H NMR (400 MHz, Chloroform-d) δ 8.99 (d, J=1.3 Hz, 1H), 7.89-7.81 (m, 2H), 7.09 (d, J=2.3 Hz, 2H), 6.50 (t, J=2.3 Hz, 1H), 3.89 (s, 6H), 3.74 (s, 2H). 13C{1H} NMR (101 MHz, CDCl3) δ 161.2, 143.3, 135.8, 135.3, 134.2, 129.0, 114.3, 105.3, 100.1, 77.3, 77.0, 76.7, 55.5. HRMS (ESI) Calculated for C14H14N4O2 [M+H]+ 271.1190, found 271.1186. λex=382 nm; λem=477 nm, Quantum Yield: 0.35.

### 2-(3,5-dimethoxyphenyl)imidazo[1,2-b]pyridazin-3-amine

Compound lot was prepared by General procedure 2. (172 mg, 0.63 mmol, 63% yield).

Yellow solid. 1H NMR (400 MHz, Chloroform-d) δ 8.29 (dd, J=4.4, 1.5 Hz, 1H), 7.88 (dd, J=9.1, 1.5 Hz, 1H), 7.16 (d, J=2.3 Hz, 2H), 6.88 (dd, J=9.1, 4.4 Hz, 1H), 6.47 (t, J=2.3 Hz, 1H), 4.45 (s, 2H), 3.91 (s, 6H). 13C{1H} NMR (126 MHz, CDCl3) δ 161.3, 142.4, 136.3, 133.6, 127.5, 124.8, 113.5, 104.2, 99.7, 77.2, 77.0, 76.7, 55.5. 6 HRMS (ESI) Calculated C₁₄H14N4O2 [M+H]+ 271.1190, found 271.1186. λex=407 nm; λem=546 nm, Quantum Yield: 0.02.

### methyl 3-amino-2-(3,5-dimethoxyphenyl)imidazo[1,2-a]pyridine-7-carboxylate

Compound 10v was prepared by General procedure 2. (240 mg, 0.73 mmol, 73% yield).

Yellow solid. 1H NMR (400 MHz, Chloroform-d) δ ¹H NMR (400 MHz, CDCl3) δ 8.31 (t, J=1.2 Hz, 1H), 7.94 (d, J=7.2 Hz, 1H), 7.42 (dd, J=7.1, 1.6 Hz, 1H), 7.11 (d, J=2.3 Hz, 2H), 6.49 (t, J=2.3 Hz, 1H), 3.98 (s, 3H), 3.90 (s, 6H), 3.70 (s, 2H). 13C{1H} NMR (101 MHz, CDCl3) δ 166.0, 161.2, 139.1, 123.7, 120.7, 120.3, 111.2, 105.2, 99.9, 77.3, 77.0, 76.7, 55.5, 52.4. HRMS (ESI) Calculated for C17H17N3O4 [M+H]+ 328.1292, found 328.1289. λex=403 nm; λem=514 nm, Quantum Yield: 0.42.

### methyl 3-amino-2-(3,5-dimethoxyphenyl)imidazo[1,2-a]pyridine-6-carboxylate

Compound 10w was prepared by General procedure 2. (148 mg, 0.45 mmol, 45% yield).

Yellow solid. 1H NMR (400 MHz, Chloroform-d) δ 8.83-8.78 (m, 1H), 7.68 (dd, J=9.4, 1.7 Hz, 1H), 7.56 (dd, J=9.4, 1.0 Hz, 1H), 7.16 (d, J=2.3 Hz, 2H), 6.49 (t, J=2.3 Hz, 1H), 3.99 (s, 3H), 3.90 (s, 6H), 3.55 (s, 2H). 13C{1H} NMR (101 MHz, CDCl3) δ 161.1, 126.3, 122.7, 116.6, 105.2, 99.9, 77.3, 77.0, 76.7, 55.5, 52.3. HRMS (ESI) Calculated for C17H17N3O4 [M+H]+ 328.1292, found 328.1290. λex=343 nm; λem=473 nm, Quantum Yield: 0.02.

### methyl 3-amino-2-(3,5-dimethoxyphenyl)imidazo[1,2-a]pyridine-5-carboxylate

Compound 10x was prepared by General procedure 2. (167 mg, 0.51 mmol, 51% yield).

Tan-Orange solid. 1H NMR (400 MHz, Chloroform-d) δ 7.86 (d, J=8.8 Hz, 1H), 7.60 (dd, J=7.1, 1.2 Hz, 1H), 7.11 (dd, J=8.8, 7.1 Hz, 1H), 7.06 (d, J=2.3 Hz, 2H), 6.50 (t, J=2.3 Hz, 1H), 4.58 (s, 2H), 4.07 (s, 3H), 3.90 (s, 6H). 13C{1H} NMR (101 MHz, CDCl3) δ 163.1, 161.3, 121.8, 120.1, 105.4, 100.1, 77.3, 77.0, 76.70, 55.5, 53.2. HRMS (ESI) Calculated for C₁₇H17N3O4 [M+H]+ 328.1292, found 328.1290. λex=400 nm; λem=N/A nm, Quantum Yield: N/A.

### 7-bromo-2-(3,5-dimethoxyphenyl)imidazo[1,2-a]pyridin-3-amine

Compound 10y was prepared by General procedure 2. (276 mg, 0.79 mmol, 79% yield).

Yellow solid. 1H NMR (400 MHz, Chloroform-d) δ 7.88 (dd, J=7.2, 0.8 Hz, 1H), 7.74 (dd, J=1.8, 0.8 Hz, 1H), 7.11 (d, J=2.3 Hz, 2H), 7.03-6.89 (m, 1H), 6.48 (t, J=2.3 Hz, 1H), 3.89 (s, 6H), 3.45 (s, 2H). 13C{1H} NMR (101 MHz, CDCl3) δ 161.1, 135.9, 122.2, 119.5, 116.8, 115.6, 105.1, 99.7, 77.3, 77.0, 76.70, 55.5. HRMS (ESI) Calculated for C15H14BrN3O2 [M+H]+ 348.0342, found 348.0339. λex=349 nm; λem=487 nm, Quantum Yield: 0.01.

Compound 10aa was prepared by General procedure 2. (132 mg, 0.63 mmol, 63% yield).

Brown solid. 1H NMR (400 MHz, Chloroform-d) δ 8.27 (dd, J=4.4, 1.5 Hz, 1H), 8.03-7.95 (m, 2H), 7.86 (dd, J=9.1, 1.6 Hz, 1H), 7.51 (t, J=7.8 Hz, 2H), 7.38-7.30 (m, 1H), 6.86 (dd, J=9.1, 4.4 Hz, 1H), 4.43 (s, 2H). 13C{1H} NMR (101 MHz, CDCl3) δ 142.3, 134.3, 133.7, 129.3, 128.9, 127.8, 127.1, 126.3, 124.7, 113.4, 77.3, 77.0, 76.7. HRMS (ESI) Calculated for C₁₂H10N4 [M+H]+ 211.0978, found 211.0978. λex=432 nm; λem=545 nm, Quantum Yield: 0.02.

### 2-(naphthalen-2-yl)imidazo[1,2-b]pyridazin-3-amine

Compound 10ab was prepared by General procedure 2. (159 mg, 0.61 mmol, 61% yield).

Brown solid. 1H NMR (400 MHz, Chloroform-d) δ 8.44-8.39 (m, 1H), 8.30 (dd, J=4.4, 1.5 Hz, 1H), 8.20 (dd, J=8.5, 1.8 Hz, 1H), 8.01-7.79 (m, 4H), 7.51 (tt, J=6.9, 5.2 Hz, 2H), 6.89 (dd, J=9.1, 4.4 Hz, 1H), 4.53 (s, 2H). 13C{1H} NMR (101 MHz, CDCl3) δ 142.4, 133.7, 132.5, 128.6, 128.1, 127.7, 126.3, 125.9, 124.8, 124.8, 124.5, 113.6, 77.3, 77.0, 76.7. HRMS (ESI) Calculated for C16H12N4 [M+H]+ 261.1135, found 261.1129. λex=438 nm; λem=543 nm, Quantum Yield: 0.04.

### 2-(4-methoxyphenyl)imidazo[1,2-b]pyridazin-3-amine

Compound 10ac was prepared by General procedure 2. (106 mg, 0.44 mmol, 44% yield).

Yellow solid. 1H NMR (400 MHz, CDCl3) δ 8.25 (dd, J=4.5, 1.5 Hz, 1H), 7.97-7.87 (m, 2H), 7.82 (dd, J=9.1, 1.5 Hz, 1H), 7.08-7.00 (m, 2H), 6.83 (dd, J=9.0, 4.5 Hz, 1H), 4.34 (s, 2H), 3.88 (s, 3H). 13C{1H} (101 MHz, CDCl3) δ 158.9, 142.1, 133.7, 128.5, 128.4, 127.6, 126.9, 124.3, 114.3, 113.0, 77.3, 77.0, 76.7, 55.3. HRMS (ESI) Calculated for C13H12N4O [M+H]+ 241.1084, found 241.1081. λex=435 nm; λem=543 nm, Quantum Yield: 0.03.

### 2-(3,4,5-trimethoxyphenyl)imidazo[1,2-b]pyridazin-3-amine

Compound 10ad was prepared by General procedure 2. (114 mg, 0.38 mmol, 38% yield).

Black-Green solid. 1H NMR (400 MHz, CDCl3) δ 8.27 (dd, J=4.4, 1.5 Hz, 1H), 7.85 (dd, J=9.1, 1.6 Hz, 1H), 7.24 (s, 2H), 6.86 (dd, J=9.1, 4.4 Hz, 1H), 4.40 (s, 2H), 3.98 (s, 6H), 3.92 (s, 3H). 13C{1H} (101 MHz, CDCl3) δ 153.7, 142.4, 137.48, 133.6, 129.98, 129.0, 128.0, 124.5, 113.6, 103.5, 77.3, 77.0, 76.7, 60.9, 56.3. HRMS (ESI) Calculated for C15H16N4O3 [M+H]+ 301.1295, found 301.1290. λex=429 nm; λem=541 nm, Quantum Yield: 0.03.

### 2-(2,4-dimethoxyphenyl)imidazo[1,2-b]pyridazin-3-amine

Compound 10ae was prepared by General procedure 2. (123 mg, 0.45 mmol, 45% yield).

Black solid. 1H NMR (400 MHz, CDCl3) δ 8.25 (dd, J=4.5, 1.5 Hz, 1H), 7.89-7.80 (m, 2H), 6.82 (dd, J=9.0, 4.5 Hz, 1H), 6.71 (dd, J=8.6, 2.4 Hz, 1H), 6.62 (d, J=2.4 Hz, 1H), 4.67 (s, 2H), 3.95 (s, 3H), 3.89 (s, 3H). 13C{1H} (101 MHz, CDCl3) δ 160.79, 156.73, 142.14, 133.56, 131.83, 130.31, 125.15, 124.26, 116.33, 112.68, 106.09, 99.29, 77.36, 77.04, 76.72, 56.14, 55.52. HRMS (ESI) Calculated for C14H14N4O2 [M+H]+ 271.1190, found 271.1185. λex=433 nm; λem=555 nm, Quantum Yield: 0.02.

### 2-(4-(diphenylamino)phenyl)imidazo[1,2-b]pyridazin-3-amine

Compound 10af was prepared by General procedure 2. (234 mg, 0.62 mmol, 62% yield).

Orange solid. 1H NMR (400 MHz, CDCl3) δ 8.31 (dd, J=4.4, 1.5 Hz, 1H), 7.92 (d, J=9.0 Hz, 1H), 7.90-7.84 (m, 2H), 7.35-7.25 (m, 5H), 7.21 (d, J=8.7 Hz, 2H), 7.17 (d, J=7.2 Hz, 3H), 7.07 (t, J=7.3 Hz, 2H), 6.90 (dd, J=9.1, 4.5 Hz, 1H), 4.40 (s, 2H). 13C{1H}13C NMR (101 MHz, CDCl3) δ 147.5, 147.1, 142.5, 129.3, 127.2, 124.5, 124.1, 123.8, 123.0, 77.3, 77.0, 76.7. HRMS (ESI) Calculated for C24H19N5 [M+H]+ 378.1713, found 378.1708. λex=438 nm; λem=544 nm, Quantum Yield: 0.05.

### 2-(4-(diethylamino)phenyl)imidazo[1,2-b]pyridazin-3-amine

Compound 10ag was prepared by General procedure 2. (129 mg, 0.46 mmol, 46% yield).

Black solid. 1H NMR (400 MHz, CDCl3) δ 8.25 (dd, J=4.5, 1.5 Hz, 1H), 7.90-7.80 (m, 3H), 6.81 (dd, J=9.0, 4.5 Hz, 3H), 4.32 (s, 2H), 3.47-3.42 (m, 4H), 1.24 (t, J=7.0 Hz, 6H). 13C{1H} (126 MHz, CDCl3) δ 141.8, 128.9, 127.6, 123.7, 77.2, 77.0, 76.7, 44.4, 12.6. HRMS (ESI) Calculated for C16H19N5 [M+H]+ 282.1713, found 282.1708. λex=444 nm; λem=572 nm, Quantum Yield: 0.06.

### methyl 3-amino-2-phenylimidazo[1,2-a]pyridine-7-carboxylate

Compound 10ah was prepared by General procedure 2. (219 mg, 0.81 mmol, 81% yield).

Yellow solid. 1H NMR (400 MHz, CDCl3) δ 8.31 (dt, J=1.6, 0.8 Hz, 1H), 8.01-7.92 (m, 3H), 7.51 (dd, J=8.4, 7.0 Hz, 2H), 7.44 (dd, J=7.1, 1.6 Hz, 1H), 7.43-7.34 (m, 1H), 3.98 (s, 3H), 3.67 (s, 2H). 13C{1H} (101 MHz, CDCl3) δ 128.9, 127.7, 127.2, 120.8, 120.1, 111.2, 77.3, 77.0, 76.7, 52.4. HRMS (ESI) Calculated for C15H13N3O2 [M+H]+ 268.1081, found 268.1077. λex=394 nm; λem=510 nm, Quantum Yield: 0.40.

### methyl 3-amino-2-(naphthalen-2-yl)imidazo[1,2-a]pyridine-7-carboxylate

Compound 10ai was prepared by General procedure 2. (245 mg, 0.77 mmol, 77% yield).

Yellow solid. 1H NMR (400 MHz, DMSO) δ 8.50 (d, J=1.8 Hz, 1H), 8.35 (dd, J=7.2, 1.0 Hz, 1H), 8.31 (s, 2H), 8.25 (dd, J=8.6, 1.8 Hz, 1H), 8.04 (dd, J=1.8, 0.9 Hz, 1H), 7.98 (t, J=7.8 Hz, 2H), 7.91 (dd, J=7.7, 1.5 Hz, 1H), 7.51 (s, 2H), 7.28 (dd, J=7.2, 1.7 Hz, 1H), 3.89 (s, 3H). 13C{1H} (101 MHz, DMSO) δ 166.0, 137.4, 133.7, 132.3, 132.2, 128.5, 128.2, 127.9, 126.6, 126.1, 125.4, 124.8, 122.3, 121.3, 119.3, 110.1, 79.7, 79.6, 79.4, 79.1, 52.6, 40.6, 40.4, 40.4, 40.2, 40.2, 40.0, 40.0, 39.8, 39.6, 39.3. HRMS (ESI) Calculated for C19H15N3O2 [M+H]+ 318.1237, found 318.1232. λex=434 nm; λem=512 nm, Quantum Yield: 0.47.

### methyl 2-([1,1′-biphenyl]-4-yl)-3-aminoimidazo[1,2-a]pyridine-7-carboxylate

Compound 10aj was prepared by General procedure 2. (268 mg, 0.78 mmol, 78% yield).

Yellow solid. 1H NMR (400 MHz, DMSO) δ 8.32 (d, J=7.3 Hz, 1H), 8.16-8.09 (m, 2H), 8.02 (t, J=1.3 Hz, 1H), 7.80-7.71 (m, 4H), 7.50 (t, J=7.7 Hz, 2H), 7.43-7.34 (m, 1H), 7.27 (dd, J=7.3, 1.7 Hz, 1H), 5.86 (d, J=7.6 Hz, 2H), 3.89 (s, 3H). 13C{1H} (101 MHz, DMSO) δ 129.4, 127.2, 127.1, 126.9, 52.6. HRMS (ESI) Calculated for C21H17N3O2 [M+H]+ 344.1394, found 344.1388. λex=403 nm; λem=517 nm, Quantum Yield: 0.45.

### methyl 3-amino-2-(4-methoxyphenyl)imidazo[1,2-a]pyridine-7-carboxylate

Compound 10ak was prepared by General procedure 2. (185 mg, 0.62 mmol, 62% yield).

Yellow solid. 1H NMR (400 MHz, CDCl3) δ 8.30 (t, J=1.3 Hz, 1H), 7.98 (dd, J=7.2, 0.9 Hz, 1H), 7.95-7.87 (m, 2H), 7.44 (dd, J=7.1, 1.6 Hz, 1H), 7.09-7.01 (m, 2H), 3.98 (s, 3H), 3.90 (s, 3H), 3.57 (s, 2H). 13C{1H} (101 MHz, CDCl3) δ 183.9, 128.5, 114.3, 111.1, 77.3, 77.0, 76.7, 55.3, 52.4. HRMS (ESI) Calculated for C16H15N3O3 [M+H]+ 298.1186, found 298.1181. λex=394 nm; λem=510 nm, Quantum Yield: 0.40.

### methyl 3-amino-2-(3,4,5-trimethoxyphenyl)imidazo[1,2-a]pyridine-7-carboxylate

Compound 10al was prepared by General procedure 2. (182 mg, 0.51 mmol, 51% yield).

Yellow solid. 1H NMR (400 MHz, CDCl3) δ 8.26 (dd, J=1.6, 0.9 Hz, 1H), 7.92 (dd, J=7.2, 1.0 Hz, 1H), 7.39 (dd, J=7.2, 1.6 Hz, 1H), 7.18 (s, 2H), 3.94 (d, J=2.8 Hz, 9H), 3.91 (s, 3H), 3.72 (s, 2H).13C{1H} (101 MHz, CDCl3) δ 165.9, 153.6, 139.0, 137.8, 134.8, 124.8, 120.7, 119.9, 111.2, 104.5, 77.3, 77.0, 76.7, 60.9, 56.3, 52.4. HRMS (ESI) Calculated for C18H19N3O5 [M+H]+ 358.1398, found 358.1393. λex=399 nm; λem=512 nm, Quantum Yield: 0.51.

### methyl 3-amino-2-(2,4-dimethoxyphenyl)imidazo[1,2-a]pyridine-7-carboxylate

Compound 10am was prepared by General procedure 2. (204 mg, 0.62 mmol, 62% yield).

Orange solid. 1H NMR (400 MHz, CDCl3) δ 8.24 (t, J=1.2 Hz, 1H), 7.89 (dd, J=7.2, 0.9 Hz, 1H), 7.75 (d, J=8.5 Hz, 1H), 7.34 (dd, J=7.2, 1.6 Hz, 1H), 6.65 (dd, J=8.5, 2.4 Hz, 1H), 6.58 (d, J=2.4 Hz, 1H), 3.94 (s, 5H), 3.89 (s, 3H), 3.87 (s, 3H). 13C{1H} (101 MHz, CDCl3) δ 166.3, 160.9, 156.7, 139.2, 132.3, 132.3, 126.6, 122.6, 120.5, 119.8, 116.0, 110.6, 105.9, 99.1, 77.3, 77.0, 76.7, 56.1, 55.5, 52.3. HRMS (ESI) Calculated for C17H17N3O4 [M+H]+ 328.1292, found 328.1289. λex=402 nm; λem=519 nm, Quantum Yield: 0.49.

### methyl 3-amino-2-(4-(diphenylamino)phenyl)imidazo[1,2-a]pyridine-7-carboxylate

Compound 10an was prepared by General procedure 2. (230 mg, 0.53 mmol, 53% yield).

Orange solid. 1H NMR (400 MHz, CDCl3) δ 8.29 (t, J=1.2 Hz, 1H), 8.01 (d, J=7.1 Hz, 1H), 7.87-7.79 (m, 2H), 7.44 (dd, J=7.1, 1.6 Hz, 1H), 7.30 (dd, J=8.5, 7.2 Hz, 4H), 7.25-7.13 (m, 6H), 7.08 (tt, J=7.2, 1.2 Hz, 2H), 3.97 (s, 3H), 3.69 (s, 2H). 13C{1H} (126 MHz, CDCl3) δ 165.8, 147.6, 147.4, 129.3, 128.0, 124.7, 124.3, 123.3, 123.2, 121.0, 111.5, 77.2, 77.0, 76.7, 52.4. HRMS (ESI) Calculated for C27H22N4O2 [M+H]+ 435.1816, found 435.1812. λex=430 nm; λem=529 nm, Quantum Yield: 0.44.

### methyl 3-amino-2-(4-(diethylamino)phenyl)imidazo[1,2-a]pyridine-7-carboxylate

Compound 10ao was prepared by General procedure 2. (140 mg, 0.41 mmol, 41% yield).

Black solid. 1H NMR (400 MHz, CDCl3) δ 8.23 (t, J=1.2 Hz, 1H), 7.89 (d, J=7.1 Hz, 1H), 7.83-7.75 (m, 2H), 7.35 (dd, J=7.1, 1.6 Hz, 1H), 6.80-6.72 (m, 2H), 3.95 (s, 3H), 3.59 (s, 2H), 3.42 (q, J=7.1 Hz, 4H), 1.22 (t, J=7.0 Hz, 6H). 13C{1H} (101 MHz, CDCl3) δ 166.2, 147.3, 139.0, 128.2, 123.6, 123.0, 120.4, 119.2, 111.8, 110.9, 77.3, 77.0, 76.7, 52.3, 44.3, 12.6. HRMS (ESI) Calculated for C19H22N4O2 [M+H]+ 339.1816, found 339.1819. λex=432 nm; λem=540 nm, Quantum Yield: 0.25.

### 2,2′-(1,4-phenylene)bis(N-(tert-butyl)imidazo[1,2-a]pyridin-3-amine)

Compound 10ap was prepared in a 5 mL MWV scandium trifluoromethanesulfonate (98.43 mg, 0.2 Eq, 200.0 μmol), pyridin-2-amine (94.12 mg, 1.0 Eq, 1.000 mmol), p-Formylbenzaldehyde (147.5 mg, 139 μL, 1.1 Eq, 1.100 mmol), MeOH (3.3 mL), and stirbar were added. The vial was capped, Propane, 2-isocyano-2-methyl-(174.6 mg, 238 μL, 2.1 Eq, 2.100 mmol) was injected, and set to run at 140° C. for 20 min, workup was the same as general procedure 2 and produced 2,2′-(1,4-phenylene)bis(N-(tert-butyl)imidazo[1,2-a]pyridin-3-amine) (281 mg, 620 μmol, 62%) as a tan solid. 1H NMR (500 MHz, CDCl3) δ 8.30 (d, J=6.9 Hz, 2H), 7.99 (s, 4H), 7.66 (d, J=8.9 Hz, 2H), 7.21 (t, J=7.8 Hz, 2H), 6.84 (t, J=6.8 Hz, 2H), 3.35 (s, 2H), 1.03 (s, 18H). 13C{1H} (126 MHz, CDCl3) δ 156.1, 128.1, 124.0, 123.9, 116.4, 112.2, 77.2, 77.0, 76.7, 56.6, 30.3. HRMS (ESI) Calculated for C28H32N6 [M+H]+ 453.2761, found 453.2755. λex=344 nm; λem=458 nm, Quantum Yield: 0.30.

### N-(tert-butyl)-2-phenylimidazo[1,2-a]pyridin-3-amine

Compound 10aq was prepared in a 5 mL MWV scandium trifluoromethanesulfonate (98.4 mg, 0.2 Eq, 200 μmol), pyridin-2-amine (94.1 mg, 1.0 Eq, 1.00 mmol), benzaldehyde (117 mg, 1.1 Eq, 1.10 mmol), MeOH (3.3 mL), and stirbar were added. The vial was capped, Propane, 2-isocyano-2-methyl-(91.4 mg, 125 μL, 1.1 Eq, 1.10 mmol) was injected, and set to run at 140° C. for 20 min, workup was the same as general procedure 2 and produced N-(tert-butyl)-2-phenylimidazo[1,2-a]pyridin-3-amine (226 mg, 850 μmol, 85%) as a brown solid. 1H NMR (500 MHz, CDCl3) δ 8.27 (d, J=6.9 Hz, 1H), 7.93-7.88 (m, 2H), 7.66 (d, J=9.1 Hz, 1H), 7.45-7.38 (m, 2H), 7.35-7.28 (m, 1H), 7.19 (t, J=7.9 Hz, 1H), 6.83 (t, J=6.8 Hz, 1H), 3.19 (s, 1H), 1.02 (s, 9H). 13C{1H}¹³C NMR (126 MHz, CDCl3) δ 128.3, 128.2, 127.7, 123.6, 116.9, 111.8, 77.2, 77.0, 76.7, 56.5, 30.3. HRMS (ESI) Calculated for C17H19N3 [M+H]+ 266.1652, found 266.1646. λex=326 nm; λem=461 nm, Quantum Yield: 0.30.

### 2-(4-methoxyphenyl)-6-phenylimidazo[1,2-a]pyridin-3-amine

Compound 10ar was prepared by General procedure 2. (224 mg, 0.71 mmol, 71% yield).

Orange solid. 1H NMR (400 MHz, CDCl3) δ 8.22 (dd, J=1.9, 1.0 Hz, 1H), 7.99-7.91 (m, 2H), 7.62 (ddd, J=8.9, 5.5, 1.3 Hz, 3H), 7.50 (dd, J=8.4, 6.8 Hz, 2H), 7.46-7.34 (m, 2H), 7.03-6.95 (m, 2H), 3.85 (s, 3H), 3.50 (s, 2H). 13C{1H} (101 MHz, CDCl3) δ 159.0, 137.6, 129.0, 128.3, 127.7, 126.9, 119.2, 116.5, 114.1, 77.3, 77.0, 76.7, 55.2. HRMS (ESI) Calculated for C20H17N3O [M+H]+ 316.1444, found 316.1436. λex=350 nm; λem=510 nm, Quantum Yield: 0.09.

### 2-(4-methoxyphenyl)-6-(piperidin-1-yl)imidazo[1,2-a]pyridin-3-amine

Compound 10as was prepared by General procedure 2. (198 mg, 0.61 mmol, 61% yield).

Black solid. 1H NMR ¹H NMR (500 MHz, CDCl3) δ 7.90-7.83 (m, 2H), 7.42-7.37 (m, 2H), 7.02-6.93 (m, 3H), 3.83 (s, 3H), 3.29 (s, 2H), 3.06-3.01 (m, 4H), 1.75 (p, J=5.6 Hz, 4H), 1.60-1.55 (m, 2H). 13C{1H}13C NMR (126 MHz, CDCl3) δ 158.7, 140.6, 128.1, 122.1, 121.1, 116.7, 114.1, 107.5, 77.2, 77.0, 76.7, 55.3, 52.1, 25.9, 24.0. HRMS (ESI) Calculated for C₁₉H22N4O [M+H]+ 323.1866, found 323.1868. λex=341 nm; λem=479 nm, Quantum Yield: 0.07.

### 7-methoxy-2-(4-methoxyphenyl)imidazo[1,2-a]pyridin-3-amine

Compound 10at was prepared by General procedure 2. (181 mg, 0.67 mmol, 67% yield).

Yellow solid. 1H NMR 1H NMR (500 MHz, CDCl3) δ 7.94-7.87 (m, 2H), 7.84 (d, J=7.4 Hz, 1H), 7.00-6.93 (m, 2H), 6.79 (d, J=2.4 Hz, 1H), 6.50 (dd, J=7.5, 2.4 Hz, 1H), 3.83 (s, 3H), 3.82 (s, 3H), 3.17 (s, 2H). 13C{1H}13C NMR (126 MHz, CDCl3) δ 158.6, 157.2, 142.3, 128.1, 127.2, 122.5, 120.3, 114.0, 106.5, 94.4, 77.2, 77.0, 76.7, 55.4, 55.3. HRMS (ESI) Calculated for C15H15N3O2 [M+H]+ 270.1237, found 270.1237. λex=344 nm; λem=500 nm, Quantum Yield: 0.10.

### 7-methoxy-2-(4-nitrophenyl)imidazo[1,2-a]pyridin-3-amine

Compound 10au was prepared by General procedure 2. (109 mg, 0.38 mmol, 38% yield).

Red solid. 1H NMR ¹H NMR (500 MHz, CDCl3) δ 8.19-8.13 (m, 2H), 7.85 (d, J=7.5 Hz, 1H), 7.72-7.66 (m, 2H), 6.88-6.77 (m, 1H), 6.56 (dd, J=7.5, 2.4 Hz, 1H), 3.85 (s, 3H), 3.27 (s, 2H). 13C{1H}¹³C NMR (126 MHz, DMSO) δ 156.8, 140.8, 140.6, 132.5, 128.5, 126.1, 124.4, 124.0, 120.0, 107.2, 106.8, 94.5, 55.9. HRMS (ESI) Calculated for C14H12N4O3 [M+H]+ 285.0982, found 285.0983. λex=360 nm; λem=N/A, Quantum Yield: N/A.

### 4-(3-amino-6-(piperidin-1-yl)imidazo[1,2-a]pyridin-2-yl)benzonitrile

Compound 10av was prepared by General procedure 2. (209 mg, 0.66 mmol, 66% yield).

Black solid. 1H NMR (500 MHz, CDCl3) δ 8.14-8.09 (m, 2H), 7.68-7.63 (m, 2H), 7.38 (d, J=9.7 Hz, 1H), 7.30 (d, J=2.2 Hz, 1H), 7.05 (dd, J=9.8, 2.2 Hz, 1H), 3.35 (s, 2H), 3.07-3.02 (m, 4H), 1.75 (p, J=5.8 Hz, 4H), 1.59 (td, J=7.1, 4.4 Hz, 2H). 13C{1H} (126 MHz, CDCl3) δ 141.0, 139.4, 138.6, 132.3, 131.2, 126.8, 124.3, 122.2, 119.3, 117.3, 109.6, 106.9, 77.2, 77.0, 76.7, 51.8, 25.8, 23.9. HRMS (ESI) Calculated for C19H19N5 [M+H]+ 318.1713, found 318.1710. λex=375 nm; λem=482 nm, Quantum Yield: 0.03.

### 2-(4-nitrophenyl)-6-(piperidin-1-yl)imidazo[1,2-a]pyridin-3-amine

Compound 10aw was prepared by General procedure 2. (95 mg, 0.28 mmol, 28% yield).

Black solid. 1H NMR (500 MHz, DMSO) δ 8.35-8.30 (m, 2H), 8.15-8.09 (m, 2H), 7.87 (d, J=2.1 Hz, 1H), 7.59-7.50 (m, 2H), 6.29 (s, 2H), 3.16-3.12 (m, 4H), 1.69 (p, J=5.6 Hz, 4H), 1.58 (h, J=4.5 Hz, 2H). 13C{1H} (126 MHz, DMSO) δ 145.56 141.9, 131.1, 126.4, 124.5, 113.9, 107.7, 50.7, 25.5, 24.0. HRMS (ESI) Calculated for C18H19N5O2 [M+H]+ 338.1612, found 338.1613. λex=390 nm; λem=N/A nm, Quantum Yield: N/A.

### 7-(pyridin-4-yl)-2-(3,4,5-trimethoxyphenyl)imidazo[1,2-a]pyridin-3-amine

Compound 10ax was prepared by adding 3,4,5-trimethoxybenzaldehyde (432 mg, 2.2 Eq, 2.20 mmol), 4-bromopyridin-2-amine (173 mg, 1 Eq, 1.00 mmol), and Scandiumtrifluoromethanesulfonate (98.4 mg, 0.200 Eq, 0.200 mmol) in dry MeOH (5 mL, 0.2 M) to a microwave-vial (MWV) with stirbar. The MWV was capped and (Trimethylsilyl nitrile) (102 mg, 129 μL, 97% Wt, 1.0 Eq, 1.00 mmol) was injected. The resulting mixture was stirred and heated at 140° C. in the microwave for 20 min. The mixture was placed in the freezer for 20 min, then filtered and washed with cold MeOH (10 mL). The solid was then placed in an MWV with stirbar, 1,1′-Bis(diphenylphosphino)ferrocene-palladium(II) dichloride (58.54 mg, 0.08 Eq, 80.00 μmol), 4-(4,4,5,5-tetramethyl-1,3,2-dioxaborolan-2-yl)pyridine (246.1 mg, 1.2 Eq, 1.200 mmol), and sodium hydrogen carbonate (344.4 mg, 4.1 Eq, 4.100 mmol). The vial was capped and purged with Argon for 15 min and injected with 1,4-Dioxane (1.0 mL, 0.1M) and further purged for 5 min. The vial was heated at 130° C. for 40 min in the microwave. The vial was diluted with DCM and filtered through celite and washed with DCM. The filtrate was concentrated and columned(0-80% HX:EtOAc). The (E)-N-(7-(pyridin-4-yl)-2-(3,4,5-trimethoxyphenyl)imidazo[1,2-a]pyridin-3-yl)-1-(3,4,5-trimethoxyphenyl)methanimine product was dissolved in EtOAc (10 mL) and treated with 1.0M HCl in EtOAc (15 mL) and stirred at rt for an hour. The suspension was filtered and the solid was dissolved in saturated NaHCO3 and DCM. The organic layer was concentrated to afford the title compound. (132 mg, 0.35 mmol, 35% yield). Orange solid. 1H NMR ¹H NMR (500 MHz, CDCl3) δ 8.71-8.66 (m, 2H), 8.07 (d, J=7.1 Hz, 1H), 7.88-7.84 (m, 1H), 7.57-7.51 (m, 2H), 7.24 (s, 2H), 7.13 (dd, J=7.2, 1.8 Hz, 1H), 3.99-3.88 (m, 9H), 3.58 (s, 2H). 13C{1H} (126 MHz, CDCl3) δ 153.6, 150.5, 145.9, 140.4, 137.8, 129.4, 123.1, 122.0, 120.8, 114.7, 110.8, 104.5, 77.2, 77.0, 76.7, 60.9, 56.3. HRMS (ESI) Calculated for C21H20N4O3 [M+H]+ 377.1608, found 377.1604. λex=392 nm; λem=545 nm, Quantum Yield: 0.23.

### 2-(4-(diethylamino)phenyl)-7-(pyridin-4-yl)imidazo[1,2-a]pyridin-3-amine

Compound 10ay was prepared by was prepared by adding 4-(diethylamino)benzaldehyde (390.0 mg, 2.2 Eq, 2.200 mmol), 4-bromopyridin-2-amine (173 mg, 1 Eq, 1.00 mmol), and Scandiumtrifluoromethanesulfonate (98.4 mg, 0.200 Eq, 0.200 mmol) in dry MeOH (5 mL, 0.2 M) to a microwave-vial (MWV) with stirbar. The MWV was capped and (Trimethylsilyl nitrile) (102 mg, 129 μL, 97% Wt, 1.0 Eq, 1.00 mmol) was injected. The resulting mixture was stirred and heated at 140° C. in the microwave for 20 min. The mixture was placed in the freezer for 20 min, then filtered and washed with cold MeOH (10 mL). The solid was then placed in an MWV with stirbar, 1,1′-Bis(diphenylphosphino)ferrocene-palladium(II) dichloride (58.54 mg, 0.08 Eq, 80.00 μmol), 4-(4,4,5,5-tetramethyl-1,3,2-dioxaborolan-2-yl)pyridine (246.1 mg, 1.2 Eq, 1.200 mmol), and sodium hydrogen carbonate (344.4 mg, 4.1 Eq, 4.100 mmol). The vial was capped and purged with Argon for 15 min and injected with 1,4-Dioxane (10.0 mL, 0.1M) and further purged for 5 min. The vial was heated at 130° C. for 40 min in the microwave. The vial was diluted with DCM and filtered through celite and washed with DCM. The filtrate was concentrated and columned(0-100% HX:EtOAc). The (E)-4-(3-((4-(diethylamino)benzylidene)amino)-7-(pyridin-4-yl)imidazo[1,2-a]pyridin-2-yl)-N,N-diethylaniline product was dissolved in EtOAc (10 mL) and treated with 1.0M HCl in EtOAc (15 mL) and stirred at rt for an hour. The suspension was filtered and the solid was dissolved in saturated NaHCO₃ and DCM. The organic layer was concentrated to afford the title compound. (100 mg, 0.28 mmol, 28% yield). Orange solid. 1H NMR (500 MHz, CDCl3) δ 8.70-8.65 (m, 2H), 8.19 (d, J=7.1 Hz, 1H), 7.93 (s, 1H), 7.85-7.78 (m, 2H), 7.52-7.48 (m, 2H), 7.12 (dd, J=7.2, 1.8 Hz, 1H), 6.66 (d, J=8.5 Hz, 2H), 3.74 (s, 2H), 3.36-3.29 (m, 4H), 1.15 (t, J=7.1 Hz, 6H). 13C{1H} (126 MHz, CDCl3) δ 150.5, 128.2, 120.8, 111.6, 77.2, 77.0, 76.7, 44.3, 12.6. HRMS (ESI) Calculated for C22H23N5 [M+H]+ 358.2026, found 358.2021. λex=435 nm; λem=581 nm, Quantum Yield: 0.06.

### 7-nitro-2-(3,4,5-trimethoxyphenyl)imidazo[1,2-a]pyridin-3-amine

Compound 10az was prepared by General procedure 2. (210 mg, 0.61 mmol, 61% yield).

Red solid. 1H NMR (500 MHz, CDCl3) δ 8.54 (d, J=2.2 Hz, 1H), 7.90 (d, J=7.5 Hz, 1H), 7.68 (dd, J=7.5, 2.2 Hz, 1H), 7.13 (s, 2H), 3.94 (s, 6H), 3.90 (s, 3H), 3.82 (s, 2H). 13C{1H} (126 MHz, CDCl3) δ 153.9, 120.5, 114.6, 106.5, 104.7, 77.2, 77.2, 77.0, 76.7, 61.0, 56.4. HRMS (ESI) Calculated for C16H16N4O5 [M+H]+ 345.1194, found 345.1193. λex=485 nm; λem=651 nm, Quantum Yield: 0.11: 10 mm slit.

### 2-(4-(diethylamino)phenyl)-7-nitroimidazo[1,2-a]pyridin-3-amine

Compound 10ba was prepared by General procedure 2. (137 mg, 0.42 mmol, 42% yield).

Black solid. 1H NMR (500 MHz, CDCl₃) δ 8.48 (d, J=2.2 Hz, 1H), 7.86 (d, J=7.5 Hz, 1H), 7.76 (d, J=8.4 Hz, 2H), 7.64 (dd, J=7.5, 2.3 Hz, 1H), 6.77 (s, 2H), 3.76 (s, 2H), 3.41 (q, J=7.1 Hz, 4H), 1.20 (t, J=7.1 Hz, 6H). 13C{1H} (126 MHz, CDCl3) δ 159.8, 150.6, 128.3, 106.3, 101.1, 77.2, 77.0, 76.7, 44.7, 12.5. HRMS (ESI) Calculated for C17H19N5O2 [M+H]+ 326.1612, found 326.1611. λex=550 nm; λem=720 nm, Quantum Yield: 0.09: 10 mm slit length in MeOH.

### (E)-1-(3,4,5-trimethoxyphenyl)-N-(2-(3,4,5-trimethoxyphenyl)imidazo[1,2-b]pyridazin-3-yl)methanimine

Compound 11e was prepared by General Procedure 5. (100 mg, 0.21 mmol, 34% yield). Bright light-yellow solid. 1H NMR (500 MHz, CDCl3) δ 9.81 (s, 1H), 8.34 (dd, J=4.4, 1.7 Hz, 1H), 7.94 (dd, J=9.1, 1.7 Hz, 1H), 7.64 (s, 2H), 7.20 (s, 2H), 7.02 (dd, J=9.1, 4.4 Hz, 1H), 3.91 (s, 7H), 3.88 (s, 6H), 3.87 (s, 3H), 3.86 (s, 2H). 13C{1H} (126 MHz, CDCl3) δ 158.1, 153.7, 153.3, 142.6, 141.2, 140.3, 138.7, 137.4, 133.0, 129.4, 129.1, 125.3, 116.8, 106.4, 105.7, 77.3, 77.0, 76.8, 61.1, 61.0, 56.4, 56.4, 56.3. HRMS (ESI) Calculated for C25H26N4O6 [M+H]+ 479.1925, found 479.1922.

### (E)-1-(3,4,5-trimethoxyphenyl)-N-(2-(3,4,5-trimethoxyphenyl)imidazo[1,2-a]pyrimidin-3-yl)methanimine

Compound 11f was prepare by adding pyrimidin-2-amine (1 eq., 0.95 mmol), 3,4,5-trimethoxybenzaldehyde (1.1 eq., 1.0 mmol), and ytterbium (III) trifluoromethanesulfonate (0.23 eq, 0.22 mmol) to a microwave-vial (MWV) with a stirbar. The MWV was capped and dry MeOH (6 mL, 0.2 M) injected followed by trimethylsilylnitrile (0.13 mL, 97% Wt., 1.1 eq, 1 mmol). The reaction was stirred and heated to 140° C. in microwave for 15 min. The solvent was distilled off in vacuo and the crude product dissolved in DCM, and dry loaded onto silica. The dry loaded crude product was purified via automated flash chromatography via a Teledyne ISCOTM (usually 0-3% MeOH/DCM) to afford the title compound (79 mg. 0.17 mmol, 17% yield). Orange solid. 1H NMR (500 MHz, CDCl3) δ 8.74 (s, 1H), 8.55 (dd, J=6.7, 2.0 Hz, 1H), 8.48 (dd, J=4.1, 2.0 Hz, 1H), 7.12 (s, 2H), 7.04 (s, 2H), 6.86 (dd, J=6.8, 4.1 Hz, 1H), 3.86 (s, 4H), 3.86 (s, 6H), 3.82 (s, 3H), 3.74 (s, 5H). 13C{1H} (126 MHz, CDCl3) δ 160.3, 153.7, 153.6, 149.9, 145.8, 141.7, 138.1, 134.5, 131.1, 130.5, 129.3, 127.4, 108.8, 105.4, 105.2, 77.3, 77.0, 76.8, 61.1, 61.0, 56.3, 56.2. HRMS (ESI) Calculated for C25H26N4O6 [M+H]+ 479.1925, found 479.1926.

### (E)-N-(5-bromo-2-(3,4,5-trimethoxyphenyl)imidazo[1,2-a]pyridin-3-yl)-1-(3,4,5-trimethoxyphenyl)methanimine

Compound 11g was prepared by adding 6-bromopyridin-2-amine (1.2 eq., 115 mg, 0.802 mmol), 3,4,5-trimethoxybenzaldehyde (1 eq., 109 mg, 0.554 mmol), and scandium (III) trifluoromethanesulfonate (0.23 eq, 62.5 mg, 0.127 mmol) to a MWV with a stirbar. The MWV was capped and MeOH (3 mL, 0.2 M) injected, followed by trimethylsilylnitrile (0.079 mL, 97% Wt., 1.1 eq., 0.609 mmol,). The reaction was stirred and heated to 140° C. in microwave for 15 min. The solvent was distilled off in vacuo and the crude product dissolved in DCM, and dry loaded onto silica. The dry loaded crude product was purified via automated flash chromatography via a Teledyne ISCOTM (usually 0-3% MeOH/DCM) to afford the title compound (53 mg, 0.095 mmol, 17% yield). Dark orange-brown solid. 1H NMR (500 MHz, CDCl3) δ 8.36 (s, 1H), 7.58 (d, J=8.7 Hz, 1H), 7.06 (s, 2H), 7.03 (dd, J=7.2, 1.3 Hz, 1H), 7.01-6.98 (m, 1H), 6.97 (s, 2H), 3.86 (s, 3H), 3.84 (s, 6H), 3.80 (s, 3H), 3.69 (s, 6H). 13C{1H} (126 MHz, CDCl3) δ 162.0, 153.7, 153.6, 144.1, 141.6, 140.3, 137.8, 132.5, 131.3, 125.3, 119.6, 116.5, 112.0, 106.2, 105.7, 105.0, 77.3, 77.0, 76.8, 61.0, 61.0, 60.4, 56.4, 56.3, 56.1. HRMS (ESI) Calculated for C26H26BrN3O6 [M+H]+ 556.1078, found 556.1074.

### (E)-1-(3,4,5-trimethoxyphenyl)-N-(6-(3,4,5-trimethoxyphenyl)imidazo[2,1-b]thiazol-5-yl)methanimine

Compound 11h was prepare by general procedure 5 (69 mg, 0.15 mmol, 24% yield). Pale orange solid. 1H NMR (500 MHz, CDCl3) δ 8.53 (s, 1H), 7.66 (d, J=4.5 Hz, 1H), 7.29 (s, 2H), 7.05 (s, 2H), 6.85 (d, J=4.5 Hz, 1H), 3.86 (d, J=2.2 Hz, 9H), 3.83 (d, J=2.5 Hz, 9H). 13C{1H} (126 MHz, CDCl3) δ 153.7, 153.3, 153.2, 141.2, 137.8, 131.9, 130.3, 118.5, 113.2, 106.7, 105.3, 105.1, 103.6, 103.2, 77.3, 77.0, 76.8, 61.1, 61.0, 60.8, 56.3, 56.2, 56.1, 52.9. HRMS (ESI) Calculated for C24H25N306S [M+H]+ 484.1537, found 484.1532.

### 2-methoxy-5-(3-methoxyphenyl)pyrido[2′,1′:2,3]imidazo[4,5-c]isoquinoline

Compound 16a was prepared by General Procedure 1. (235 mg, 0.66 mmol, 66% yield).

Tan solid. 1H NMR (400 MHz, CDCl3) δ 9.00-8.92 (m, 1H), 8.22-8.10 (m, 2H), 7.88 (d, J=9.2 Hz, 1H), 7.60-7.45 (m, 2H), 7.41-7.31 (m, 2H), 7.23 (dd, J=9.2, 2.6 Hz, 1H), 7.10 (dd, J=8.2, 2.6 Hz, 1H), 7.03 (t, J=6.7 Hz, 1H), 4.12 (s, 3H), 3.93 (s, 3H). 13C{1H} (101 MHz, CDCl3) δ 161.3, 159.6, 154.3, 146.3, 141.1, 135.4, 132.3, 130.8, 129.3, 129.1, 124.3, 122.7, 121.4, 118.8, 117.6, 115.6, 114.3, 111.5, 101.0, 77.3, 77.2, 77.0, 76.7, 55.9, 55.4. HRMS (ESI) Calculated for C22H17N3O2 [M+H]+ 356.1394, found 356.1392. λex=361 nm; λem=417 nm, Quantum Yield: 0.05.

### 5-(2,3-dimethoxyphenyl)-1,2-dimethoxypyrido[2′,1′:2,3]imidazo[4,5-c]isoquinoline

Compound 16b was prepared by General Procedure 1. (392 mg, 0.94 mmol, 94% yield).

Black solid. 1H NMR (400 MHz, CDCl3) δ 8.93 (dt, J=6.8, 1.2 Hz, 1H), 7.95 (dt, J=9.2, 1.1 Hz, 1H), 7.70 (d, J=9.2 Hz, 1H), 7.51-7.45 (m, 1H), 7.31 (s, 1H), 7.28 (s, 1H), 7.27-7.23 (m, 1H), 7.13 (dd, J=8.2, 1.6 Hz, 1H), 6.99 (td, J=6.8, 1.1 Hz, 1H), 4.25 (s, 3H), 4.08 (s, 3H), 4.00 (s, 3H), 3.63 (s, 3H). 13C{1H} (101 MHz, CDCl3) δ 153.3, 152.9, 152.3, 147.4, 146.2, 134.3, 128.0, 126.3, 124.0, 123.8, 123.3, 123.1, 118.4, 113.8, 113.5, 112.9, 111.3, 77.3, 77.0, 76.7, 61.4, 61.3, 56.7, 56.0. HRMS (ESI) Calculated for C24H21N3O4 [M+H]+ 416.1605, found 416.1603. λex=366 nm; λem=418 nm, Quantum Yield: 0.34.

### 2,3,4-trimethoxy-5-(3,4,5-trimethoxyphenyl)pyrido[2′,1′:2,3]imidazo[4,5-c]isoquinoline

Compound 16c was prepared by General Procedure 1. (253 mg, 0.53 mmol, 53% yield).

Tan solid. 1H NMR (400 MHz, CDCl3) δ 8.92 (dt, J=6.9, 1.2 Hz, 1H), 7.98 (s, 1H), 7.81 (dt, J=9.2, 1.1 Hz, 1H), 7.55-7.45 (m, 1H), 6.98 (td, J=6.7, 1.1 Hz, 1H), 6.81 (s, 2H), 4.17 (s, 3H), 3.95 (s, 6H), 3.91 (s, 6H), 3.49 (s, 3H). 13C{1H} (101 MHz, CDCl3)) δ 157.0, 152.2, 151.8, 151.5, 146.6, 142.3, 139.5, 137.4, 134.6, 131.1, 129.1, 128.6, 124.4, 117.5, 117.0, 111.2, 106.2, 97.8, 77.3, 77.0, 76.7, 61.2, 61.1, 60.9, 56.4, 56.1. HRMS (ESI) Calculated for C26H25N3O6 [M+H]+ 476.1816, found 476.1815. λex=367 nm; λem=428 nm, Quantum Yield: 0.01.

### 5-(3,5-dimethoxyphenyl)-2,4-dimethoxypyrido[2′,1′:2,3]imidazo[4,5-c]isoquinoline

Compound 16d was prepared by General Procedure 1. (399 mg, 0.96 mmol, 96% yield).

Tan solid. 1H NMR (400 MHz, CDCl3) δ 8.93 (dd, J=6.9, 1.3 Hz, 1H), 7.88-7.76 (m, 2H), 7.56-7.47 (m, 1H), 6.99 (td, J=6.8, 1.1 Hz, 1H), 6.69 (d, J=2.3 Hz, 2H), 6.66-6.52 (m, 2H), 4.11 (s, 3H), 3.86 (s, 6H), 3.64 (s, 3H). 13C{1H} (101 MHz, CDCl3) δ 162.4, 159.7, 159.4, 152.7, 146.6, 146.4, 135.2, 133.7, 129.1, 124.5, 117.5, 113.7, 111.3, 106.9, 99.8, 99.5, 93.8, 77.3, 77.0, 76.7, 56.0, 55.4. HRMS (ESI) Calculated for C24H21N3O4 [M+H]+ 416.1605, found 416.1606. λex=364 nm; λem=415 nm, Quantum Yield: 0.01.

### 7-(naphthalen-2-yl)benzo[h]pyrido[2′,1′:2,3]imidazo[4,5-c]isoquinoline

Compound 16e was prepared by General Procedure 1. (269 mg, 0.68 mmol, 68% yield).

Tan solid. 1H NMR (400 MHz, CDCl3) δ 9.02 (dt, J=6.9, 1.2 Hz, 1H), 8.90 (d, J=8.8 Hz, 1H), 8.32-8.29 (m, 1H), 8.25 (d, J=8.7 Hz, 1H), 8.04-7.90 (m, 6H), 7.72-7.56 (m, 4H), 7.52 (ddd, J=8.0, 6.9, 1.1 Hz, 1H), 7.15 (ddd, J=8.5, 6.9, 1.5 Hz, 1H), 7.08 (t, J=6.8 Hz, 1H). 13C{1H} (101 MHz, CDCl3) δ 129.0, 128.6, 128.5, 128.26, 128.1, 127.9, 126.4, 124.7, 120.6, 77.3, 77.0, 76.7. HRMS (ESI) Calculated for C28H17N3 [M+H]+ 396.1495, found 396.1492. λex=389 nm; λem=442 nm, Quantum Yield: 0.01.

### 2,3,4-trimethoxy-5-(3,4,5-trimethoxyphenyl)pyridazino[6′,1′:2,3]imidazo[4,5-c]isoquinoline

Compound 16f was prepared by general procedure 4 (16 mg, 0.034 mmol, 16% yield). Pale brown solid. ¹H NMR (500 MHz, CDCl3) δ 8.52-8.47 (m, 1H), 8.12 (d, J=9.3 Hz, 1H), 7.95 (s, 1H), 7.28 (dd, J=9.3, 4.2 Hz, 1H), 6.72 (s, 2H), 4.11 (s, 3H), 3.88 (s, 6H), 3.84 (s, 3H), 3.81 (s, 6H), 3.43 (s, 3H). 13C{1H} (126 MHz, CDCl3) δ 157.5, 155.2, 152.2, 152.0, 142.8, 142.5, 141.1, 139.5, 137.5, 135.5, 130.6, 128.8, 126.0, 120.7, 117.3, 106.3, 97.7, 77.3, 77.0, 76.8, 61.4, 61.1, 61.0, 56.6, 56.2. HRMS (ESI) Calculated for C25H24N4O6 [M+H]+ 477.1769, found 477.1769. λex=295 nm; λem=488 nm, Quantum Yield: 0.07.

### 2,3,4-trimethoxy-5-(3,4,5-trimethoxyphenyl)pyrimido[2′,1′:2,3]imidazo[4,5-c]isoquinoline

Compound 16g was prepared by General procedure 4. (8.00 mg, 0.02 mmol, 8%).

Light yellow-brown solid. ¹H NMR (500 MHz, CDCl3) δ 9.19 (d, J=6.8 Hz, 1H), 8.85 (s, 1H), 8.13 (s, 1H), 7.08-7.01 (m, 1H), 6.75 (s, 2H), 4.15 (s, 3H), 3.93 (s, 3H), 3.92 (s, 3H), 3.87 (s, 6H), 3.46 (s, 3H). 13C{1H} (126 MHz, CDCl3) δ 157.4, 154.6, 153.6, 152.3, 151.6, 143.0, 139.2, 137.6, 132.2, 117.5, 108.0, 106.1, 104.9, 98.8, 77.3, 77.0, 76.8, 61.4, 61.1, 61.0, 56.6, 56.4, 56.2. HRMS (ESI) Calculated for C25H24N4O6 [M+H]+ 477.1769, found 477.1765. λex=296 nm; λem=510 nm, Quantum Yield: 0.07.

### 8-bromo-2,3,4-trimethoxy-5-(3,4,5-trimethoxyphenyl)pyrido[2′,1′:2,3]imidazo[4,5-c]isoquinoline

Compound 16h was prepared by General procedure 4. (14 mg, 0.025 mmol, 27% yield). Pale orange-brown solid. ¹H NMR (500 MHz, CDCl3) δ 8.03 (s, 1H), 7.85 (s, 1H), 7.37 (s, 1H), 7.21 (d, J=7.0 Hz, 1H), 6.90 (s, 2H), 4.17 (s, 3H), 3.96 (s, 3H), 3.94 (s, 3H), 3.88 (s, 6H), 3.45 (s, 3H). 13C{1H} (126 MHz, CDCl3) δ 156.8, 152.1, 152.0, 151.3, 150.8, 142.8, 139.0, 137.6, 128.9, 122.2, 117.7, 116.3, 107.5, 107.1, 98.2, 77.3, 77.0, 76.8, 61.7, 61.5, 61.4, 61.3, 61.0, 56.6, 56.2, 29.7. HRMS (ESI) Calculated for C26H24BrN3O6 [M+H]+ 554.0921, found 554.0919. λex=265 nm; λem=553 nm, Quantum Yield: 0.002.

### 2,3,4-trimethoxy-5-(3,4,5-trimethoxyphenyl)thiazolo[2′, 3′: 2,3]imidazo[4,5-c]isoquinoline

Compound 16i was prepared by adding (E)-1-(3,4,5-trimethoxyphenyl)-N-(6-(3,4,5-trimethoxyphenyl)imidazo[2,1-b]thiazol-5-yl)methanimine (1 eq., 0.12 mmol) and copper (II) trifluoromethanesulfonate (1 eq., 0.12 mmol) to a MWV with a stirbar. The MWV was capped and dry 1,2-dichloroethane (2 mL, 0.06 M) injected. The reaction was stirred and heated to 120° C. in microwave for 20 min. The solvent was distilled off in vacuo and the crude product dissolved in DCM then washed with saturated sodium bicarbonate (aq). The organic layer was collected, dried over either sodium sulfate (anhyd.) or magnesium sulfate (anhyd.). The crude product in DCM was then dry loaded onto silica and purified via automated flash chromatography via a Teledyne ISCOTM (usually 0-3% MeOH/DCM) to afford the title compound.

(31 mg, 0.064 mmol, 54% yield). Dark brown-gray solid. 1H NMR (500 MHz, CDCl₃) δ 8.05 (d, J=4.5 Hz, 1H), 7.77 (s, 1H), 6.90 (d, J=4.5 Hz, 1H), 6.71 (s, 2H), 4.09 (s, 3H), 3.89 (s, 6H), 3.85 (s, 6H), 3.43 (s, 3H). 13C{1H} (126 MHz, CDCl₃) δ 157.1, 152.3, 151.2, 151.2, 141.8, 139.5, 137.3, 128.6, 117.7, 116.1, 111.7, 106.1, 96.8, 77.3, 77.1, 76.8, 61.2, 61.1, 60.9, 56.4, 56.1. HRMS (ESI) Calculated for C24H23N306S [M+H]+ 482.1380, found 482.1378.

### 2,4-dimethoxy-5-phenylpyrido[2′,1′:2,3]imidazo[4,5-c]isoquinoline

Compound 18a was prepared by General procedure 5. (176 mg, 0.495 mmol, 99% yield).

Tan solid. 1H NMR (400 MHz, CDCl₃) δ 9.14 (d, J=6.7 Hz, 1H), 8.43 (d, J=2.3 Hz, 1H), 8.39 (d, J=9.0 Hz, 1H), 8.04 (t, J=8.0 Hz, 1H), 7.47 (s, 6H), 6.70 (d, J=2.2 Hz, 1H), 4.21 (s, 3H), 3.58 (s, 3H).

13C{1H} (101 MHz, CDCl₃) δ 164.32, 159.41, 157.87, 143.14, 140.48, 135.54, 132.85, 129.35, 128.36, 127.92, 127.25, 125.22, 121.02, 116.32, 114.68, 113.74, 101.85, 96.80, 77.38, 77.06, 76.74, 57.15, 55.45. (ESI) Calculated for C22H17N3O2 [M+H]+ 356.1394, found 356.1393. λex=365 nm; λem=422 nm, Quantum Yield: 0.01.

### 2,4-dimethoxy-5-(4-nitrophenyl)pyrido[2′,1′:2,3]imidazo[4,5-c]isoquinoline

Compound 18b was prepared by General procedure 5. (164 mg, 0.41 mmol, 82% yield).

Yellow solid. 1H NMR (500 MHz, DMSO) δ 9.30 (d, J=6.8 Hz, 1H), 8.37-8.31 (m, 2H), 8.27-8.16 (m, 2H), 7.73 (d, J=8.2 Hz, 2H), 7.70-7.63 (m, 1H), 7.60 (d, J=6.5 Hz, 1H), 6.90 (d, J=2.7 Hz, 1H), 4.04 (s, 3H), 3.58 (s, 3H).

13C{1H} (126 MHz, DMSO) δ 163.9, 159.4, 147.1, 130.2, 126.6, 122.7, 122.4, 113.7, 101.4, 94.8, 56.6, 56.3, 40.4, 40.4, 40.3, 40.2, 40.1, 40.0, 39.9, 39.9, 39.8, 39.6, 39.4. (ESI) Calculated for C₂₂H16N4O4 [M+H]+ 401.1244, found 401.1241. λex=370 nm; λem=N/A nm, Quantum Yield: 0.N/A.

### methyl (E)-2,4-dimethoxy-5-styrylpyrido[2′,1′:2,3]imidazo[4,5-c]isoquinoline-10-carboxylate

Compound 18c was prepared by adding methyl 3-amino-2-(3,5-dimethoxyphenyl) imidazo[1,2-a]pyridine-7-carboxylate (1 eq., 0.21 mmol), cinnamaldehyde (0.03 mL, 99% Wt., 1.1 eq., 0.24 mmol), and copper (II) trifluoromethanesulfonate (1 eq., 0.21 mmol) were added to a MWV with a stirbar. The MWV was capped and dry 1,2-dichloroethane (1 mL, 0.2 M) injected. Reaction was stirred and heated to 100° C. until completion. The solvent was distilled off in vacuo and the crude product dissolved in DCM then washed with saturated sodium bicarbonate (aq). The organic layer was collected, dried over either sodium sulfate (anhyd.) or magnesium sulfate (anhyd.). The crude product in DCM was then dry loaded onto silica and purified via automated flash chromatography via a Teledyne ISCOTM (usually 0-3% MeOH/DCM) to afford the title compound (68 mg, 0.15 mmol, 72% yield). Orange solid. 1H NMR (500 MHz, CDCl₃) δ 8.76-8.58 (m, 1H), 8.39 (d, J=15.4 Hz, 1H), 8.31 (s, 1H), 7.66 (d, J=15.3 Hz, 1H), 7.54 (d, J=7.3 Hz, 2H), 7.47 (s, 1H), 7.39-7.28 (m, 3H), 7.25 (d, J=7.4 Hz, 1H), 6.51 (s, 1H), 3.95 (s, 3H), 3.91 (s, 6H). 13C{1H} (126 MHz, CDCl₃) δ 165.3, 160.1, 137.6, 137.6, 134.1, 129.7, 128.7, 128.1, 127.3, 110.8, 100.4, 77.3, 77.0, 76.8, 56.1, 52.9. HRMS (ESI) Calculated for C₂₆H21N3O4 [M+H]+ 440.1605 found 440.1603. λex=325 nm; λem=510 nm, Quantum Yield: 0.19.

### TD-DFT Calculations

All geometry optimizations, TDDFT computations (S. J. A. van Gisbergen, J. G. Snijders and E. J. Baerends, Implementation of time-dependent density functional response equations, Computer Physics Communications 118, 119 (1999) and molecular orbital and natural transition orbital (NTO) visualizations were carried out with the Amsterdam Modeling Suite (AMS) versions 2021.102 through 2022.104 (2021.102, SCM, Theoretical Chemistry, Vrije Universiteit, Amsterdam, The Netherlands, http://www.scm.com. R. Ruger, M. Franchini, T. Trnka, A. Yakovlev, E. van Lenthe, P. Philipsen, T. van Vuren, B. Klumpers, T. Soini) on the UA Research Computing High-Performance Computer. Calculations were performed with the hybrid B3LYP-D4 functional and with the range-separated, coulomb-attenuated method hybrid CAMY-B3LYP-D4 functional, the latter both with and without the Tamm-Dancoff approximation (Hirata, S.; Head-Gordon, M. Time-Dependent Density Functional Theory within the Tamm-Dancoff Approximation. Chem. Phys. Lett. 1999, 314 (3), 291-299). The Tamm-Dancoff approximation was necessary for the determination of the NTOs. In all cases, the basis set was a triple-zeta valence with two polarization functions and without a frozen core. Solvation considerations were made using a Conductor like Screening Model (COSMO) where the solvent dielectric value is taken from the SCM website description of COSMO (E=32.6 for methanol) (G. te Velde, F. M. Bickelhaupt, E. J. Baerends, C. Fonseca Guerra, S. J. A. van Gisbergen, J. G. Snijders and T. Ziegler, Chemistry with ADF, Journal of Computational Chemistry 22, 931 (2001)) and the atomic radii were from MM3 (Allinger, N. L.; Zhou, X.; Bergsma, J. Molecular Mechanics Parameters. Journal of Molecular Structure: THEOCHEM 1994, 312 (1), 69-83). Relativistic corrections were made with the zeroth-order relativistic approximation (ZORA) also found within the ADF package within the AMS Modeling Suite.

### Results

This example describes the development of a 4-center-3-component reaction by simple modification of aldehyde stoichiometry to afford 14, which is primed for cyclization and oxidation, delivering fluorescent tetracycles in a mere two steps containing three points of diversity (Scheme 2) (Levi, L.; Müller, T. J. J. Multicomponent Syntheses of Functional Chromophores. Chemical Society Reviews. 2016, pp 2825-2846). Building on the fluorescent properties of 16a, the increased conjugation in new chemo-types 14 and 15 was contemplated to heighten emission efficiency maximums which are known to change in different π conjugated molecules (amaguchi, Y.; Matsubara, Y.; Ochi, T.; Wakamiya, T.; Yoshida, Z. I. How the π Conjugation Length Affects the Fluorescence Emission Efficiency. J. Am. Chem. Soc. 2008, 130 (42), 13867-13869). It was thus hypothesized reaction of 2-cyanomethylpyridine 11, aromatic aldehydes 12, and TMS-CN 13 extended by one step by the addition of a second equivalent of aldehyde 12 would form conjugated imines 14 in a modified but functionally unique MCR (Scheme 2; FIG. 2).

It was found that imine formation 14a quenched fluorescence (Scheme 2). In addition, aza-Friedel-Crafts intramolecular cyclization and concomitant oxidation to the indolizine tetracycles 15a (Scheme 2) resulted in a return of fluorescence revealing a tunability of green to blue fluorescence on moving from primary amine 16a to 15a (Scheme 2).

The reaction conditions were developed for the 4CR-3C reaction building upon previous reports for the synthesis of arrays of amino-indolizines using aromatic aldehydes Martinez-Ariza et al., supra; Masquelin, T.; Bui, H.; Brickley, B.; Stephenson, G.; Schwerkoske, J.; Hulme, C. Sequential Ugi/Strecker Reactions via Microwave Assisted Organic Synthesis: Novel 3-Center-4-Component and 3-Center-5-Component Multi-Component Reactions. Tetrahedron Lett. 2006, 47 (17), 2989-2991). Initial optimization efforts (Table 1) evaluated the effects of molarity, time, cyanide source, and temperature on the model reaction between 2-cyanomethylpyridine 11, 3-methoxybenzaldehyde 12a, and a cyanide source 13a or 13b to form (E)-3-((3-methoxybenzylidene)amino)-2-(3-methoxyphenyl)indolizine-1-carbonitrile 14a. Utilizing TMSCN (700c, 8 h, Entry 1) afforded 14a in a good, isolated yield of 65%. Further optimization increased the yield to 80% (Entry 2). The attempts to use acetyl cyanide led to decreased yields and TMSCN proved optimal. The scope of chemistry was also expanded to include the development of a 5CR-3C reaction, comprised of a sequential Knoevenagel cycloaddition-Strecker reaction (Table 2). Unlike the product profile with aromatic aldehydes, which are more stable, employing alkyl aldehydes afforded two products in each of two trial reactions, namely the imines 14b (19%) and 14c (18%), and Knoevenagel-Strecker products 17a (13%) and 17b (29%).

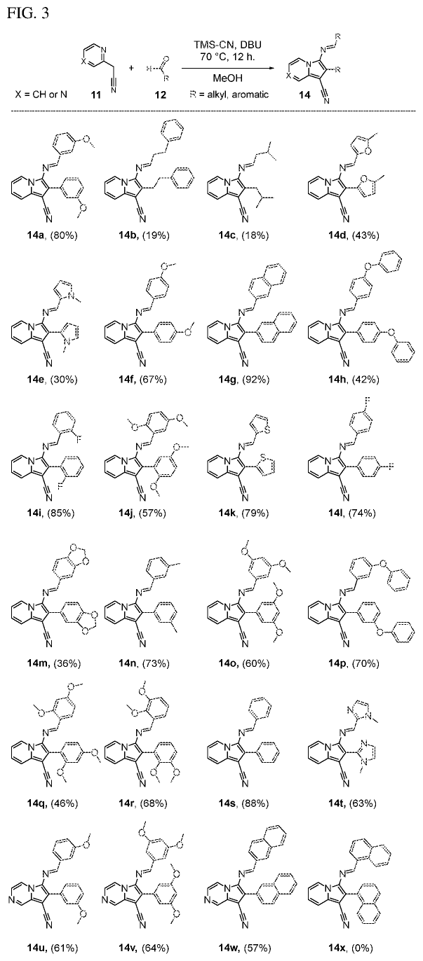

The latter derived from the 5CR-3C reaction were fluorescent having identical excitation and emission maximums. The scope of the 4CR-3C reaction utilizing aromatic aldehydes is exemplified in Scheme 3 (FIG. 3) with 21 examples, 14a-14w with isolated yields ranging from 30-92%. Some sterically hindered and/or poorly soluble aldehydes were unproductive in final product formation. For example, 2-naphthaldehyde was amenable to high yields of 14g (92% yield), yet no product 14x from use of 1-naphthaldehyde was detected. The latter was found to only afford the corresponding Knoevenagel condensation product, which precipitated out of solution. As such, it was contemplated that greater steric constraints with 1-napthaldeyhde greatly reduced the rate of the [4+1]cycloaddition. Imine formation also proceeded well with heteroaromatic aldehydes, exemplified by furan 14d, pyrrole 14e and imid-azole 14t containing products.

The reaction was also compatible with cyano-methyl pyrazines 14u-w. Aza-Friedel-Crafts reactions may proceed via intermolecular or intramolecular 1,2-addition of aromatic groups to imines, where an unoxidized secondary amine is typically generated (Terada, M.; Sorimachi, K.; Am, J.; Jia,) Y.-X; Zhong, J.; Zhu, S.-F.; Zhang, C.-M.; Zhou, Q.-L.; Zhang; G.-W; Wang, L.; Nie, J.; Ma, J.-A. Chapter 4 Enantioselective Aza-Friedel-Crafts Reaction of Naphthols with Imines 4.1 Introduction. Angew. Chem. Int. Ed 2007, 9, 1413.; Li, Y.; Xu, M. H. Lewis-Acid-Catalyzed Intramolecular Aza-Friedel-Crafts Reaction of N-Tert-Butanesulfinyl Imines: Efficient Synthesis of Optically Active 9-Aminofluorene Derivatives. Asian J. Org. Chem. 2013, 2 (1), 50-53). Imines 14 are thus primed to undergo an aza-Friedel-Crafts cyclization, where gratifyingly the fully oxidized indolizine was commonly observed as product after reaction optimization depicted in Table 3.

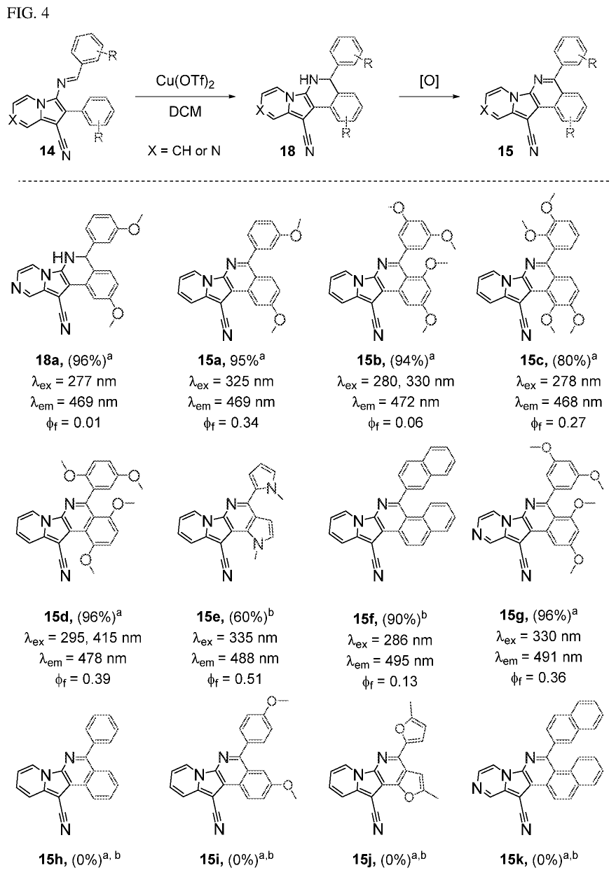

Cyclization of (E)-3-((3-methoxybenzylidene)amino)-2-(3-methoxyphenyl)indolizine-1-carbonitrile 14a to 18, was promoted with catalytic Cu(OTf)2 or Sc(OTf)3, followed by rapid oxidation to the desired tetracycle 15a, Table 3 (Kobayashi, S.; Sugiura, M.; Kitagawa, H.; Lam, W. W. L. Rare-Earth Metal Triflates in Organic Synthesis. Chem. Rev. 2002, 102 (6), 2227-2302). Reaction progress was initially monitored up to 12 hours at room temperature with modest isolated yields of 15a. Slightly improved yields were seen by performing the reaction at higher temperature (Entries 5 & 6). On increasing the stoichiometry of either Lewis acid, acceptable yields were obtained, with copper triflate (1.0 equiv., 50° C., 12 h) proving optimal. The result was consistent with prior literature indicating that Cu(OTf)2 is superior to Sc(OTf)3 in analogous trans-formations (Kobayashi et al., supra). During work-up, Cu(OTf)2 mediated reactions formed a precipitate that would not dissolve in water or a variety of organic solvents. Reports from others indicate this observation was due to the formation of copper complexes driven by copper-nitrile ligate formation (Rach, S. F.; Kuhn, F. E. Nitrile Ligated Transition Metal Complexes with Weakly Coordinating Counteranions and Their Catalytic Applications. Chem. Rev. 2009, 109 (5), 2061-2080; Trevani, L. N.; Roberts, J. C.; Tremaine, P. R. Copper(II)-Ammonia Complexation Equilibria in Aqueous Solutions at Temperatures from 30 to 250° C. by Visible Spectroscopy. J. Solution Chem. 2001, 30 (7), 585-622). Optimal work-up conditions proved to be the treatment of crude product with ammonia in methanol enabling the breakdown of the complex, and as such a very simple overall purification process. Thus, reaction scope was explored (Scheme 4; FIG. 4). Only one example, the pyrazine 18a, was isolated and found to be stable in the non-oxidized form, presumably due to the electron withdrawing inductive effects of the pyrazine nitrogen atom. This phenomenon of stable un-oxidized tetracycle was not observed with 15g, thought due to two of the four aryl methoxy substituents counteracting loss of electron density at the secondary amine by the pyrazine nitrogen.

In general and akin to other aza-Friedel-Craft cyclizations, only indolizines derived from meta-methoxy substituted aldehydes (15a-15d, 15g) underwent cyclization and oxidation via copper triflate mediated conditions. Conversely, the tetracycle 15i was not detected from the 4-methoxybenzaldehyde derived imine 14f. In most cases, Cu(OTf)2 was sufficient to mediate cyclization (18a, 15a-15d, 15g), however products 15e and 15f required more forcing conditions and neat sulfuric acid at 90° C. was employed. Under the latter conditions cyclization was also amenable to 2-substituted pyrrolo-aldehydes embedded in the indolizine of 14e to afford the unique tetracycle 15e.

The excitation wavelength of the tetracyclic indolizines ranged from 277 nm to 415 nm with two of the molecules 15b and 15d having two absorption maxima at 280, 330 nm, and 295, 415 nm respectively. The emission wave-length maxima ranged from 468 nm to 495 nm (blue to green) with the single and dimethoxy substituted indolizines 15a-15d emitting within 10 nm of one another at 468-478 nm. The quantum yields varied from 0.01 in 18a to 0.51 in 15e (FIG. 5). The fluorescence of the uniquely unoxidized 18a was not observable by the naked eye with a low quantum yield likely due to the disruption of conjugation in the molecule.

Comparing the fluorescence of the previously published amine 16a derived from 2-naphthaldehyde to the equivalent fully oxidized tetracyclic 15f, it was observed that excitation maxima decrease (292 nm to 286 nm), emission maxima decrease (505 nm to 495 nm) and quantum yield increases more than six-fold (0.02-0.13).13 Although solvent effects may be a factor, these tetracyclic indolizines have magnitudes higher quantum yield and range of fluorescent color versus existing indolizines.

TABLE 1
Optimization of Knoevenagel [4 + 1] Cycloaddition-Condensation.

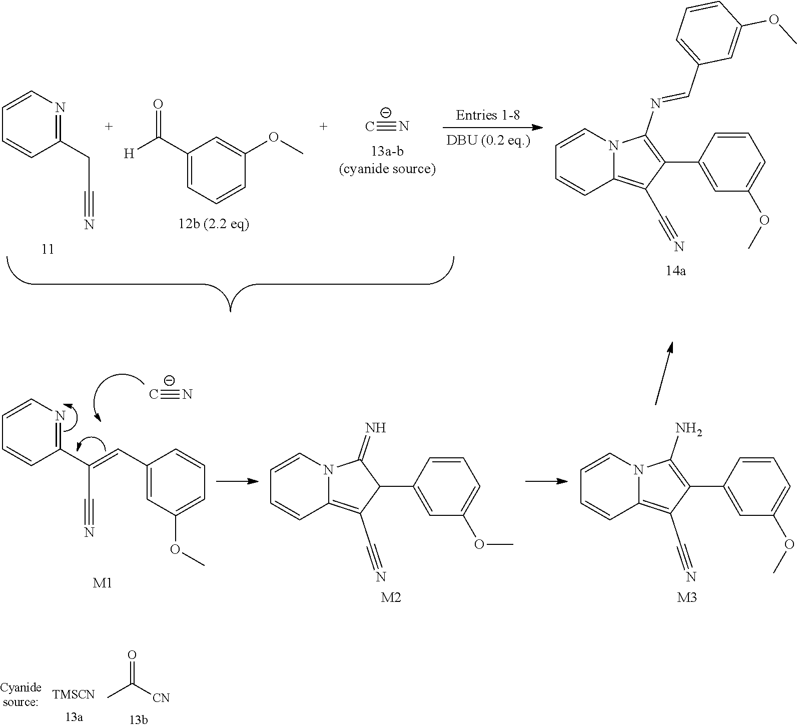

| Entry^{a} | Cyanide | MeOH | Tº C. | Time | Yield (%) |
|---|---|---|---|---|---|
| 1 | 13a | 0.2M | 70 | 8 h | 65 |
| 2 | 13a | 0.2M | 70 | 12 h | 80 |
| 3 | 13b | 0.2M | 70 | 12 h | 58 |
| 4 | 13a | 0.1M | 70 | 12 h | 63 |
| 5 | 13a | 0.2M | 70 | 24 h | 72 |
| 6 | 13a | 0.2M | Rt | 12 h | 53 |
| 7 | 13a | 0.2M | 60 | 12 h | 68 |
| 8 | 13a | 0.3M | 70 | 12 h | 59 |

^{a}Scale: 0.25 mmol

TABLE 2
Knoevenagel [4 + 1] Cycloaddition-Condensation and Strecker-products with alkyl
aldehydes.

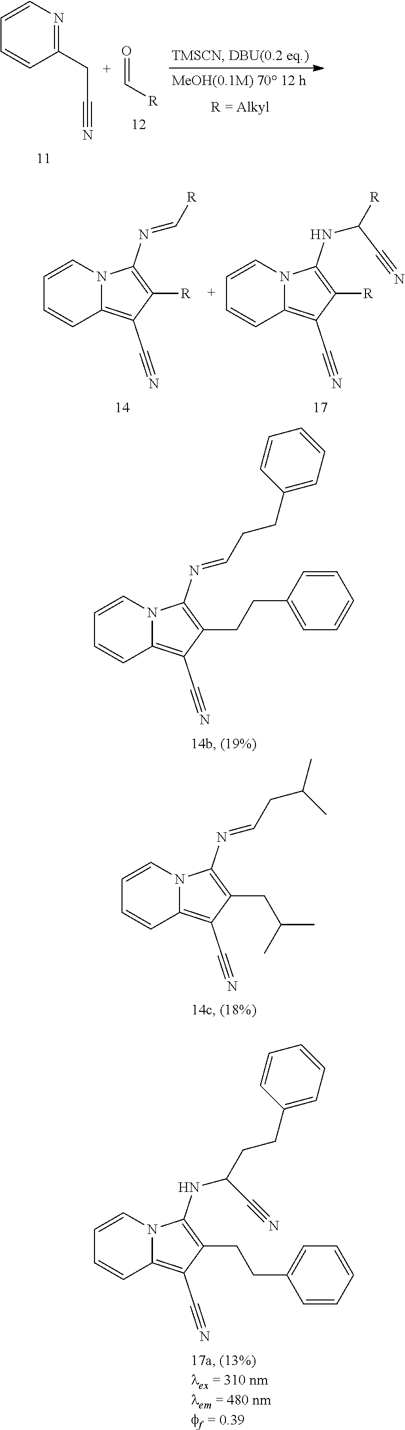

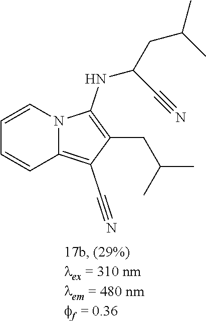 [SMILES: CC(C)Cc1c(C#N)c2ccccn2c1NC(C#N)CC(C)C]

| Entry^{a} | RCHO (eq.) | TMSCN (eq.) | Yield (%) |
|---|---|---|---|
| 14b | 2.2 | 1.0 | 19 |
| 14c | 2.2 | 1.0 | 18 |
| 17a | 4.0 | 3.0 | 13 |
| 17b | 4.0 | 3.0 | 29 |

TABLE 3
Optimization of Aza-Friedel-Crafts-Intramolecular Cyclization-Oxidation.

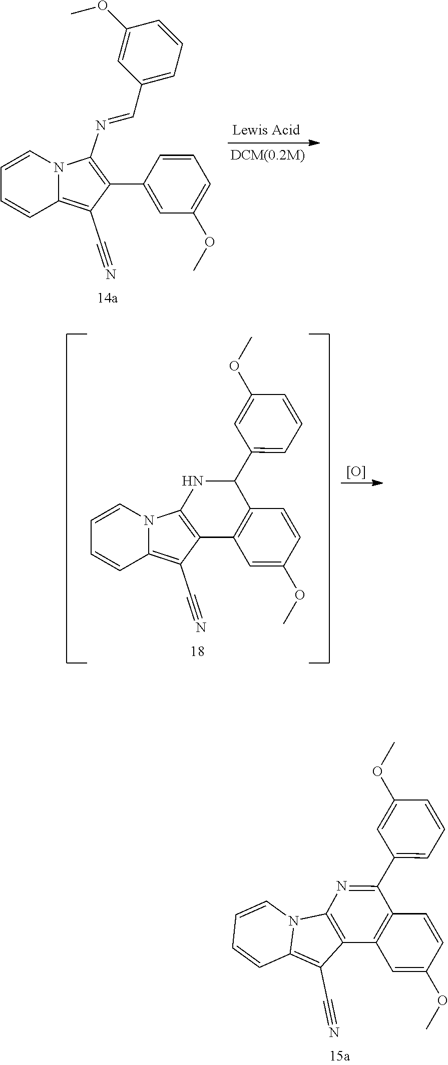

| Entry^{a} | Lewis Acid (eq.) | T (° C.) | Time (h) | Yield (%) |
|---|---|---|---|---|
| 1 | Cu(OTf)₂(0.1) | 25 | 6 | 22 |
| 2 | Sc(OTf)₃(0.1) | 25 | 6 | 12 |
| 3 | Cu(OTf)₂(0.1) | 25 | 12 | 35 |
| 4 | Sc(OTf)₃(0.1) | 25 | 12 | 27 |
| 5 | Cu(OTf)₂(0.1) | 50 | 12 | 56 |
| 6 | Sc(OTf)₃(0.1) | 50 | 12 | 43 |
| 7 | Cu(OTf)₂(0.5) | 50 | 12 | 72 |
| 8 | Sc(OTf)₃(0.5) | 50 | 12 | 57 |
| 9 | Cu(OTf)₂(1.0) | 50 | 12 | 95 |
| 10 | Sc(OTf)₃(1.0) | 50 | 12 | 68 |
| 11 | Cu(OTf)₂(1.0) | 50 | 6 | 88 |

^{a}Scale: 0.25 mmol

### Example 2

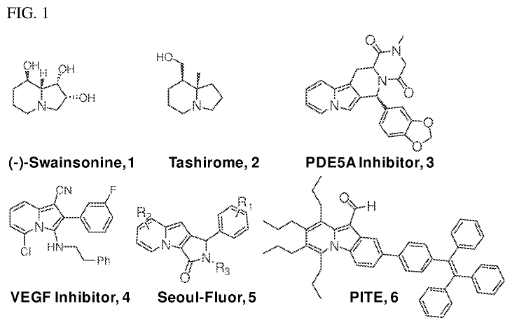

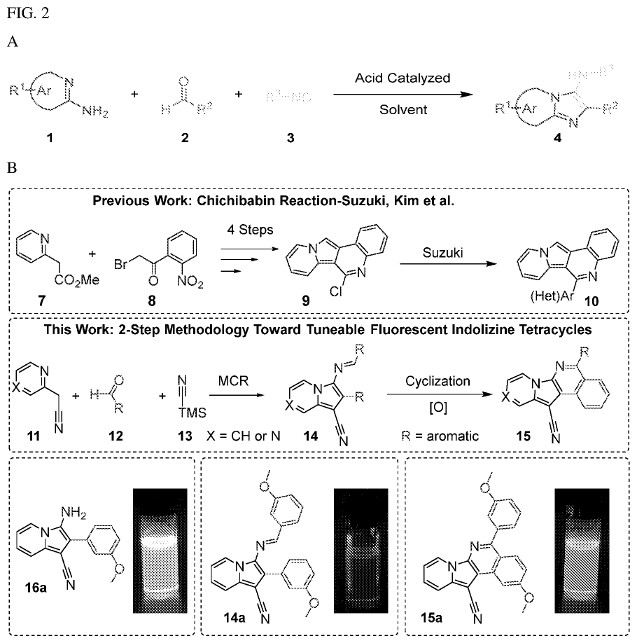

Synthesis of pyrido[2′,1′:2,3]imidazo[4,5-c]isoquinolines and imidazo[1,2-a]pyridin-3-amines stem from previous work (FIG. 1). Before working on the one-pot synthesis of the substituted pyrido[2′,1′:2,3]imidazo[4,5-c]isoquinolines, 1id was cyclized as proof of concept (Table 4). To achieve this, similar reaction conditions were incorporated from a previous study (Bedard, N.; Foley, C.; Davis, G. J.; Jewett, J. C.; Hulme, C. Sequential Knoevenagel [4+1]Cycloaddition-Condensation-Aza-Friedel-Crafts Intramolecular Cyclization: A 4-Center-3-Component Reaction Toward Tunable Fluorescent Indolizine Tetracycles. J. Org. Chem 2021, 86, 17550-17559.). Initially, reaction time was increased modestly with slight increases in yield (Entry 1-3). Catalyst loading was then increased since it was hypothesized that increasing the Lewis acid loading would further improve yield, which was confirmed (Entry 4-7). Near quantitative results were achieved by switching the solvent from dichloromethane (DCM) to Additionally, a one-pot method of synthesis to increase efficiency in the synthesis of 16d was explored (Table 5). Early experimentation found the original Lewis acid copper triflate (Cu(OTf)₂) failed to furnish the desired product 16d under different solvent types (Entry 10-12). It was noted that the formation of the cyclized product occurred only after the addition of the Bronsted acid, and acetic acid (Entry 13). This phenomenon is due to acetic acid's ability to promote aza-Friedel-Crafts reactions (Hatano, M.; Mochizuki, T.; Nishikawa, K.; Ishihara, K. Enantioselective Aza-Friedel-Crafts Reaction of Indoles with Ketimines Catalyzed by Chiral Potassium Binaphthyldisulfonates. ACS Catalysis 2018, 8 (1), 349-353). I was further speculated that increasing the acid's strength would augment the yield of 16d. As such, sulfuric acid was incorporated into the conditions (Entry 14-15). The use of sulfuric acid diminished the yields, which was attributed to its ability to interfere with imine formation 11d (Table 4). To bypass this possible interference, the order of addition of sulfuric acid was changed to after the imine formation, thereby keeping the reaction in one pot. This resulted in excellent yields of over 95% (Entry 16-18).

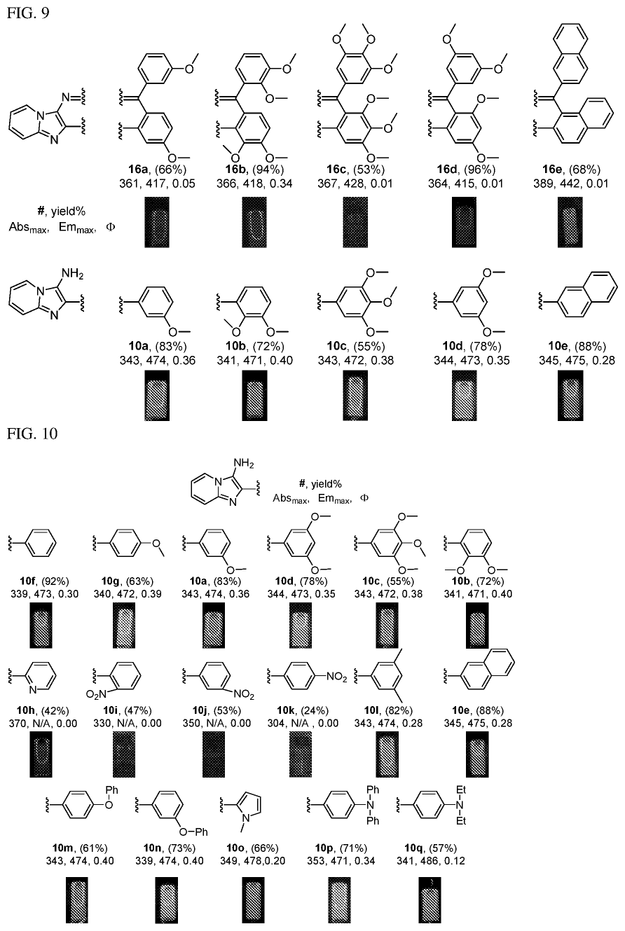

During fluorescent characterizations, a significant color change was noticed in the fluorescence of the cyclized products 16a-e compared to the free amine 10a-e (FIG. 9). In almost every molecule the fluorescence wavelength and quantum yield decreased. This was counterproductive to the goal of a high-quantum-yield-red fluorophore, so the free-amine-containing molecules 10 (Scheme 2; FIG. 1) were prioritized.

To determine the substituent effects on fluorescence the aldehyde input 8 was varied (FIG. 10) on amino-imidazopyridines 10 synthesized with previously reported conditions (Scheme 2). Starting first with a conventional benzene 10f (FIG. 11) that gave a modest quantum yield of 0.30 and an emission wavelength (Emₘₐₓ) of 473 nm, the simple introduction of a 4-methoxy group 10g increased the quantum yield by 30% to 0.39. However, this did not affect the fluorescence wavelength by an appreciable amount. Furthermore, the introduction of one or more methoxy groups in the benzene ring consistently increased the quantum yield without affecting the fluorescence wavelength. The introduction of methyl groups (101) or naphthalene (10e) decreased quantum yield while not affecting the Emₘₐₓ. Conversely introducing an electron-withdrawing group (EWG) in 10h-k consistently quenched fluorescence. It was not until the introduction of a para-N-ethyl group with 10q that significant increases in Emₘₐₓ were achieved, however, the quantum yield was decreased.

Next, a variety of aminopyridine analogues 1 were synthesized (Scheme 1;

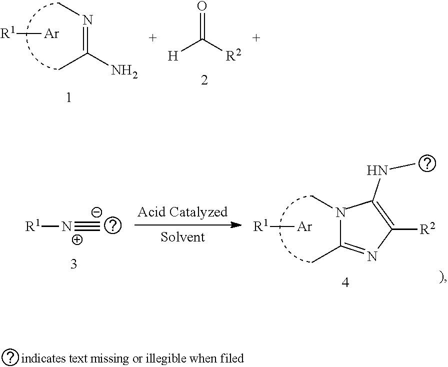

and it was found that these compounds displayed diverse fluorescent characters (FIG. 11). Notably, a substantial red-shift was observed when comparing pyridine 10d to pyrimidine 10r where the Emₘₐₓ increased significantly from 478 nm to 553 nm. This red shift corresponded with a dramatic decrease in quantum yield from 0.35 to 0.02 respectively. Synthesis of pyrazine 10s returned fluorescence quantum yield, but only the Absₘₐₓ was increased compared to 10d. Pyridazine 10t displayed similar properties to 10r with an increase in Emax and a decrease in quantum yield, while pyrimidine 10u did not react to form the product. The introduction of a methoxy-ester had mixed results on fluorescence with the para 10v increasing both Emₘₐₓ and quantum yield, while the meta 10w decreased the quantum yield, and the ortho 10x quenched fluorescence altogether.

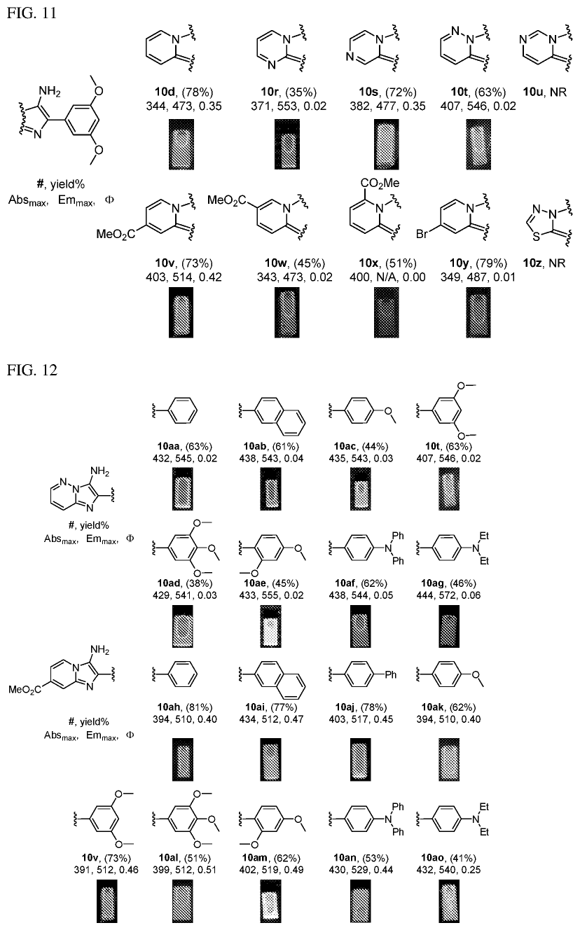

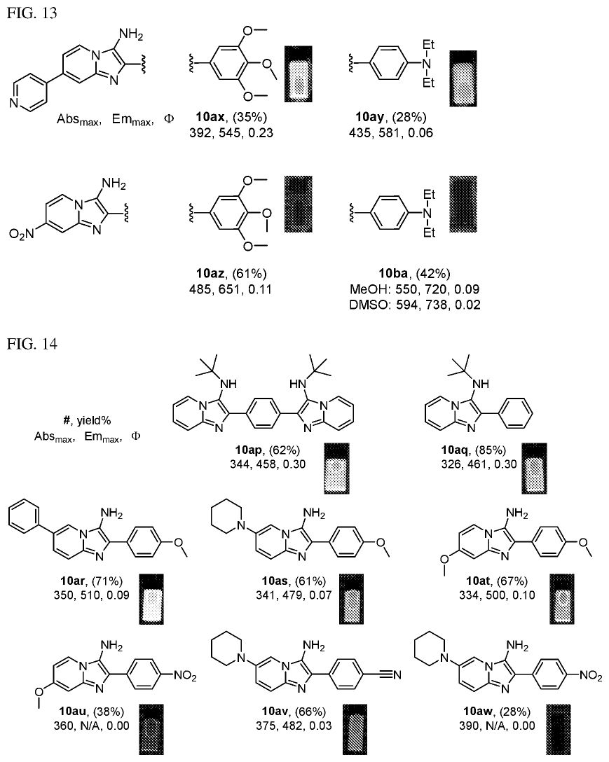

It was concluded that the aldehyde variations of the pyridazine core of 10t should be synthesized since it had a higher Emₘₐₓ with the goal to further increase the Emₘₐₓ and improve the quantum yield (FIG. 12). While the quantum yield and Emₘₐₓ were increased with the introduction of the para-N-diethyl 10ag compared to the other pyridazines, they remained rather low at 0.06 and 572 nm. After these efforts failed to increase the quantum yield of the pyridazine core of 10t, it was decided to start with the high quantum yield molecule of the meta methoxy-ester 10v and optimize its aldehyde variants (FIG. 12). The Emₘₐₓ stayed roughly similar throughout the aldehyde variations in 10ah-am at ˜510 nm. Like FIG. 10, the introduction of more methoxy groups increased the quantum yield to the highest so far at 0.51φ with 10al. The first fluorescence color change was seen from green to yellow by the aldehyde substituent of para N-ethyl group with 10ao. Furthermore, it retained a moderate quantum yield of 0.25 making it the best compound so far. These results indicated that the para position of the aminopyridine 7 (Scheme 2) was the optimal place for modifying since it was possible to increase the Emₘₐₓ while keeping a good quantum yield. Noting that the introduction of an EWG in the para position gave the best results, various EWG's were introduced (FIG. 13). The introduction of a 4-pyridine in the para position with a trimethoxy aldehyde 10ax provided a similar fluorescent profile to 10ao. When the aldehyde was swapped to the para N-ethyl group with 10ay, another color change was observed from yellow to orange at 581 nm. However, the quantum yield was decreased to 0.06. When the strength of the electron-withdrawing moiety was further increased from the para 4-pyridine to the para-nitro 10az, another color change was observed, and red fluorescence was accomplished at 651 nm with just the trimethoxy aldehyde substituent in 10az. When the aldehyde was changed to the para N-ethyl group in 10ba, Emₘₐₓ jumped from the visible range to the near-infrared (NIR) range at 720 nm. It should be noted that while the para nitro compounds had the highest Emₘₐₓ, the fluorescence had to be measured at an increased slit width of 10 mm compared to the rest of the molecules at 2.5 mm.

From there, different heterocyclic scaffolds and pairings of EDG:EWG were done to further explore the heterocyclic rings fluorescent properties (FIG. 14). 10ap, an analogue of Dömling's 3aa without the phenyl linker (Zheng, Q.; Li, X.; Kurpiewska, K.; Dömling, A. Synthesis of Tunable Fluorescent Imidazole-Fused Heterocycle Dimers. Organic Letters 2022, 24 (28), 5014-5017.), showed enhanced quantum yield (0.30) with comparable excitation and emission. One explanation for this drastic increase in quantum yield is due to the steric hindrance, previously reported to play an effect in fluorescent quenching (Chatterjee, S.; Basu, S.; Ghosh, N.; Chakrabarty, M. Steric Effect on Fluorescence Quenching. Spectrochimica Acta—Part A: Molecular and Biomolecular Spectroscopy 2005, 61 (9), 2199-2201), of 3aa. This was seen in previous work where the introduction of a conjugated phenyl alkyne in 41 increased the quantum yield over just the phenyl in 4c, however, the substitution of the phenyl for alkyl in 4q quenched fluorescence altogether (Martinez-Ariza, G.; Mehari, B. T.; Pinho, L. A. G.; Foley, C.; Day, K.; Jewett, J. C.; Hulme, C. Synthesis of Fluorescent Heterocycles: Via a Knoevenagel/[4+1]-Cycloaddition Cascade Using Acetyl Cyanide. Organic and Biomolecular Chemistry 2017, 15 (29), 6076-6079). An EDG methoxy was substituted with deleterious effects on fluorescence with 10at; the Emmax and quantum yield were both decreased to 500 nm and 0.10. The introduction of an EDG on the aminopyridine was repeated with 10as-aw to see its effect on fluorescence and in all these molecules the fluorescent properties were poor.

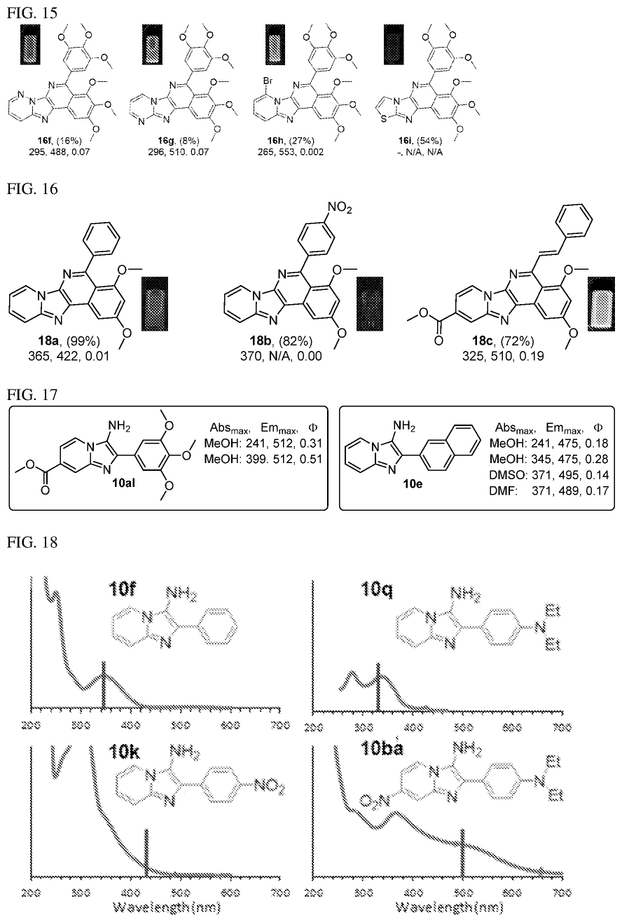

Taking into consideration the fluorescent effects of changing the aminopyridine, the same approach was taken on the fully cyclized products 16f-16i (FIG. 15). In the case of 16f, the fluorescence emission increased compared to the basic cyclized aminopyridine 16c, but it had a lower fluorescence emission compared to its amine version 10ad. This reconfirmed the previous discovery that the cyclized versions had poorer fluorescent capabilities.

The final investigation into the fluorescent properties of the cyclized products involved changing the electronics of the system in order to favorably affect the fluorescence (Table 6). This was done by the introduction of different aldehydes, making molecules during the condensation-cyclization step with excellent yields (Entry 20,24). With the introduction of an electron neutral benzaldehyde 18a, the fluorescence wavelength slightly decreased from the trimethoxy cyclized product 16c and the quantum yield stayed the same at 0.01 (FIG. 16). Similar to the amine version 10k with an EWG, the introduction of an EWG quenched the fluorescence fully in 18b. When a cinnamaldehyde moiety was introduced in 18c, the fluorescence pattern was followed with the wavelength and quantum yield decreasing compared to the amine version 10v.

Upon the initial fluorescence investigation, it was found that the amino-imidazopyridines had two Absₘₐₓ values (FIG. 17). The fluorescence quantum yield was measured on both Absₘₐₓ values and consistently the higher Absₘₐₓ value provided higher quantum yields (10al, 10e). The fluorescence was also measured in different solvents to examine the solvent effects. In general, the emission spectra shift to a higher wavelength as the polarity is increased,29 which was observed. The most polar solvent, DMSO, produced the highest wavelength emission while DMF and methanol followed the general trends. Methanol generated double the quantum yield compared to DMSO and almost double that of DMF with 10e. It was for this reason that the fluorescence measurements were run with methanol.

Time-Dependent Density Functional Theory (TD-DFT) computations were used to study a representative sample of the molecules. Molecule 10f is the basic imidazopyridine-phenyl molecule without substituents. Molecule 10q has an electron donor NEt3 substituent on the para position of the phenyl ring and, in contrast, 10k has an electron-withdrawing NO2 substituent on this position. Molecule 10ba retains the NEt2 substituent on the para position of the phenyl ring and adds a NO2 group on the opposite side of the molecule at the para position of the imidazopyridine group. Computations were carried out with the range-separated hybrid (RSH) density functional CAMY-B3LYP (Seth, M.; Ziegler, T. Range-Separated Exchange Functionals with Slater-Type Functions. Journal of Chemical Theory and Computation 2012, 8 (3), 901-907). CAMY-B3LYP is the coulomb-attenuating method functional that is the Slater-type orbital counterpart of CAM-B3LYP (Yanai, T.; Tew, D. P.; Handy, N. C. A New Hybrid Exchange-Correlation Functional Using the Coulomb-Attenuating Method (CAM-B3LYP). Chemical Physics Letters 2004, 393 (1-3), 51-57), which has been shown to compare well among the best range-separated hybrid density functionals (Salzner, U.; Aydin, A. Improved Prediction of Properties of 7-Conjugated Oligomers with Range-Separated Hybrid Density Functionals. Journal of Chemical Theory and Computation 2011, 7 (8), 2568-2583). The basis set was a triple-zeta valence plus double polarization set of Slater-type functions as defined in the Amsterdam Modeling Suite (AMS) (Chatterjee, S.; Basu, S.; Ghosh, N.; Chakrabarty, M. Steric Effect on Fluorescence Quenching. Spectrochimica Acta—Part A: Molecular and Biomolecular Spectroscopy 2005, 61 (9), 2199-2201; Martinez-Ariza, G.; Mehari, B. T.; Pinho, L. A. G.; Foley, C.; Day, K.; Jewett, J. C.; Hulme, C. Synthesis of Fluorescent Heterocycles: Via a Knoevenagel/[4+1]-Cycloaddition Cascade Using Acetyl Cyanide. Organic and Biomolecular Chemistry 2017, 15 (29), 6076-6079. FIG. 18 shows the calculated first absorption wavelengths for these molecules overlaid on the experimental absorption spectra. The wavelengths calculated for the first absorption bands fall within the envelopes of the experimentally observed absorption bands. Molecules 10f and 10q have well-defined first absorption bands and the agreements between the calculated and observed first absorption band positions are very good. The nitro-substituted molecules 10k and 10ba have broad first absorption bands with long tails to low energy. The calculated absorption energy for 10k is at the onset of the absorption band. For comparison, B3LYP functional computations drastically overestimate the initial absorption wavelengths of 10k and 10ba by about 150 nm. Common functionals such as B3LYP without range-separated potentials often overestimate the wavelengths of excitations with large amounts of charge-transfer character (Dreuw, A.; Head-Gordon, M. Single-Reference Ab Initio Methods for the Calculation of Excited States of Large Molecules. Chemical Reviews. November 2005, pp 4009-4037; Korzdorfer, T.; Bredas, J. L. Organic Electronic Materials: Recent Advances in the Dft Description of the Ground and Excited States Using Tuned Range-Separated Hybrid Functionals. Accounts of Chemical Research 2014, 47 (11), 3284-3291). This error traces to incorrect long-range behavior of the exchange-correlation energy. The agreement with experiment can be improved by tuning the parameters that define the range-separated potential for each molecule, but for these molecules the CAMY-B3LYP functional accounts sufficiently well for the initial absorptions without adjusting the parameters from those recommended in Seth et al (Seth, M.; Ziegler, T. Range-Separated Exchange Functionals with Slater-Type Functions. Journal of Chemical Theory and Computation 2012, 8 (3), 901-907).

The optimized structures for each of these molecules show that the lowest energy conformer is a structure with the imidazopyridine and phenyl portions out of the plane with each other by 24-33°. This twist reduces the overlap between the pi orbitals of each side of the molecule and, when the excitation corresponds to a charge transfer from one side of the molecule to the other, weakens the spatial overlap between the ground and excited electronic states. Strong charge transfer can also lead to larger geometry relaxations. Both factors tend to reduce the oscillator strength for emission and lengthen the excited state lifetime. Fluorescence tends to favor excited states with shorter lifetimes so that radiative decay can occur before the longer timescales of alternative relaxation pathways.

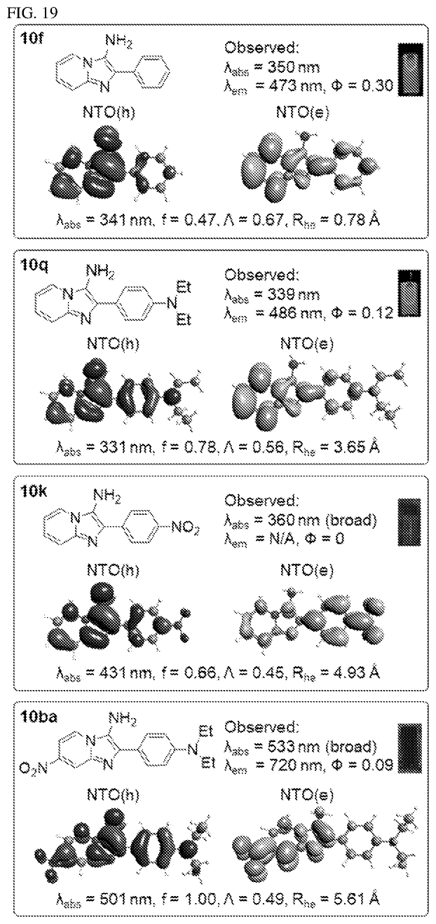

FIG. 19 compares the electron distributions of the natural transition orbitals (NTOs) for these molecules.35 These NTOs average 98% pure HOMO to LUMO excitations. For the parent molecule 10f, the computations show the electron distribution associated with the occupied NTO orbital involved in the excitation (NTO(h) in FIG. 19) is localized largely in the pi-system of the imidazopyridine portion of the molecule. The virtual NTO orbital involved in the excitation (NTO(e) in FIG. 19) has significant character throughout the molecule. FIG. 19 lists two indicators of the degree of charge transfer associated with the excitation (more in the SI). One indicator is the spatial overlap between these orbitals (symbol A) (Peach, M. J. G.; Benfield, P.; Helgaker, T.; Tozer, D. J. Excitation Energies in Density Functional Theory: An Evaluation and a Diagnostic Test. The Journal of Chemical Physics 2008, 128 (4), 44118), and the second indicator is the distance between the charge centroids of the orbitals (Guido, C. A.; Cortona, P.; Mennucci, B.; Adamo, C. On the Metric of Charge Transfer Molecular Excitations: A Simple Chemical Descriptor. Journal of Chemical Theory and Computation 2013, 9 (7), 3118-3126), which is interpreted as the hole-electron distance in the excited state (symbol Rhe). Among this group of molecules, the spatial overlap of the NTO orbitals for 10f is the largest, and the hole-electron separation is the shortest (Rhe=0.78 Å).

With the p-NEt2 substitution on the phenyl group in molecule 10q, the orbital energies are less stable as expected from the donor ability of NEt2, but there is little shift in the excitation energy because both orbitals become less stable. The electron distribution of NTO(h) shifts toward the phenyl portion of the molecule and the electron distribution of NTO(e) shifts toward the imidazopyridine portion of the molecule (FIG. 19). The charge transfer character increases relative to 10f as indicated by the smaller spatial overlap of the orbitals (Λ=0.58) and the increased hole-electron separation (Rhe=3.65 Å). The oscillator strength of the first excitation of 10q (0.78) is calculated to be greater than the oscillator strength of 10f (0.47), consistent with the greater charge transfer associated with the excitation. The wavelength of the absorption does not change significantly, but the fluorescence intensity decreases with the greater charge-transfer character associated with the excitation.

The effect of the p-nitro substituent on the phenyl ring is shown with molecule 10k. Most significantly, the NTO(e) orbital now has electron distribution almost exclusively on the phenyl-p-NO2 portion of the molecule due to the electron withdrawing ability of the NO2 group. The smaller spatial overlap (Λ=0.45) and the longer hole-electron distance (Rhe=4.93 Å) are indicative of the large amount of charge transfer associated with this excitation. This molecule did not show any observable fluorescence intensity. Placing the nitro substituent on the imidazopyridine portion of the molecule in 10ba resulted in a large shift of the absorption to longer wavelength from that of 10q. As noted above, modification of the para position of the imidazopyridine moiety proved to be the most effective for substantially tuning the fluorescence wavelength of these molecules. The shift, in this case, is due entirely to a large 1.7 eV stabilization of the LUMO, which has a substantial character on the p-NO₂ addition. In contrast, the HOMO shows little shift with the nitro substitution, consistent with the small character of this orbital on the substituent carbon atom. Like 10k, this molecule shows a large amount of charge transfer in the excitation (Λ=0.49. Rhe=5.61 Å), but the charge transfer is in the opposite direction from 10k, being to the imidazopyridine portion of the molecule rather than to the phenyl portion of the molecule. Like 10k, the fluorescence intensity is low. These results indicate that the fluorescence intensity tends to be low if the calculated spatial overlap of the NTOs is small and the hole-electron separation is large.

TABLE 4
Optimization of Aza-Friedel-Crafts-Intramolecular Cyclization-Oxidation.

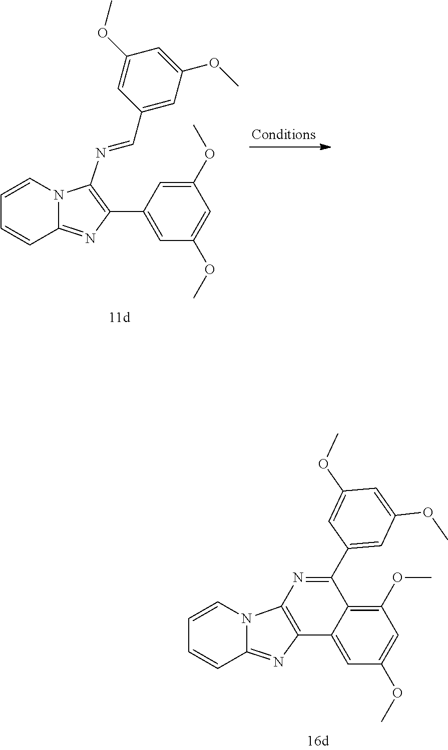 [SMILES: COc1cc(/C=N/c2c(-c3cc(OC)cc(OC)c3)nc3ccccn23)cc(OC)c1 ; COc1cc(OC)cc(-c2nc3c(nc4ccccn43)c3cc(OC)cc(OC)c23)c1]

| | LA | T | | Time | Yield |
|---|---|---|---|---|---|
| Entry^{a} | (eq.) | (° C.) | Solvent (M) | (min) | (%) |
| 1 | Cu(OTf)₂(0.5) | 100 | DCM(0.1) | 5 | 20 |
| 2 | Cu(OTf)₂(0.5) | 100 | DCM(0.1) | 10 | 28 |
| 3 | Cu(OTf)₂(0.5) | 100 | DCM(0.1) | 20 | 28 |
| 4 | Cu(OTf)₂(1.0) | 100 | DCM(0.1) | 5 | 68 |
| 5 | Cu(OTf)₂(1.0) | 100 | DCM(0.1) | 10 | 83 |
| 6 | Cu(OTf)₂(1.0) | 100 | DCM(0.1) | 20 | 91 |
| 7 | Cu(OTf)₂(2.0) | 100 | DCM(0.1) | 20 | 93 |
| 8 | Cu(OTf)₂(1.0) | 120 | DCE(0.1) | 20 | 99 |
| 9 | Cu(OTf)₂(2.0) | 120 | DCE(0.1) | 20 | 99 |

TABLE 5
One-Pot Optimizations Toward Fluorescent Pyrido[2′,1′:2,3]imidazo[4,5-c]isoquinolines

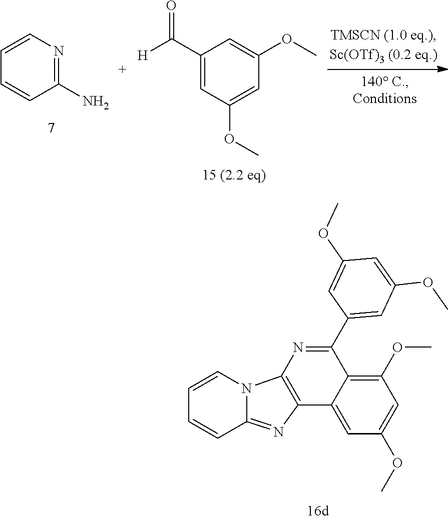 [SMILES: Nc1ccccn1 ; COc1cc(C=O)cc(OC)c1 ; COc1cc(OC)cc(-c2nc3c(nc4ccccn43)c3cc(OC)cc(OC)c23)c1]

| | Acid | | | Yield |
|---|---|---|---|---|
| Entry^{a} | (eq.) | Solvent (0.3M) | Time (min) | (%) |
| 10 | Cu(OTf)₂ (1.0) | MeOH | 20 | 0 |
| 11 | Cu(OTf)₂ (1.0) | DCM^{b} | 20 | 0 |
| 12 | Cu(OTf)₂ (1.0) | ACN | 20 | 0 |
| 13 | — | AcOH | 20 | 28 |
| 14 | H₂SO₄ (30 eq.) | AcOH | 20 | 6 |
| 15 | H₂SO₄ (50 eq.) | MeOH | 20 | 0 |
| 16 | H₂SO₄ (50 eq.) | MeOH | 20, 5^{c} | 81 |
| 17 | H₂SO₄ (50 eq.) | MeOH | 20, 10^{c} | 87 |
| 18 | H₂SO₄ (50 eq.) | MeOH | 20, 20^{c} | 96 |

TABLE 6
Asymmetric Aldehyde Variation

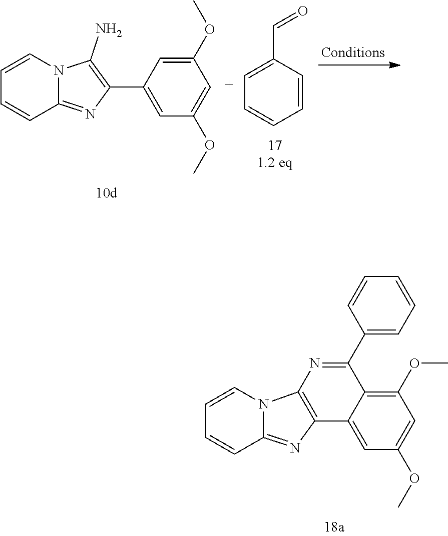

| | Acid | Solvent | Time | |
|---|---|---|---|---|
| Entry^{a} | (eq.) | (0.1M) | (min) | Yield (%) |
| 19 | Cu(OTf)₂ (1.0) | DCM | 5 | 89 |
| 20 | Cu(OTf)₂ (1.0) | DCM | 10 | 95 |
| 21 | Cu(OTf)₂ (1.0) | DCM | 20 | 99 |
| 22 | H₂SO₄ (50 eq.) | MeOH | 5 | 41 |
| 23 | H₂SO₄ (50 eq.) | MeOH | 10 | 72 |
| 24 | H₂SO₄ (50 eq.) | MeOH | 20 | 99 |

### Example 3

Xray structure report.

### Experimental

The single crystal XRD of C₂₄H₂₅N₅ [mo_NB_IT_1_1_0ma]was studied on a Bruker Kappa APEX-II diffractometer. The crystal was kept at 100.0 K during the data collection. Using the Olex2 (Dolomanov, O. V., Bourhis, L. J., Gildea, R. J, Howard, J. A. K. & Puschmann, H. (2009), J. Appl. Cryst. 42, 339-341) environment, the structure was solved with the ShelXT (Sheldrick, G. M. (2015). Acta Cryst. A71, 3-8) structure solution program using Intrinsic Phasing and refined with the ShelXL (Sheldrick, G. M. (2015). Acta Cryst. C71, 3-8) refinement package using Least Squares minimization.

### Crystal Structure Determination of [Mo_NB_IT_1_1_0Ma]

Crystal Data for C24H25N5 (M=383.49 g/mol): monoclinic, space group P2₁/c (no. 14), a=17.4919 (13) Å, b=9.2308 (7) Å, c=12.5168 (9) Å, β=98.011 (4)°, V=2001.3 (3) Å³, Z=4, T=100 K, μ(MoKα)=0.078 mm⁻¹, Dcalc=1.273 g/cm³, 19648 reflections measured (4.704°≤2Θ≤50.028°), 3529 unique (Rᵢₙₜ=0.0443, R_{sigma}=0.0296) which were used in all calculations. The final R₁ was 0.0407 (I>2σ(I)) and wR₂ was 0.0942 (all data).

### Refinement Model Description

Number of restraints—0, number of constraints—unknown.

Details:
1. Fixed Uiso
   At 1.2 times of:
      All C(H) groups
   At 1.5 times of:
      All C(H,H,H) groups
2.a Aromatic/amide H refined with riding coordinates:
   C8(H8), C18(H18), C15(H15), C7(H7), C19(H19), C5(H5 Å), C17(H17), C16(H16), C1(H1), C2(H2), C4(H4), C3(H3)
2.b Idealized Me refined as rotating group:
   C20(H20 Å,H20B,H20C), C22(H22 Å,H22B,H22C), C24(H24 Å,H24B,H24C), C23(H23 Å,H23B,
   H23C)

### Results

TABLE 24
Crystal data and structure refinement for mo_NB_IT_1_1_0ma.

| | |
|---|---|
| Identification code | mo_NB_IT_1_1_0ma |
| Empirical formula | C₂₄H₂₅N₅ |
| Formula weight | 383.49 |
| Temperature/K | 100 |
| Crystal system | monoclinic |
| Space group | P2₁/c |
| a/Å | 17.4919(13) |
| b/Å | 9.2308(7) |
| c/Å | 12.5168(9) |
| α/° | 90 |
| β/° | 98.011(4) |
| γ/° | 90 |
| Volume/Å³ | 2001.3(3) |
| Z | 4 |
| ρ_{calc}g/cm³ | 1.273 |
| μ/mm⁻¹ | 0.078 |
| F(000) | 816.0 |
| Crystal size/mm³ | 0.45 × 0.17 × 0.04 |
| Radiation | MoKα (λ = 0.71073) |
| 2Θ range for data | 4.704 to 50.028 |
| collection/° | |
| Index ranges | −20 ≤ h ≤ 20, −10 ≤ k ≤ 10, −14 ≤ l ≤ 14 |
| Reflections collected | 19648 |
| Independent reflections | 3529 [Rᵢₙₜ = 0.0443, R_{sigma} = 0.0296] |
| Data/restraints/ | 3529/0/270 |
| parameters | |
| Goodness-of-fit on F² | 1.059 |
| Final R indexes | R₁ = 0.0407, wR₂ = 0.0888 |
| [I >= 2σ (I)] | |
| Final R indexes | R₁= 0.0540, wR₂ = 0.0942 |
| [all data] | |
| Largest diff. peak/ | 0.20/−0.20 |
| hole/e Å⁻³ | |

### Molecular Structure.

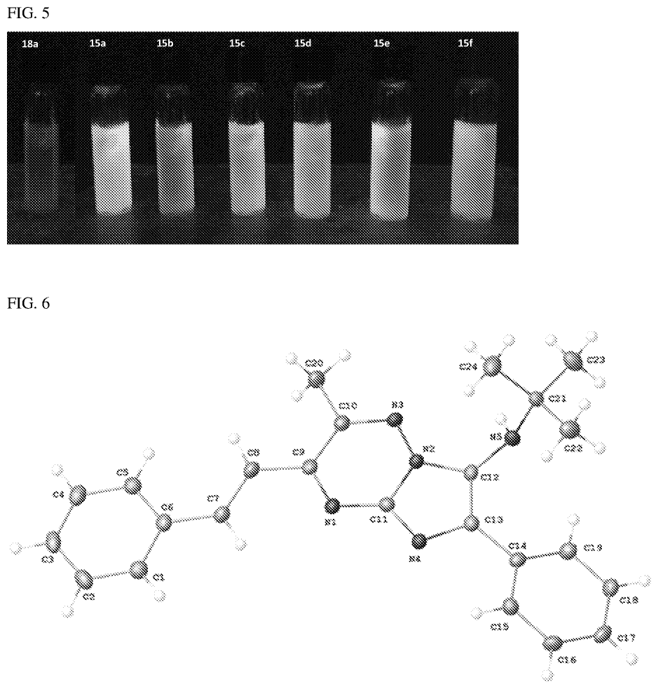

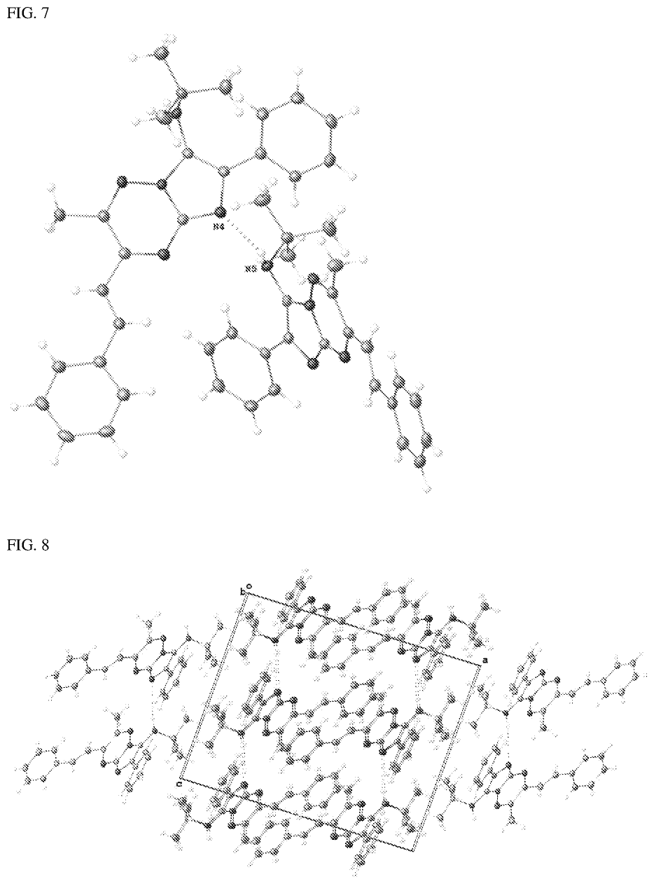

There are 4 molecules per unit cell, 1 molecule per asymmetric unit. The thermal displacement ellipsoids are at a 50% probability level (FIG. 6). There is a hydrogen bond between N5-H5 and N4 of the neighboring molecule (FIG. 7). Unit cell and packing. View almost along axis b (FIG. 8).

TABLE 25
Unit cell and packing. View almost along axis b.

| Unit cell | View almost | | | | |
|---|---|---|---|---|---|
| and packing. | along axis b. | x | y | z | U(eq) |
| Unit cell | View almost | 7541.0(8) | 3445.0(14) | 4191.9(10) | 14.7(3) |
| and packing. | along axis b. | | | | |
| Unit cell | View almost | 7466.9(8) | 2038.6(15) | 5635.5(11) | 17.0(3) |
| and packing. | along axis b. | | | | |
| Unit cell | View almost | 8321.2(8) | 1994.7(16) | 3107.9(11) | 16.5(3) |
| and packing. | along axis b. | | | | |
| Unit cell | View almost | 6764.4(8) | 4283.8(15) | 5487.6(11) | 17.3(3) |
| and packing. | along axis b. | | | | |
| Unit cell | View almost | 7361.4(8) | 4547.6(15) | 3484.1(10) | 16.6(3) |
| and packing. | along axis b. | | | | |
| Unit cell | View almost | 7955.3(9) | 2206.9(18) | 4024.7(13) | 15.1(3) |
| and packing. | along axis b. | | | | |
| Unit cell | View almost | 7246.4(9) | 3282.9(18) | 5168.6(12) | 15.5(4) |
| and packing. | along axis b. | | | | |
| Unit cell | View almost | 7879.2(9) | 1337.1(18) | 4918.3(12) | 15.9(4) |
| and packing. | along axis b. | | | | |
| Unit cell | View almost | 8094.5(9) | −195.4(18) | 5104.8(13) | 16.5(4) |
| and packing. | along axis b. | | | | |
| Unit cell | View almost | 6867.5(9) | 5487.1(18) | 3789.6(13) | 17.3(4) |
| and packing. | along axis b. | | | | |
| Unit cell | View almost | 4939.0(9) | 7109.5(18) | 6180.9(13) | 18.0(4) |
| and packing. | along axis b. | | | | |
| Unit cell | View almost | 5980.2(10) | 6399.3(19) | 5073.0(13) | 19.8(4) |
| and packing. | along axis b. | | | | |
| Unit cell | View almost | 6558.6(9) | 5361.3(18) | 4806.0(13) | 17.2(4) |
| and packing. | along axis b. | | | | |
| Unit cell | View almost | 8456.9(10) | −2536.6(19) | 4454.4(14) | 21.2(4) |
| and packing. | along axis b. | | | | |
| Unit cell | View almost | 8046.9(9) | −828.8(19) | 6110.6(13) | 19.1(4) |
| and packing. | along axis b. | | | | |
| Unit cell | View almost | 5549.1(9) | 6175.7(19) | 5865.1(13) | 18.3(4) |
| and packing. | along axis b. | | | | |
| Unit cell | View almost | 9168.4(9) | 2367.8(18) | 3190.4(13) | 17.7(4) |
| and packing. | along axis b. | | | | |
| Unit cell | View almost | 8295.6(10) | −1075.1(19) | 4275.1(13) | 19.5(4) |
| and packing. | along axis b. | | | | |
| Unit cell | View almost | 4645.3(10) | 8308.8(19) | 5574.1(14) | 20.8(4) |
| and packing. | along axis b. | | | | |
| Unit cell | View almost | 8406.3(10) | −3155.8(19) | 5455.4(14) | 21.3(4) |
| and packing. | along axis b. | | | | |
| Unit cell | View almost | 8198.9(10) | −2298.1(19) | 6277.2(14) | 21.8(4) |
| and packing. | along axis b. | | | | |
| Unit cell | View almost | 4628.2(10) | 6779(2) | 7126.1(13) | 20.9(4) |
| and packing. | along axis b. | | | | |
| Unit cell | View almost | 4053.6(10) | 7634(2) | 7470.9(14) | 23.2(4) |
| and packing. | along axis b. | | | | |
| Unit cell | View almost | 6619.0(10) | 6687.6(19) | 3005.3(14) | 23.6(4) |
| and packing. | along axis b. | | | | |
| Unit cell | View almost | 4066.6(10) | 9160(2) | 5910.0(14) | 22.8(4) |
| and packing. | along axis b. | | | | |
| Unit cell | View almost | 3773.9(10) | 8830(2) | 6865.3(14) | 23.1(4) |
| and packing. | along axis b. | | | | |
| Unit cell | View almost | 9643.6(10) | 1271(2) | 3915.4(14) | 24.7(4) |
| and packing. | along axis b. | | | | |
| Unit cell | View almost | 9334.1(11) | 3895(2) | 3636.7(16) | 27.8(4) |
| and packing. | along axis b. | | | | |
| Unit cell | View almost | 9373.7(10) | 2265(2) | 2046.2(14) | 27.1(4) |
| and packing. | along axis b. | | | | |

TABLE 26
Fractional Atomic Coordinates (×10⁴) and Equivalent Isotropic
Displacement Parameters (Å² × 10³) for mo_NB_IT_1_1_0ma.
U_{eq} is defined as ⅓ of the trace of the orthogonalized U_{IJ} tensor.

| Atom | x | y | z | U(eq) |
|---|---|---|---|---|
| N2 | 7541.0(8) | 3445.0(14) | 4191.9(10) | 14.7(3) |
| N4 | 7466.9(8) | 2038.6(15) | 5635.5(11) | 17.0(3) |
| N5 | 8321.2(8) | 1994.7(16) | 3107.9(11) | 16.5(3) |
| N1 | 6764.4(8) | 4283.8(15) | 5487.6(11) | 17.3(3) |
| N3 | 7361.4(8) | 4547.6(15) | 3484.1(10) | 16.6(3) |
| C12 | 7955.3(9) | 2206.9(18) | 4024.7(13) | 15.1(3) |
| C11 | 7246.4(9) | 3282.9(18) | 5168.6(12) | 15.5(4) |
| C13 | 7879.2(9) | 1337.1(18) | 4918.3(12) | 15.9(4) |
| C14 | 8094.5(9) | −195.4(18) | 5104.8(13) | 16.5(4) |
| C10 | 6867.5(9) | 5487.1(18) | 3789.6(13) | 17.3(4) |
| C6 | 4939.0(9) | 7109.5(18) | 6180.9(13) | 18.0(4) |
| C8 | 5980.2(10) | 6399.3(19) | 5073.0(13) | 19.8(4) |
| C9 | 6558.6(9) | 5361.3(18) | 4806.0(13) | 17.2(4) |
| C18 | 8456.9(10) | −2536.6(19) | 4454.4(14) | 21.2(4) |
| C15 | 8046.9(9) | −828.8(19) | 6110.6(13) | 19.1(4) |
| C7 | 5549.1(9) | 6175.7(19) | 5865.1(13) | 18.3(4) |
| C21 | 9168.4(9) | 2367.8(18) | 3190.4(13) | 17.7(4) |
| C19 | 8295.6(10) | −1075.1(19) | 4275.1(13) | 19.5(4) |
| C5 | 4645.3(10) | 8308.8(19) | 5574.1(14) | 20.8(4) |
| C17 | 8406.3(10) | −3155.8(19) | 5455.4(14) | 21.3(4) |
| C16 | 8198.9(10) | −2298.1(19) | 6277.2(14) | 21.8(4) |
| C1 | 4628.2(10) | 6779(2) | 7126.1(13) | 20.9(4) |
| C2 | 4053.6(10) | 7634(2) | 7470.9(14) | 23.2(4) |
| C20 | 6619.0(10) | 6687.6(19) | 3005.3(14) | 23.6(4) |
| C4 | 4066.6(10) | 9160(2) | 5910.0(14) | 22.8(4) |
| C3 | 3773.9(10) | 8830(2) | 6865.3(14) | 23.1(4) |
| C22 | 9643.6(10) | 1271(2) | 3915.4(14) | 24.7(4) |
| C24 | 9334.1(11) | 3895(2) | 3636.7(16) | 27.8(4) |
| C23 | 9373.7(10) | 2265(2) | 2046.2(14) | 27.1(4) |

TABLE 27
Anisotropic Displacement Parameters (Å² ×
10³) for mo_NB_IT_1_1_0ma. The Anisotropic
displacement factor exponent takes the form: −2π²[h²a*²U₁₁ + 2hka*b*U₁₂ + . . . ].

| Atom | U₁₁ | U₂₂ | U₃₃ | U₂₃ | U₁₃ | U₁₂ |
|---|---|---|---|---|---|---|
| N2 | 16.6(7) | 13.6(7) | 13.8(7) | 0.5(6) | 2.1(5) | 0.7(6) |
| N4 | 18.9(7) | 14.9(8) | 17.6(7) | −0.4(6) | 3.8(6) | 0.1(6) |
| N5 | 18.4(7) | 18.5(8) | 12.7(7) | 1.7(6) | 2.6(6) | 1.3(6) |
| N1 | 18.0(7) | 14.7(8) | 19.6(7) | −1.4(6) | 3.6(6) | −0.4(6) |
| N3 | 18.5(7) | 13.8(8) | 17.6(7) | 2.2(6) | 2.5(6) | 0.0(6) |
| C12 | 15.9(8) | 13.6(9) | 15.4(8) | −2.8(7) | 0.7(6) | 0.0(7) |
| C11 | 16.4(8) | 15.4(9) | 14.8(8) | −0.9(7) | 2.2(6) | −2.2(7) |
| C13 | 15.2(8) | 16.4(9) | 15.5(8) | −2.2(7) | 0.8(6) | −0.9(7) |
| C14 | 15.5(8) | 14.8(9) | 18.8(8) | −0.1(7) | 0.9(6) | −2.1(7) |
| C10 | 16.6(8) | 15.4(9) | 19.9(9) | −0.3(7) | 2.3(7) | −0.5(7) |
| C6 | 17.6(8) | 17.0(9) | 18.9(8) | −4.4(7) | 0.9(7) | −1.6(7) |
| C8 | 22.2(9) | 14.6(9) | 22.8(9) | 1.0(7) | 3.3(7) | 1.3(7) |
| C9 | 16.7(8) | 14.9(9) | 20.0(9) | −2.2(7) | 3.0(7) | −2.3(7) |
| C18 | 25.2(9) | 17.4(9) | 21.4(9) | −4.7(7) | 4.7(7) | −1.1(7) |
| C15 | 20.2(9) | 20.1(9) | 17.3(8) | −1.0(7) | 3.7(7) | 0.8(7) |
| C7 | 20.1(9) | 15.7(9) | 18.6(9) | −0.6(7) | 0.9(7) | 0.6(7) |
| C21 | 16.8(8) | 17.0(9) | 20.0(8) | 1.3(7) | 5.3(7) | 1.5(7) |
| C19 | 23.0(9) | 18.0(9) | 16.9(8) | −1.4(7) | 1.0(7) | −1.6(7) |
| C5 | 20.6(9) | 20.2(10) | 21.9(9) | −2.5(7) | 4.2(7) | −2.1(7) |
| C17 | 21.7(9) | 13.8(9) | 27.5(9) | 1.2(7) | 0.2(7) | 0.0(7) |
| C16 | 25.7(9) | 20.8(10) | 19.2(9) | 4.9(7) | 3.8(7) | −1.8(8) |
| C1 | 20.3(9) | 20.8(10) | 20.9(9) | −1.6(7) | 0.7(7) | −0.8(7) |
| C2 | 20.9(9) | 28.4(11) | 21.0(9) | −7.6(8) | 5.8(7) | −4.1(8) |
| C20 | 25.7(10) | 21.1(10) | 25.0(9) | 5.5(8) | 7.5(8) | 6.2(8) |
| C4 | 20.8(9) | 18.5(9) | 27.9(10) | −4.3(8) | −1.0(7) | 0.6(7) |
| C3 | 17.0(9) | 22.8(10) | 29.5(10) | −11.8(8) | 2.7(7) | −0.5(8) |
| C22 | 20.7(9) | 26.2(10) | 26.8(10) | 5.3(8) | 2.5(7) | 0.8(8) |
| C24 | 23.3(9) | 22.1(10) | 38.4(11) | −5.3(8) | 5.7(8) | −1.4(8) |
| C23 | 23.4(9) | 35.2(12) | 24.0(10) | 2.9(8) | 7.8(8) | 4.1(8) |

TABLE 28
Bond Lengths for mo_NB_IT_1_1_0ma.

| Atom | Atom | Length/Å | Atom | Atom | Length/Å |
|---|---|---|---|---|---|
| N2 | N3 | 1.3572(18) | C6 | C7 | 1.468(2) |
| N2 | C12 | 1.385(2) | C6 | C5 | 1.399(2) |
| N2 | C11 | 1.399(2) | C6 | CI | 1.403(2) |
| N4 | C11 | 1.322(2) | C8 | C9 | 1.466(2) |
| N4 | C13 | 1.388(2) | C8 | C7 | 1.343(2) |
| N5 | C12 | 1.403(2) | C18 | C19 | 1.390(3) |
| N5 | C21 | 1.511(2) | C18 | C17 | 1.391(2) |
| N1 | C11 | 1.348(2) | C15 | C16 | 1.392(2) |
| N1 | C9 | 1.327(2) | C21 | C22 | 1.526(2) |
| N3 | C10 | 1.318(2) | C21 | C24 | 1.529(2) |
| C12 | C13 | 1.398(2) | C21 | C23 | 1.527(2) |
| C13 | C14 | 1.474(2) | C5 | C4 | 1.392(2) |
| C14 | C15 | 1.401(2) | C17 | C16 | 1.386(2) |
| C14 | C19 | 1.401(2) | C1 | C2 | 1.392(2) |
| C10 | C9 | 1.454(2) | C2 | C3 | 1.389(3) |
| C10 | C20 | 1.504(2) | C4 | C3 | 1.398(3) |

TABLE 29
Bond Angles for mo_NB_IT_1_1_0ma.

| Atom | Atom | Atom | Angle/° | Atom | Atom | Atom | Angle/° |
|---|---|---|---|---|---|---|---|
| N3 | N2 | C12 | 126.78(13) | C5 | C6 | C1 | 118.26(16) |
| N3 | N2 | C11 | 124.79(13) | C1 | C6 | C7 | 118.77(15) |
| C12 | N2 | C11 | 107.94(13) | C7 | C8 | C9 | 123.00(16) |
| C11 | N4 | C13 | 105.39(13) | N1 | C9 | C10 | 121.44(15) |
| C12 | N5 | C21 | 117.84(13) | N1 | C9 | C8 | 118.52(15) |
| C9 | N1 | C11 | 116.62(14) | C10 | C9 | C8 | 119.98(15) |
| C10 | N3 | N2 | 113.92(13) | C19 | C18 | C17 | 120.48(16) |
| N2 | C12 | N5 | 123.14(14) | C16 | C15 | C14 | 120.47(16) |
| N2 | C12 | C13 | 104.06(13) | C8 | C7 | C6 | 127.79(16) |
| C13 | C12 | N5 | 132.77(15) | N5 | C21 | C22 | 109.71(14) |
| N4 | C11 | N2 | 111.04(14) | N5 | C21 | C24 | 111.67(14) |
| N4 | C11 | N1 | 128.36(15) | N5 | C21 | C23 | 106.16(13) |
| N1 | C11 | N2 | 120.47(14) | C22 | C21 | C24 | 109.54(14) |
| N4 | C13 | C12 | 111.44(14) | C22 | C21 | C23 | 109.66(14) |
| N4 | C13 | C14 | 119.32(14) | C23 | C21 | C24 | 110.05(15) |
| C12 | C13 | C14 | 128.84(15) | C18 | C19 | C14 | 120.60(16) |
| C15 | C14 | C13 | 119.79(15) | C4 | C5 | C6 | 120.68(16) |
| C15 | C14 | C19 | 118.47(16) | C16 | C17 | C18 | 119.34(16) |
| C19 | C14 | C13 | 121.59(15) | C17 | C16 | C15 | 120.64(16) |
| N3 | C10 | C9 | 122.67(15) | C2 | C1 | C6 | 121.32(16) |
| N3 | C10 | C20 | 115.91(14) | C3 | C2 | C1 | 119.71(16) |
| C9 | C10 | C20 | 121.40(15) | C5 | C4 | C3 | 120.25(17) |
| C5 | C6 | C7 | 122.96(15) | C2 | C3 | C4 | 119.76(16) |

TABLE 30
Hydrogen Bonds for mo_NB_IT_1_1_0ma.

| D | H | A | d(D—H)/Å | d(H—A)/Å | d(D—A)/Å | D—H—A/° |
|---|---|---|---|---|---|---|
| N5 | H5 | N4¹ | 0.900(19) | 2.51(2) | 3.3640(19) | 157.9(16) |

TABLE 31
Torsion Angles for mo_NB_IT_1_1_0ma.

| A | B | C | D | Angle/° | A | B | C | D | Angle/° |
|---|---|---|---|---|---|---|---|---|---|
| N2 | N3 | C10 | C9 | 1.2(2) | C13 | N4 | C11 | N2 | −3.29(17) |
| N2 | N3 | C10 | C20 | −176.98(14) | C13 | N4 | C11 | N1 | 172.45(16) |
| N2 | C12 | C13 | N4 | −2.86(17) | C13 | C14 | C15 | C16 | 175.73(15) |
| N2 | C12 | C13 | C14 | 169.65(15) | C13 | C14 | C19 | C18 | −176.56(16) |
| N4 | C13 | C14 | C15 | −14.9(2) | C14 | C15 | C16 | C17 | 0.6(3) |
| N4 | C13 | C14 | C19 | 160.50(15) | C6 | C5 | C4 | C3 | 0.4(3) |
| N5 | C12 | C13 | N4 | 179.18(16) | C6 | C1 | C2 | C3 | 0.7(3) |
| N5 | C12 | C13 | C14 | −8.3(3) | C9 | N1 | C11 | N2 | 3.6(2) |
| N3 | N2 | C12 | N5 | 6.8(2) | C9 | N1 | C11 | N4 | −171.81(16) |
| N3 | N2 | C12 | C13 | −171.41(14) | C9 | C8 | C7 | C6 | −177.88(15) |
| N3 | N2 | C11 | N4 | 174.01(14) | C18 | C17 | C16 | C15 | −0.4(3) |
| N3 | N2 | C11 | N1 | −2.1(2) | C15 | C14 | C19 | C18 | −1.1(2) |
| N3 | C10 | C9 | N1 | 0.3(2) | C7 | C6 | C5 | C4 | −179.97(16) |
| N3 | C10 | C9 | C8 | −176.78(15) | C7 | C6 | C1 | C2 | 179.40(15) |
| C12 | N2 | N3 | C10 | 170.56(15) | C7 | C8 | C9 | N1 | −12.3(2) |
| C12 | N2 | C11 | N4 | 1.62(18) | C7 | C8 | C9 | C10 | 164.90(16) |
| C12 | N2 | C11 | N1 | −174.51(14) | C21 | N5 | C12 | N2 | 95.82(18) |
| C12 | N5 | C21 | C22 | 70.46(19) | C21 | N5 | C12 | C13 | −86.5(2) |
| C12 | N5 | C21 | C24 | −51.19(19) | C19 | C14 | C15 | C16 | 0.2(2) |
| C12 | N5 | C21 | C23 | −171.13(15) | C19 | C18 | C17 | C16 | −0.5(3) |
| C12 | C13 | C14 | C15 | 173.09(16) | C5 | C6 | C7 | C8 | 9.4(3) |
| C12 | C13 | C14 | C19 | −11.5(3) | C5 | C6 | C1 | C2 | −1.3(2) |
| C11 | N2 | N3 | C10 | −0.4(2) | C5 | C4 | C3 | C2 | −1.1(3) |
| C11 | N2 | C12 | N5 | 178.99(14) | C17 | C18 | C19 | C14 | 1.3(3) |
| C11 | N2 | C12 | C13 | 0.79(16) | C1 | C6 | C7 | C8 | −171.41(17) |
| C11 | N4 | C13 | C12 | 3.87(18) | C1 | C6 | C5 | C4 | 0.8(2) |
| C11 | N4 | C13 | C14 | −169.45(14) | C1 | C2 | C3 | C4 | 0.5(3) |
| C11 | N1 | C9 | C10 | −2.8(2) | C20 | C10 | C9 | N1 | 178.46(15) |
| C11 | N1 | C9 | C8 | 174.39(14) | C20 | C10 | C9 | C8 | 1.3(2) |

TABLE 32
Hydrogen Atom Coordinates (Å × 10⁴) and Isotropic
Displacement Parameters (Å² × 10³) for mo_NB_IT_1_1_0ma.

| Atom | x | y | z | U(eq) |
|---|---|---|---|---|
| H8 | 5906.88 | 7270.01 | 4667.07 | 24 |
| H18 | 8602.62 | −3116.82 | 3889.2 | 25 |
| H15 | 7909.9 | −251.52 | 6683.01 | 23 |
| H7 | 5650.98 | 5309.45 | 6269.67 | 22 |
| H19 | 8322.05 | −669.02 | 3583.77 | 23 |
| H5A | 4842.79 | 8544.6 | 4926.25 | 25 |
| H17 | 8512.96 | −4156.45 | 5574.5 | 26 |
| H16 | 8160.08 | −2716.85 | 6960.9 | 26 |
| H1 | 4813.32 | 5956.51 | 7539.21 | 25 |
| H2 | 3853.81 | 7399.29 | 8117.31 | 28 |
| H20A | 6784.17 | 7620.89 | 3332.47 | 35 |
| H20B | 6055.56 | 6677.34 | 2824.7 | 35 |
| H20C | 6855.3 | 6546.87 | 2347.5 | 35 |
| H4 | 3869.82 | 9967.94 | 5488.61 | 27 |
| H3 | 3385.06 | 9422.03 | 7099.37 | 28 |
| H22A | 9478.71 | 1280.03 | 4632.14 | 37 |
| H22B | 10191.72 | 1527.99 | 3978.75 | 37 |
| H22C | 9565.69 | 301.17 | 3600.71 | 37 |
| H24A | 9026.29 | 4597.98 | 3172.98 | 42 |
| H24B | 9883.93 | 4113.85 | 3654.07 | 42 |
| H24C | 9198.18 | 3952.81 | 4369.09 | 42 |
| H23A | 9237.04 | 1300.81 | 1750.99 | 41 |
| H23B | 9929.05 | 2424.31 | 2062.21 | 41 |
| H23C | 9086.71 | 3002.2 | 1591.08 | 41 |
| H5 | 8066(11) | 2480(20) | 2547(15) | 21(5) |

### Example 4

### Additional Compounds

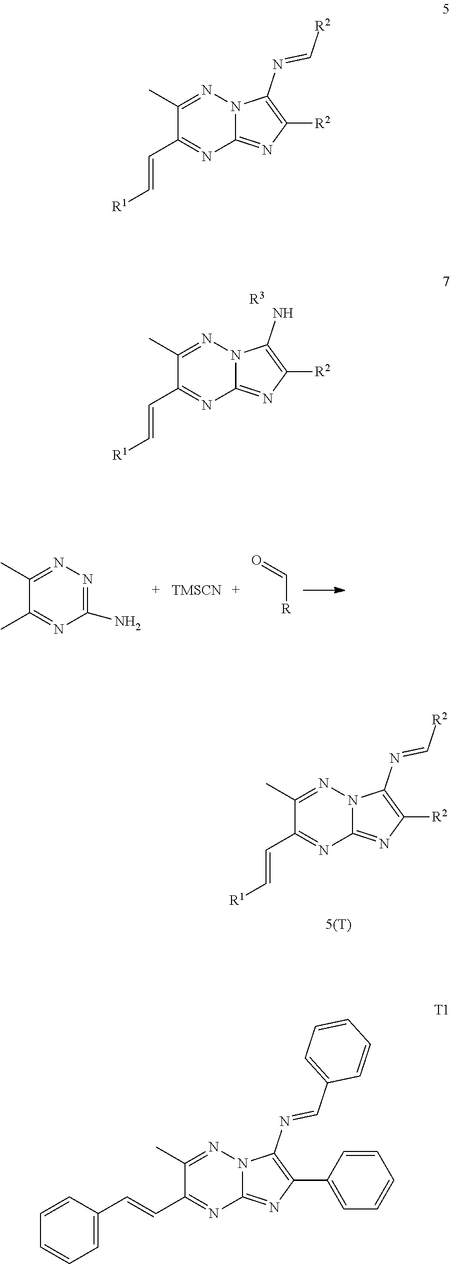

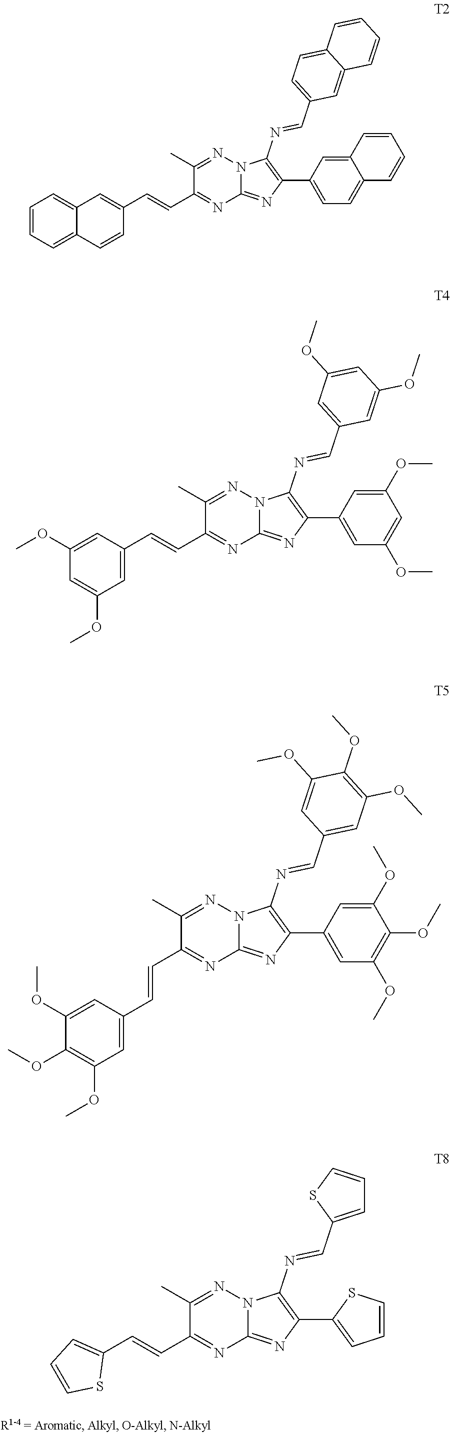

General Procedure 1: Modified GBB-Condensation to afford 5(T) analogs

A mixture of aldehyde (3.1 equiv., 3.1 mmol), 5,6-dimethyl-1,2,4-triazin-3-amine (124 mg, 1 Eq, 1.00 mmol), and Scandiumtrifluoromethanesulfonate (98.4 mg, 0.200 Eq, 0.200 mmol) in dry MeOH (3.3 mL, 0.3 M) was added to a microwave-vial (MWV) with stirbar. The MWV was capped and (Trimethylsilylnitrile) (109 mg, 145 μL, 1.1 Eq, 1.10 mmol) was injected. The resulting mixture was stirred and heated at 140° C. in microwave for 20 min. The solvent was evaporated in vacuo and the crude product was dissolved in DCM, dry loaded onto silica, and purified via automated flash chromatography using a Teledyne ISCOTM (0-40% EtOAc/Hexane) to afford the title compound.

### (E)-N-(2-methyl-6-phenyl-3-((E)-styryl)imidazo[1,2-b][1,2,4]triazin-7-yl)-1-phenylmethanimine

Compound T1 was prepared by General Procedure 1. (158 mg, 0.38 mmol, 38% yield).

Red solid. 1H NMR (400 MHz, CDCl₃) δ 9.93 (s, 1H), 8.67-8.54 (m, 2H), 8.26 (d, J=15.5 Hz, 1H), 8.02 (dd, J=6.6, 2.9 Hz, 2H), 7.69-7.62 (m, 2H), 7.54 (dd, J=7.0, 1.6 Hz, 5H), 7.49-7.41 (m, 4H), 7.25 (d, J=15.3 Hz, 1H), 2.82 (s, 3H). 13C{1H} NMR (101 MHz, CDCl₃) δ 129.1, 128.9, 128.8, 128.7, 128.4, 127.9, 77.3, 77.0, 76.7, 20.0.

### (E)-N-(2-methyl-6-(naphthalen-2-yl)-3-((E)-2-(naphthalen-2-yl)vinyl)imidazo[1,2-b][1,2,4]triazin-7-yl)-1-(naphthalen-2-yl)methanimine

Compound T2 was prepared by General Procedure 1. (170 mg, 0.33 mmol, 33% yield).

Red solid. 1H NMR (400 MHz, CDCl₃) δ 10.04 (s, 1H), 9.23 (s, 1H), 8.84 (d, J=9.0 Hz, 1H), 8.38 (d, J=15.3 Hz, 1H), 8.30 (d, J=8.6 Hz, 1H), 8.24 (s, 1H), 8.04-7.80 (m, 10H), 7.75 (d, J=8.7 Hz, 1H), 7.60-7.48 (m, 6H), 7.27 (d, J=13.7 Hz, 1H), 2.83 (s, 3H).

### (E)-1-(3,5-dimethoxyphenyl)-N-(6-(3,5-dimethoxyphenyl)-3-((E)-3,5-dimethoxystyryl)-2-methylimidazo[1,2-b][1,2,4]triazin-7-yl)methanimine

Compound T4 was prepared by General Procedure 1. (162 mg, 0.28 mmol, 28% yield).

Red solid. 1H NMR (400 MHz, CDCl₃) δ 9.74 (s, 1H), 8.11 (d, J=15.4 Hz, 1H), 7.91 (d, J=2.3 Hz, 2H), 7.19-7.11 (m, 3H), 6.76 (d, J=2.2 Hz, 2H), 6.60 (t, J=2.3 Hz, 1H), 6.54 (t, J=2.3 Hz, 1H), 6.50 (t, J=2.2 Hz, 1H), 3.91 (d, J=2.3 Hz, 12H), 3.86 (s, 6H), 2.80 (s, 3H). 13C{1H}NMR (101 MHz, CDCl₃) δ 161.0, 106.2, 105.9, 77.33, 77.0, 76.7, 55.6, 55.4, 20.0.

### (E)-N-(2-methyl-6-(3,4,5-trimethoxyphenyl)-3-((E)-3,4,5-trimethoxystyryl)imidazo[1,2-b][1,2,4]triazin-7-yl)-1-(3,4,5-trimethoxyphenyl)methanimine

Compound T5 was prepared by General Procedure 1. (199 mg, 0.29 mmol, 29% yield).

Red solid. 1H NMR (400 MHz, DMSO) δ 9.86 (s, 1H), 8.01 (d, J=15.5 Hz, 1H), 7.84 (s, 2H), 7.47 (d, J=15.4 Hz, 1H), 7.36 (s, 2H), 7.20 (s, 2H), 3.92 (s, 6H), 3.90 (d, J=3.0 Hz, 12H), 3.79 (s, 3H), 3.77 (s, 3H), 3.74 (s, 3H), 2.86 (s, 3H).

### (E)-N-(2-methyl-6-(thiophen-2-yl)-3-((E)-2-(thiophen-2-yl)vinyl)imidazo[1,2-b][1,2,4]triazin-7-yl)-1-(thiophen-2-yl)methanimine

Compound T8 was prepared by General Procedure 1. (108 mg, 0.X mmol, X % yield).

Red solid. 1H NMR ¹H NMR (500 MHz, CDCl₃) δ 8.27 (d, J=15.0 Hz, 1H), 8.15 (d, J=3.5 Hz, 1H), 7.51 (dd, J=7.0, 4.3 Hz, 2H), 7.47 (d, J=5.0 Hz, 1H), 7.34 (d, J=4.8 Hz, 1H), 7.24 (s, 2H), 7.16 (dd, J=5.0, 3.5 Hz, 1H), 7.14-7.11 (m, 1H), 7.05 (dd, J=4.9, 3.5 Hz, 1H), 6.90 (d, J=15.1 Hz, 1H), 2.69 (s, 3H).

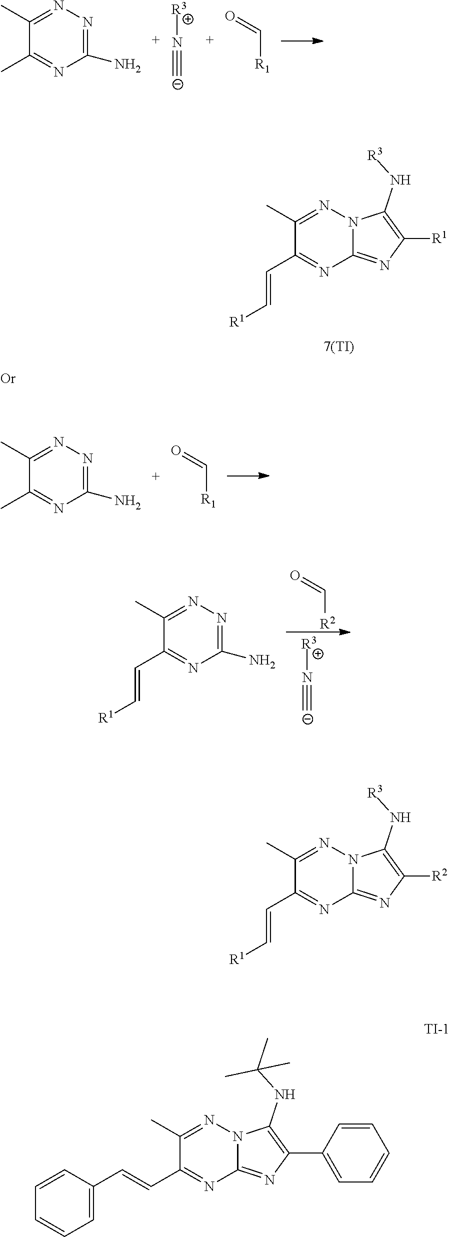

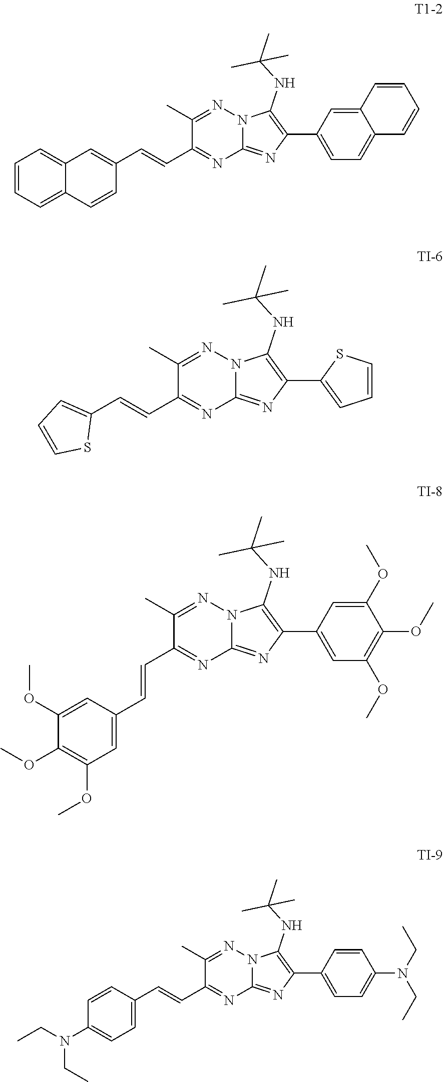

### General Procedure 2: GBB-Condensation to Afford 7(TI) Analogs

A mixture of aldehyde (4.0 equiv., 2.0 mmol), 5,6-dimethyl-1,2,4-triazin-3-amine (62.1 mg, 1 Eq, 0.50 mmol), and Scandiumtrifluoromethanesulfonate (49.2 mg, 0.200 Eq, 0.100 mmol) in dry DCE (2.5 mL, 0.2 M) was added to a microwave-vial (MWV) with stirbar. The MWV was capped and Propane, 2-isocyano-2-methyl-(58.2 mg, 79.3 μL, 1.4 Eq, 700 μmol) was injected. The resulting mixture was stirred and heated at 170° C. in the microwave for 20 min. The solvent was evaporated in vacuo and the crude product was dissolved in DCM, dry loaded onto silica, and purified via automated flash chromatography using a Teledyne ISCOTM (0-40% EtOAc/Hexane) to afford the title compound.

### (E)-N-(tert-butyl)-2-methyl-6-phenyl-3-styrylimidazo[1,2-b][1,2,4]triazin-7-amine

Compound TI-I was prepared by General Procedure 2. (82 mg, 0.215 mmol, 43% yield).

Red solid. 1H NMR (500 MHz, CDCl₃) δ 8.40-8.35 (m, 2H), 8.21 (d, J=15.6 Hz, 1H), 7.66-7.59 (m, 2H), 7.46-7.30 (m, 7H), 7.21 (d, J=15.6 Hz, 1H), 2.73 (s, 3H), 1.14 (s, 9H). 13C{1H}NMR (126 MHz, CDCl₃) δ 145.63, 139.12, 138.26, 136.09, 134.31, 129.46, 128.93, 128.14, 128.09, 128.06, 127.78, 126.63, 120.19, 57.49, 30.47, 19.77.

### (E)-N-(tert-butyl)-2-methyl-6-(naphthalen-2-yl)-3-(2-(naphthalen-2-yl)vinyl)imidazo[1,2-b][1,2,4]triazin-7-amine

Compound TI-2 was prepared by General Procedure 2. (75 mg, 0.155 mmol, 31% yield).

Red solid. 1H NMR (500 MHz, CDCl₃) δ 8.93 (d, J=1.6 Hz, 1H), 8.57 (dd, J=8.5, 1.7 Hz, 1H), 8.34 (d, J=15.4 Hz, 1H), 7.97 (s, 1H), 7.95-7.91 (m, 1H), 7.88 (d, J=8.7 Hz, 2H), 7.82 (t, J=7.0 Hz, 3H), 7.76 (dd, J=8.6, 1.7 Hz, 1H), 7.51-7.46 (m, 4H), 7.28 (d, J=15.4 Hz, 1H), 3.42 (s, 1H), 2.73 (s, 3H), 1.15 (s, 9H). 13C{1H} NMR (126 MHz, CDCl₃) δ 133.94, 133.56, 133.41, 133.26, 128.67, 128.59, 127.79, 127.65, 127.55, 126.98, 126.72, 126.26, 126.06, 125.80, 123.54, 77.28, 77.03, 76.77, 57.60, 30.52, 19.85.

### (E)-N-(tert-butyl)-2-methyl-6-(thiophen-2-yl)-3-(2-(thiophen-2-yl)vinyl)imidazo[1,2-b][1,2,4]triazin-7-amine

Compound TI-6 was prepared by General Procedure 2. (49.4 mg, 0.125 mmol, 25% yield).

Red solid. 1H NMR (500 MHz, CDCl₃) δ 8.27 (dd, J=15.1, 4.3 Hz, 1H), 7.94 (d, J=3.6 Hz, 1H), 7.55-7.46 (m, 1H), 7.34 (dt, J=6.0, 3.8 Hz, 2H), 7.25 (d, J=2.9 Hz, 1H), 7.12-7.02 (m, 2H), 6.92 (dd, J=15.2, 9.6 Hz, 1H), 2.66 (s, 3H), 1.26 (s, 9H). 13C{1H} NMR (126 MHz, CDCl₃) δ 141.74, 130.84, 130.30, 129.05, 128.49, 128.29, 127.84, 127.48, 127.40, 126.29, 125.21, 119.25, 77.29, 77.04, 76.78, 57.35, 30.76, 19.66.

### (E)-N-(tert-butyl)-2-methyl-6-(3,4,5-trimethoxyphenyl)-3-(3,4,5-trimethoxystyryl)imidazo[1,2-b][1,2,4]triazin-7-amine

Compound TI-8 was prepared by General Procedure 2. (87 mg, 0.155 mmol, 31% yield).

Red solid. 1H NMR (500 MHz, CDCl₃) δ 8.10 (d, J=15.4 Hz, 1H), 7.78 (s, 2H), 7.04 (d, J=15.4 Hz, 1H), 6.81 (s, 2H), 3.94 (s, 6H), 3.91 (s, 6H), 3.88 (s, 3H), 3.88 (s, 3H), 3.37 (s, 1H), 2.71 (s, 3H), 1.18 (s, 9H). 13C{1H} NMR (126 MHz, CDCl₃) δ 153.54, 152.96, 139.77, 138.98, 138.16, 131.63, 126.21, 119.28, 105.18, 105.07, 77.29, 77.04, 76.79, 61.04, 60.97, 57.50, 56.25, 56.22, 30.66, 19.82.

### (E)-N-(tert-butyl)-6-(4-(diethylamino)phenyl)-3-(4-(diethylamino)styryl)-2-methylimidazo[1,2-b][1,2,4]triazin-7-amine

Compound TI-9 was prepared by General Procedure 2. (50 mg, 0.095 mmol, 19% yield).

Purple solid. 1H NMR (500 MHz, CDCl₃) δ 8.28 (d, J=8.4 Hz, 2H), 8.09 (d, J=15.3 Hz, 1H), 7.50-7.44 (m, 2H), 6.91 (d, J=15.3 Hz, 1H), 6.71 (s, 2H), 6.65 (d, J=8.8 Hz, 2H), 3.39 (q, J=7.1 Hz, 8H), 3.28 (s, 1H), 2.63 (s, 3H), 1.21-1.15 (m, 21H). 13C{1H} NMR (126 MHz, CDCl₃) δ 139.65, 129.58, 129.14, 111.48, 111.04, 77.29, 77.04, 76.78, 57.16, 44.51, 30.57, 19.75, 12.66.

### Example 5

### Additional Compounds

### Procedures

### General Procedure 1:

A mixture of aldehyde (4.0 Eq, 1.0 mmol), 5,6-dimethyl-1,2,4-triazin-3-amine (31 mg, 1 Eq, 0.25 mmol), and Scandiumtrifluoromethanesulfonate (25 mg, 0.2 Eq, 50 μmol) in DCE (2.5 mL, 0.1 M) was added to a microwave-vial with stir bar. The MWV was capped and isonitrile (1.4 Eq, 0.35 mmol) was injected. The resulting mixture was stirred and heated at 170° C. in the microwave for 20 min. The solvent was evaporated in vacuo and the crude product was dissolved in DCM, dry loaded onto silica, and purified via automated flash chromatography using a Teledyne ISCO™ (0-60% EtOAc/Hexane) to afford the title compound.

### General Procedure 2:

A mixture of aldehyde (4.0 Eq, 1.0 mmol), 5,6-dimethyl-1,2,4-triazin-3-amine (31 mg, 1 Eq, 0.25 mmol), and Scandiumtrifluoromethanesulfonate (25 mg, 0.2 Eq, 50 μmol) in MeOH (2.5 mL, 0.1 M) was added to a microwave-vial with stir bar. The MWV was capped and isonitrile (1.4 Eq, 0.35 mmol) was injected. The resulting mixture was stirred and heated at 170° C. in the microwave for 20 min. The solvent was evaporated in vacuo and the crude product was dissolved in DCM, dry loaded onto silica, and purified via automated flash chromatography using a Teledyne ISCO™ (0-60% EtOAc/Hexane) to afford the title compound.

### General Procedure 3:

In a 5 mL Biotage microwave vial, a stir bar, (E)-1-(3,5-dimethoxyphenyl)-N-(6-(3,5-dimethoxyphenyl)-3-((E)-3,5-dimethoxystyryl)-2-methylimidazo[1,2-b][1,2,4]triazin-7-yl)methanimine (0.15 g, 1 Eq, 0.25 mmol) and Copper(II)trifluoromethanesulfonate (90 mg, 1 Eq, 0.25 mmol) were added. DCE (2.5 mL, 0.10 M) was injected, the vial was sealed and heated at 120° C. for 20 min. Upon completion, the reaction was treated with 5 mL of 7N Ammonia in MeOH concentrated then columned 0-10% DCM:MeOH to afford the title compound.

### General Procedure 4:

A mixture of aldehyde (4.0 Eq, 1.0 mmol), 5,6-dimethyl-1,2,4-triazin-3-amine (31 mg, 1 Eq, 0.25 mmol), and Scandiumtrifluoromethanesulfonate (25 mg, 0.2 Eq, 50 μmol) in DCE (2.5 mL, 0.1 M) was added to a microwave-vial with stir bar. The MWV was capped and isonitrile (1.4 Eq, 0.35 mmol) was injected. The resulting mixture was stirred and heated at 100° C. for 12 h. The solvent was evaporated in vacuo and the crude product was dissolved in DCM, dry loaded onto silica, and purified via automated flash chromatography using a Teledyne ISCO™ (0-60% EtOAc/Hexane) to afford the title compound.

### Compound Characterization

(E)-N-(tert-butyl)-2-methyl-6-phenyl-3-styrylimidazo[1,2-b][1,2,4]triazin-7-amine Compound 42 was prepared by General procedure 1. (41 mg, 0.11 mmol, 43% yield). Red solid. ¹H NMR (500 MHz, CDCl₃) δ 8.40-8.35 (m, 2H), 8.21 (d, J=15.6 Hz, 1H), 7.66-7.59 (m, 2H), 7.46-7.30 (m, 7H), 7.21 (d, J=15.6 Hz, 1H), 2.73 (s, 3H), 1.14 (s, 9H). ¹³C NMR (126 MHz, CDCl₃) δ 145.63, 139.12, 138.26, 136.09, 134.31, 129.46, 128.93, 128.14, 128.09, 128.06, 127.78, 126.63, 120.19, 57.49, 30.47, 19.77. λex=453 nm; λem=636 nm, Quantum Yield: 0.11.

(E)-N-(tert-butyl)-2-methyl-6-(naphthalen-2-yl)-3-(2-(naphthalen-2-yl)vinyl)imidazo[1,2-b][1,2,4]triazin-7-amine Compound 43 was prepared by General procedure 1. (39 mg, 0.08 mmol, 32% yield). Red solid. ¹H NMR (500 MHz, CDCl₃) δ 8.93 (d, J=1.6 Hz, 1H), 8.57 (dd, J=8.5, 1.7 Hz, 1H), 8.34 (d, J=15.4 Hz, 1H), 7.97 (s, 1H), 7.95-7.91 (m, 1H), 7.88 (d, J=8.7 Hz, 2H), 7.82 (t, J=7.0 Hz, 3H), 7.76 (dd, J=8.6, 1.7 Hz, 1H), 7.51-7.46 (m, 4H), 7.28 (d, J=15.4 Hz, 1H), 3.42 (s, 1H), 2.73 (s, 3H), 1.15 (s, 9H). ¹³C NMR (126 MHz, CDCl₃) δ 133.94, 133.56, 133.41, 133.26, 128.67, 128.59, 127.79, 127.65, 127.55, 126.98, 126.72, 126.26, 126.06, 125.80, 123.54, 77.28, 77.03, 76.77, 57.60, 30.52, 19.85. λex=485 nm; λem=643 nm, Quantum Yield: 0.22.

(E)-N-(tert-butyl)-6-(4-methoxyphenyl)-3-(4-methoxystyryl)-2-methylimidazo[1,2-b][1,2,4]triazin-7-amine Compound 44 was prepared by General procedure 1. (54 mg, 0.12 mmol, 49% yield). Red solid. 1H NMR (500 MHz, DMSO) δ 8.30 (d, J=8.3 Hz, 2H), 7.90 (d, J=15.6 Hz, 1H), 7.79 (d, J=8.2 Hz, 2H), 7.34 (d, J=15.6 Hz, 1H), 7.05-7.00 (m, 4H), 3.82 (d, J=6.8 Hz, 6H), 3.17 (d, J=5.3 Hz, 1H), 2.72 (s, 3H), 1.11 (s, 9H). 13C NMR (126 MHz, DMSO) δ 160.99, 154.20, 152.86, 143.49, 132.04, 129.17, 114.92, 54.58, 47.94, 40.50, 40.33, 40.16, 40.00, 39.83, 39.66, 39.50, 30.86, 29.25. λex=486 nm; λem=643 nm, Quantum Yield: 0.27.

(E)-N-(tert-butyl)-2-methyl-6-(4-nitrophenyl)-3-(4-nitrostyryl)imidazo[1,2-b][1,2,4]triazin-7-amine Compound 45 was prepared by General procedure 1. (18 mg, 0.038 mmol, 15% yield).

Red solid. 1H NMR (500 MHz, DMSO) δ 8.63 (d, J=8.5 Hz, 2H), 8.32 (dd, J=17.3, 8.4 Hz, 4H), 8.14 (d, J=8.3 Hz, 2H), 8.07 (d, J=16.0 Hz, 1H), 7.75 (d, J=15.7 Hz, 1H), 4.87 (s, 1H), 2.78 (s, 3H), 1.15 (s, 9H). 13C NMR (126 MHz, DMSO) δ 146.77, 129.49, 128.38, 126.59, 124.52, 124.08, 57.08, 40.49, 40.33, 40.25, 40.16, 40.08, 39.99, 39.82, 39.66, 39.49, 30.78. λex=505 nm; λem=655 nm, Quantum Yield: 0.11.

(E)-N-(tert-butyl)-6-(3,5-dimethoxyphenyl)-3-(3,5-dimethoxystyryl)-2-methylimidazo[1,2-b][1,2,4]triazin-7-amine Compound 46 was prepared by General procedure 1. (58 mg, 0.12 mmol, 46% yield). Red solid. 1H NMR (500 MHz, DMSO) δ 7.89 (d, J=15.5 Hz, 1H), 7.60 (d, J=2.4 Hz, 2H), 7.49 (d, J=15.6 Hz, 1H), 7.01 (d, J=2.2 Hz, 2H), 6.56 (s, 1H), 6.48 (d, J=2.5 Hz, 1H), 4.59 (s, 1H), 3.82 (d, J=3.0 Hz, 12H), 2.75 (s, 3H), 1.15 (s, 9H). 13C NMR (126 MHz, DMSO) δ 161.25, 160.66, 138.27, 137.21, 106.49, 105.65, 100.66, 56.48, 55.87, 55.65, 40.50, 40.43, 40.34, 40.26, 40.17, 40.09, 40.00, 39.92, 39.83, 39.67, 39.50, 30.94, 20.04. λex=461 nm; λem=638 nm, Quantum Yield: 0.19.

(E)-N-(tert-butyl)-2-methyl-6-(thiophen-2-yl)-3-(2-(thiophen-2-yl)vinyl)imidazo[1,2-b][1,2,4]triazin-7-amine Compound 47 was prepared by General procedure 1. (49 mg, 0.13 mmol, 25% yield). Red solid. ¹H NMR (500 MHz, CDCl₃) δ 8.27 (dd, J=15.1, 4.3 Hz, 1H), 7.94 (d, J=3.6 Hz, 1H), 7.55-7.46 (m, 1H), 7.34 (dt, J=6.0, 3.8 Hz, 2H), 7.25 (d, J=2.9 Hz, 1H), 7.12-7.02 (m, 2H), 6.92 (dd, J=15.2, 9.6 Hz, 1H), 2.66 (s, 3H), 1.26 (s, 9H). ¹³C NMR (126 MHz, CDCl₃) δ 141.74, 130.84, 130.30, 129.05, 128.49, 128.29, 127.84, 127.48, 127.40, 126.29, 125.21, 119.25, 77.29, 77.04, 76.78, 57.35, 30.76, 19.66. λex=513 nm; λem=647 nm, Quantum Yield: 0.33.

(E)-N-(tert-butyl)-2-methyl-6-(3,4,5-trimethoxyphenyl)-3-(3,4,5-trimethoxystyryl)imidazo[1,2-b][1,2,4]triazin-7-amine Compound 48 was prepared by General procedure 1. (44 mg, 0.078 mmol, 31% yield).

Red solid. 1H NMR (500 MHz, CDCl₃) δ 8.10 (d, J=15.4 Hz, 1H), 7.78 (s, 2H), 7.04 (d, J=15.4 Hz, 1H), 6.81 (s, 2H), 3.94 (s, 6H), 3.91 (s, 6H), 3.88 (s, 3H), 3.88 (s, 3H), 3.37 (s, 1H), 2.71 (s, 3H), 1.18 (s, 9H). 13C NMR (126 MHz, CDCl₃) δ 153.54, 152.96, 139.77, 138.98, 138.16, 131.63, 126.21, 119.28, 105.18, 105.07, 77.29, 77.04, 76.79, 61.04, 60.97, 57.50, 56.25, 56.22, 30.66, 19.82. λex=506 nm; λem=633 nm, Quantum Yield: 0.28.

(E)-N-(tert-butyl)-6-(4-(diethylamino)phenyl)-3-(4-(diethylamino)styryl)-2-methylimidazo[1,2-b][1,2,4]triazin-7-amine Compound 49 was prepared by General procedure 1. (24 mg, 0.045 mmol, 18% yield). Red solid. 1H NMR (500 MHz, CDCl₃) δ 8.28 (d, J=8.4 Hz, 2H), 8.09 (d, J=15.3 Hz, 1H), 7.50-7.44 (m, 2H), 6.91 (d, J=15.3 Hz, 1H), 6.71 (s, 2H), 6.65 (d, J=8.8 Hz, 2H), 3.39 (q, J=7.1 Hz, 8H), 3.28 (s, 1H), 2.63 (s, 3H), 1.21-1.15 (m, 21H). 13C NMR (126 MHz, CDCl₃) δ 139.65, 129.58, 129.14, 111.48, 111.04, 77.29, 77.04, 76.78, 57.16, 44.51, 30.57, 19.75, 12.66. λex=566 nm; λem=653 nm, Quantum Yield: 0.45.

### (E)-N-(2-methyl-6-phenyl-3-((E)-styryl)imidazo[1,2-b][1,2,4]triazin-7-yl)-1-phenylmethanimine

Compound 58 was prepared by General procedure 2. (22 mg, 0.053 mmol, 21% yield). Red solid. 1H NMR (400 MHz, CDCl₃) δ 9.93 (s, 1H), 8.67-8.54 (m, 2H), 8.26 (d, J=15.5 Hz, 1H), 8.02 (dd, J=6.6, 2.9 Hz, 2H), 7.69-7.62 (m, 2H), 7.54 (dd, J=7.0, 1.6 Hz, 5H), 7.49-7.41 (m, 4H), 7.25 (d, J=15.3 Hz, 1H), 2.82 (s, 3H). 13C NMR (101 MHz, CDCl₃) δ 144.13, 129.18, 128.99, 128.87, 128.74, 128.41, 127.98, 119.78, 77.33, 77.02, 76.70, 20.00. λex=510 nm; λem=601 nm, Quantum Yield: 0.04.

(E)-N-(2-methyl-6-(naphthalen-2-yl)-3-((E)-2-(naphthalen-2-yl)vinyl)imidazo[1,2-b][1,2,4]triazin-7-yl)-1-(naphthalen-2-yl)methanimine Compound 59 was prepared by General procedure 2. (65 mg, 0.12 mmol, 46% yield). Red solid. 1H NMR (400 MHz, CDCl₃) δ 10.04 (s, 1H), 9.23 (s, 1H), 8.84 (d, J=9.0 Hz, 1H), 8.38 (d, J=15.3 Hz, 1H), 8.30 (d, J=8.6 Hz, 1H), 8.24 (s, 1H), 8.04-7.80 (m, 10H), 7.75 (d, J=8.7 Hz, 1H), 7.60-7.48 (m, 6H), 7.27 (d, J=13.7 Hz, 1H), 2.83 (s, 3H). 13C NMR (126 MHz, CDCl₃) δ 133.74, 132.98, 132.51, 129.44, 128.36, 127.83, 127.30, 127.00, 126.25, 123.67, 118.68, 77.28, 77.03, 76.77, 29.71. λex=526 nm; λem=601 nm, Quantum Yield: 0.04.

(E)-1-(4-methoxyphenyl)-N-(6-(4-methoxyphenyl)-3-((E)-4-methoxystyryl)-2-methylimidazo[1,2-b][1,2,4]triazin-7-yl)methanimine Compound 60 was prepared by General procedure 2. (35 mg, 0.069 mmol, 28% yield). Red solid. 1H NMR (500 MHz, CDCl₃) δ 9.75 (s, 1H), 8.59-8.53 (m, 2H), 8.15 (d, J=15.4 Hz, 1H), 7.88 (d, J=8.7 Hz, 2H), 7.60-7.52 (m, 2H), 7.05-6.97 (m, 5H), 6.91-6.87 (m, 2H), 3.88 (s, 3H), 3.87 (s, 3H), 3.82 (s, 3H), 2.74 (s, 3H). 13C NMR (126 MHz, CDCl₃) δ 156.67, 130.74, 129.81, 114.49, 114.39, 114.01, 77.27, 77.02, 76.76, 55.50, 55.41, 55.35, 20.04. λex=563 nm; λem=609 nm, Quantum Yield: 0.19.

(E)-1-(3,5-dimethoxyphenyl)-N-(6-(3,5-dimethoxyphenyl)-3-((E)-3,5-dimethoxystyryl)-2-methylimidazo[1,2-b][1,2,4]triazin-7-yl)methanimine Compound 62 was prepared by General procedure 2. (25 mg, 0.043 mmol, 17% yield). Red solid. 1H NMR (400 MHz, CDCl₃) δ 9.74 (s, 1H), 8.11 (d, J=15.4 Hz, 1H), 7.91 (d, J=2.3 Hz, 2H), 7.19-7.11 (m, 3H), 6.76 (d, J=2.2 Hz, 2H), 6.60 (t, J=2.3 Hz, 1H), 6.54 (t, J=2.3 Hz, 1H), 6.50 (t, J=2.2 Hz, 1H), 3.91 (d, J=2.3 Hz, 12H), 3.86 (s, 6H), 2.80 (s, 3H). 13C NMR (101 MHz, CDCl₃) δ 161.07, 160.73, 157.47, 127.26, 106.28, 105.98, 77.33, 77.02, 76.70, 55.62, 55.48, 20.06. λex=506 nm; λem=589 nm, Quantum Yield: 0.10.

(E)-N-(2-methyl-6-(thiophen-2-yl)-3-((E)-2-(thiophen-2-yl)vinyl)imidazo[1,2-b][1,2,4]triazin-7-yl)-1-(thiophen-2-yl)methanimine Compound 63 was prepared by General procedure 2. (52 mg, 0.12 mmol, 48% yield). Red solid. 1H NMR (500 MHz, CDCl₃) δ 9.88 (s, 1H), 8.27 (d, J=15.0 Hz, 1H), 8.15 (d, J=3.5 Hz, 1H), 7.51 (dd, J=7.0, 4.3 Hz, 2H), 7.47 (d, J=5.0 Hz, 1H), 7.34 (d, J=4.8 Hz, 1H), 7.24 (s, 2H), 7.16 (dd, J=5.0, 3.5 Hz, 1H), 7.14-7.11 (m, 1H), 7.05 (dd, J=4.9, 3.5 Hz, 1H), 6.90 (d, J=15.1 Hz, 1H), 2.69 (s, 3H). 13C NMR (126 MHz, CDCl₃) δ 160.98, 154.17, 147.02, 141.01, 134.31, 131.06, 128.70, 128.47, 128.37, 128.20, 127.82, 119.02, 77.29, 77.03, 76.78, 18.39. λex=572 nm; λem=615 nm, Quantum Yield: 0.19.

(E)-N-(2-methyl-6-(3,4,5-trimethoxyphenyl)-3-((E)-3,4,5-trimethoxystyryl)imidazo[1,2-b][1,2,4]triazin-7-yl)-1-(3,4,5-trimethoxyphenyl)methanimine Compound 64 was prepared by General procedure 2. (22 mg, 0.033 mmol, 13% yield). Red solid. 1H NMR (500 MHz, CDCl₃) δ 9.76 (s, 1H), 8.16 (d, J=15.4 Hz, 1H), 7.88 (s, 2H), 7.24 (s, 2H), 7.07 (d, J=15.4 Hz, 1H), 6.84 (s, 2H), 3.99-3.89 (m, 27H), 2.80 (s, 3H). 13C NMR (126 MHz, CDCl₃) δ 153.74, 153.58, 153.23, 132.85, 131.40, 127.62, 118.93, 106.74, 105.94, 105.28, 77.28, 77.02, 76.77, 61.07, 61.03, 60.99, 56.47, 56.39, 56.25, 20.07. λex=533 nm; λem=611 nm, Quantum Yield: 0.10.

(E)-N-benzyl-2-methyl-6-phenyl-3-styrylimidazo[1,2-b][1,2,4]triazin-7-amine Compound 74 was prepared by General procedure 1. (9 mg, 0.02 mmol, 9% yield). Red solid. ¹H NMR (500 MHz, DMSO) δ 8.01 (d, J=7.6 Hz, 2H), 7.88 (d, J=15.6 Hz, 1H), 7.82 (d, J=7.5 Hz, 2H), 7.48-7.42 (m, 5H), 7.42-7.31 (m, 2H), 7.28-7.19 (m, 4H), 7.17 (dq, J=6.0, 3.3 Hz, 1H), 6.00 (t, J=7.1 Hz, 1H), 4.45 (d, J=6.9 Hz, 2H), 2.74 (d, J=2.2 Hz, 3H). ¹³C NMR (126 MHz, DMSO) δ 146.75, 144.12, 140.44, 137.68, 136.46, 136.06, 134.45, 129.68, 129.37, 128.96, 128.60, 128.31, 128.23, 128.01, 127.40, 127.33, 122.30, 49.54, 40.48, 40.32, 40.15, 39.98, 39.82, 39.65, 39.48, 20.04. λex=498 nm; λem=636 nm, Quantum Yield: 0.24.

(E)-N-benzyl-2-methyl-6-(naphthalen-2-yl)-3-(2-(naphthalen-2-yl)vinyl)imidazo[1,2-b][1,2,4]triazin-7-amine Compound 75 was prepared by General procedure 1. (25 mg, 0.05 mmol, 19% yield). Red solid. ¹H NMR (500 MHz, DMSO) δ 8.48 (s, 1H), 8.27 (s, 1H), 8.21 (d, J=8.7 Hz, 1H), 8.08 (dd, J=16.2, 12.3 Hz, 2H), 7.99 (s, 4H), 7.96-7.89 (m, 3H), 7.64 (d, J=15.6 Hz, 1H), 7.60-7.55 (m, 2H), 7.53 (s, 1H), 7.30 (d, J=7.6 Hz, 2H), 7.24 (t, J=7.4 Hz, 2H), 7.18 (d, J=7.4 Hz, 1H), 6.18 (t, J=6.7 Hz, 1H), 4.57-4.48 (m, 2H), 2.81 (s, 3H). ¹³C NMR (126 MHz, DMSO) δ 128.66, 128.31, 127.45, 125.95, 124.64, 49.67, 40.49, 40.41, 40.32, 40.25, 40.16, 40.08, 39.99, 39.82, 39.65, 39.49, 20.15. λex=529 nm; λem=646 nm, Quantum Yield: 0.34.

(E)-N-benzyl-6-(4-methoxyphenyl)-3-(4-methoxystyryl)-2-methylimidazo[1,2-b][1,2,4]triazin-7-amine Compound 76 was prepared by General procedure 1. (19 mg, 0.04 mmol, 16% yield). Red solid. ¹H NMR (500 MHz, DMSO) δ 7.99 (d, J=8.2 Hz, 2H), 7.83 (d, J=15.0 Hz, 1H), 7.77 (d, J=8.1 Hz, 2H), 7.32 (d, J=15.0 Hz, 2H), 7.24 (q, J=8.1 Hz, 4H), 7.17 (d, J=9.2 Hz, 2H), 7.03 (s, 2H), 5.78 (d, J=7.7 Hz, 1H), 4.41 (d, J=6.8 Hz, 2H), 3.82 (d, J=8.3 Hz, 6H), 2.71 (s, 3H). ¹³C NMR (126 MHz, DMSO) δ 143.60, 129.93, 128.59, 128.29, 114.87, 55.79, 49.75, 40.51, 40.34, 40.27, 40.17, 40.09, 40.00, 39.84, 39.67, 39.50, 31.89. λex=499 nm; λem=639 nm, Quantum Yield: 0.24.

(E)-5-(3,5-dimethoxyphenyl)-10-(3,5-dimethoxystyryl)-2,4-dimethoxy-9-methyl-[1,2,4]triazino[3′, 2′: 2,3]imidazo[4,5-c]isoquinolines Compound 79 was prepared by General procedure 3. (141 mg, 0.24 mmol, 95% yield). Red solid. ¹H NMR (500 MHz, CDCl₃) δ 8.66 (s, 1H), 8.49 (d, J=15.2 Hz, 1H), 7.34 (d, J=15.2 Hz, 1H), 6.87 (d, J=2.2 Hz, 2H), 6.68 (d, J=2.1 Hz, 1H), 6.62 (d, J=2.3 Hz, 2H), 6.53 (dt, J=4.6, 2.2 Hz, 2H), 4.21 (d, J=2.0 Hz, 3H), 3.85 (s, 6H), 3.81 (s, 6H), 3.61 (s, 3H), 2.96 (d, J=2.0 Hz, 3H). ¹³C NMR (126 MHz, CDCl₃) δ 160.19, 127.48, 126.56, 125.54, 105.78, 76.25, 76.00, 75.75, 54.62, 54.51, 28.69, -0.00. λex=498 nm; λem=608 nm, Quantum Yield: 0.18.

(E)-2,3,4-trimethoxy-9-methyl-5-(3,4,5-trimethoxyphenyl)-10-(3,4,5-trimethoxystyryl)-[1,2,4]triazino[3′, 2′: 2,3]imidazo[4,5-c]isoquinolines Compound 80 was prepared by General procedure 3. (160 mg, 0.24 mmol, 95% yield). Red solid. ¹H NMR (500 MHz, CDCl₃) δ 8.62 (s, 1H), 8.31 (d, J=15.2 Hz, 1H), 7.31 (d, J=15.4 Hz, 1H), 6.88 (s, 2H), 6.79 (s, 2H), 3.97-3.83 (m, 27H), 2.99 (s, 3H). ¹³C NMR (126 MHz, CDCl₃) δ 152.55, 151.32, 76.26, 76.00, 75.75, 60.11, 59.95, 55.35, 55.22, 28.69, 0.00. λex=499 nm; λem=604 nm, Quantum Yield: 0.25.

methyl (E)-2-(7-(tert-butylamino)-3-(2-(methoxycarbonyl)styryl)-2-methylimidazo[1,2-b][1,2,4]triazin-6-yl)benzoate Compound 84 was prepared by General procedure 4. (26 mg, 0.0.053 mmol, 21% yield). Red solid. ¹H NMR (500 MHz, CDCl₃) δ 8.82 (d, J=15.3 Hz, 1H), 7.98-7.93 (m, 1H), 7.89 (dd, J=15.7, 7.7 Hz, 2H), 7.68 (d, J=7.7 Hz, 1H), 7.62 (t, J=7.5 Hz, 1H), 7.56 (t, J=7.6 Hz, 1H), 7.46 (dt, J=16.0, 7.6 Hz, 2H), 7.08 (d, J=15.3 Hz, 1H), 3.97 (s, 3H), 3.80 (s, 3H), 2.77 (s, 3H), 1.44-1.40 (m, 1H), 0.97 (s, 9H). ¹³C NMR (126 MHz, CDCl₃) δ 167.39, 132.22, 132.08, 131.83, 130.91, 130.63, 130.17, 127.89, 127.38, 77.28, 77.02, 76.77, 56.96, 52.81, 52.48, 30.73, 29.96, 20.06.

methyl (E)-2-(7-(butylamino)-3-(2-(methoxycarbonyl)styryl)-2-methylimidazo[1,2-b][1,2,4]triazin-6-yl)benzoate Compound 85 was prepared by General procedure 4. (22 mg, 0.045 mmol, 18% yield). Red solid. ¹H NMR (500 MHz, CDCl₃) δ 8.74 (d, J=15.4 Hz, 1H), 8.56-8.48 (m, 2H), 7.95-7.87 (m, 1H), 7.82 (td, J=7.5, 1.3 Hz, 1H), 7.72-7.65 (m, 1H), 7.60 (ddd, J=8.2, 7.2, 1.2 Hz, 1H), 7.58-7.48 (m, 1H), 7.40 (td, J=7.6, 1.2 Hz, 1H), 7.11 (d, J=15.4 Hz, 1H), 3.94 (s, 3H), 3.88 (s, 3H), 2.77 (s, 3H), 1.83 (ddt, J=9.9, 7.6, 3.7 Hz, 2H), 1.50 (hept, J=7.3 Hz, 2H), 1.23 (s, 3H), 1.00 (t, J=7.4 Hz, 3H). ¹³C NMR (126 MHz, CDCl₃) δ 166.34, 159.94, 132.20, 131.15, 130.89, 129.98, 129.75, 129.36, 129.01, 128.34, 128.05, 127.80, 127.27, 126.69, 124.04, 121.79, 76.26, 76.00, 75.75, 51.84, 51.58, 42.06, 30.90, 28.68, 18.99, 12.88, 0.00.

### (E)-6-methyl-5-(2-(naphthalen-2-yl)vinyl)-1,2,4-triazin-3-amine

Compound 87 was prepared by General procedure 1. (16 mg, 0.075 mmol, 30% yield). Orange solid. ¹H NMR (500 MHz, CDCl₃) δ 8.23 (d, J=15.5 Hz, 1H), 8.01-7.97 (m, 1H), 7.89-7.80 (m, 3H), 7.76 (dd, J=8.6, 1.8 Hz, 1H), 7.55-7.46 (m, 2H), 7.16 (d, J=15.5 Hz, 1H), 5.26 (s, 2H), 2.68 (s, 3H). ¹³C NMR (126 MHz, CDCl₃) δ 147.46, 141.40, 134.18, 133.43, 132.91, 129.95, 128.79, 128.60, 127.84, 127.30, 126.82, 123.56, 120.44, 77.28, 77.03, 76.77, 18.56.

N-(tert-butyl)-2,3-dimethyl-6-(naphthalen-2-yl)imidazo[1,2-b][1,2,4]triazin-7-amine Compound 88 was prepared by General procedure 1. (25 mg, 0.085 mmol, 34% yield). Red solid. ¹H NMR (500 MHz, CDCl₃) δ 8.86-8.82 (m, 1H), 8.51 (dd, J=8.6, 1.7 Hz, 1H), 7.94-7.77 (m, 3H), 7.49-7.41 (m, 2H), 2.60 (s, 3H), 2.56 (s, 3H), 1.13 (s, 9H). ¹³C NMR (126 MHz, CDCl₃) δ 138.94, 133.42, 133.10, 128.58, 127.62, 127.48, 127.18, 126.06, 125.96, 125.86, 77.28, 77.03, 76.78, 57.33, 30.45, 22.71, 19.86.

### Results

While optimizing the fluorescent properties of amino-imidazopyridines 13, it was noticed that changing the amino-pyridine 11, had the greatest effect on fluorescence. A scan was run on relevant aminopyridine analogs 11 and 5,6-dimethyl-1,2,4-triazin-3-amine 14 was chosen as a candidate. While checking the reaction for completion via TLC, it was noticed that there was constantly a nonpolar red fluorescence spot in the reactions. Upon isolation, characterization, and literature review, it was revealed that this dimethyl triazine 14 undergoes aldehyde condensation at the 5 position of the triazine. Upon this discovery, it was quickly realized that this condensation in conjunction with the GBB reaction (FIG. 2A) afforded new scaffolds. However, to fully explore these scaffolds, optimization of the reaction was performed (Table 15). The yield drastically increased as the time was increased Entry 1-5, however it remained low at 28% even after 140 minutes at 140° C. (MW).

Next, a solvent screen was run with methanol (MeOH), ethanol (EtOH), dichloroethane (DCE), acetonitrile (ACN), chloroform, dioxane (DiOx), tetrahydrofuran (THF), trifluoroethanol (TFE), and dichloromethane (DCM) (Table 16, Entry 3, 6-13). The 40-minute reaction time was chosen to decrease microwave reactor time and the general trend was that more nonpolar solvents produced a better yield with DCE being the best at 42% (Entry 7). TFE was the worst at only 9% (Entry 12).

From there a series of optimizations were done (Table 17). Increasing the temperature (Entry 1, 14) resulted in an increased yield from 3% to 15%. Swapping solvents from MeOH to DCE (Entry 14, 15), increased the yields as well at higher temperatures. Increasing the equivalents of the tertbutylisocyanide (t-BuNC) from 1.1 to 2.0 decreased the yield in both MeOH (Entry 14, 16) and DCE (Entry 15, 17). While increasing the aldehyde equivalents to 4 increased the yield in both MeOH and DCE (Entry 14, 18 and 15, 19). Lastly, increasing both the equivalents of the t-BuNC and aldehyde (Entry 20, 21) did not increase the yield to an appreciable amount.

A catalyst screen was run with various catalysts (Table 18). Piperidine, acetic acid (AcOH)16 and ytterbium triflate (Yb(OTf)₃) (Ghashghaei, O.; Pedrola, M.; Seghetti, F.; Martin, V. V; Zavarce, R.; Babiak, M.; Novacek, J.; Hartung, F.; Rolfes, K. M.; Haarmann-Stemmann, T.; Lavilla, R. Extended Multicomponent Reactions with Indole Aldehydes: Access to Unprecedented Polyheterocyclic Scaffolds, Ligands of the Aryl Hydrocarbon Receptor. https://doi.org/10.1002/anie.202011253) gave modest yields for this reaction (Entry 21-23). While sulfuric acid (H₂SO₄), H₂SO₄ with Scandium triflate (Sc(OTf)₃), and NH₄Cl (Nenajdenko, V. G.; Reznichenko, A. L.; Balenkova, E. S. The Synthesis of Aminoimidazo[1,22a]Azinecarboxylic Acid Esters from Ethyl Glyoxylate; 2007; Vol. 56) (Entry 24-27) gave poor to no yields. The best catalyst was Sc(OTf)3 (Blackburn, C.; Guan, B.; Fleming, P.; Shiosaki, K.; Tsai, S. Parallel Synthesis of 3-Aminoimidazo[1,2-a]Pyridines and Pyrazines by a New Three-Component Condensation. Tetrahedron Letters 1998, 39 (22), 3635-3638) (Entry 28) with 35%. At this point, it was clear that Sc(OTf)3 was the best catalyst and DCE was the best solvent. A few more optimizations were run on the molarity of DCE (Table 18, Entry 19, 28-30) and it was found that 0.TM was better than 0.2M. The equivalents of t-BuNC were increased to 1.4 (Entry 31) and an all-time high yield of 43% was achieved. Temperature and time studies were done as well (Entry 32-33) without increasing the yield.

From there the equivalents of the Lewis Acid Sc(OTf)₃ were varied (Table 19). However, 0.2 equivalents (Entry 31) reigned supreme garnering a 43% yield. After the optimization of equivalents of starting materials, time, and temperature, some different solvents were revisited (Table 21). Ratios of DCE:MeOH (Entry 38-40), toluene (Entry 41), and neat conditions (Entry 42) were explored all with deleterious effects on the yield.

Upon optimization of the time, temperature, solvent, equivalents, and catalyst, different aldehydes (2) were screened (Scheme 2). In most cases, the yields decreased with 43, 45, 47-49, but 46 increased. Upon investigation of the fluorescent properties of the molecules, they were all found to fluoresce red (>630 nm). The quantum yield of the molecules was low to moderate with 42 and 45 having the lowest quantum yield at 0.11 and 49 having the highest at 0.45. Once the aldehyde analogs were synthesized with t-BuNC, the isocyanide replacement TMSCN 52 was incorporated (Scheme 3) (Guchhait, S. K.; Chaudhary, V.; Madaan, C. A Chemoselective Ugi-Type Reaction in Water Using TMSCN as a Functional Isonitrile Equivalent: Generation of Heteroaromatic Molecular Diversity. Organic and Biomolecular Chemistry 2012, 10 (46), 9271-9277). The initial reaction conditions (Entry 43, Table 22) used from the previous optimizations (Entry 31, Table 19) garnered a 5% yield which proved inefficient at making the fully conjugated product 53. A short methodology screen was conducted with 46% being the highest yield (Entry 53).

Following the optimization of the isocyanide replacement TMSCN, an aldehyde screen and fluorescent measurements were taken (Scheme 4). The TMSCN proved overall to be worse for the conversion to the product compared to t-BuNC. For note, the 4-nitro 61 failed to garner any product. This could be due to the weaker strength of TMSCN. Fluorescent properties were similar to the tert-butylisocyanide congener in 42-49 (Scheme 2) in regard to the Absₘₐₓ and Emₘₐₓ which were in the 500s and 600s nm respectively. However, the quantum yield was generally lower.

The isocyanide screen was continued with aryl-isocyanides (Scheme 5). The 4-methoxy 68 and meta-morpholino 69 congeners proved ineffectual in garnering the desired product 67 One explanation of this is that due to their aryl structure, the isocyanide was weaker than TMSCN. Once the isocyanide was swapped from aryl to benzyl 72, the desired product was furnished again (Scheme 6). Out of the isocyanides that did produce products, 73 had the worst yields. However, the quantum yields were overall higher than the other products (Scheme 2+4). Like previous work (Bedard, N.; Foley, C.; Davis, G. J.; Jewett, J. C.; Hulme, C. Sequential Knoevenagel [4+1] Cycloaddition-Condensation-Aza-Friedel-Crafts Intramolecular Cyclization: A 4-Center-3-Component Reaction Toward Tunable Fluorescent Indolizine Tetracycles. J. Org. Chem 2021, 86, 17550-17559), when the imine 77 was subjected to Cu(OTf)₂, intramolecular cyclization occurred forming the fully conjugated 78 (Scheme 7). For both 79 and 80, Absₘₐₓ and Emₘₐₓ were decreased compared to the uncyclized version 77, while their quantum yields were increased.

Further investigation into scaffolds 81 that were similar to ours and possessed antimitotic effects (Meng, T.; Wang, W.; Zhang, Z.; Ma, L.; Zhang, Y.; Miao, Z.; Shen, J. Synthesis and Biological Evaluation of 6H-Pyrido[2′,1′:2,3]Imidazo[4,5-c]Isoquinolin-5(6H)-Ones as Antimitotic Agents and Inhibitors of Tubulin Polymerization. Bioorganic and Medicinal Chemistry 2014, 22 (2), 848-855) was conducted with various isocyanides (Scheme 8). While facile cyclization has been previously reported with aminopyridindes (Meng, T.; Wang, W.; Zhang, Z.; Ma, L.; Zhang, Y.; Miao, Z.; Shen, J. Synthesis and Biological Evaluation of 6H-Pyrido[2′,1′:2,3]Imidazo[4,5-c]Isoquinolin-5(6H)-Ones as Antimitotic Agents and Inhibitors of Tubulin Polymerization. Bioorganic and Medicinal Chemistry 2014, 22 (2), 848-855), the imidazotriazines 84 and 85 did not undergo intramolecular cyclization with a variety of isocyanides including t-Bu and n-Bu. Most surprising was the failure of n-Bu since it is not as sterically hindered. Further optimizations were conducted (Table 23), however, none were successful in producing 86.

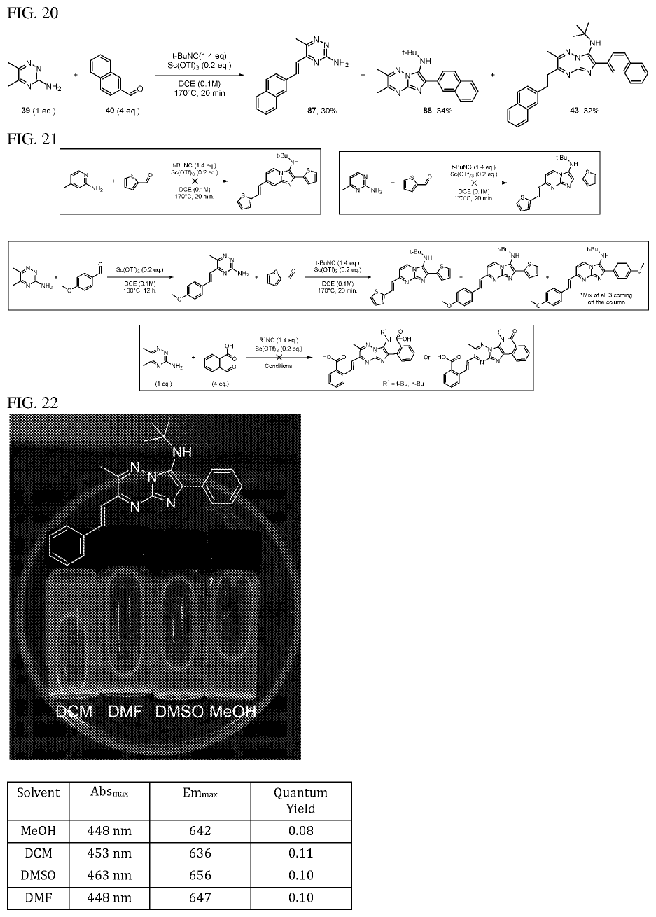

Given the ambiguity of the location of the condensation on the methyl on either the 5 or 6 positions, the structure of the product was confirmed by X-ray crystallography (FIG. 6) which confirmed the aldehyde's condensation on the 5-methyl. The 5-methyl condensation is in concordance with the previously reported position which was based on ultraviolet spectra.8 We hypothesized that the 5-methyl condensation event can happen before or after the GBB reaction as both the condensed aminotriazine 87 and the noncondensed GBB product 88 were isolated in the reaction (FIG. 20). Further explorations of the reaction's feasibility were investigated with different amidine starting materials and it was found that only the 5,6-dimethyl-1,2,4-triazin-3-amine underwent condensation (FIG. 21). Based on this information, a plausible mechanism was constructed (Scheme 9). In accordance with the reported literature,8 it is hypothesized that the triazine 89 first condenses with the aldehyde 90 forming 91. This amidine condensed product 91 then undergoes LA-mediated imine formation22 forming 92. 92 then undergoes [4+1] cycloaddition forming 93+94 which then undergoes a 1, 3 H-shift forming 95 that is in equilibrium with 96.

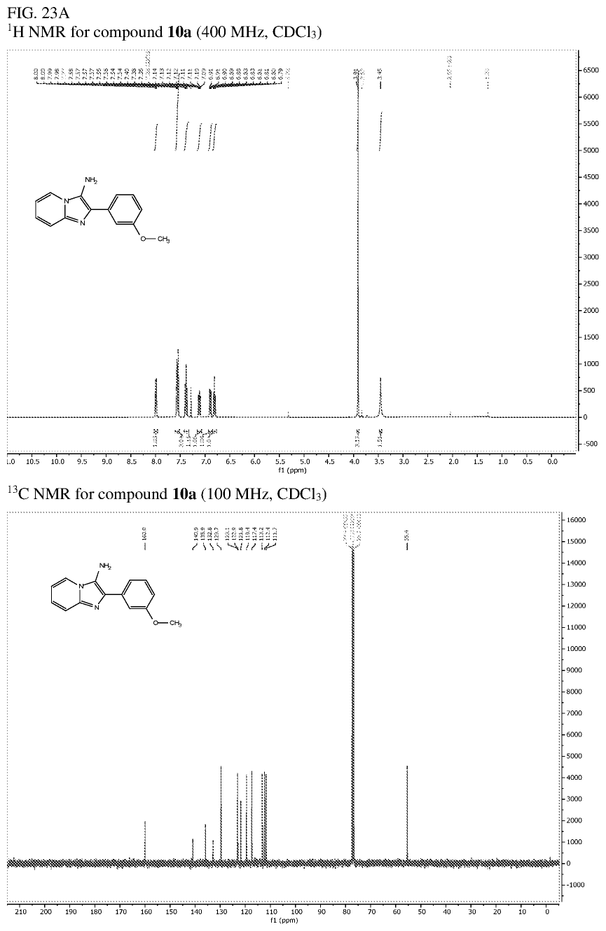

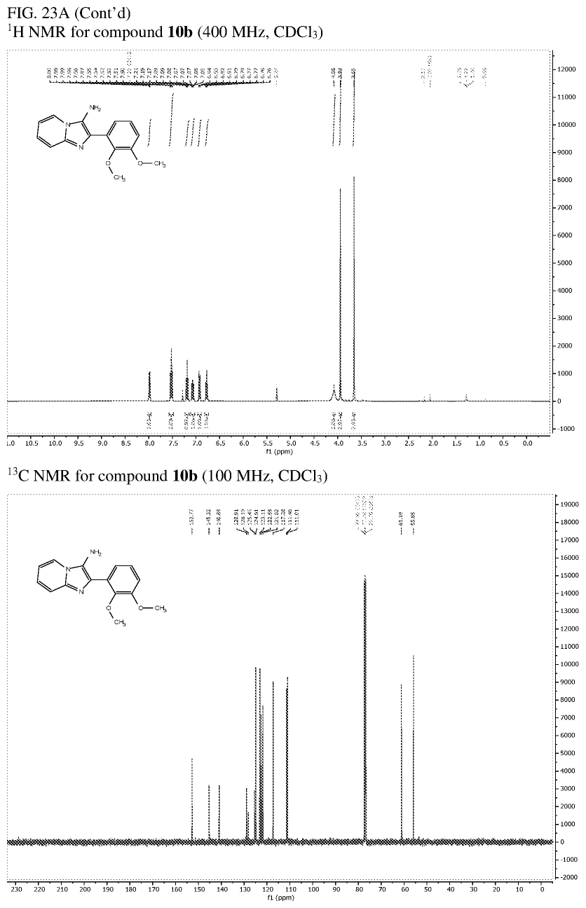

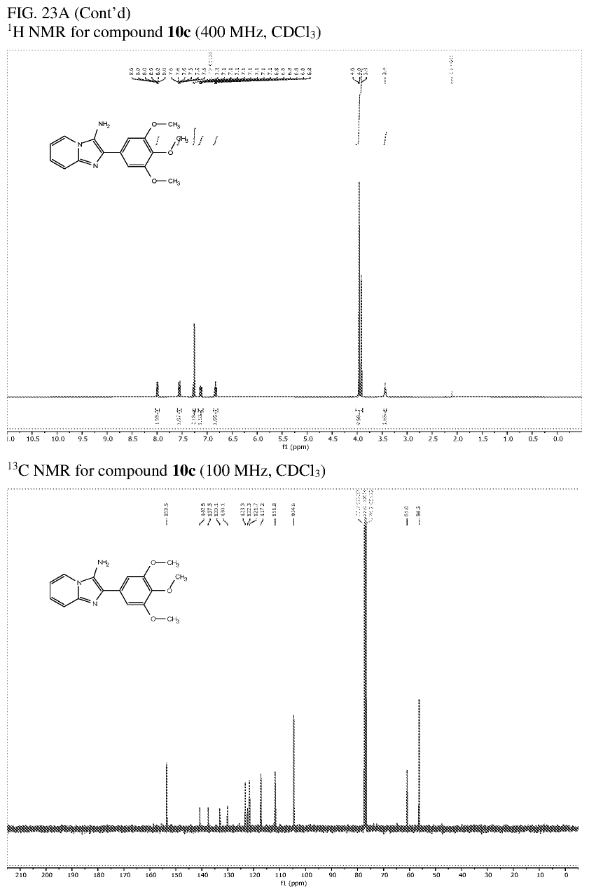

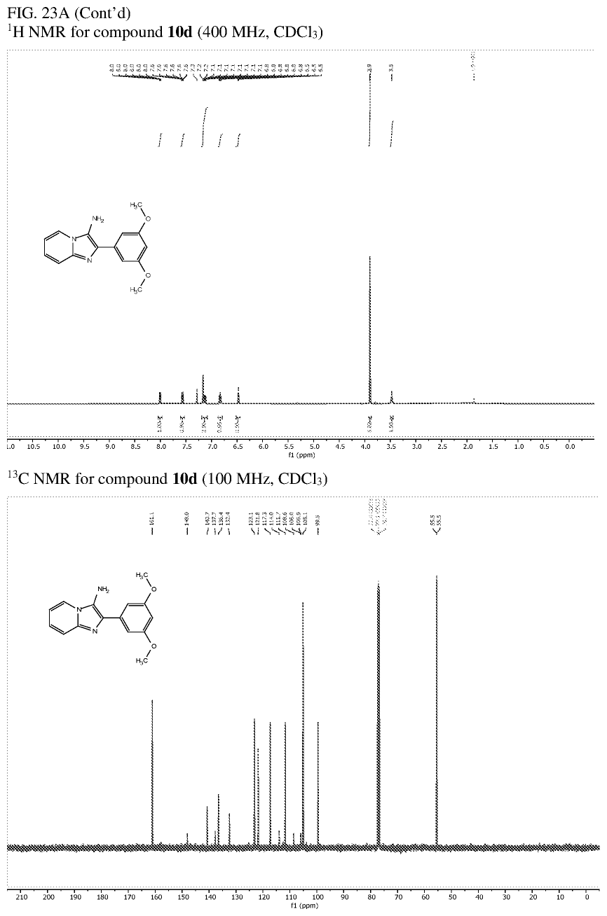

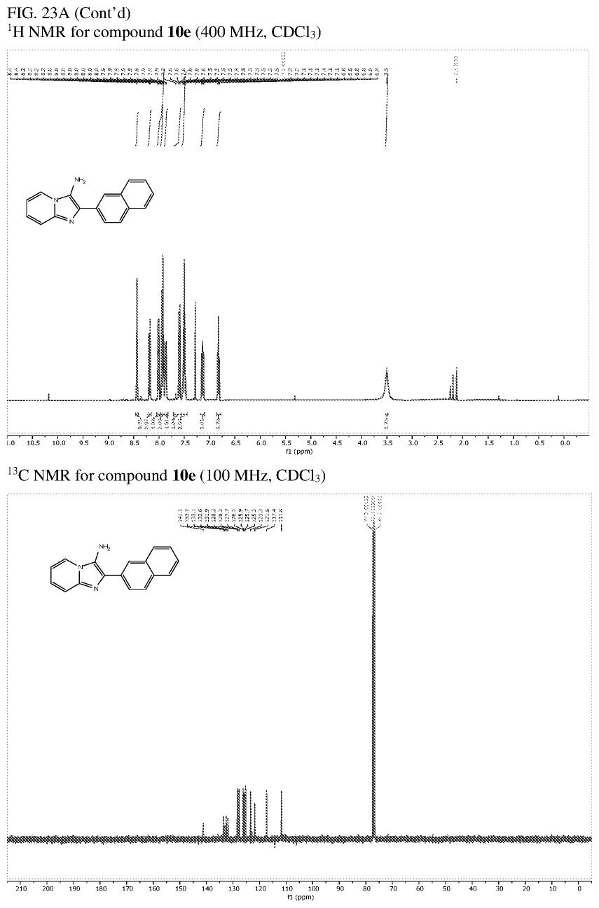

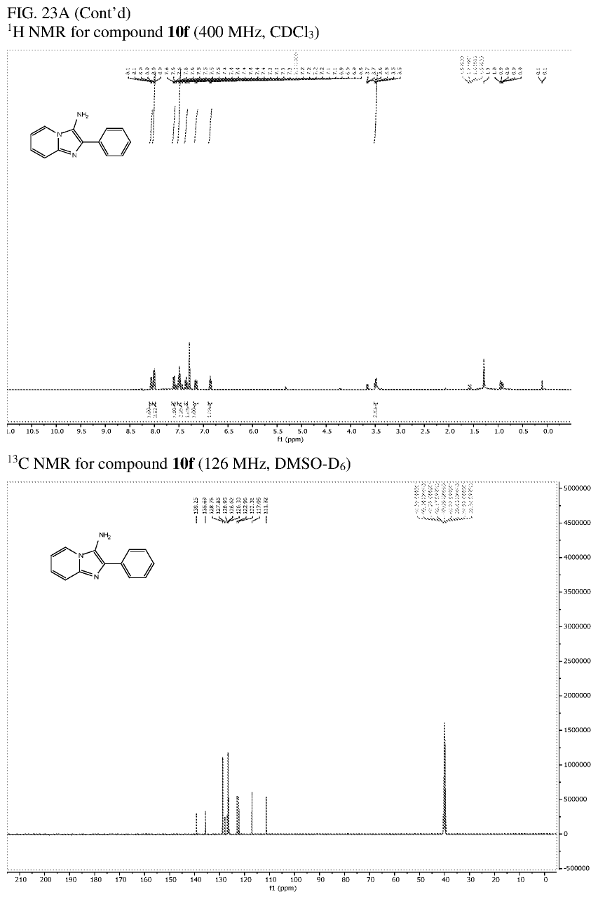

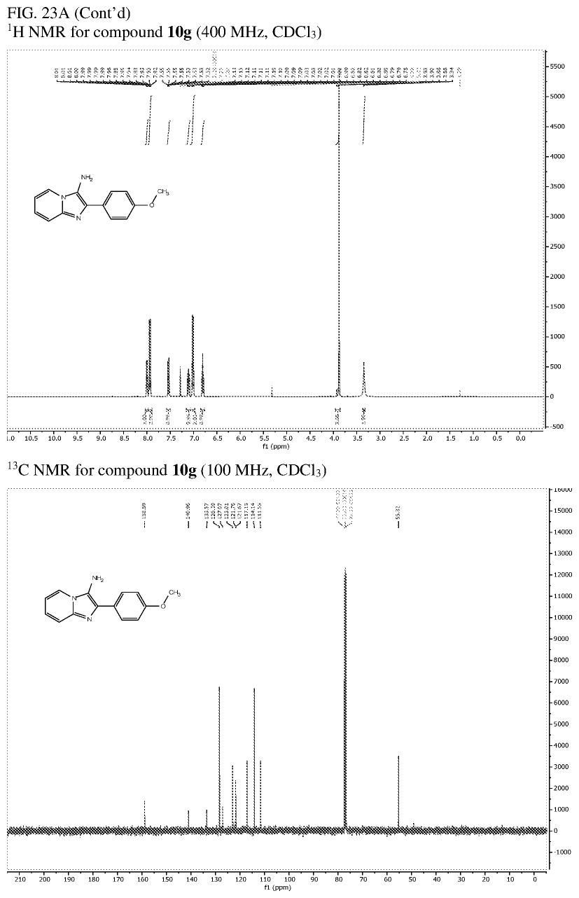

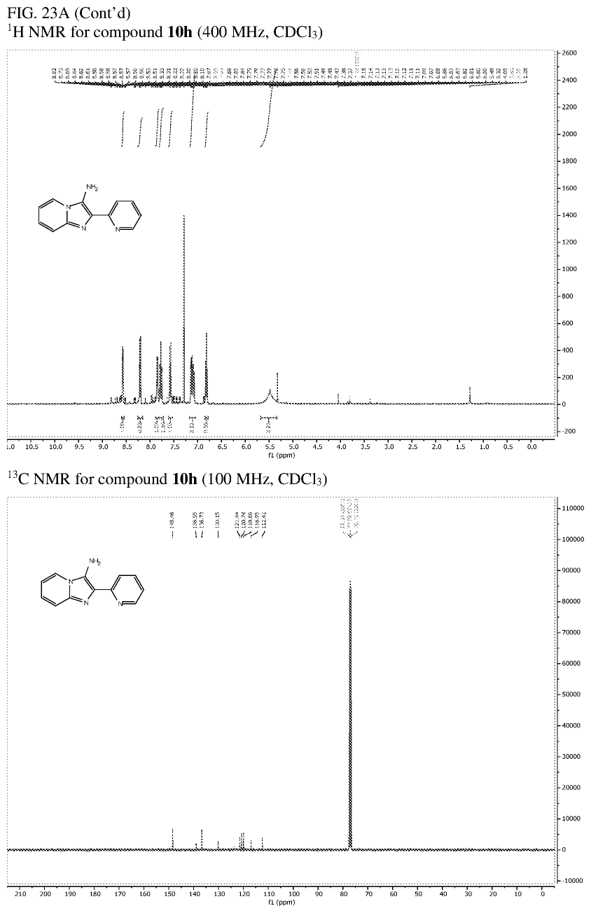

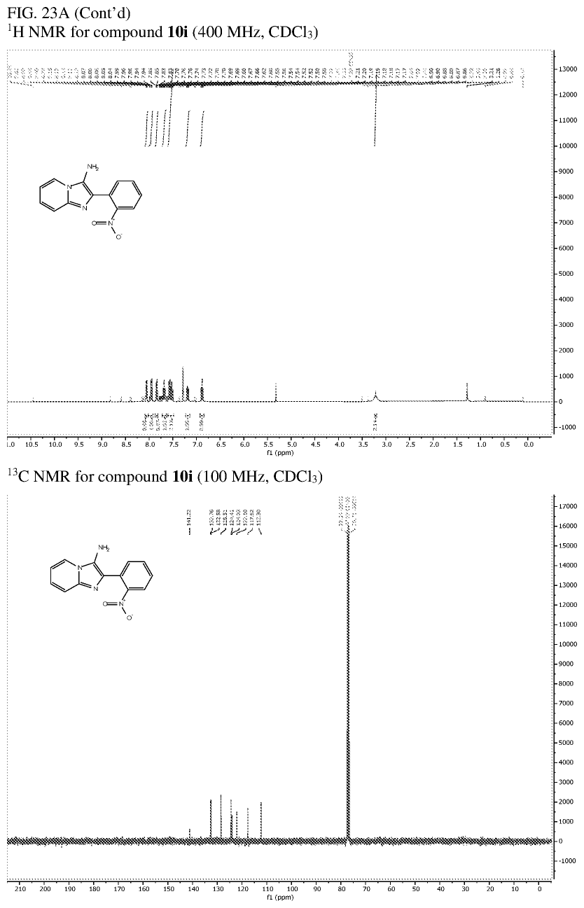

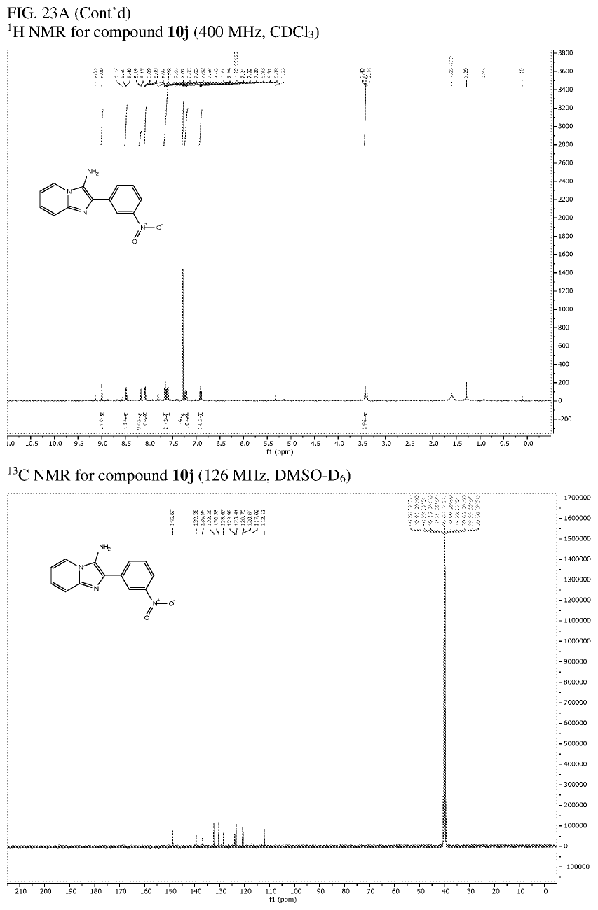

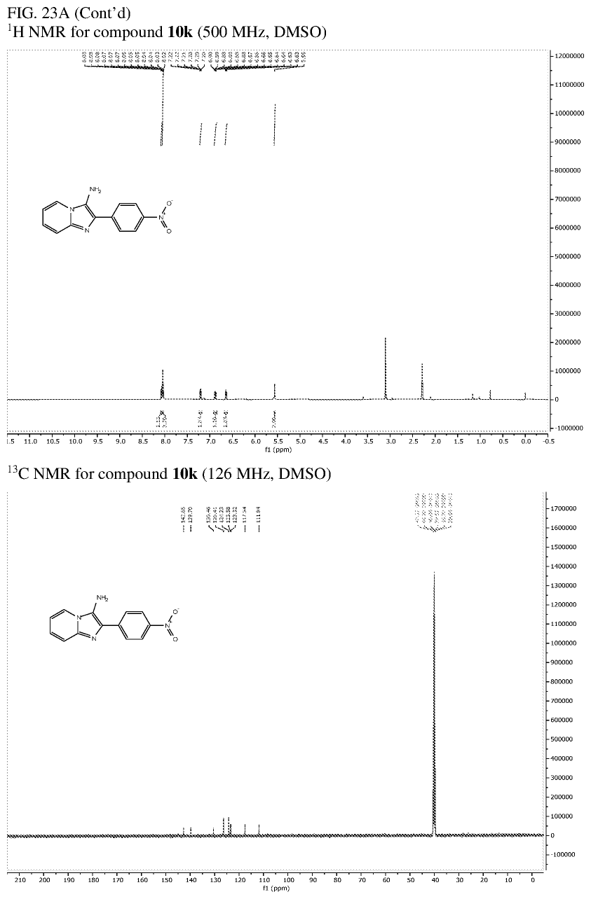

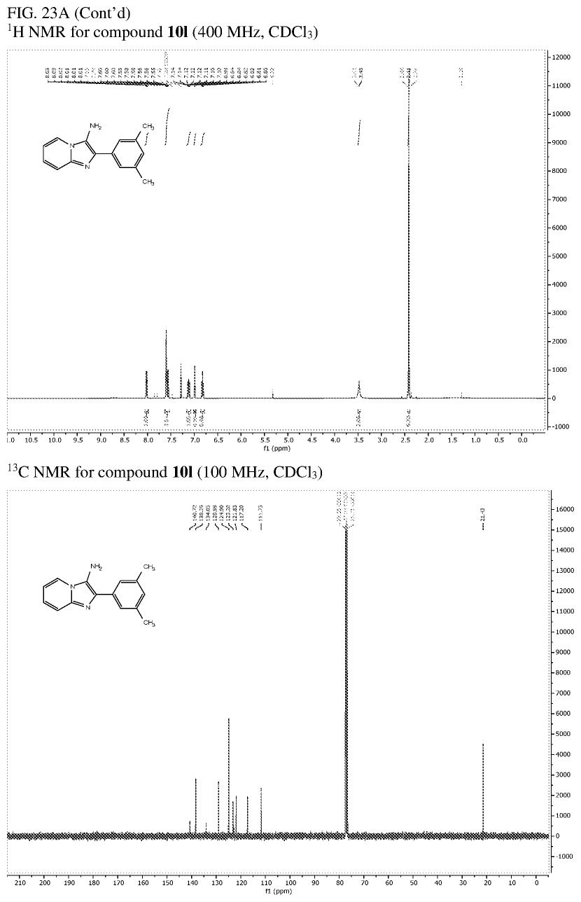

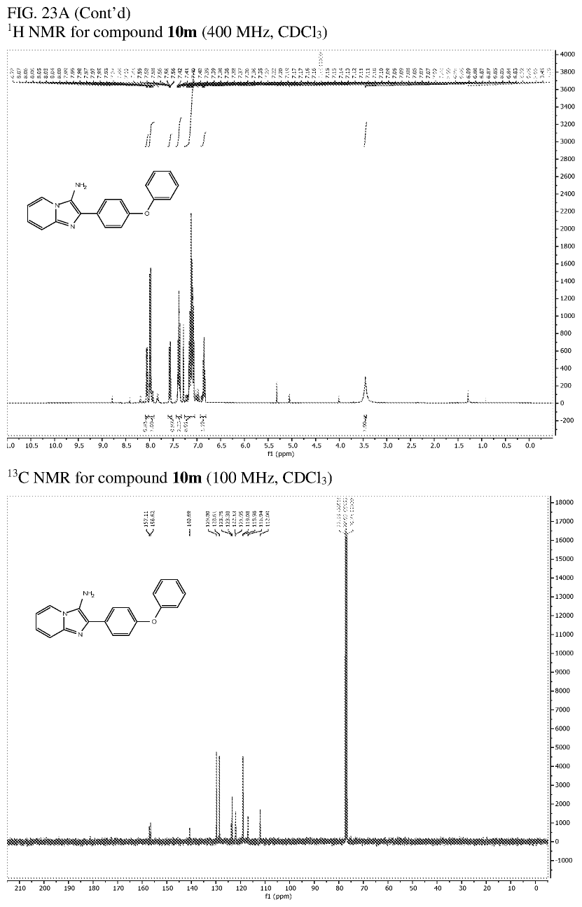

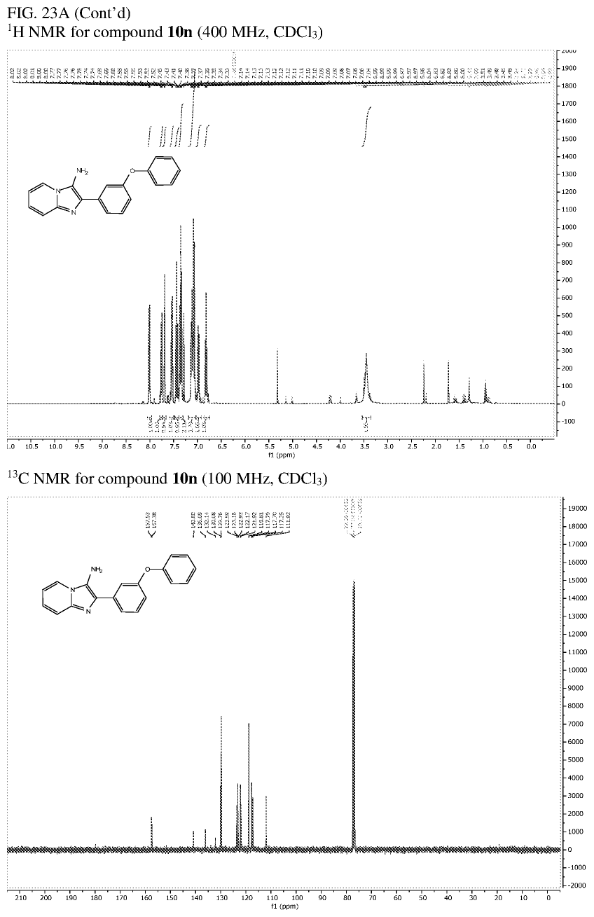

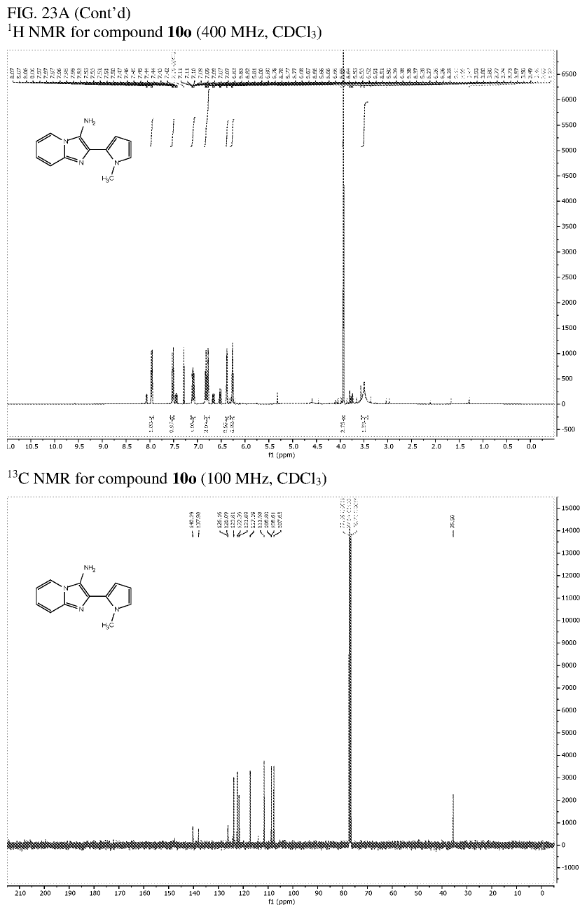

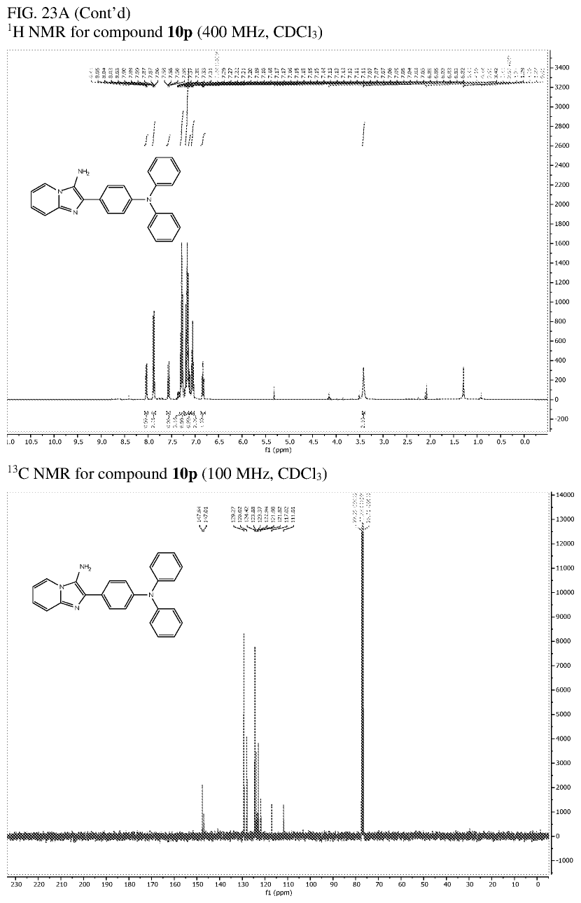

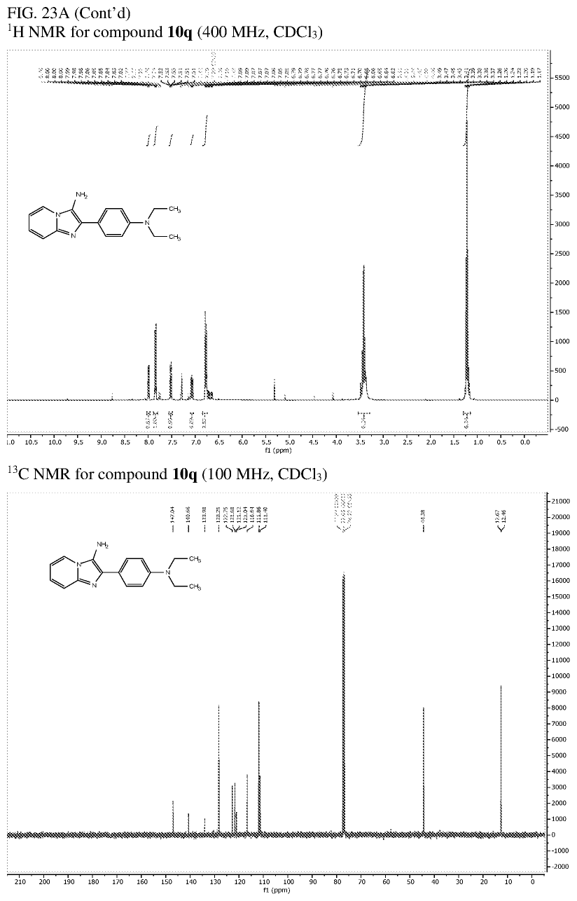

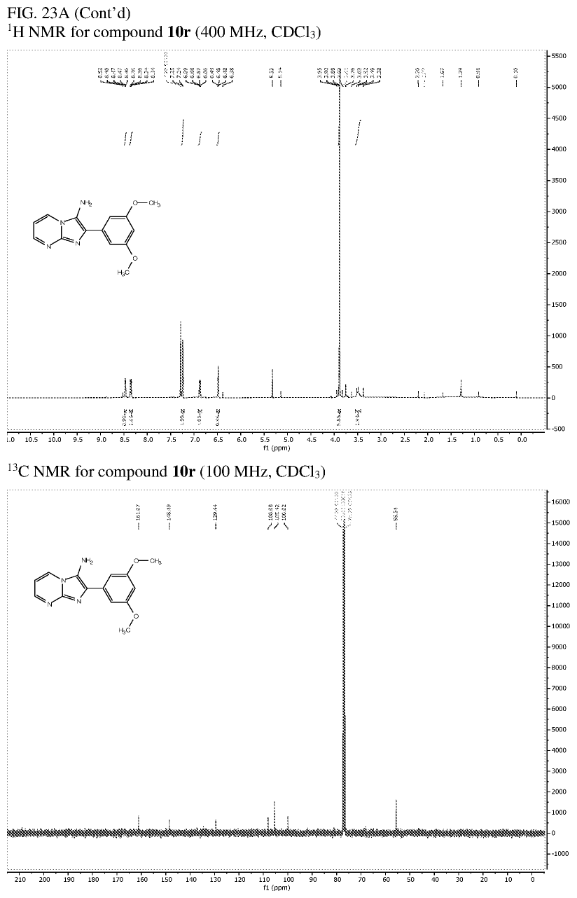

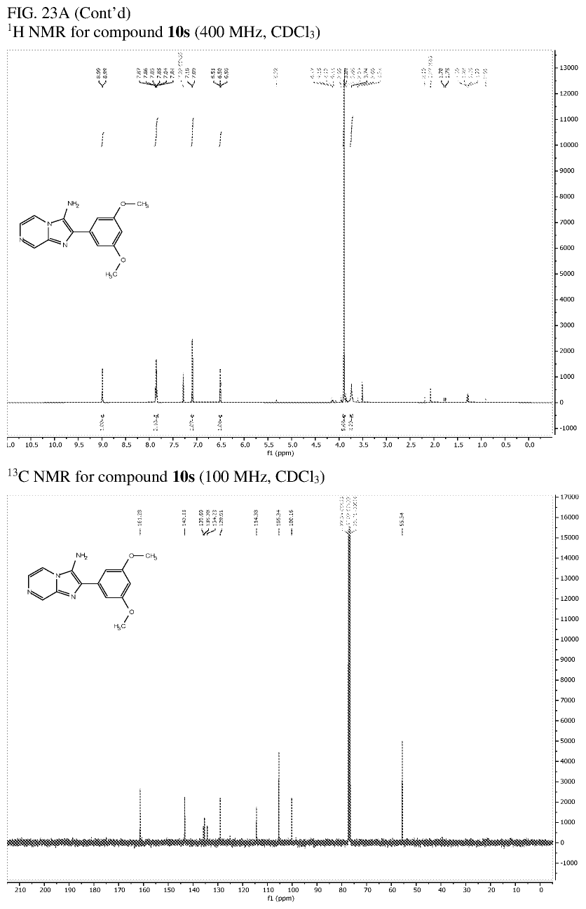

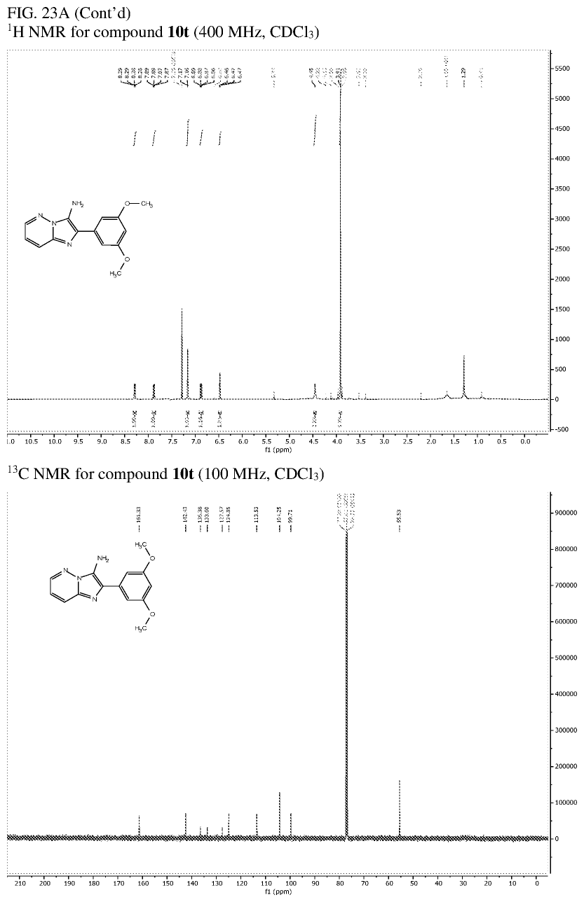

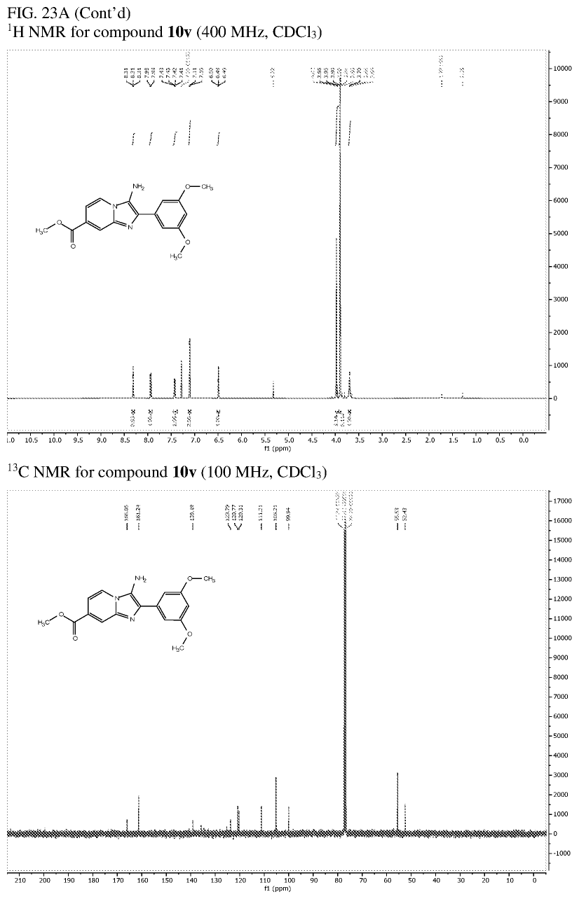

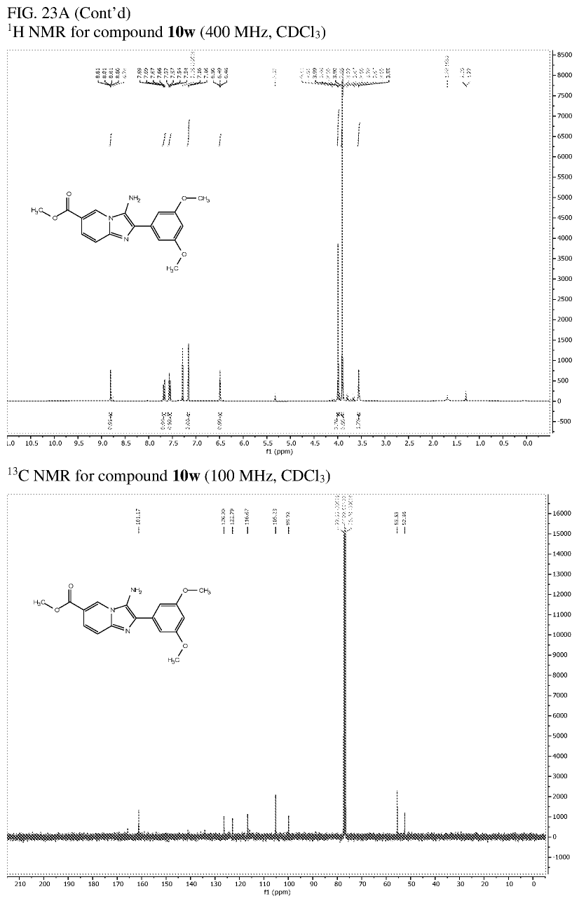

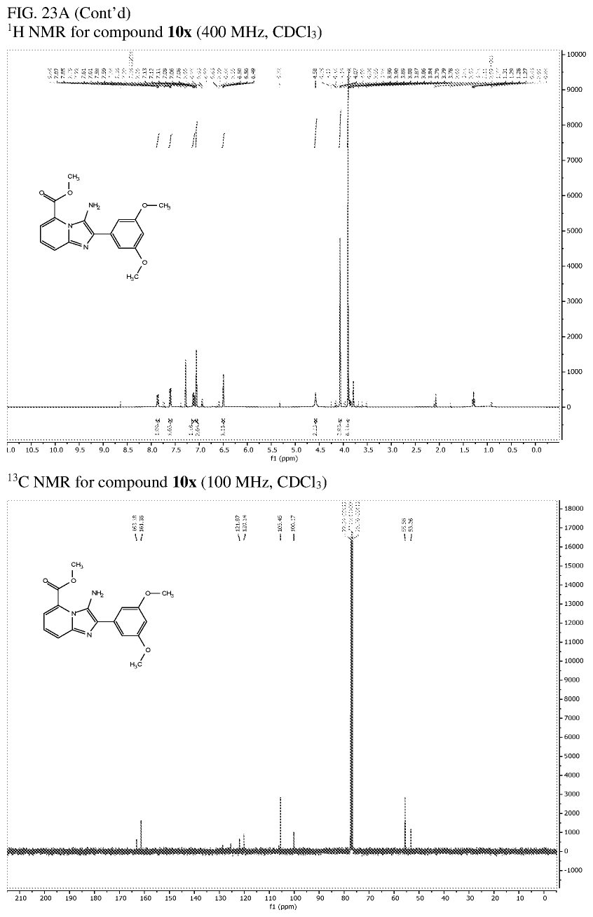

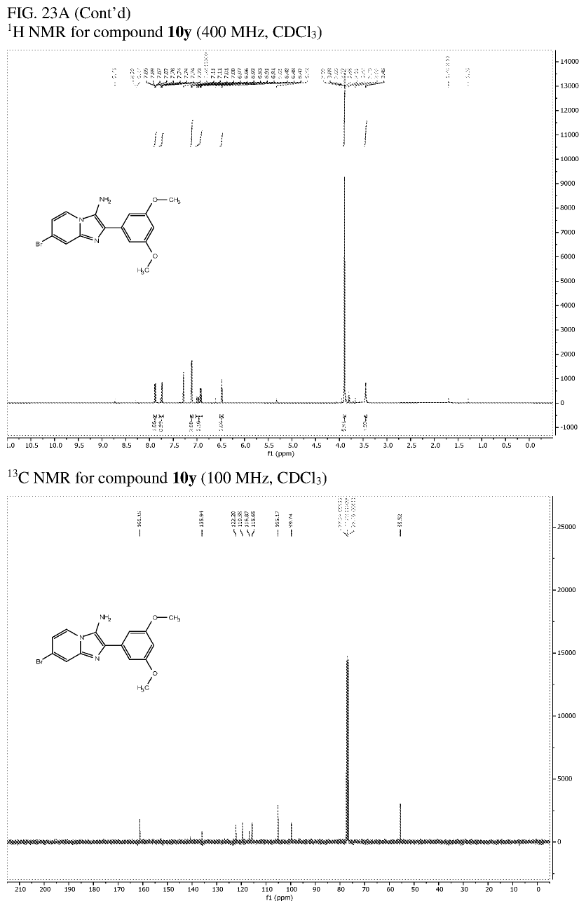

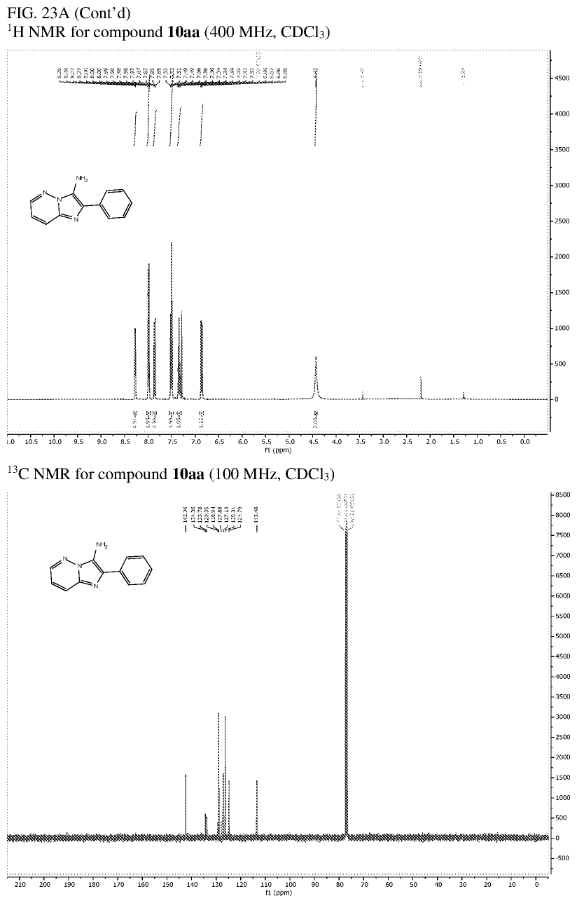

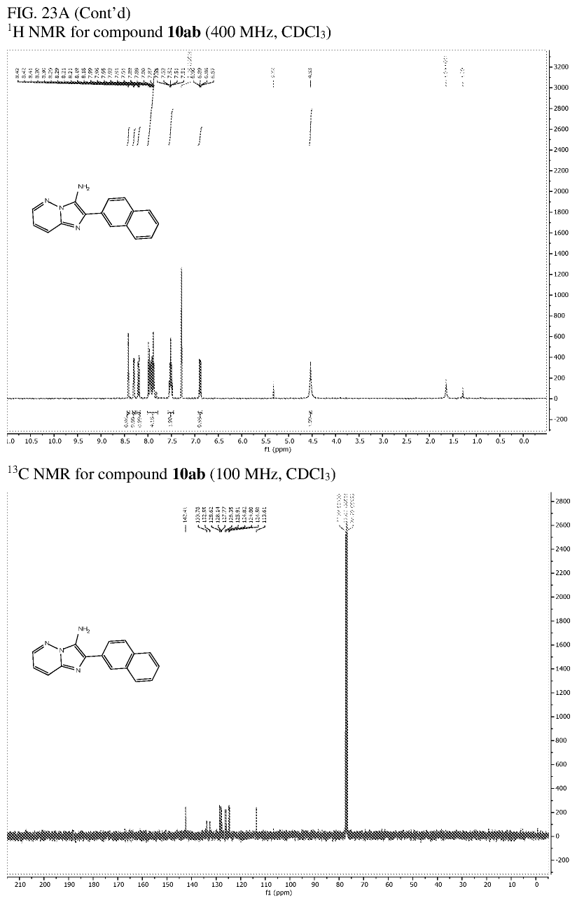

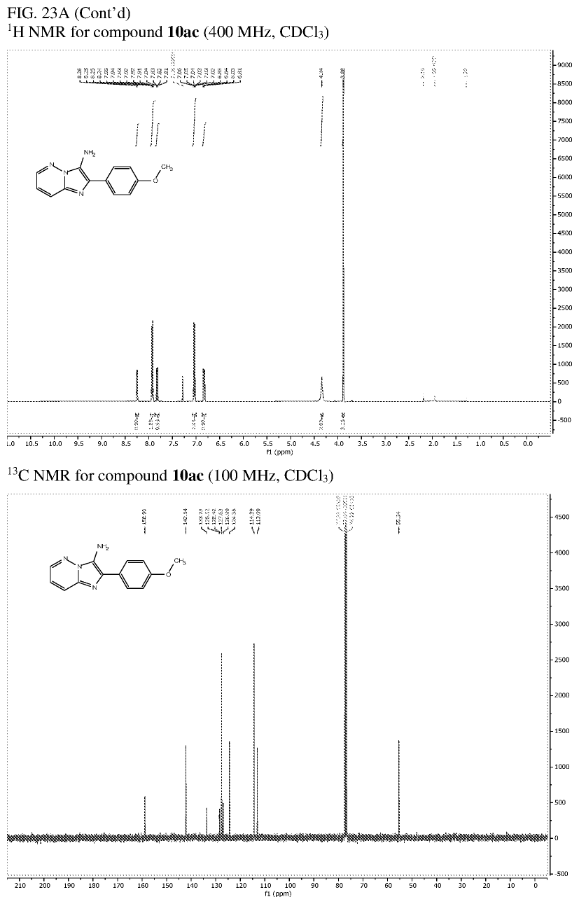

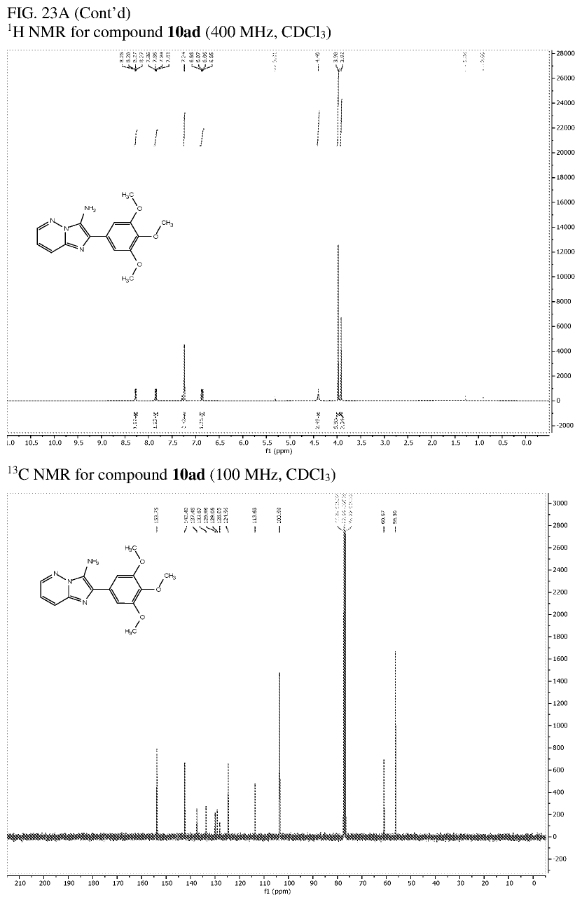

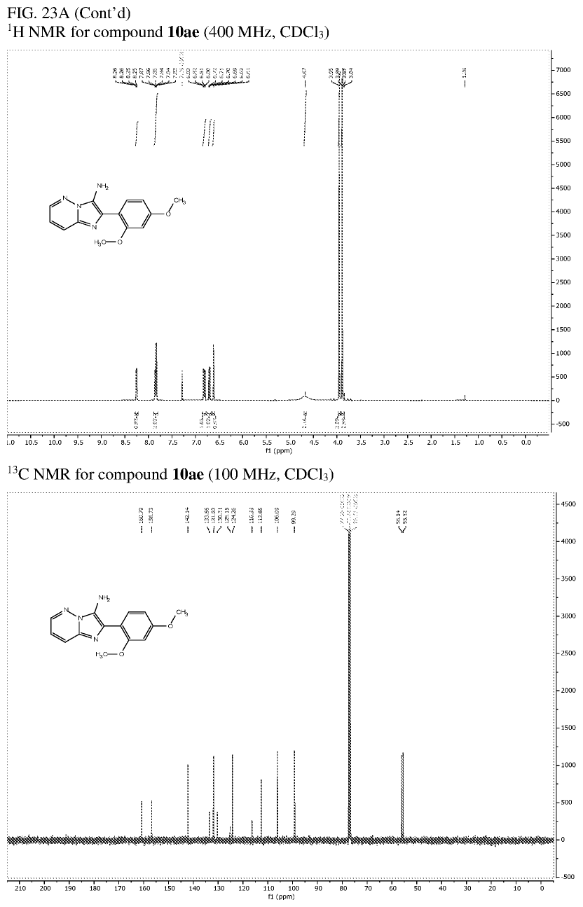

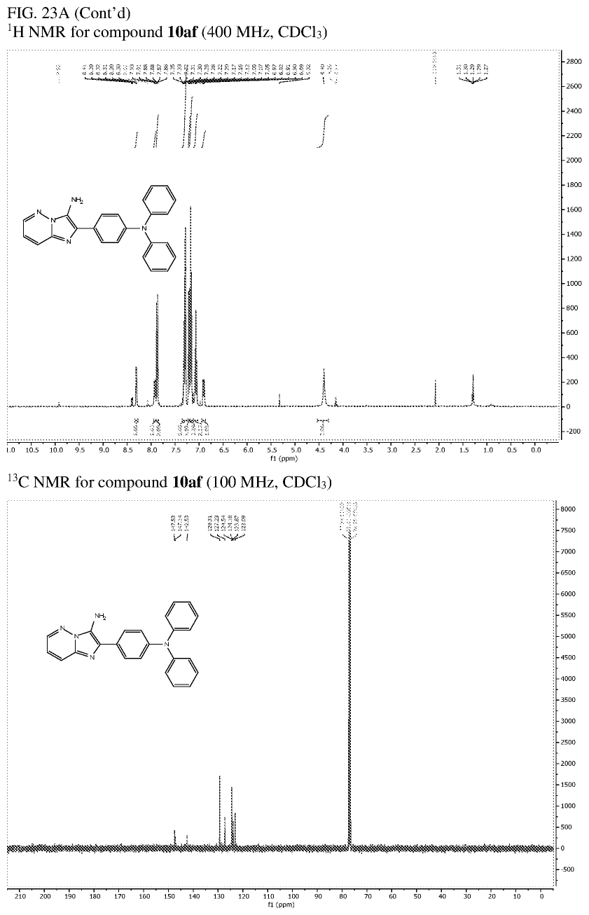

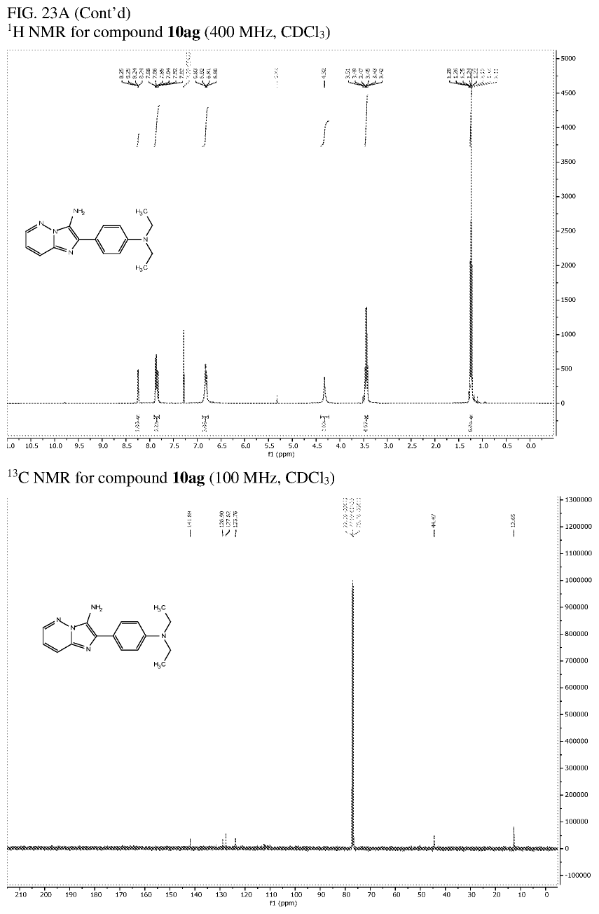

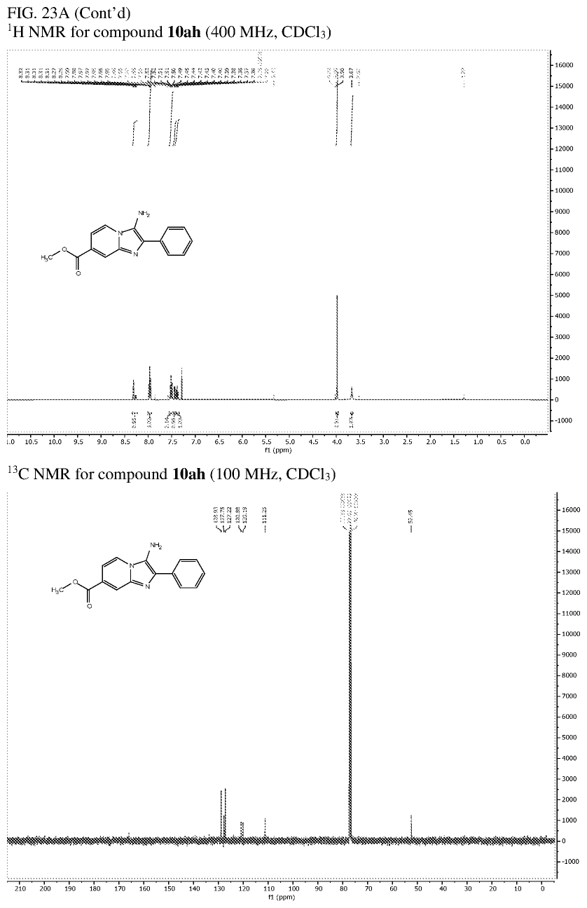

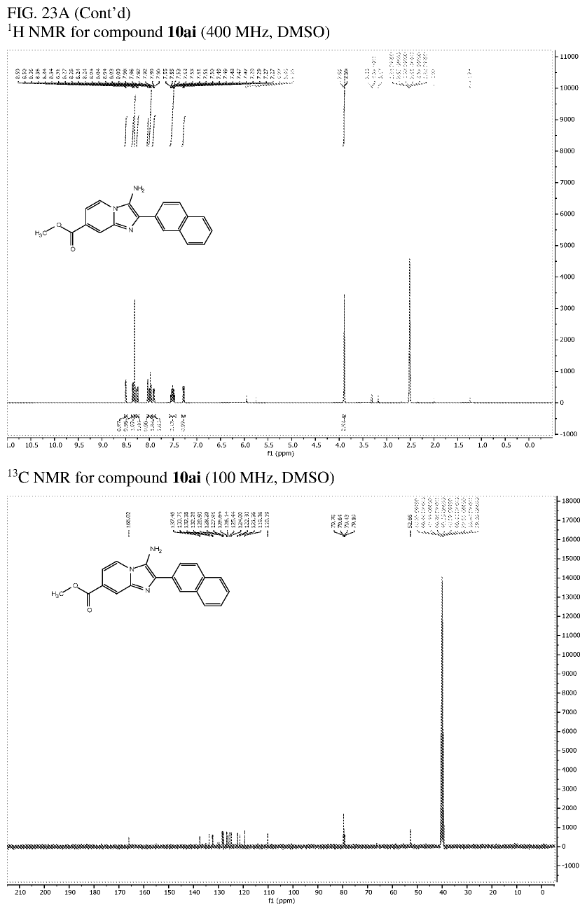

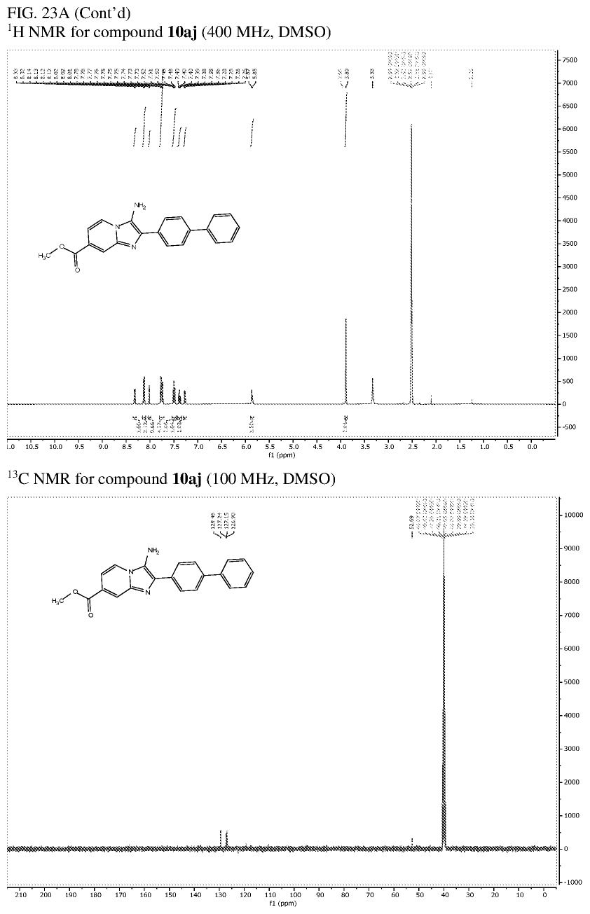

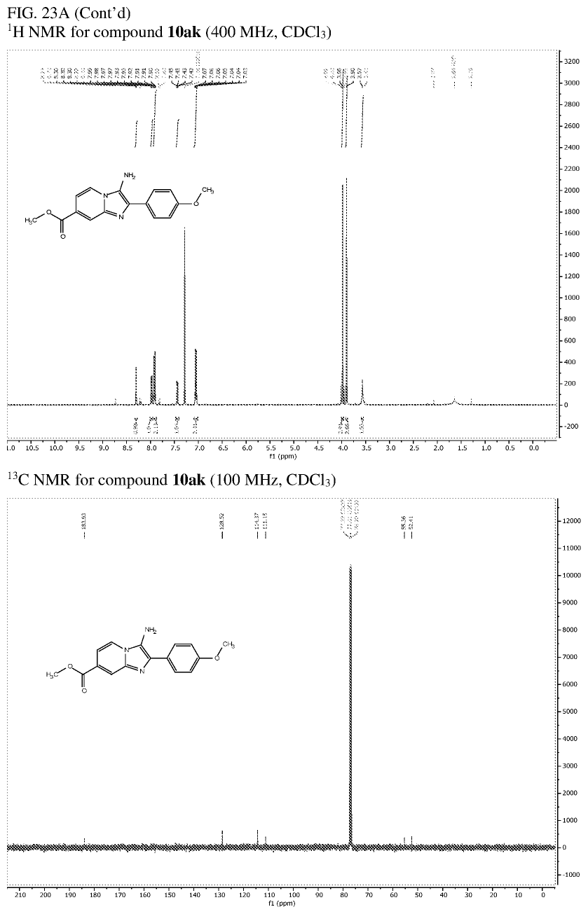

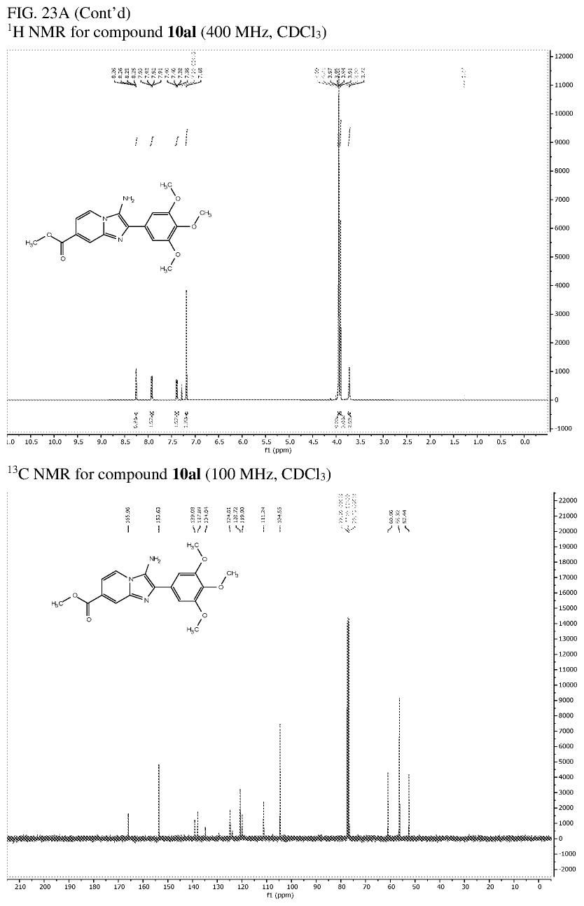

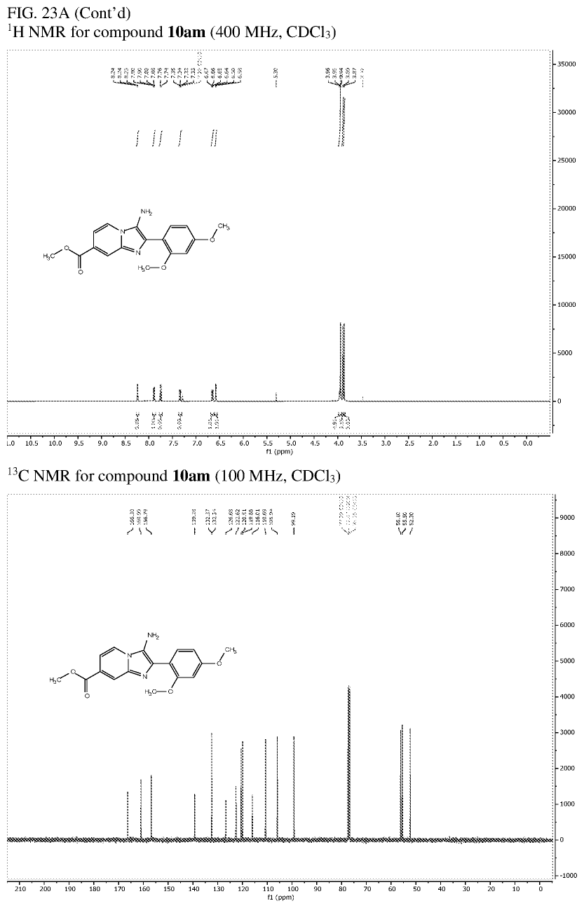

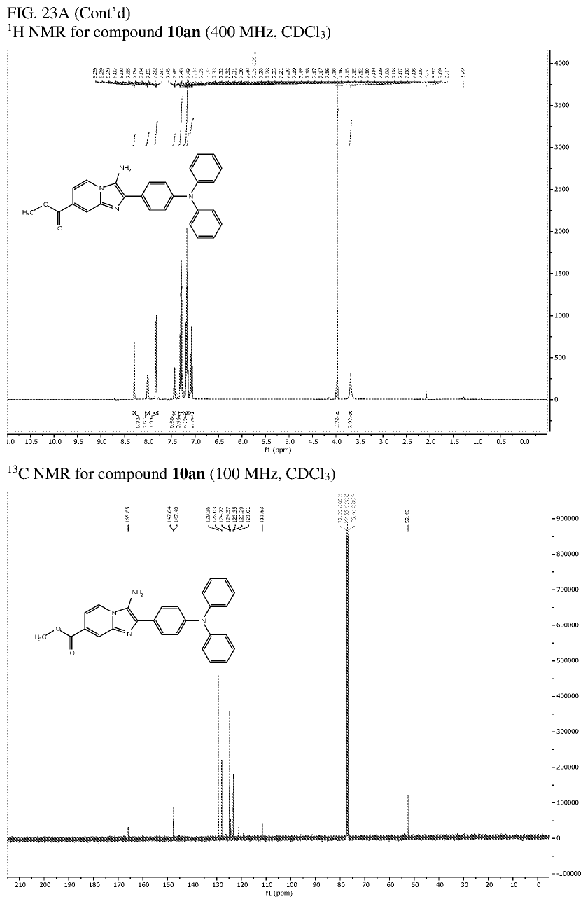

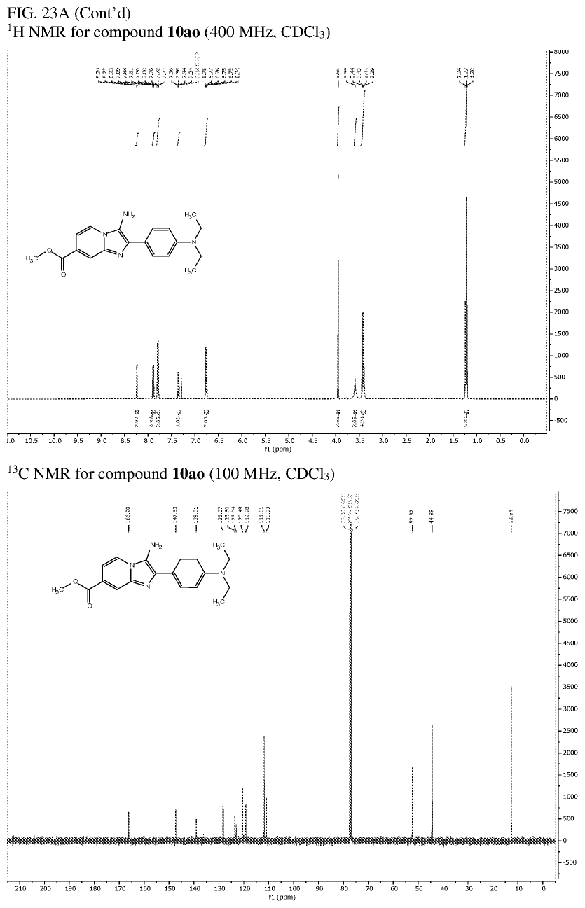

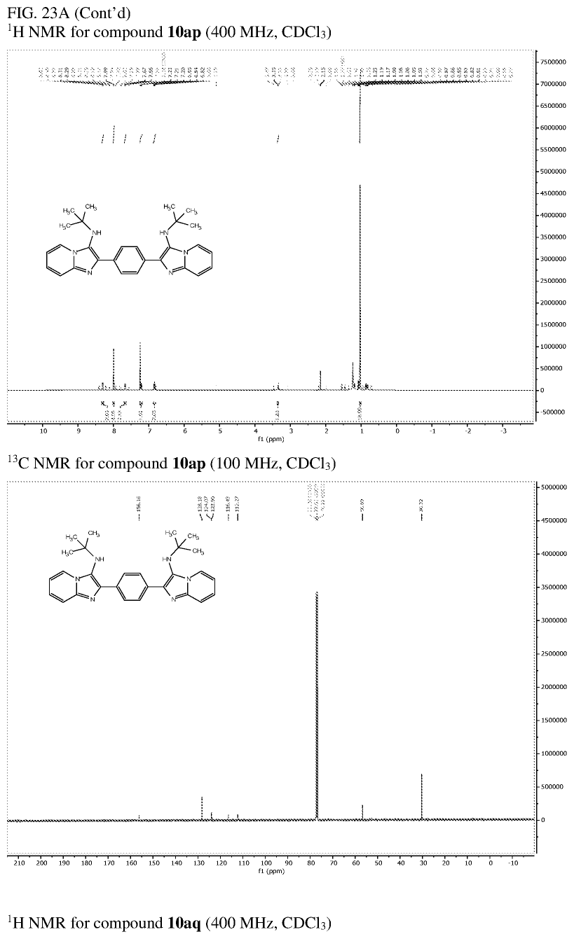

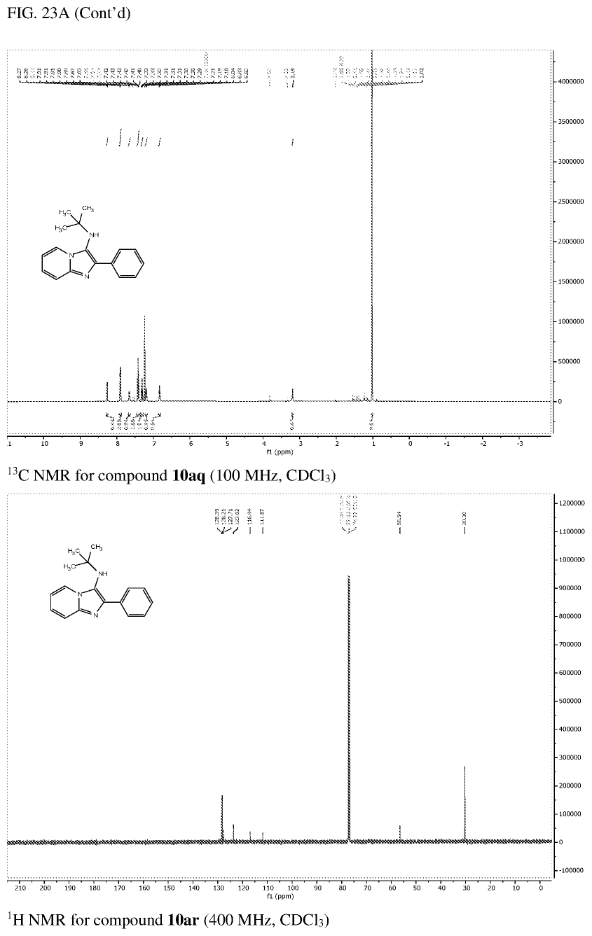

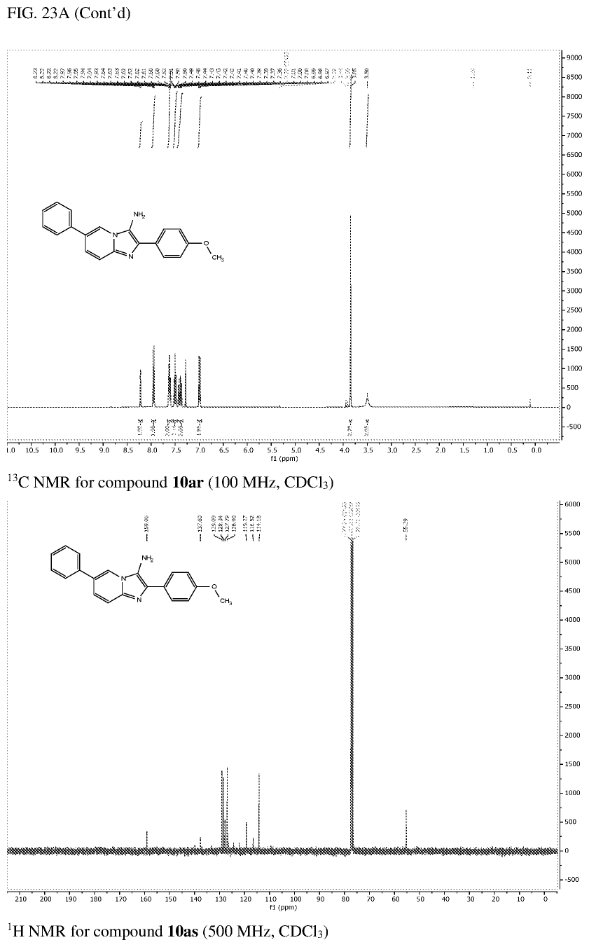

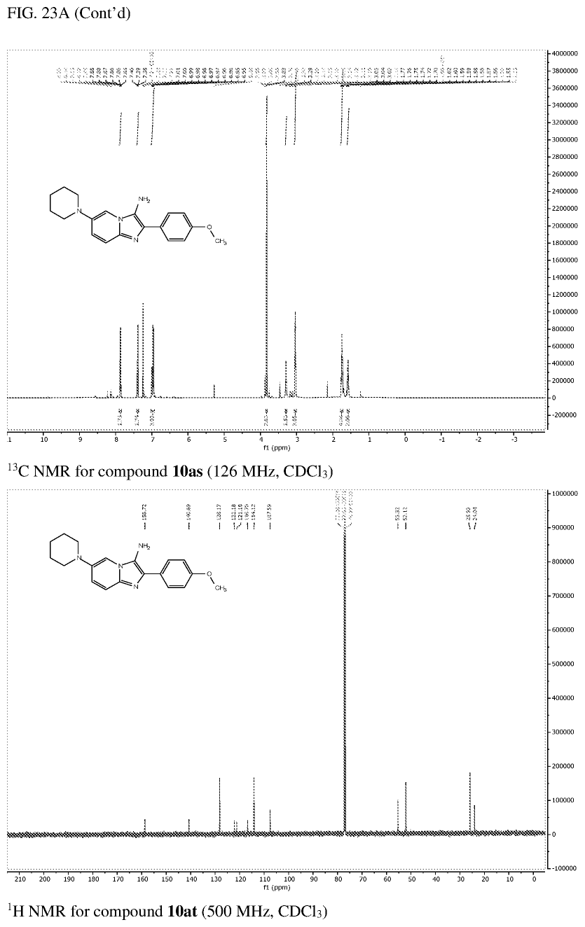

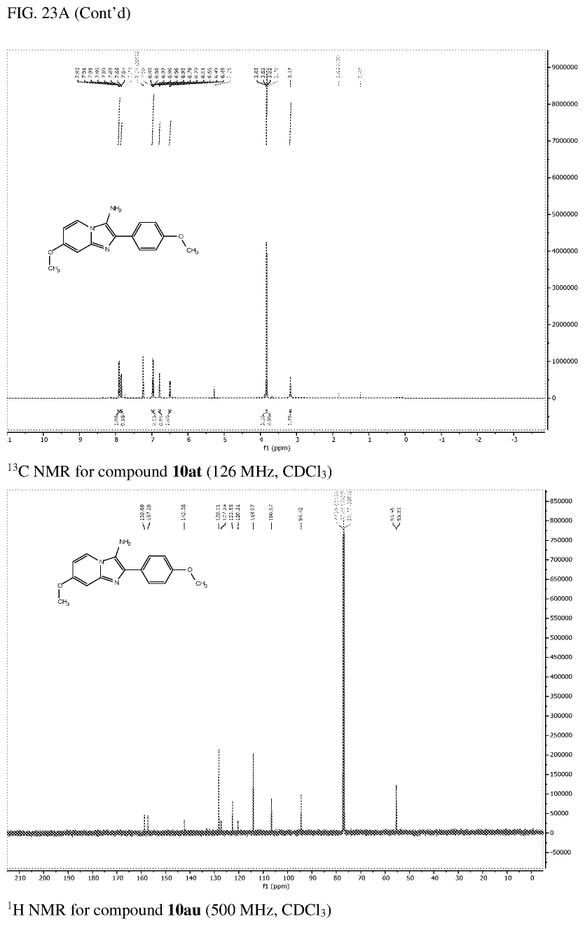

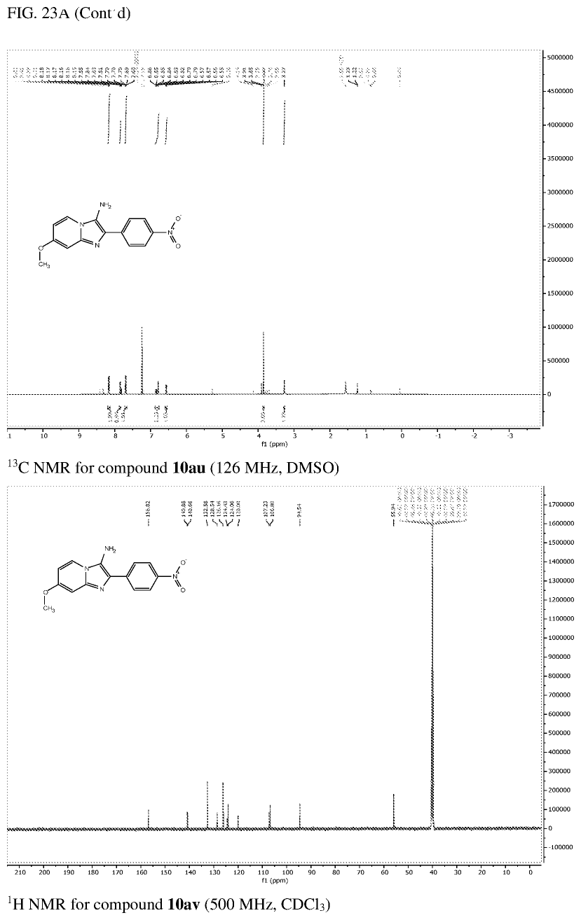

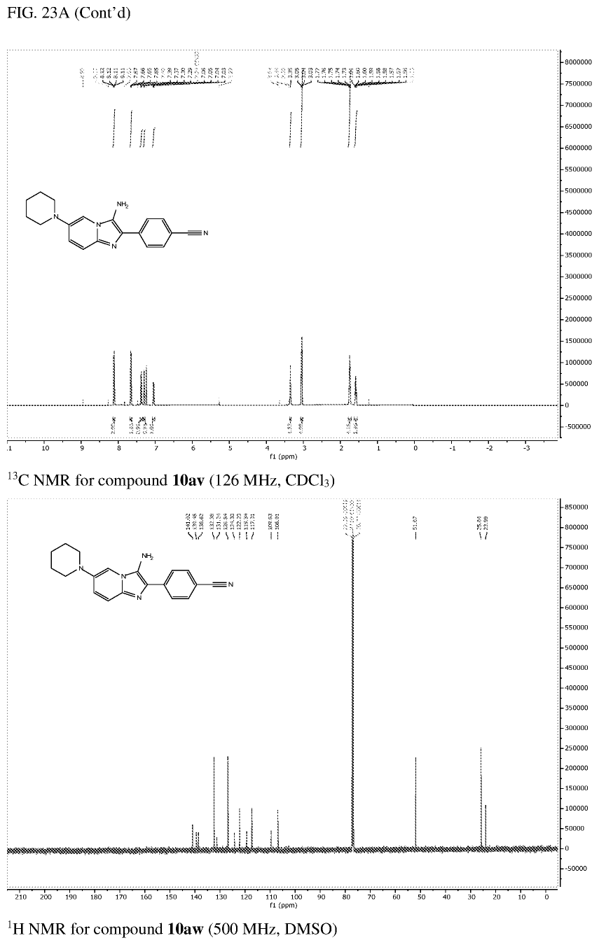

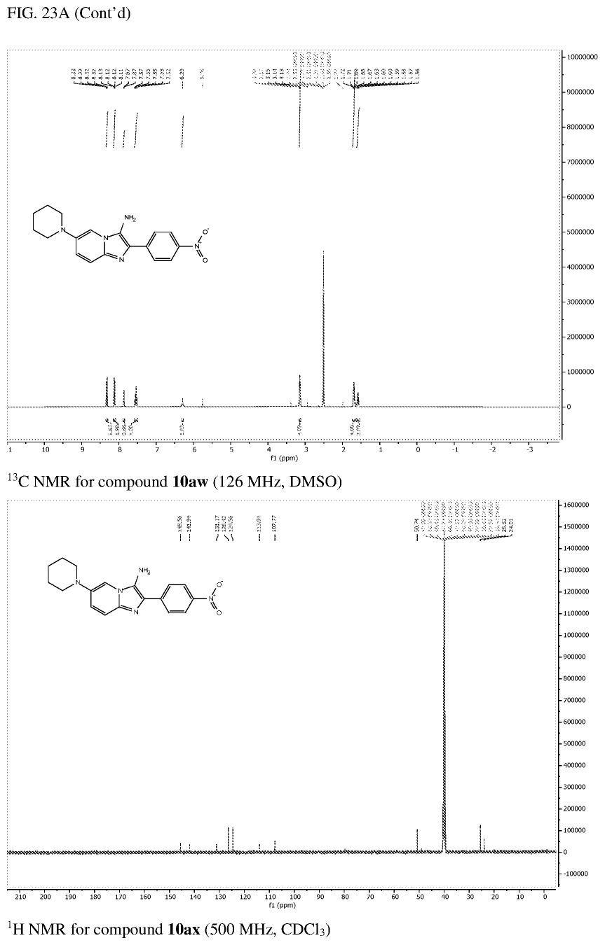

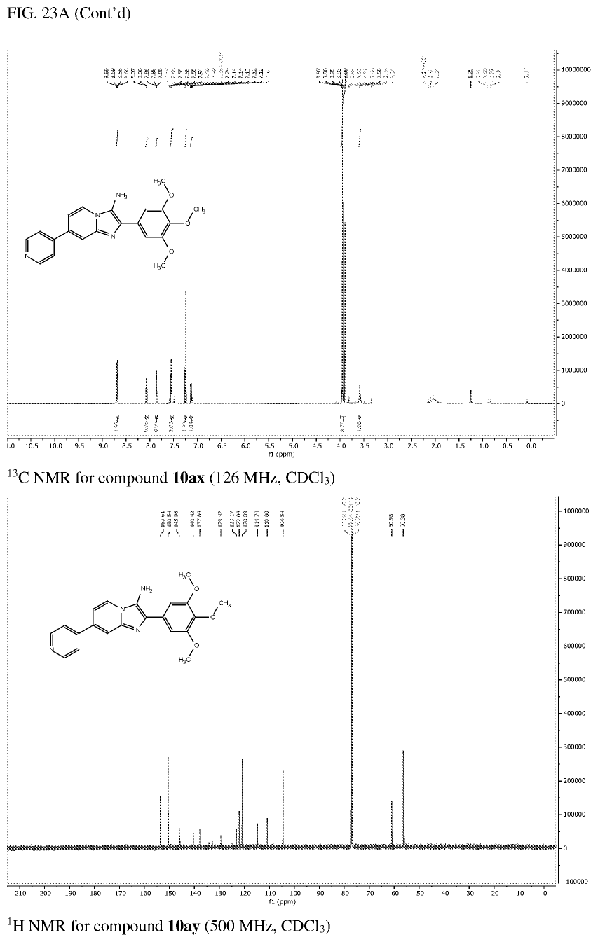

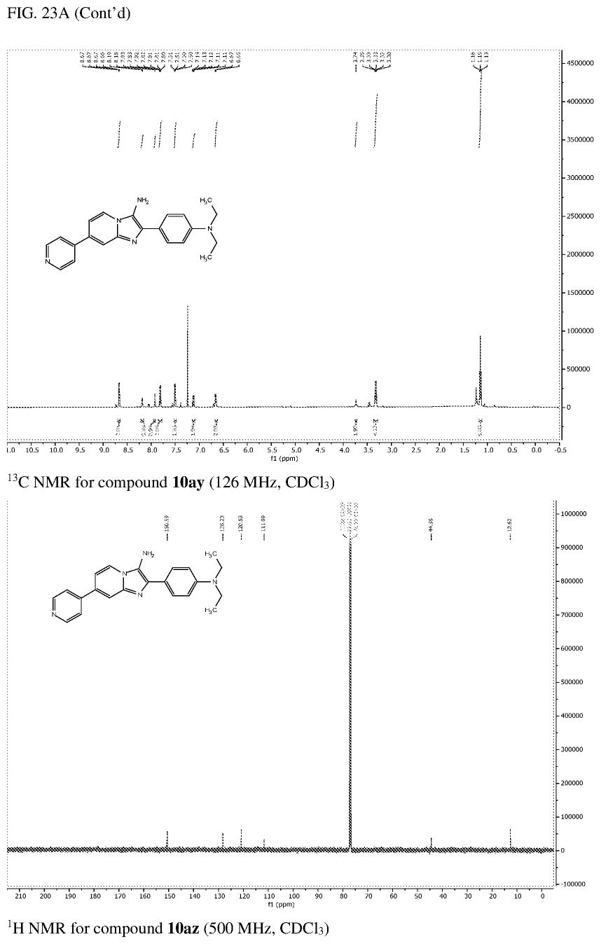

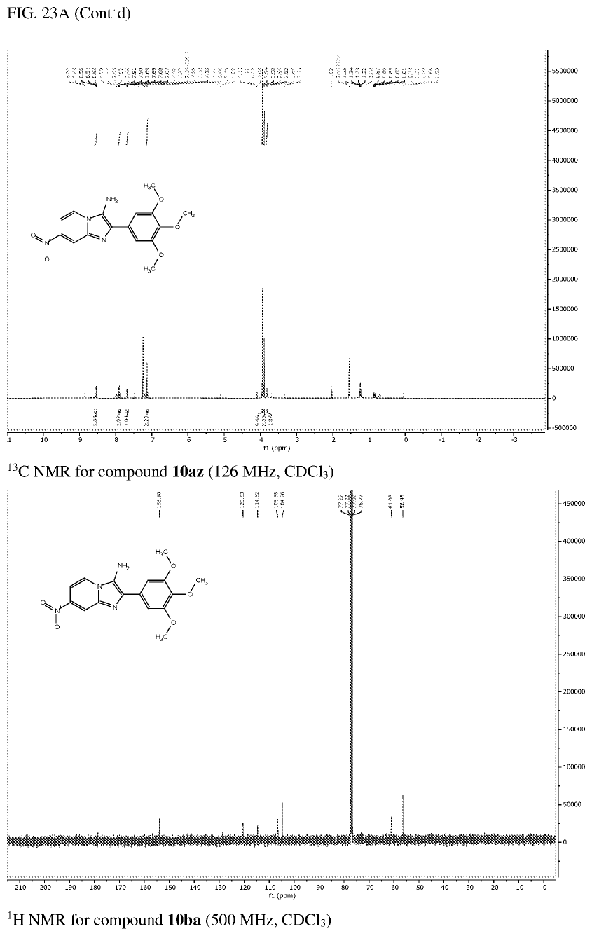

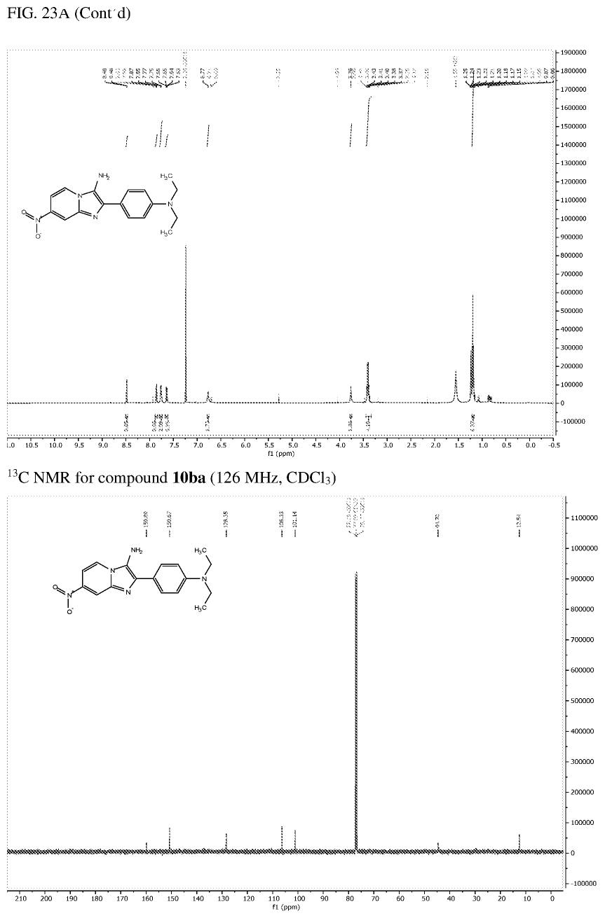

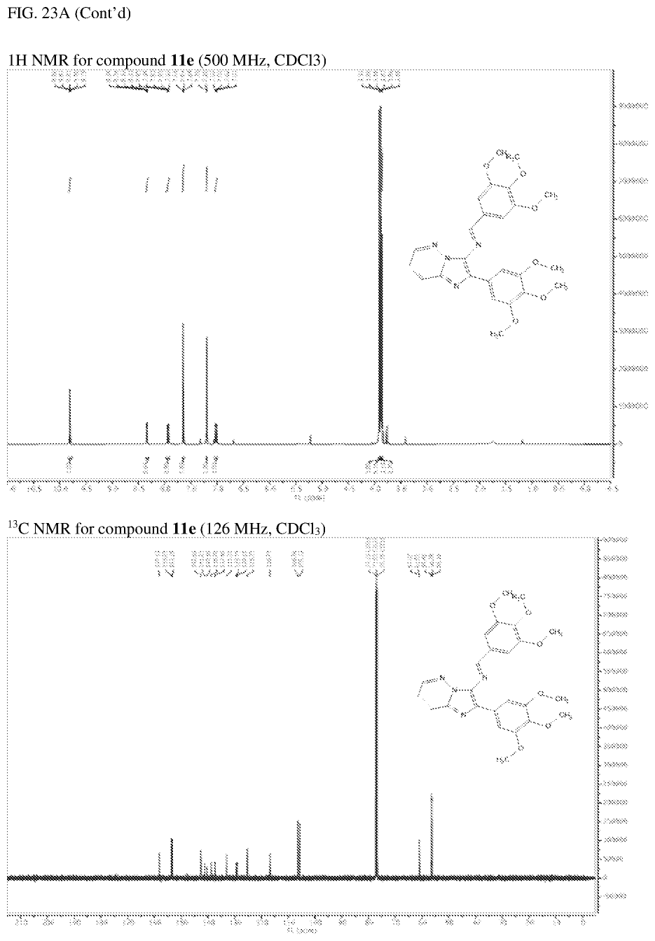

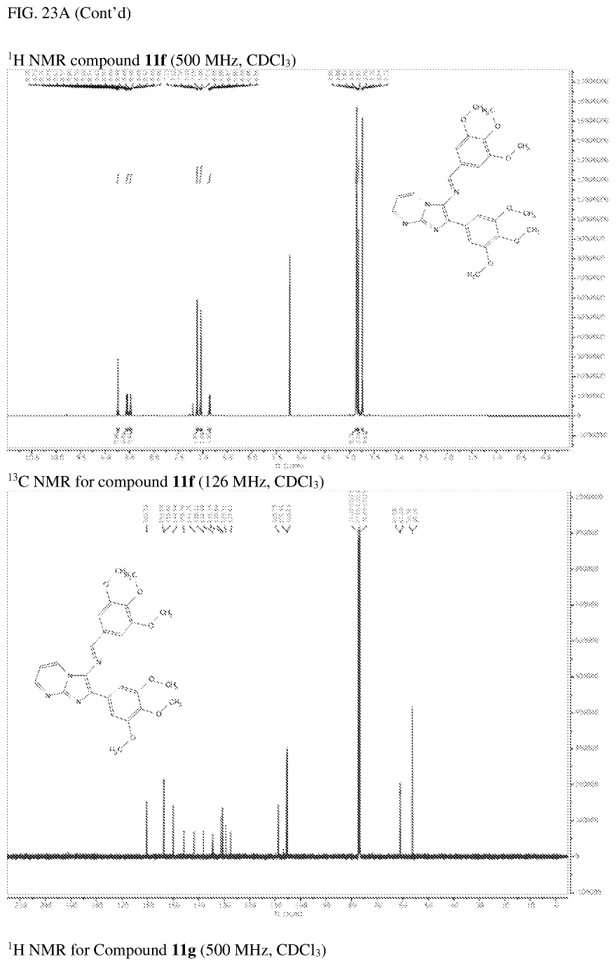

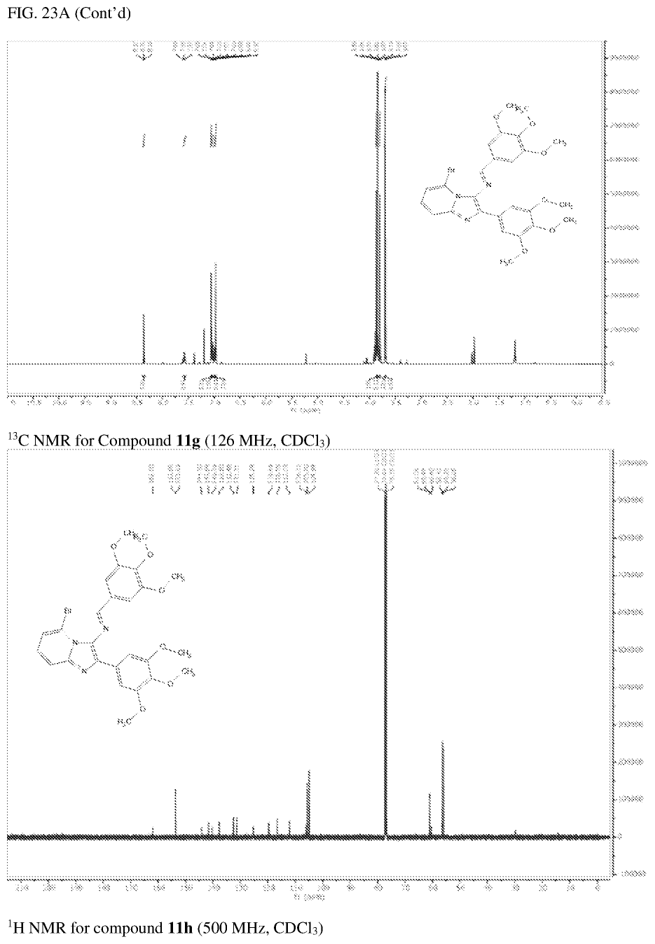

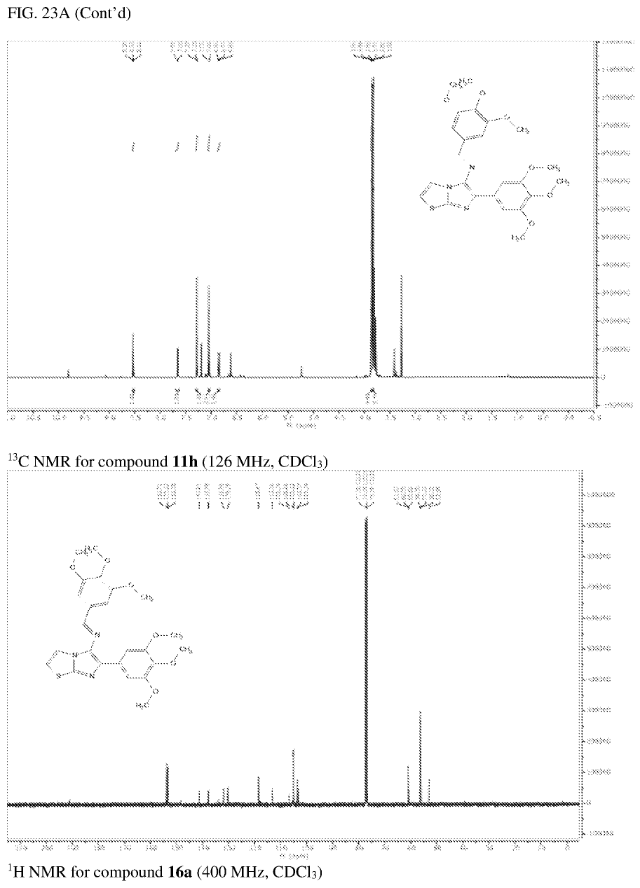

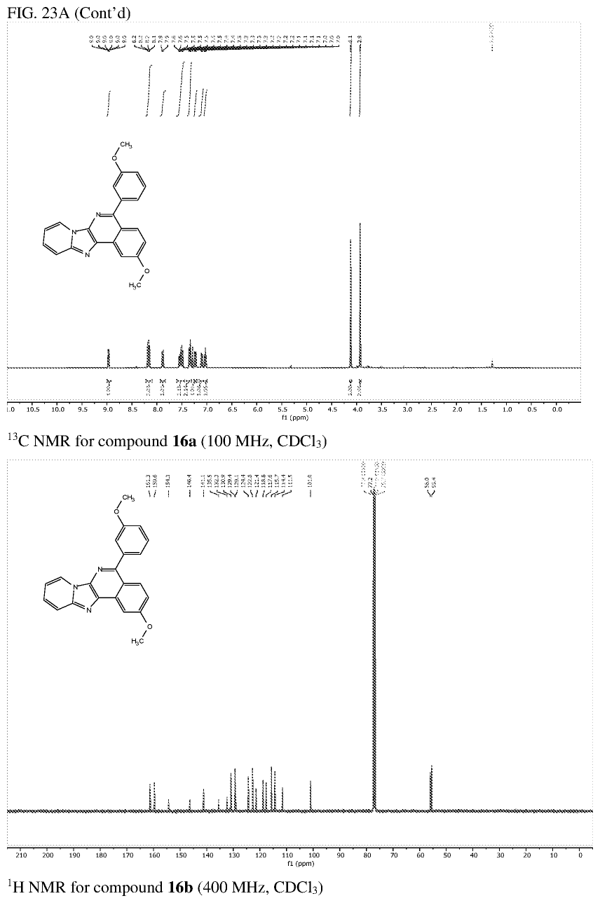

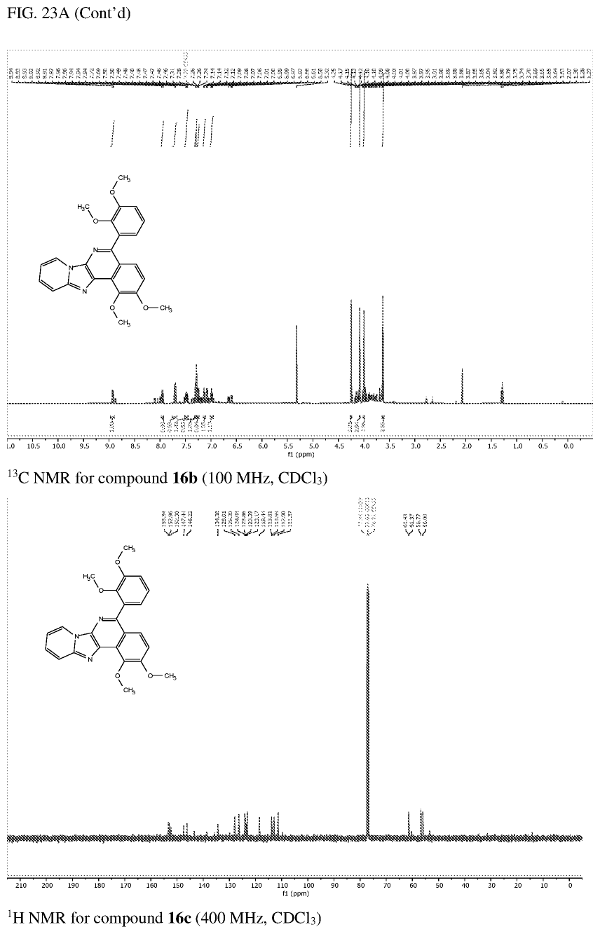

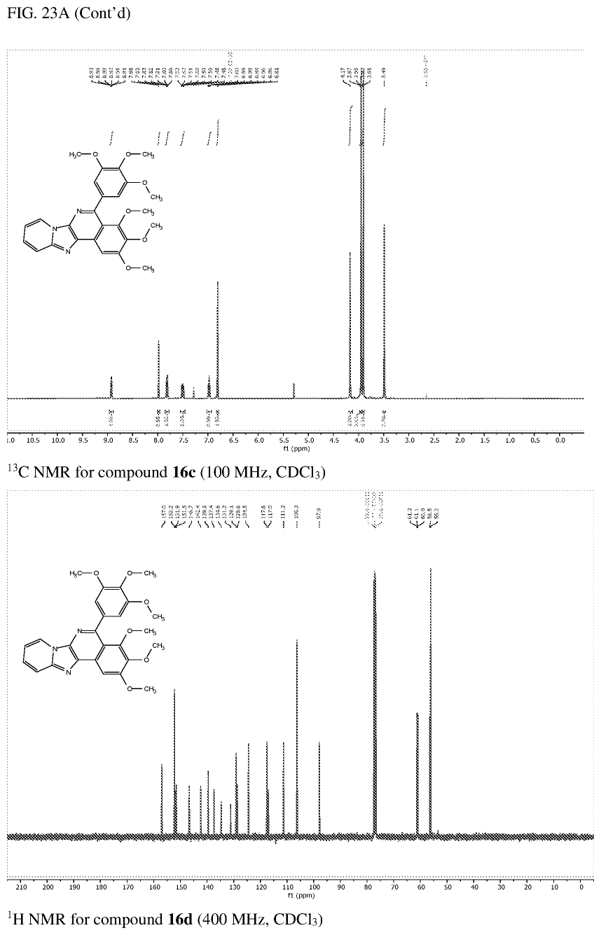

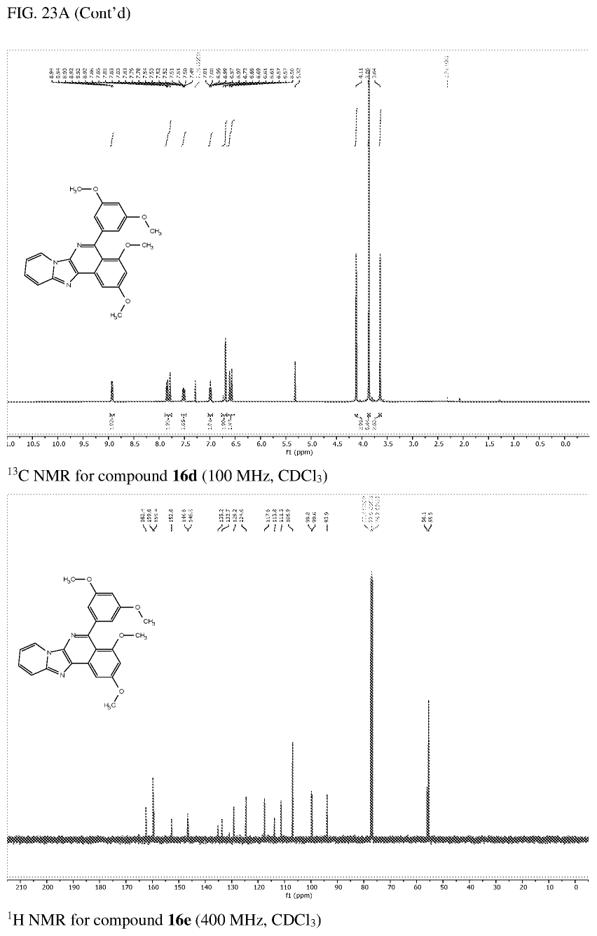

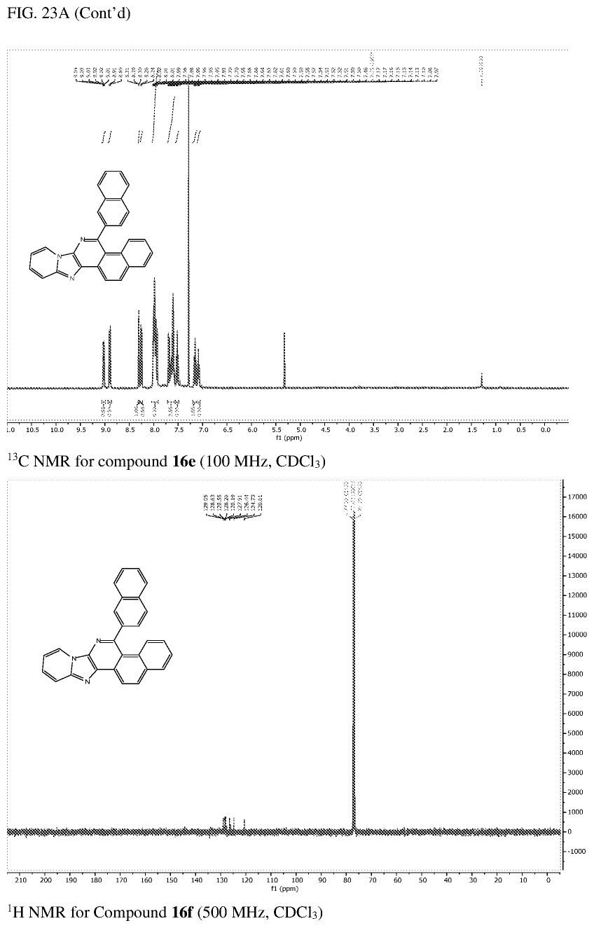

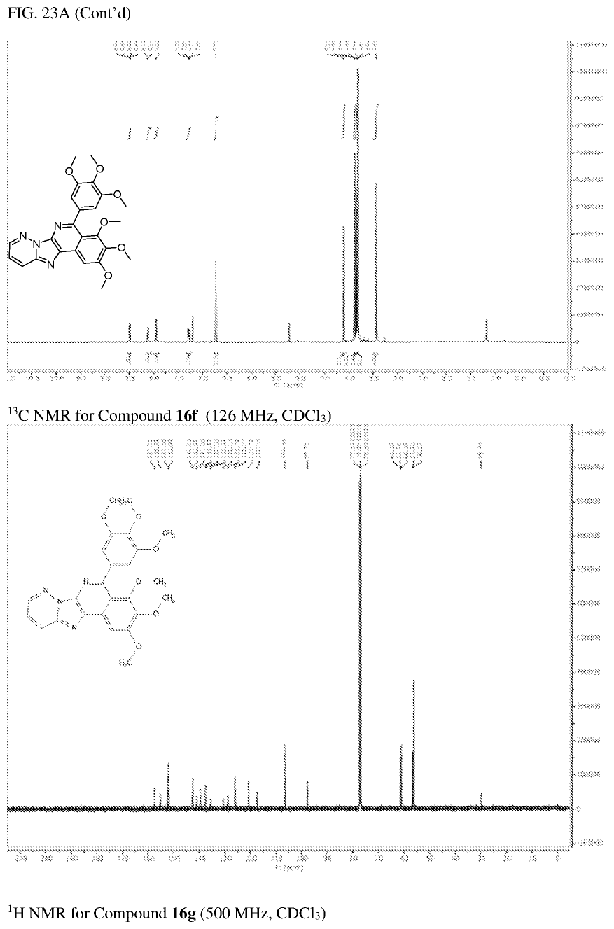

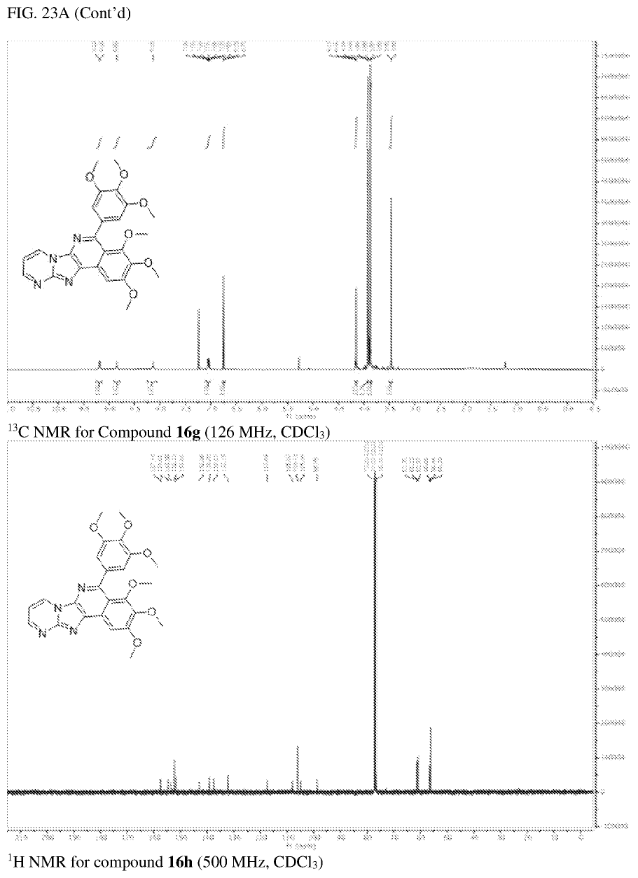

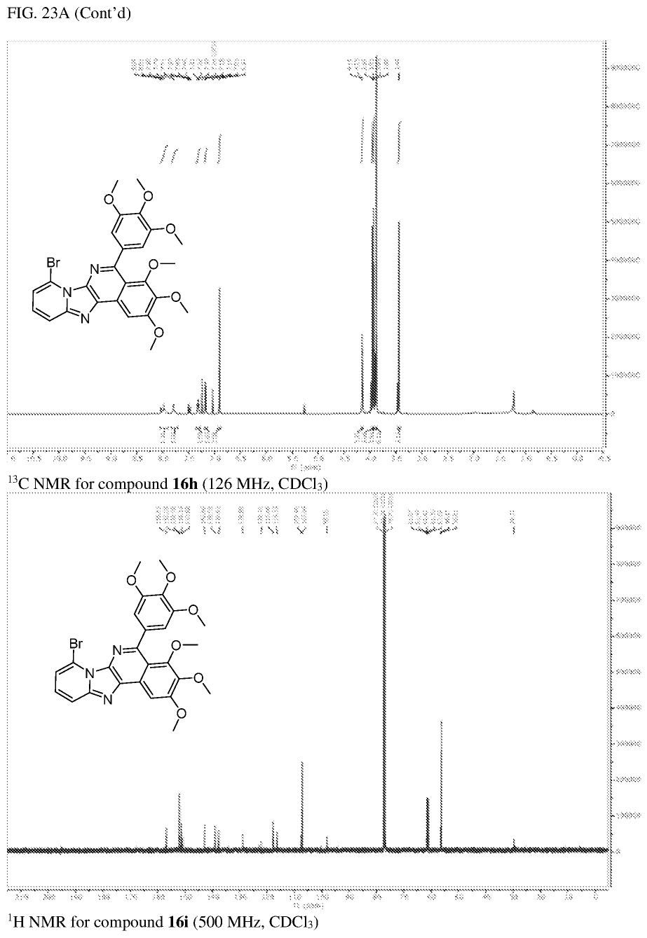

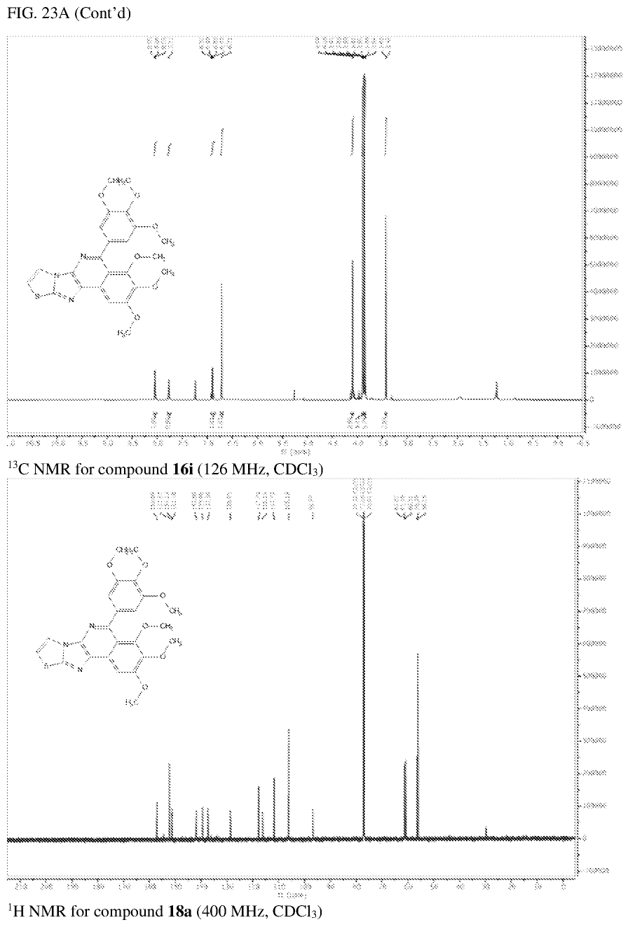

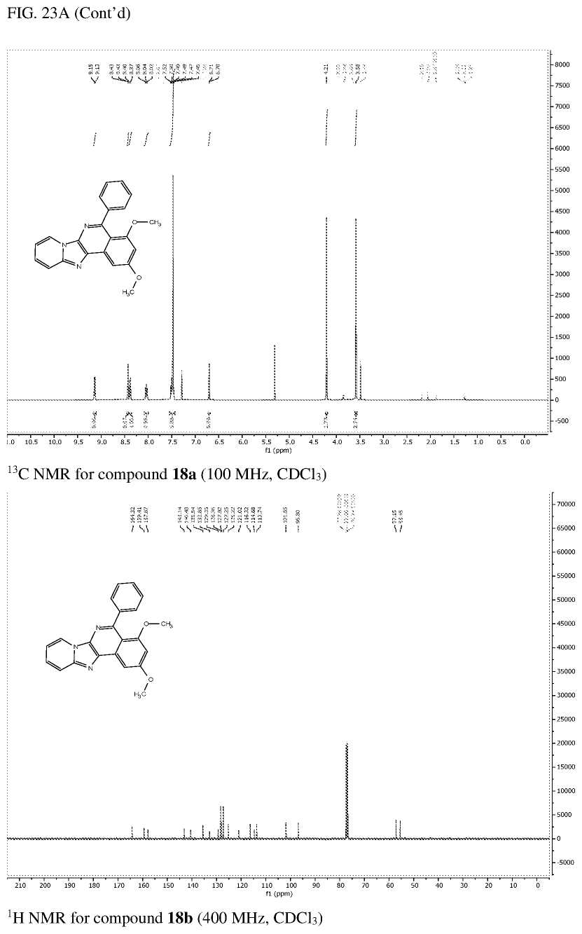

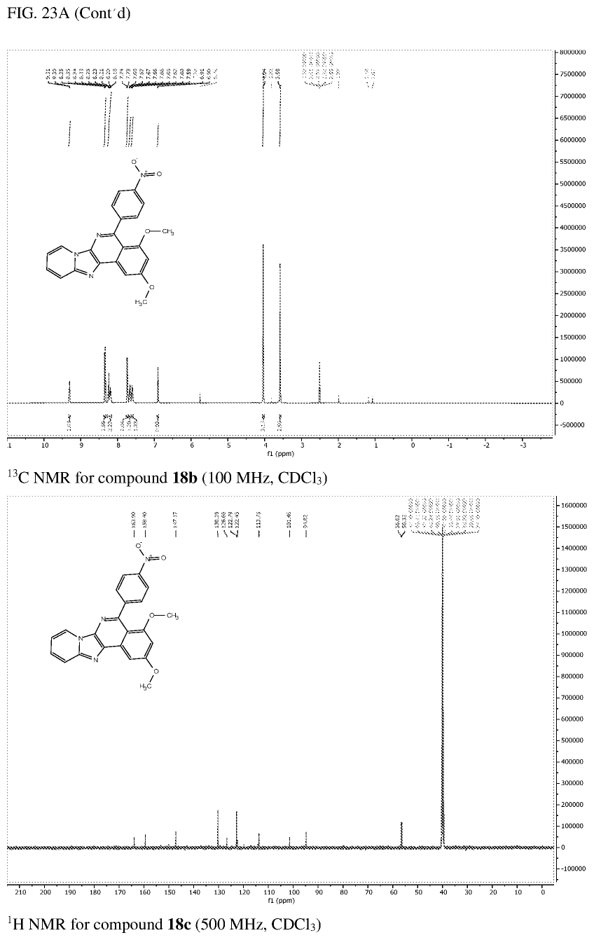

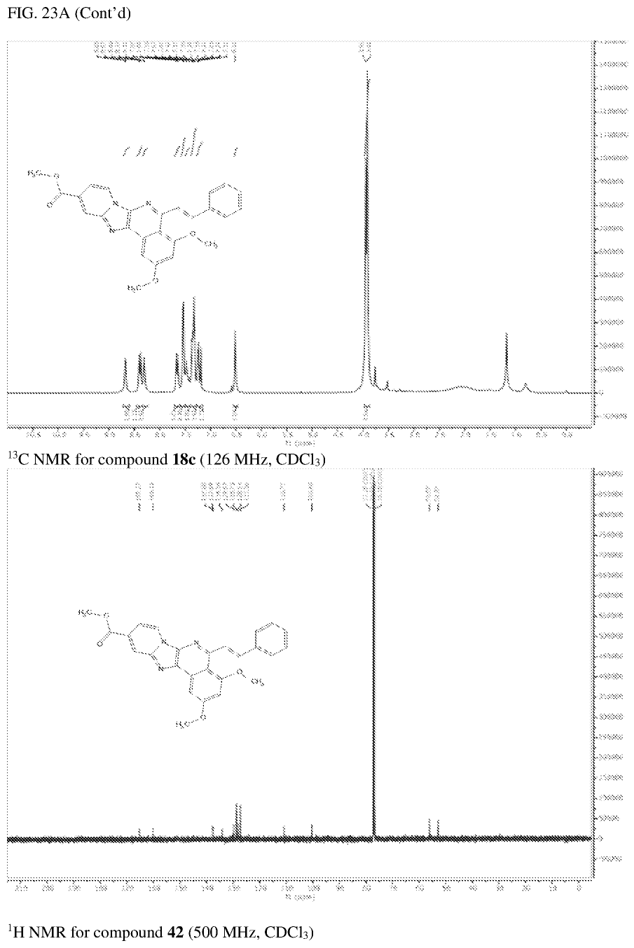

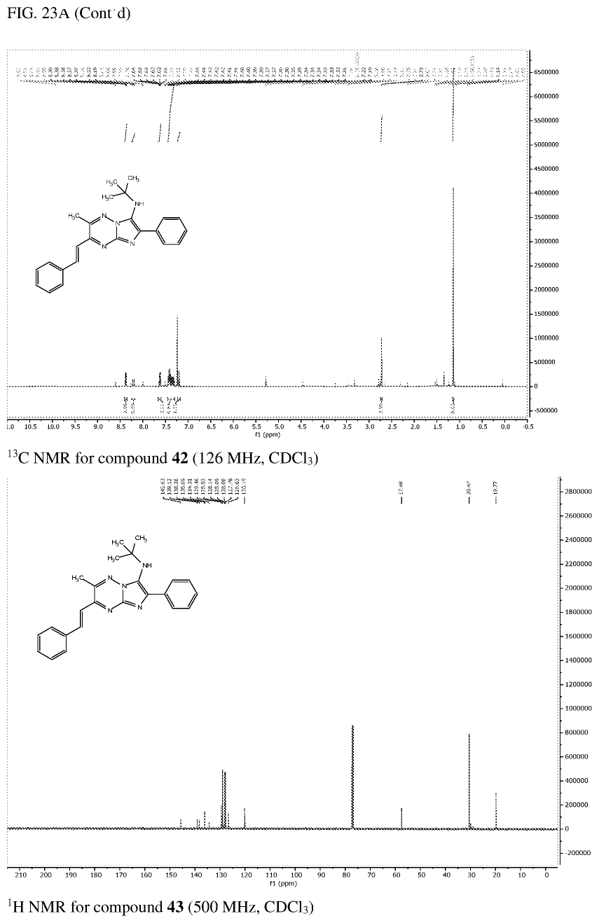

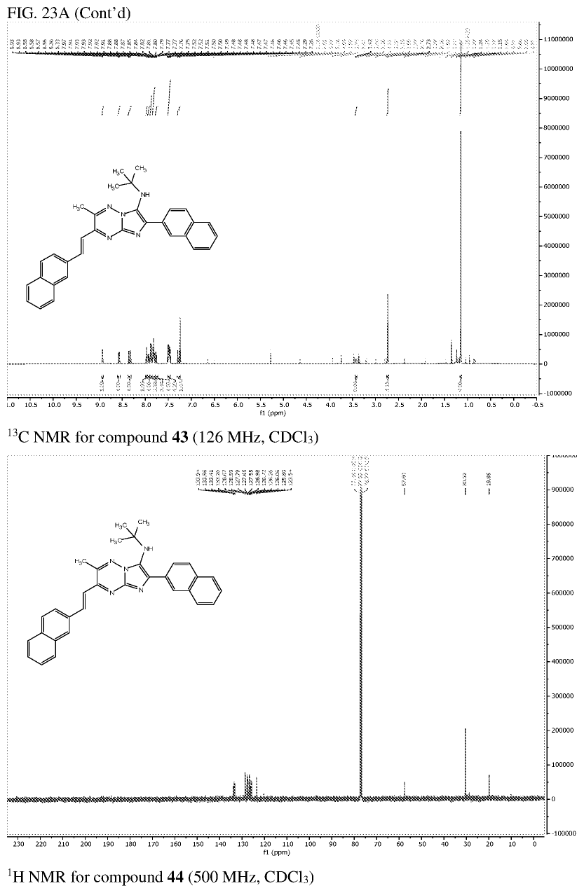

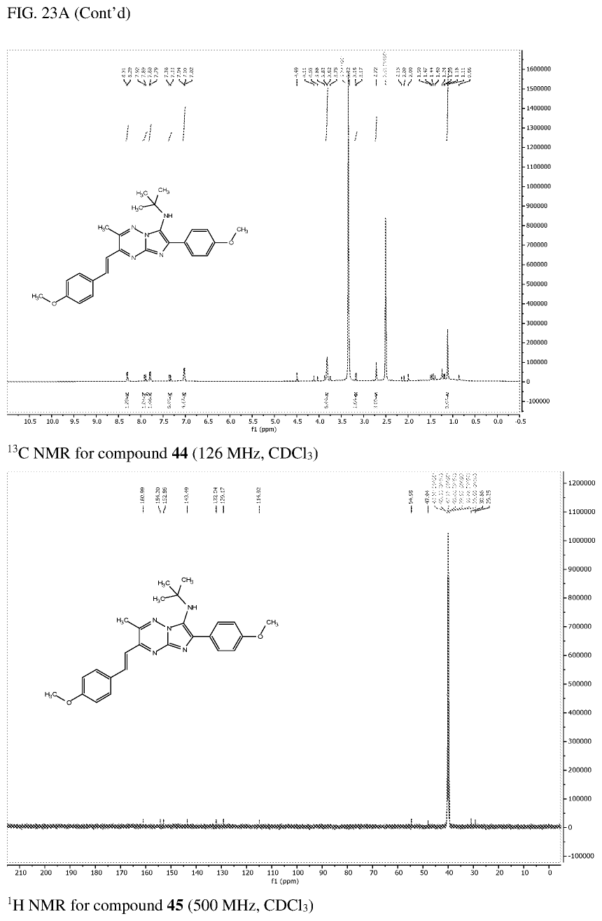

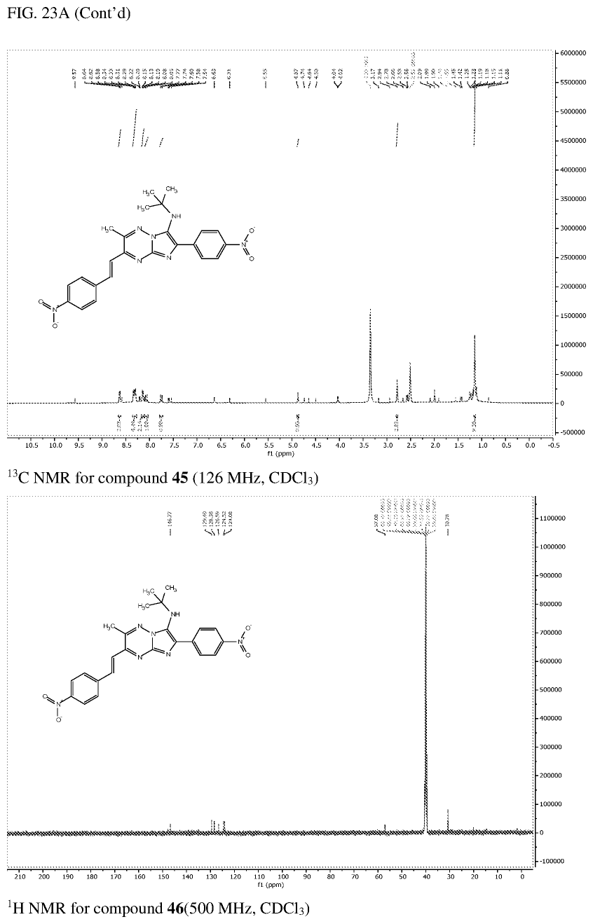

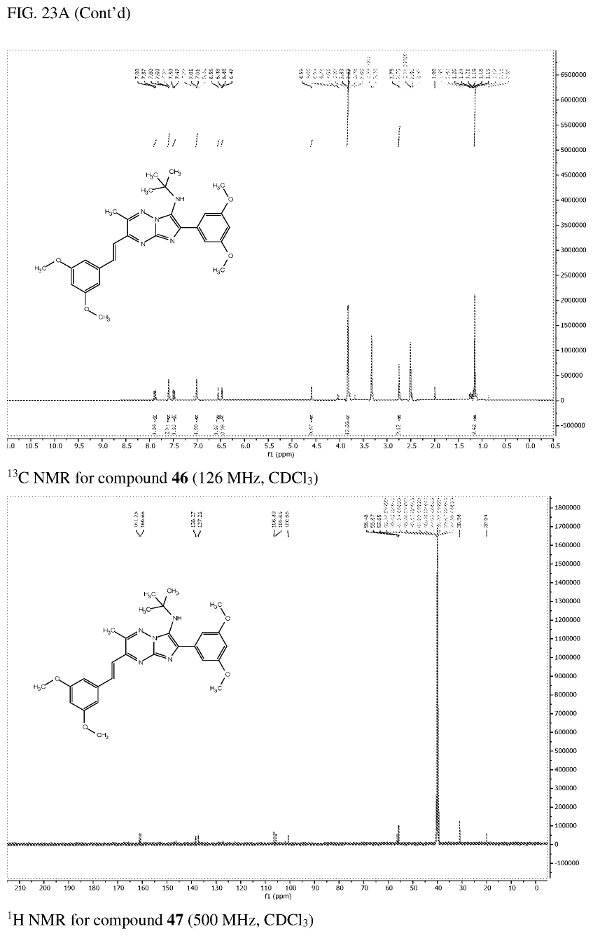

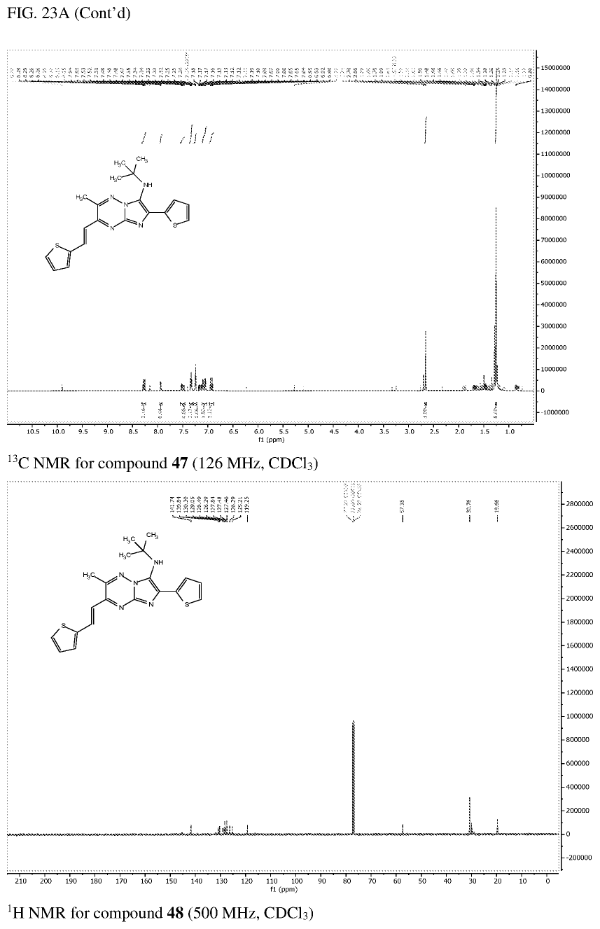

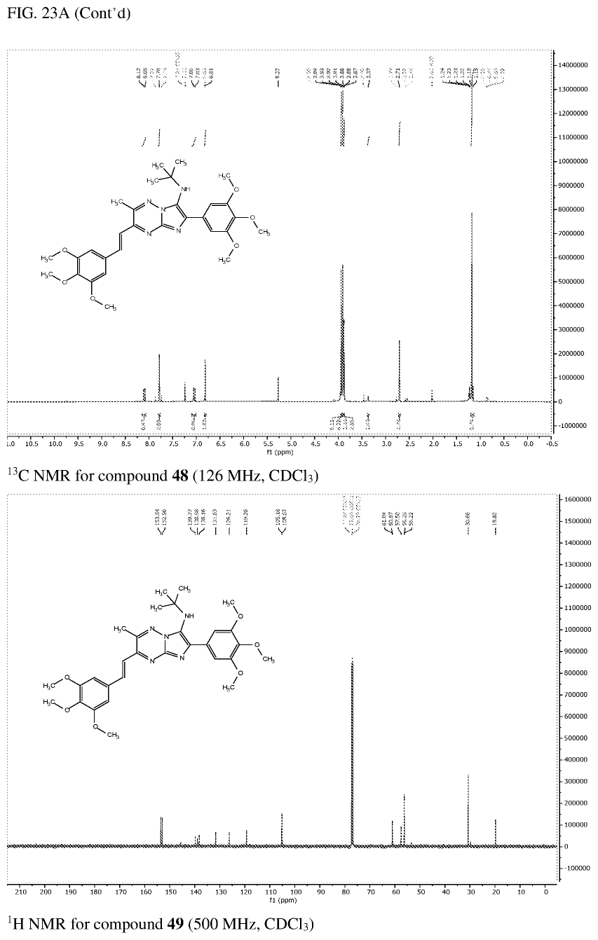

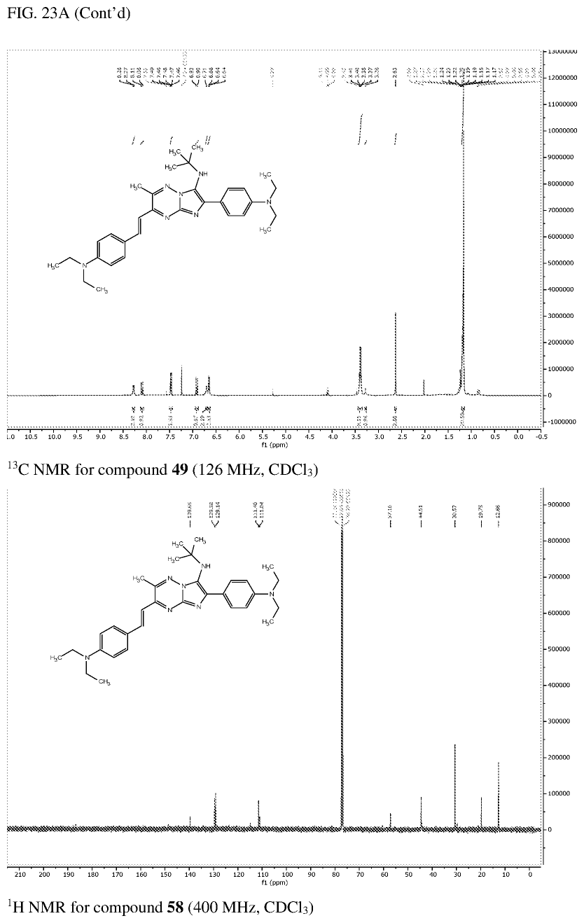

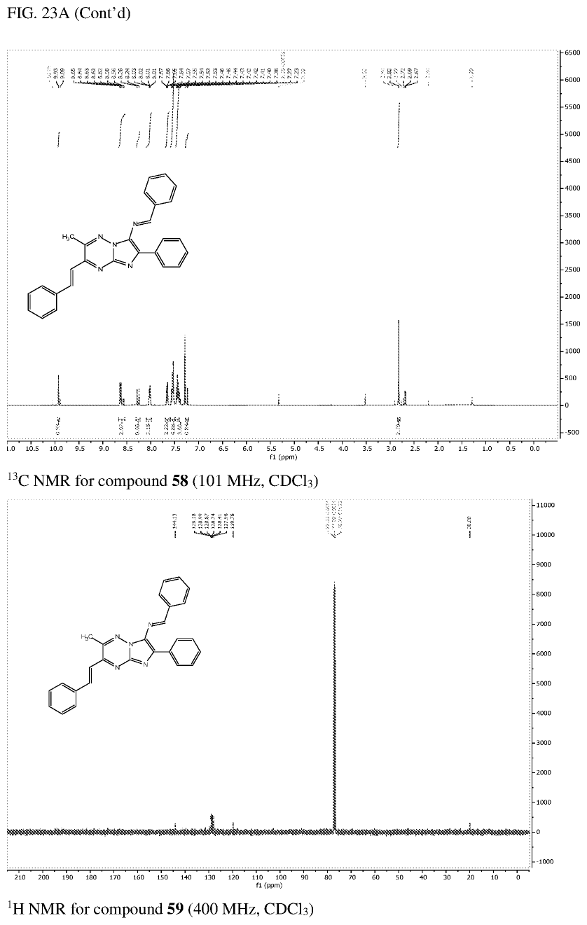

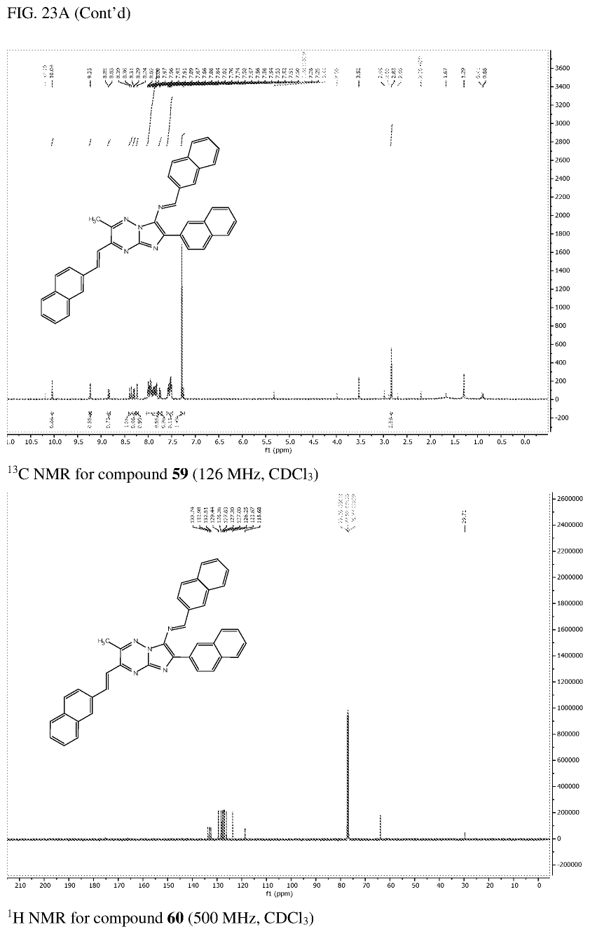

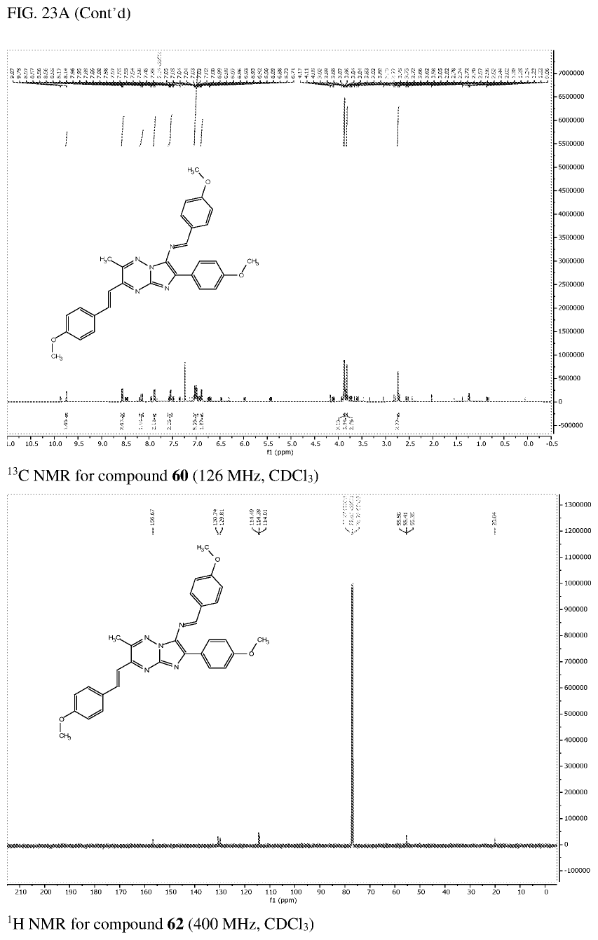

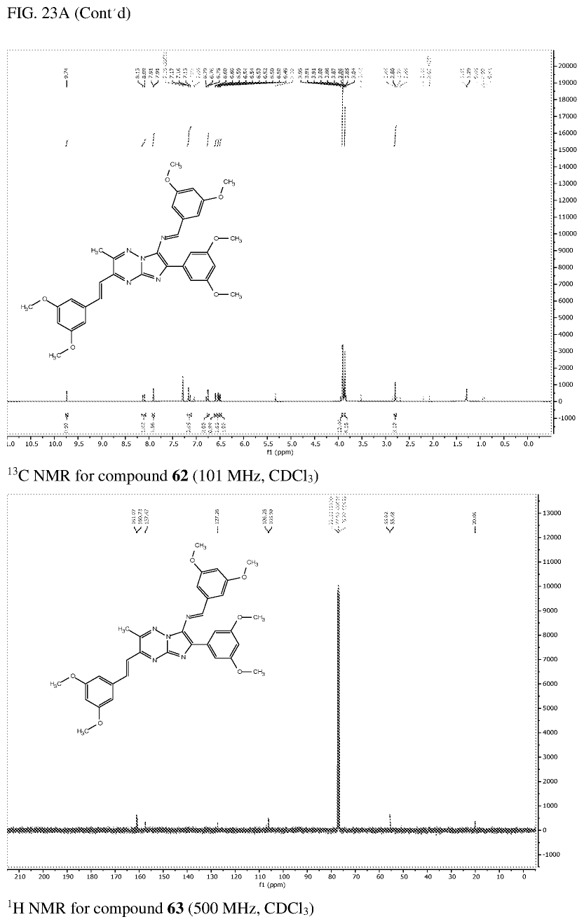

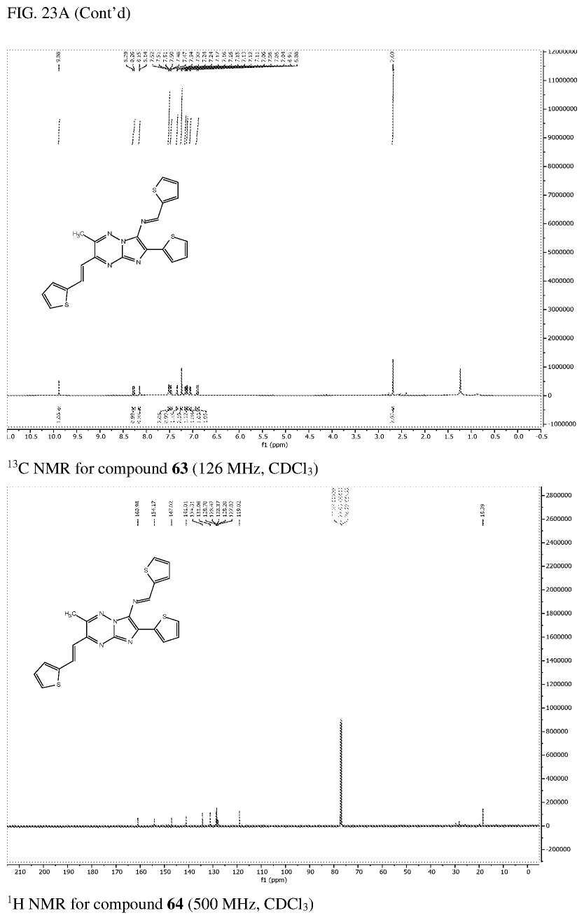

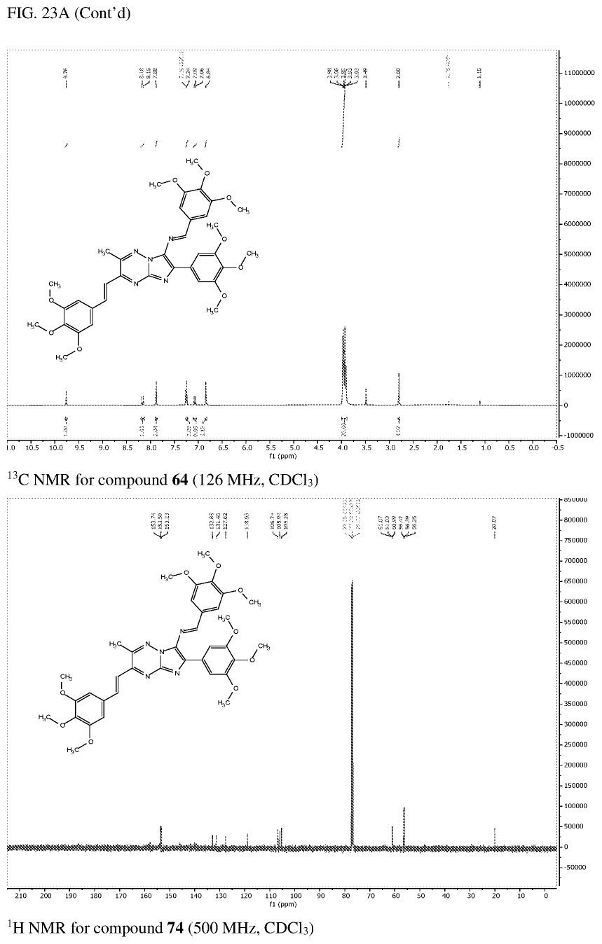

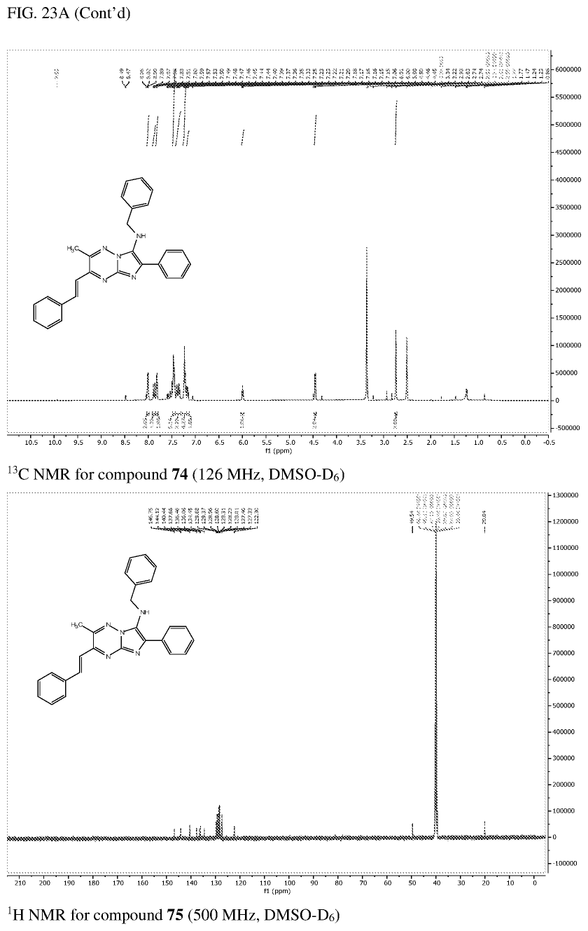

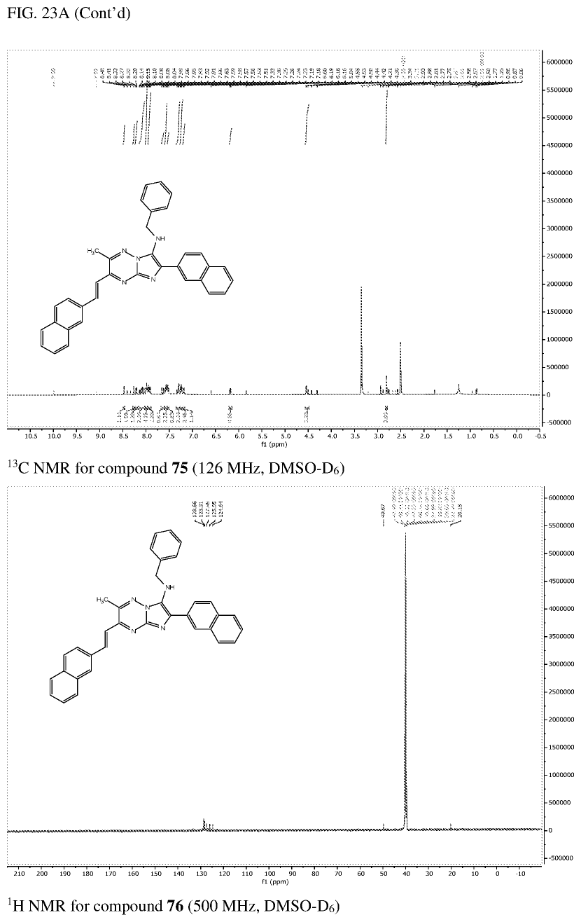

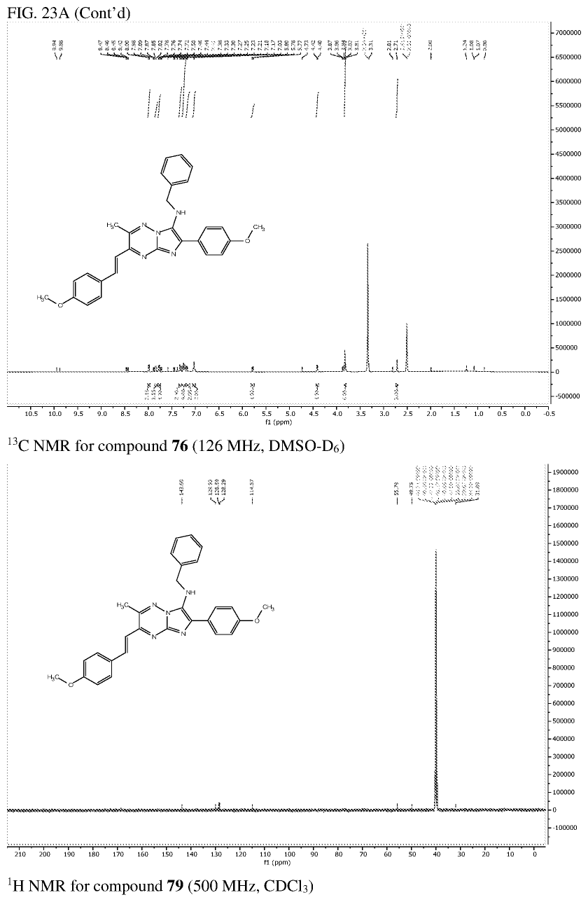

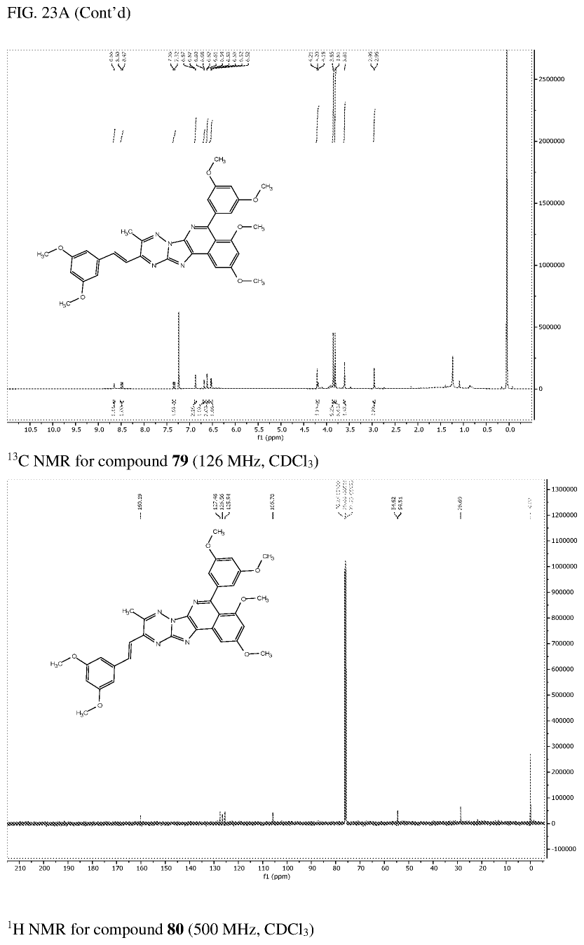

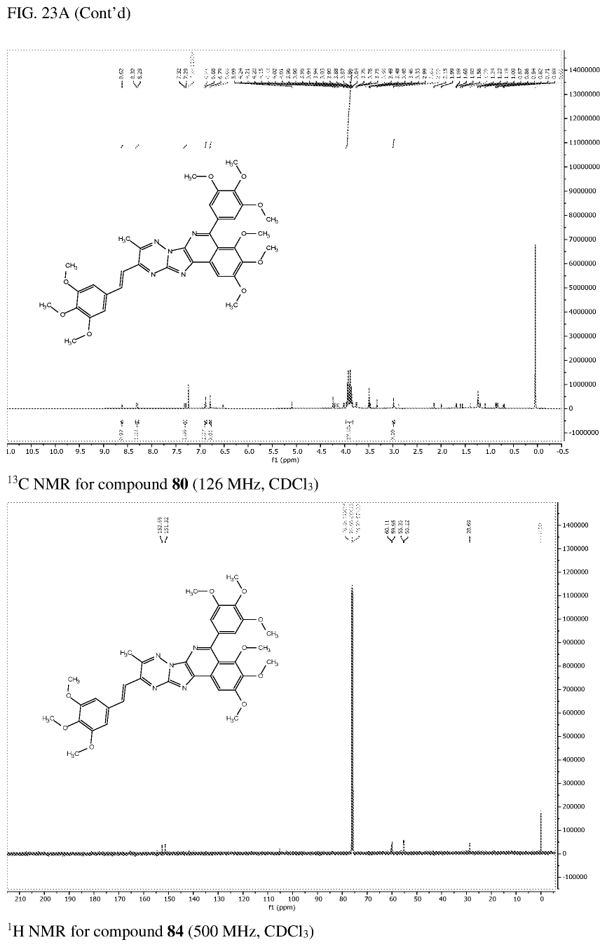

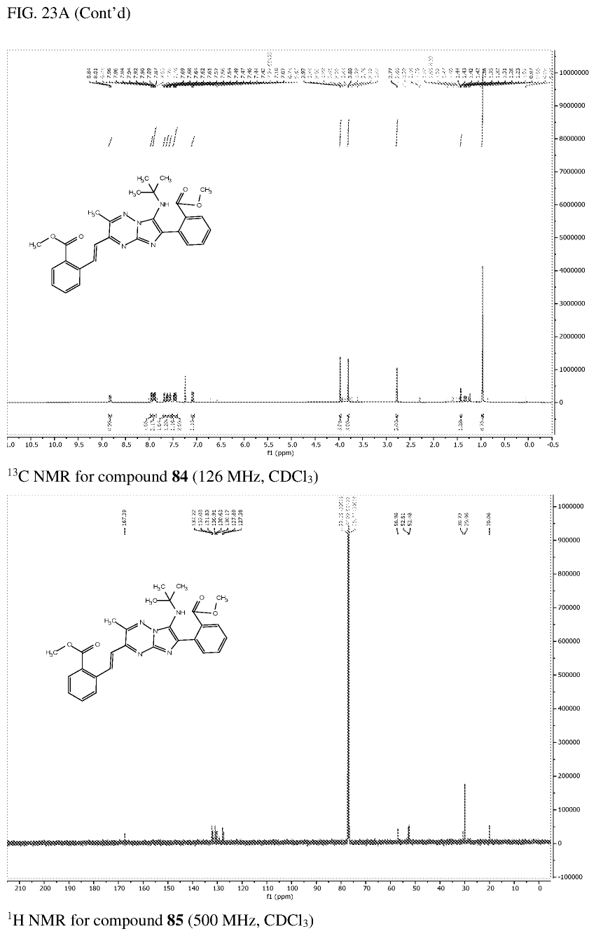

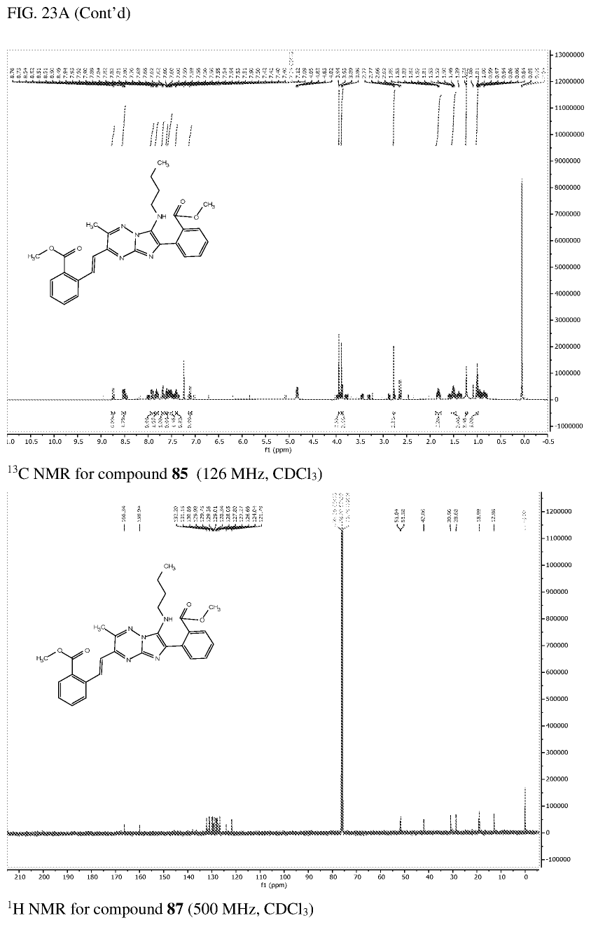

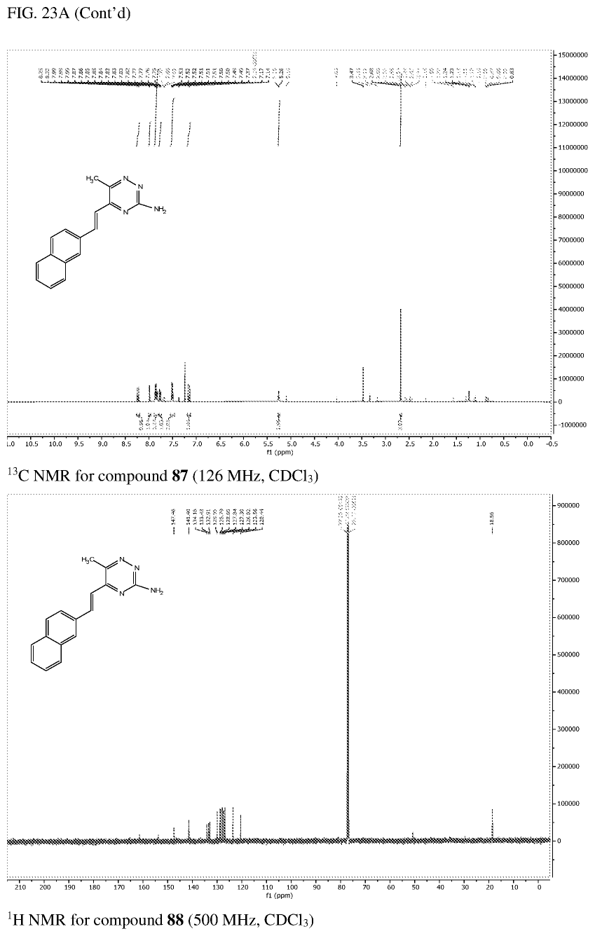

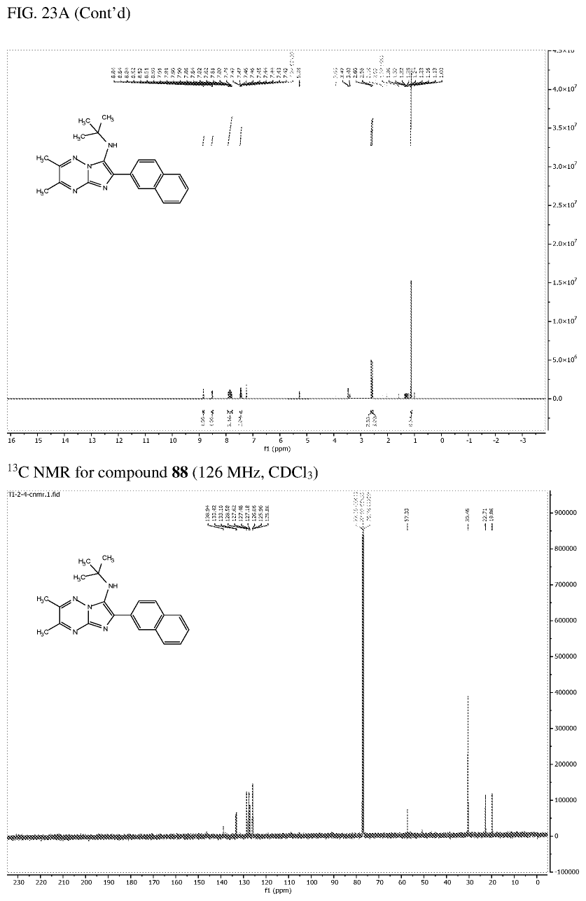

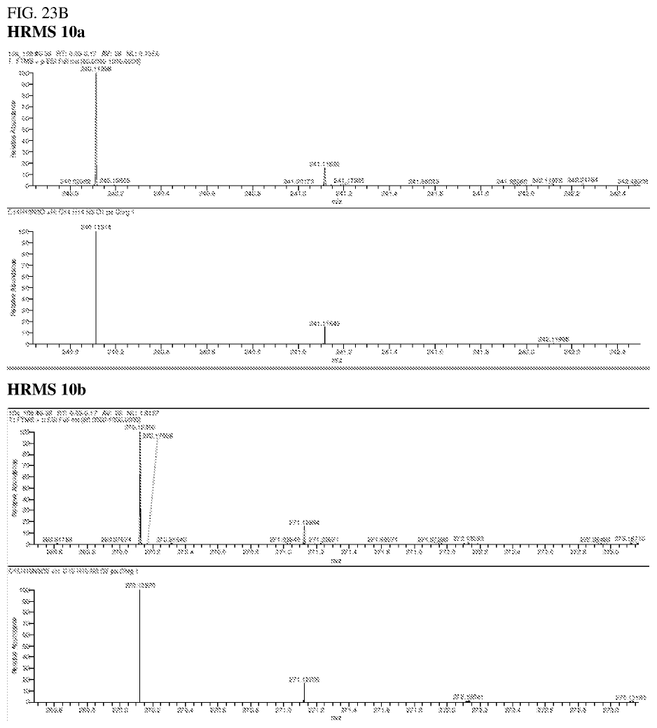

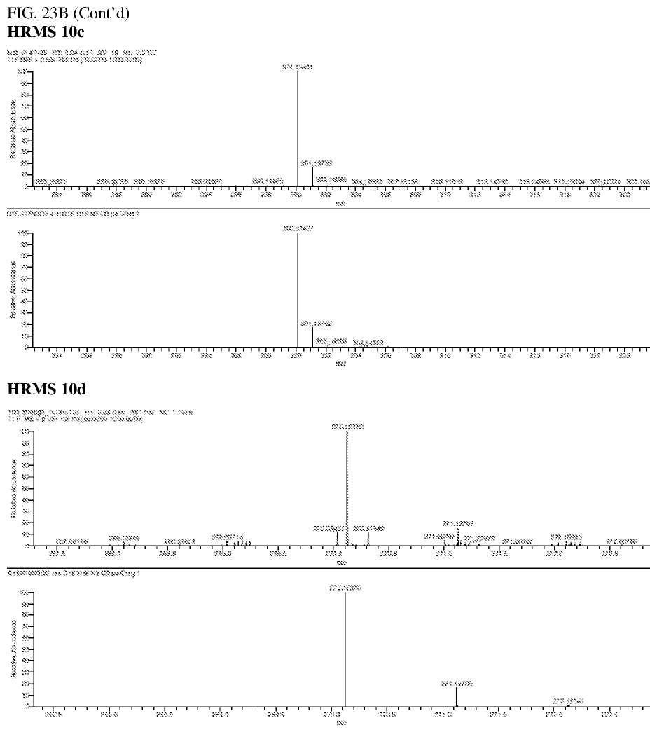

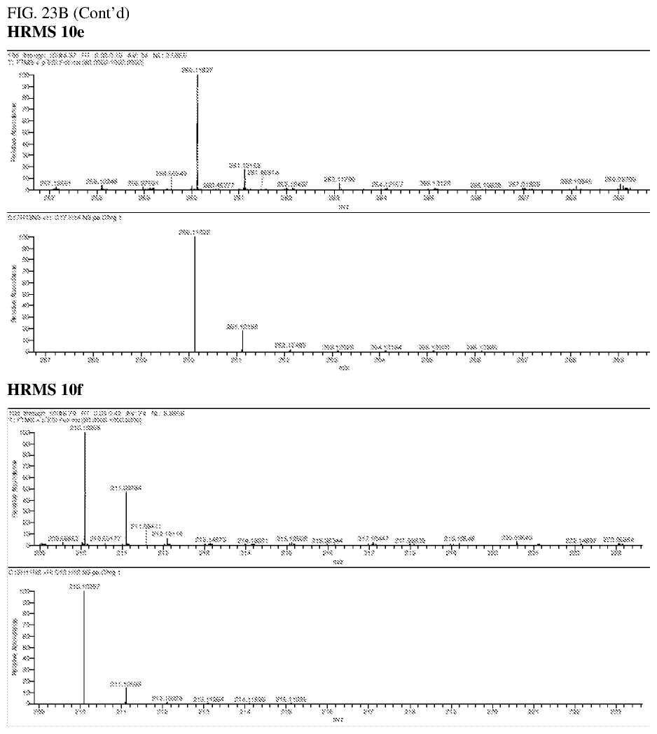

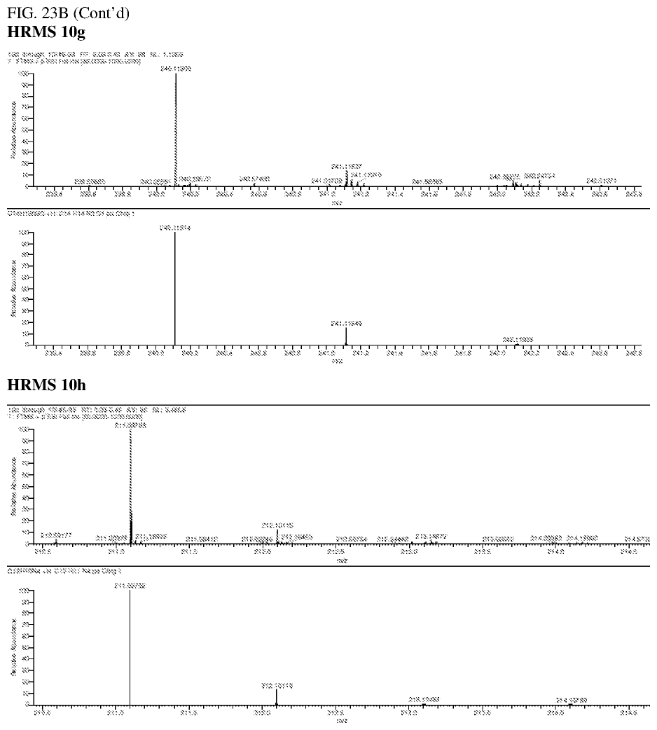

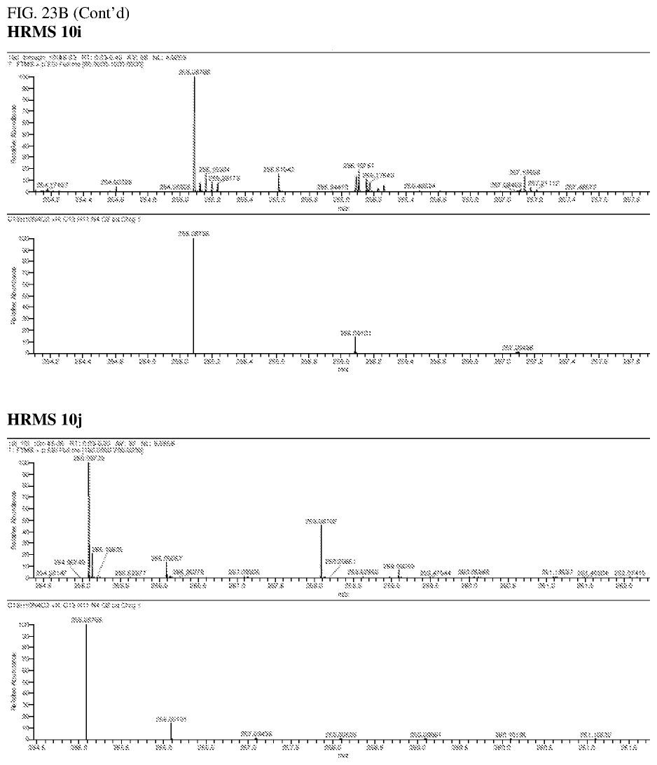

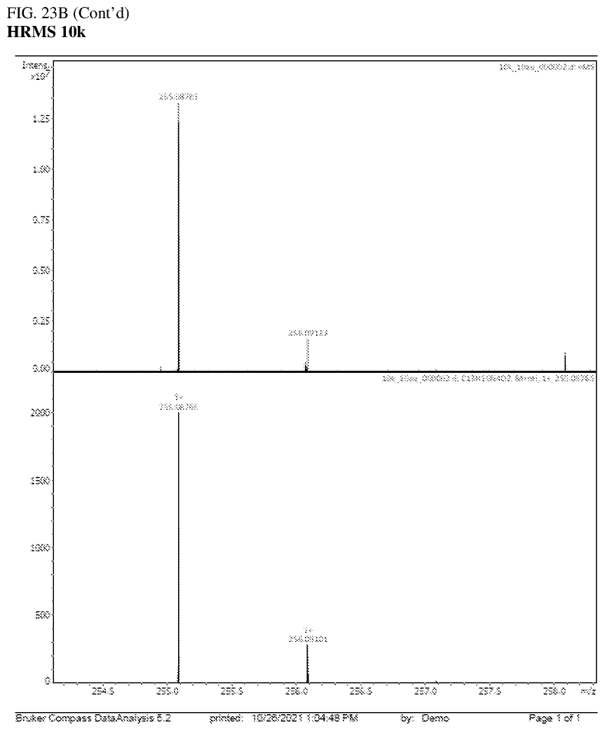

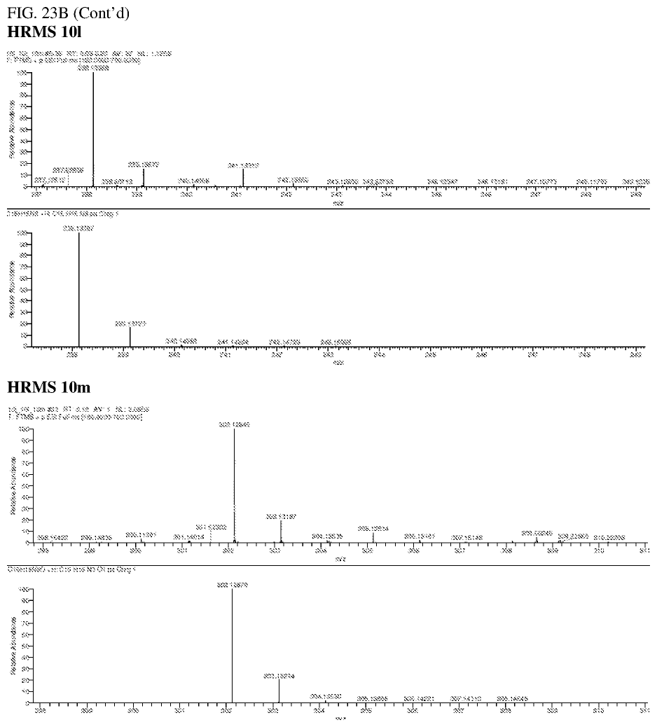

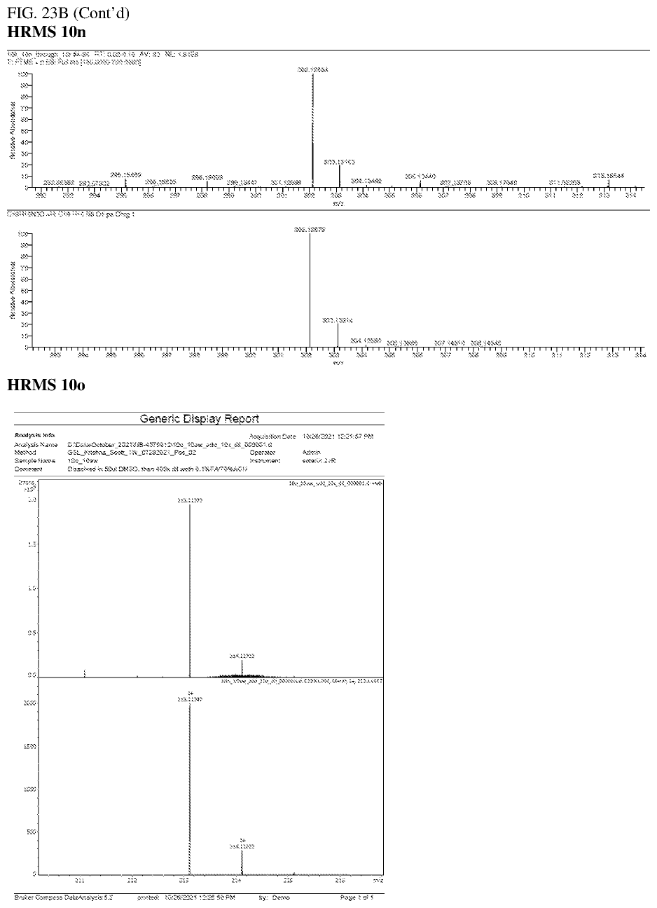

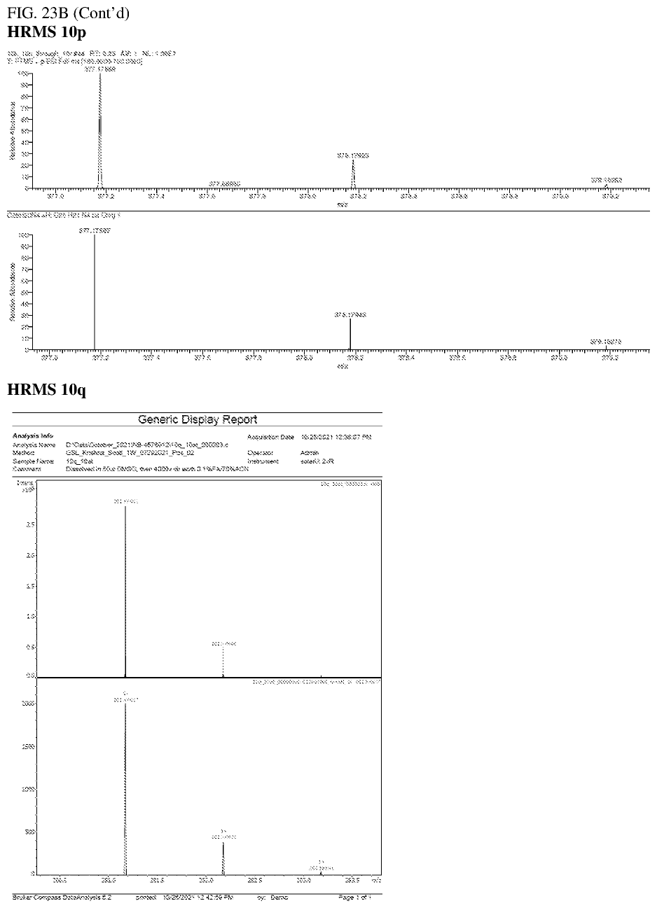

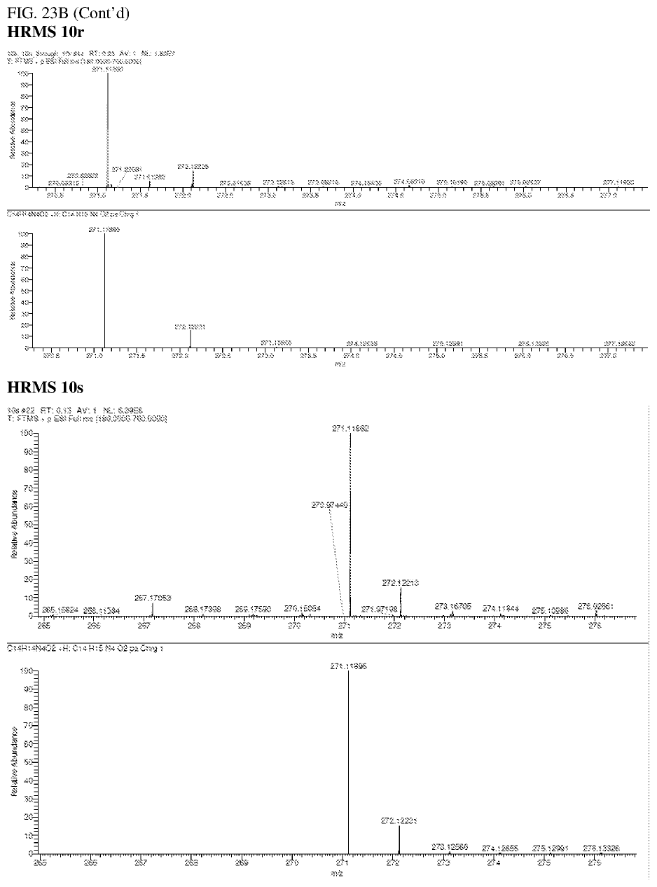

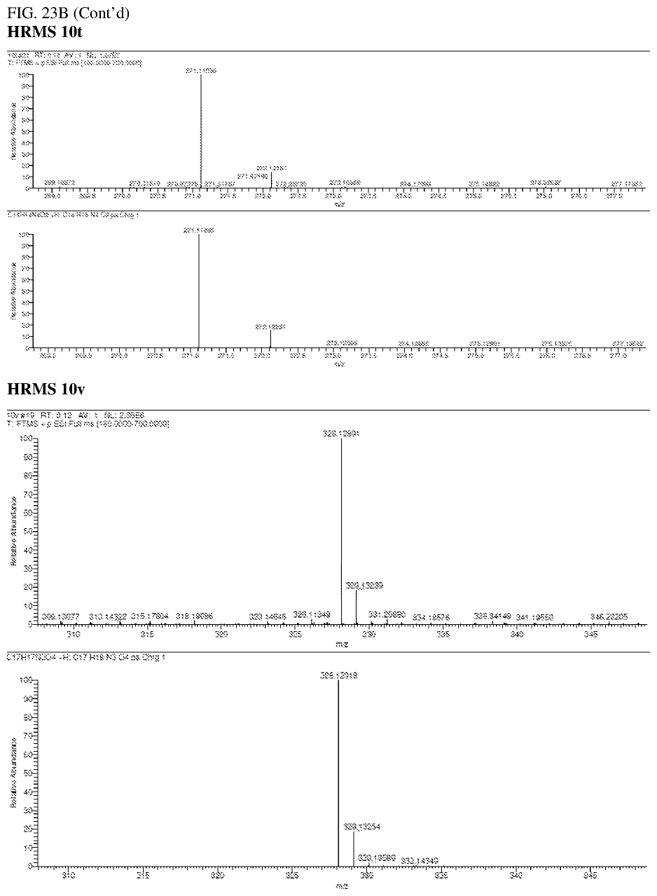

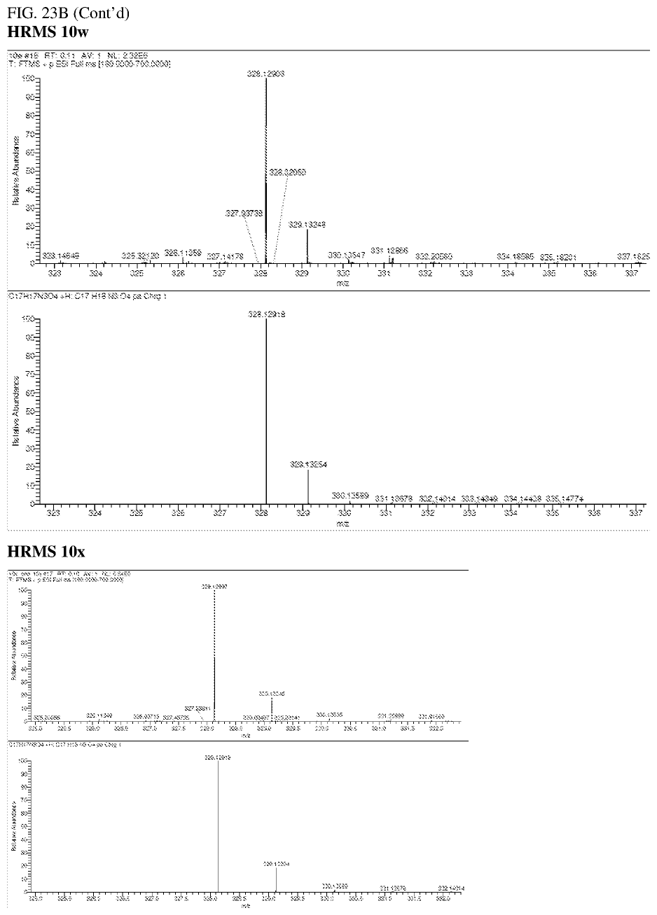

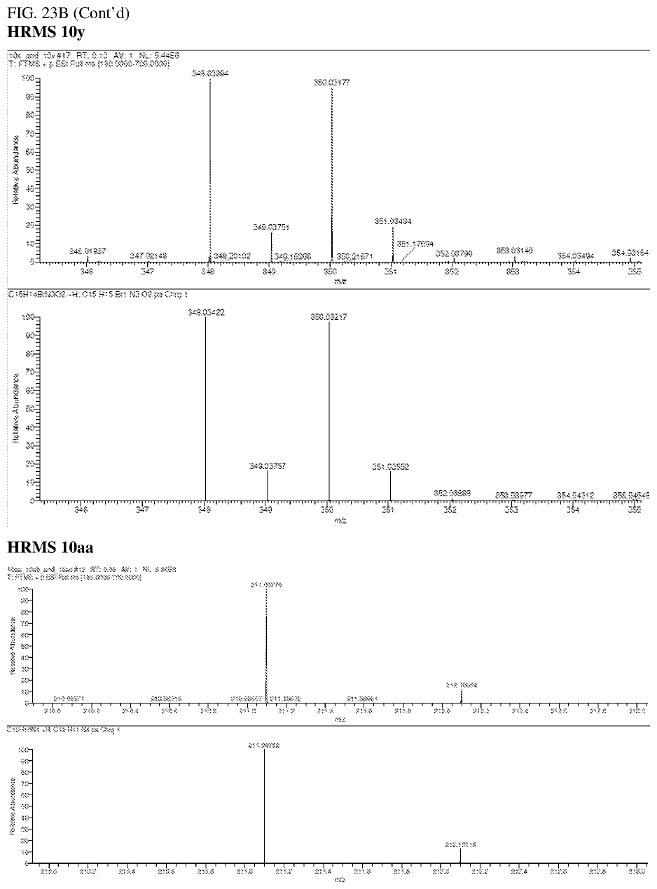

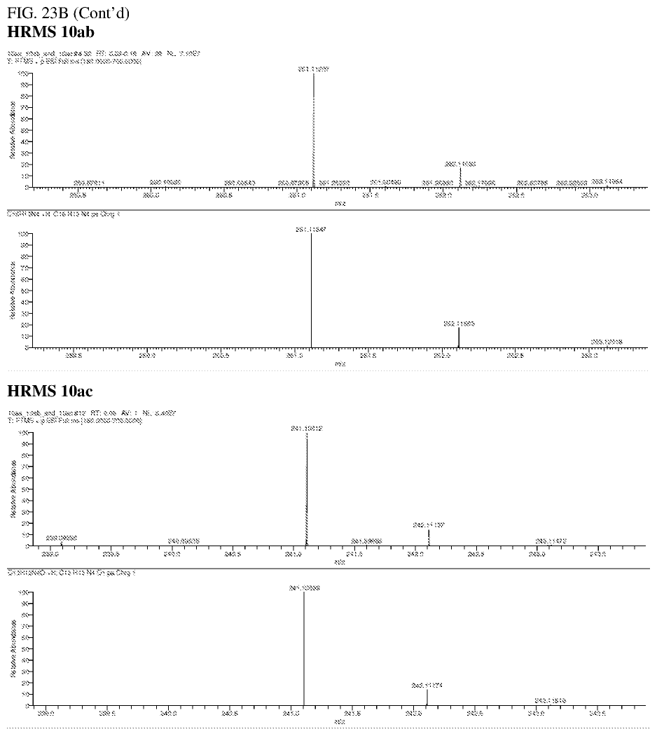

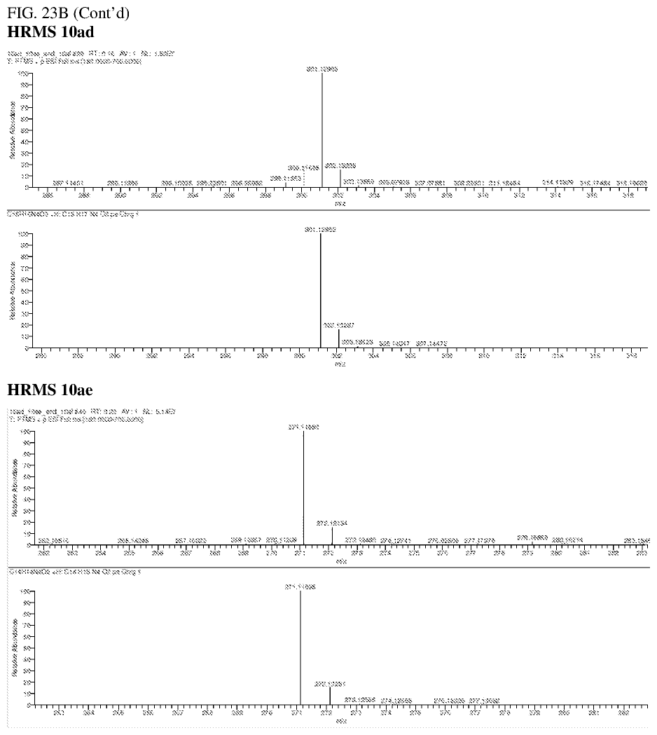

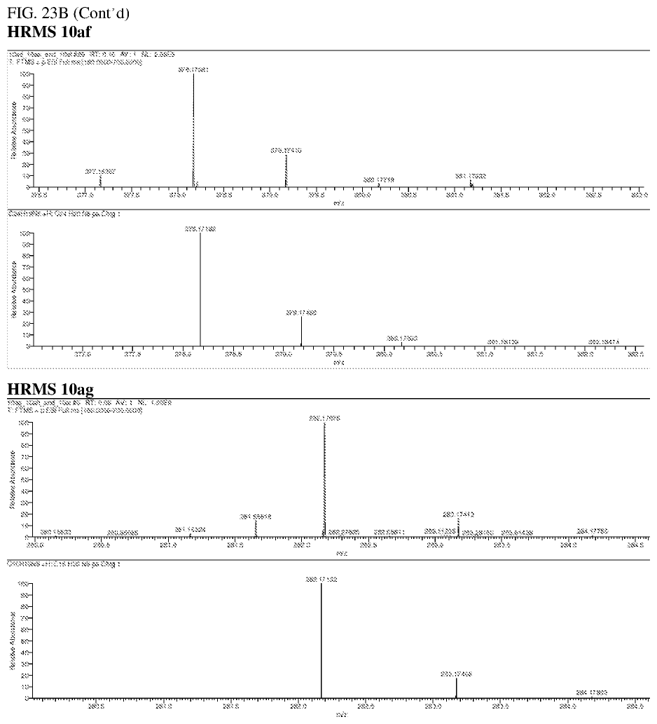

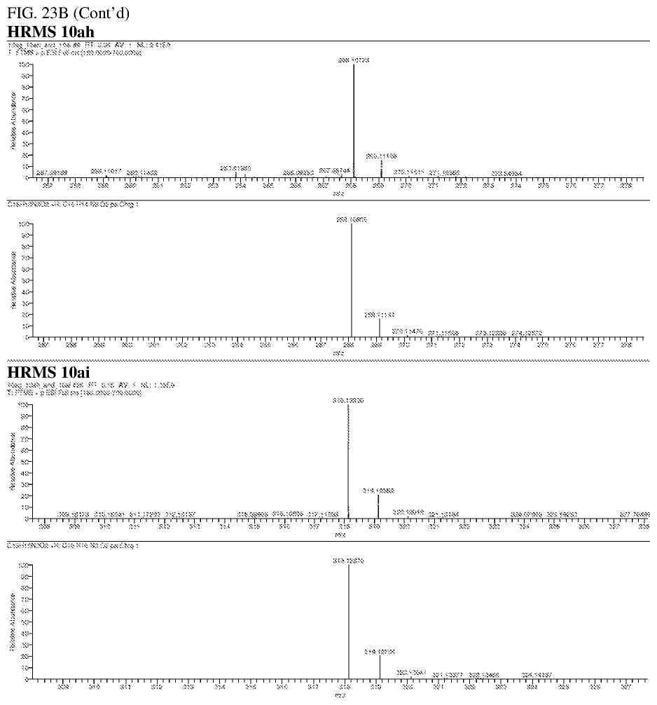

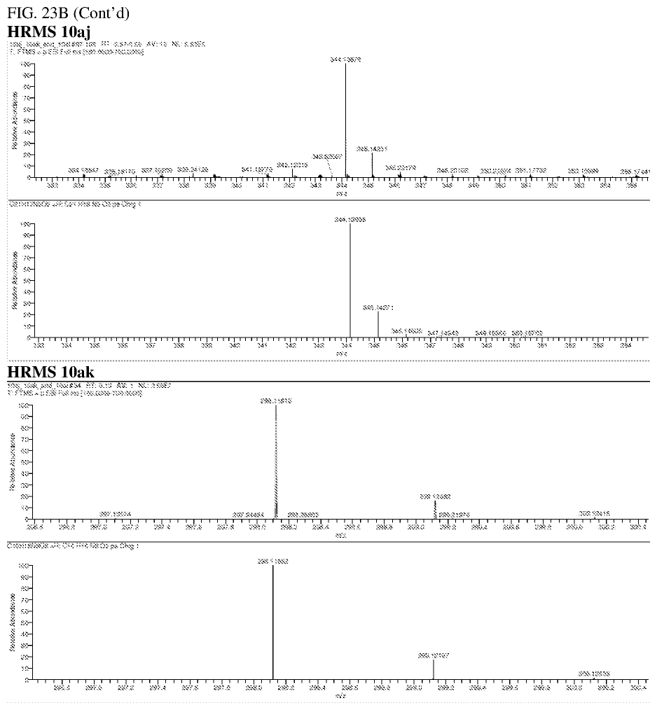

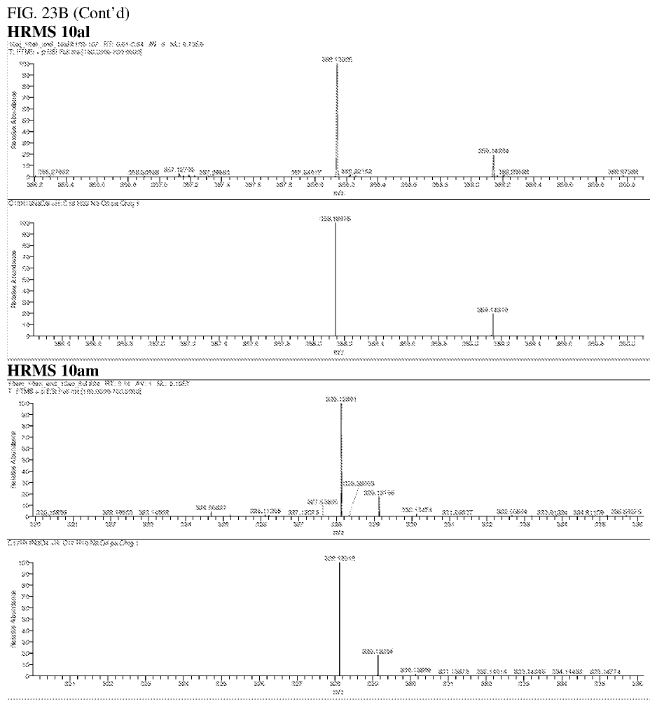

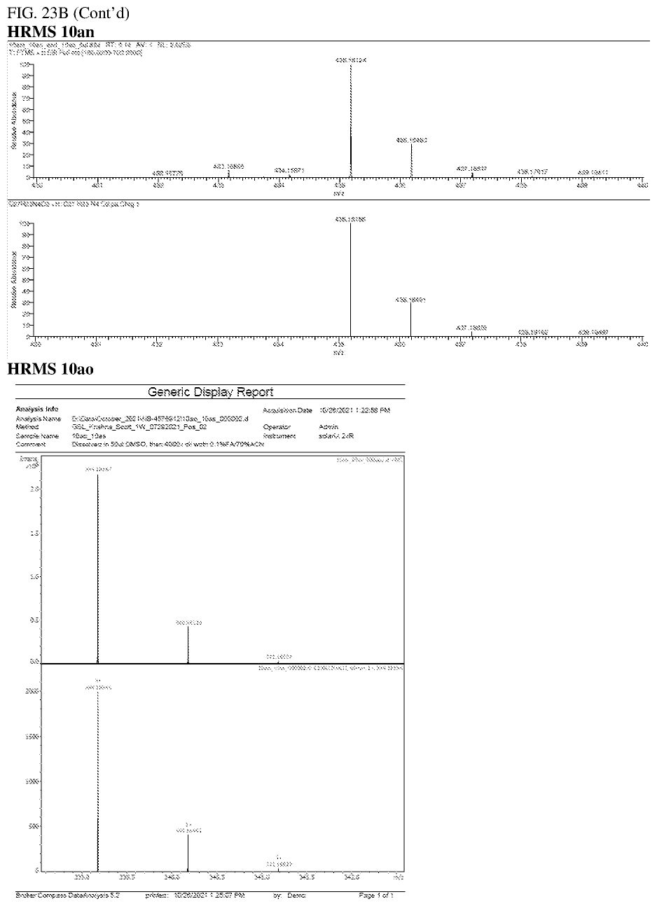

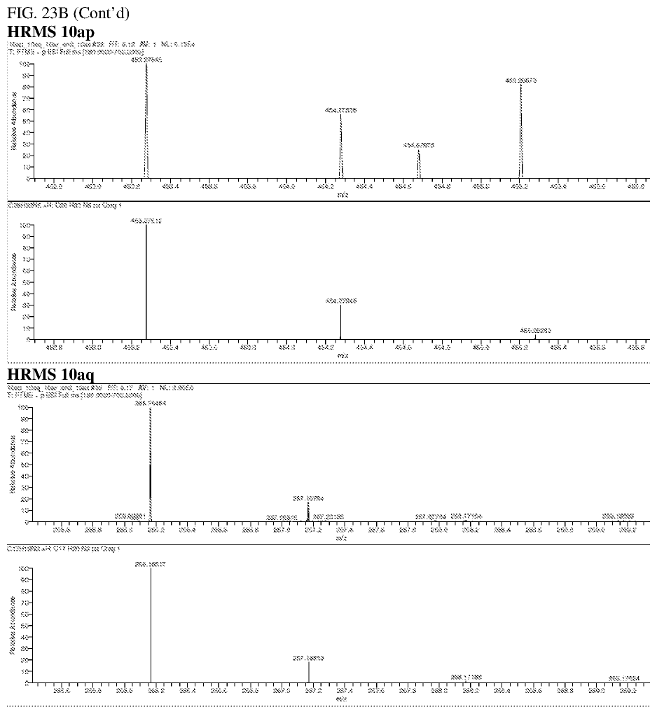

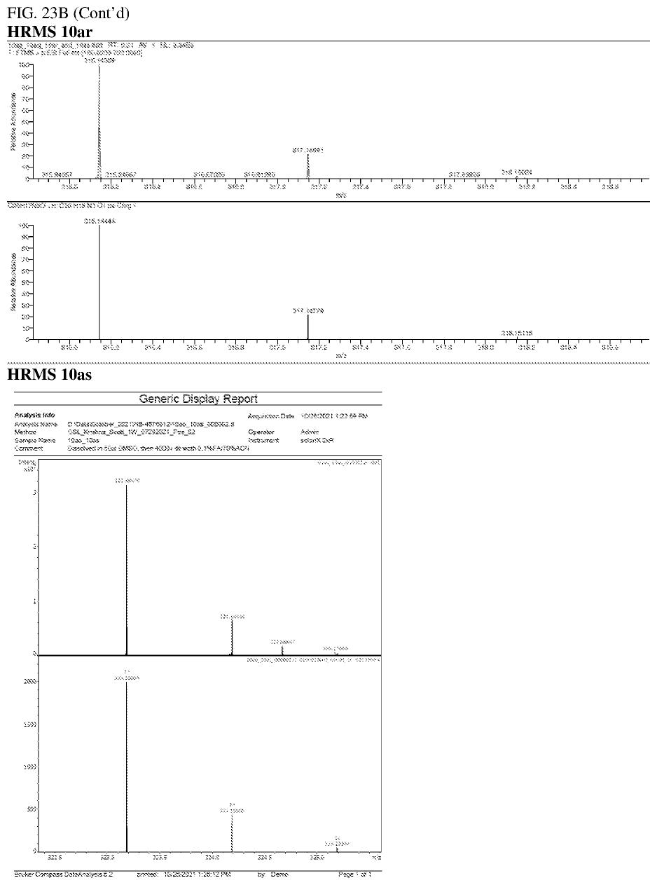

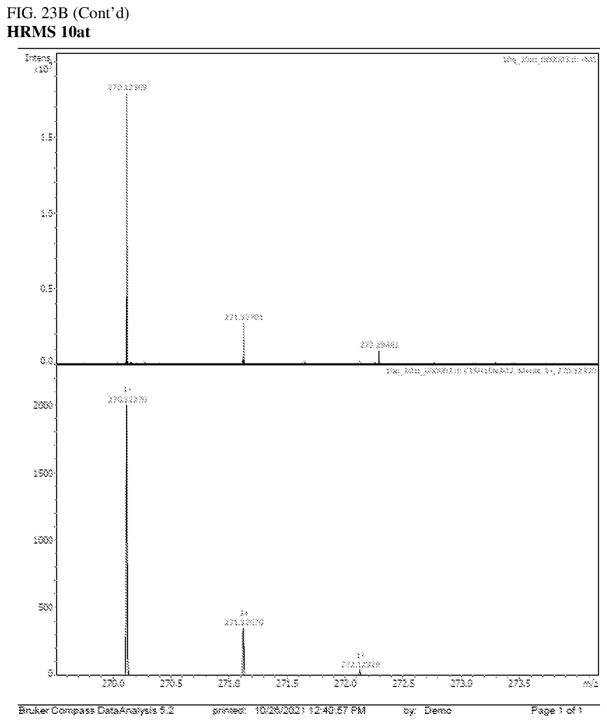

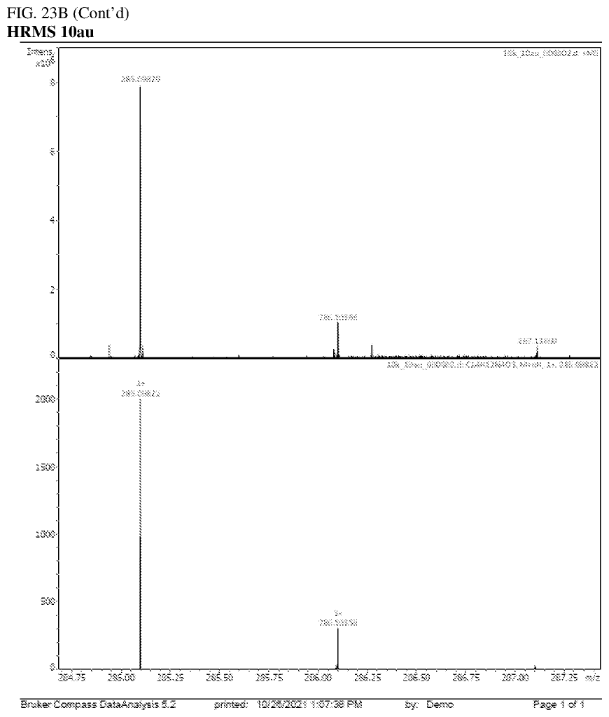

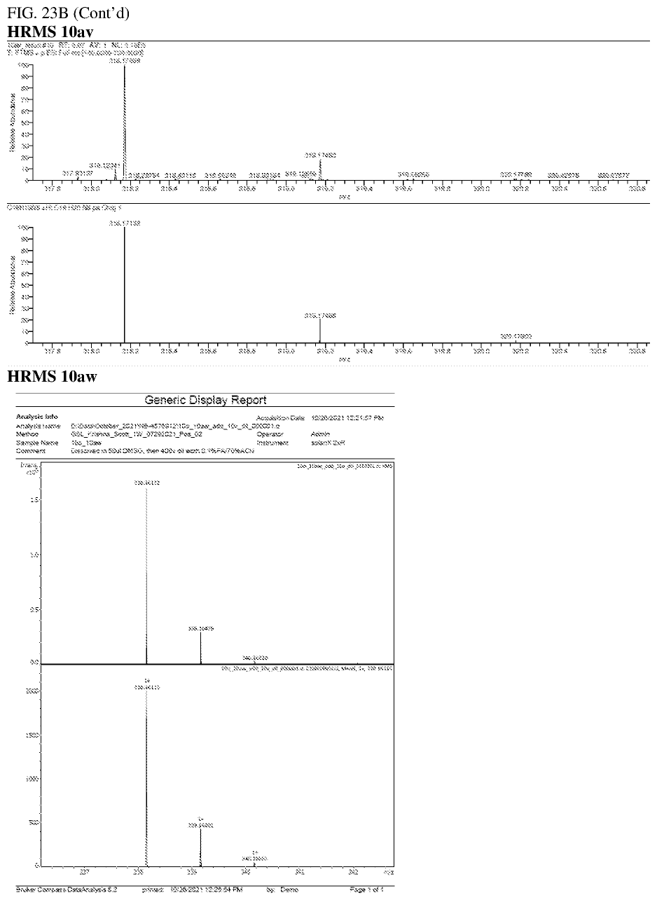

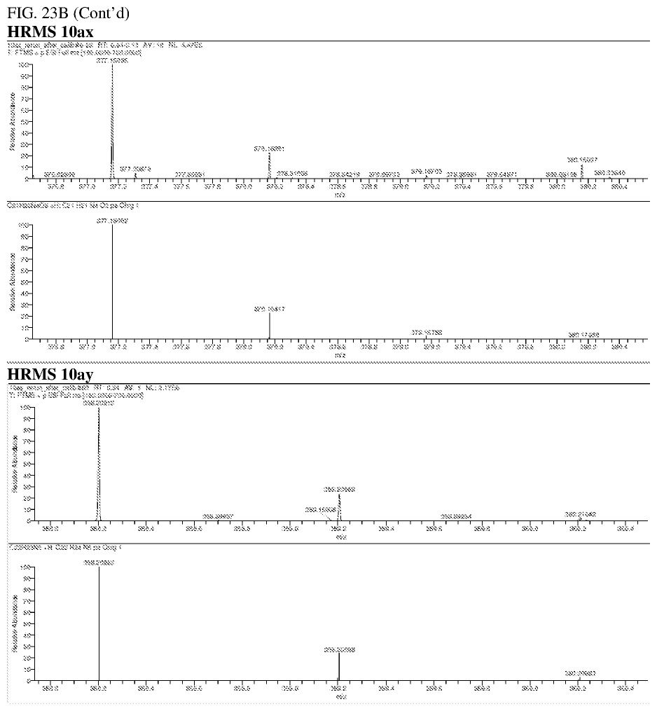

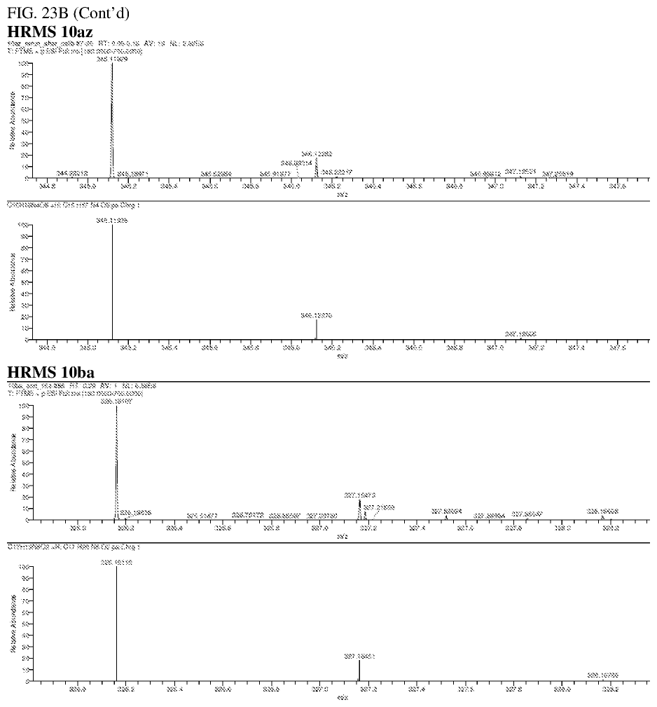

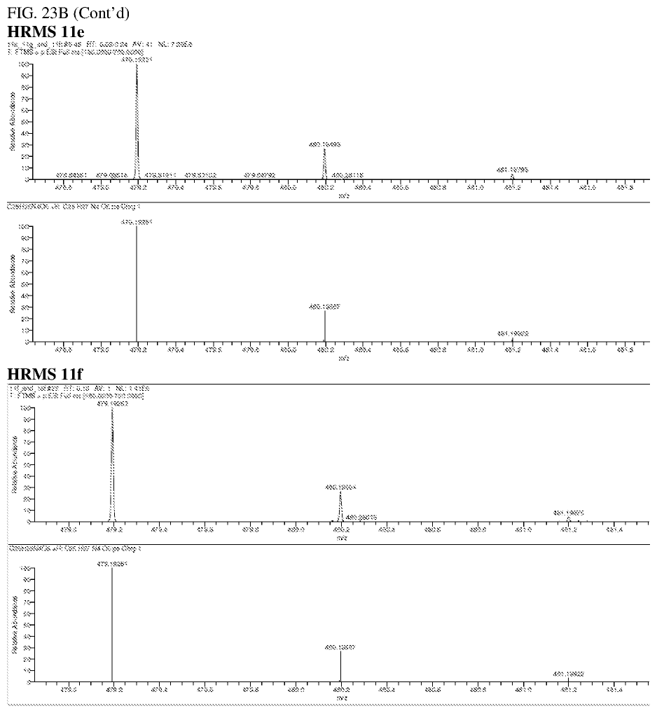

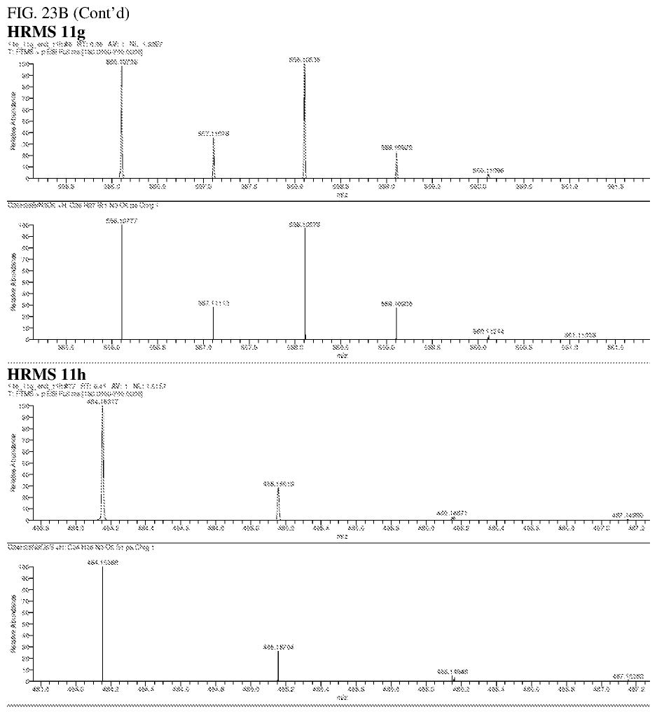

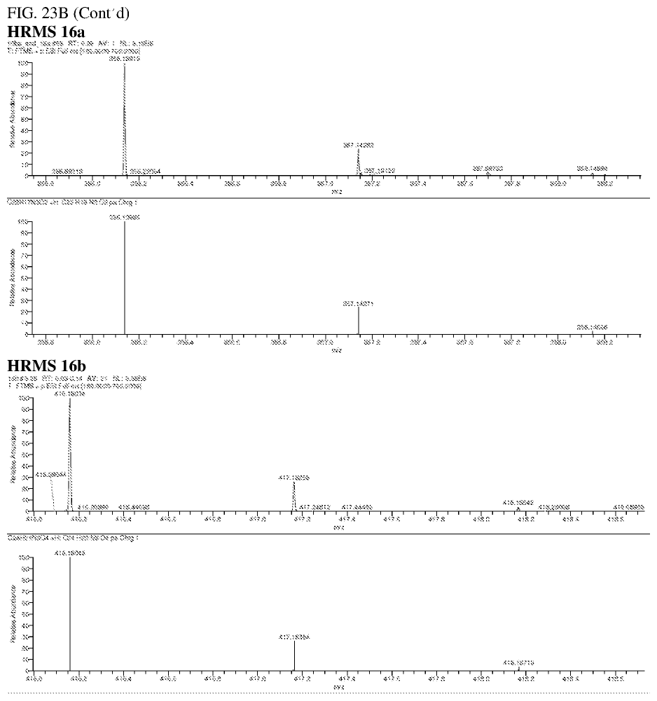

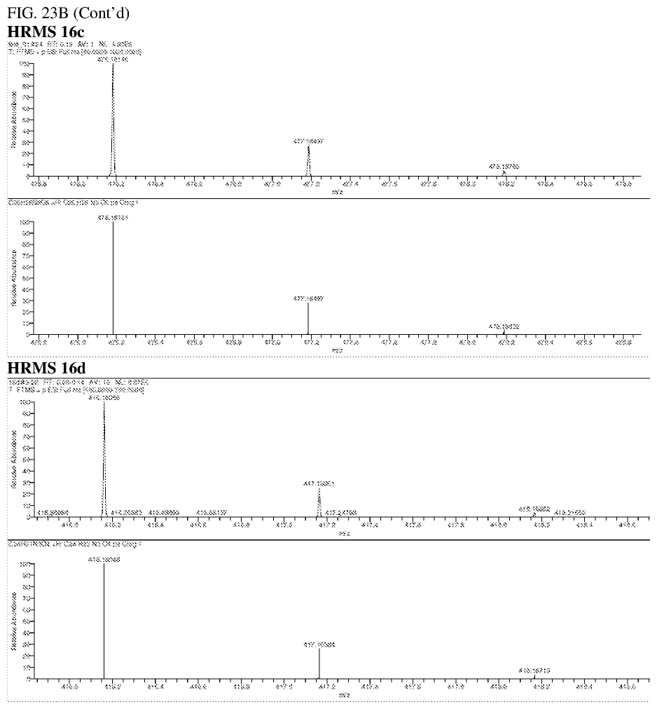

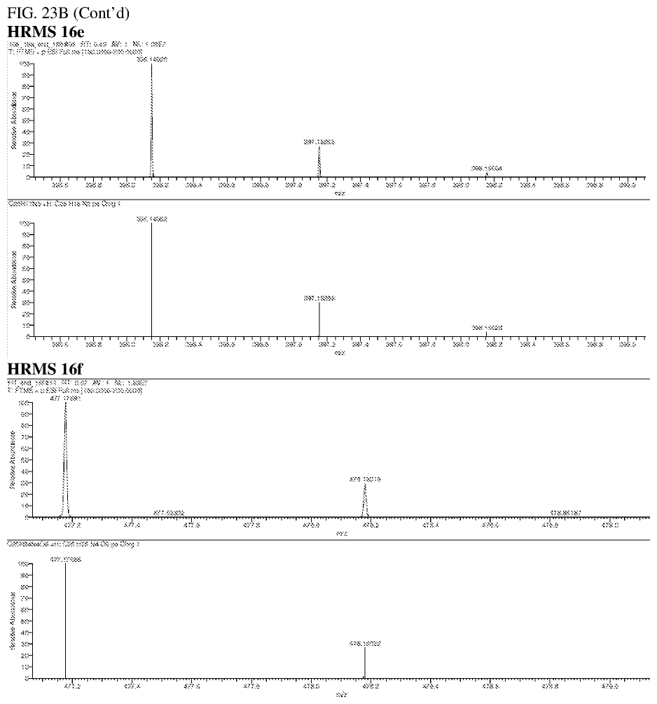

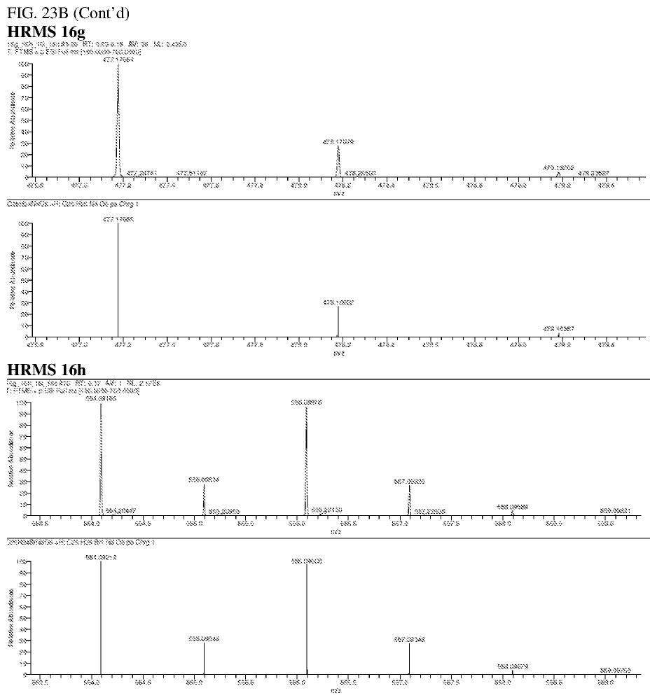

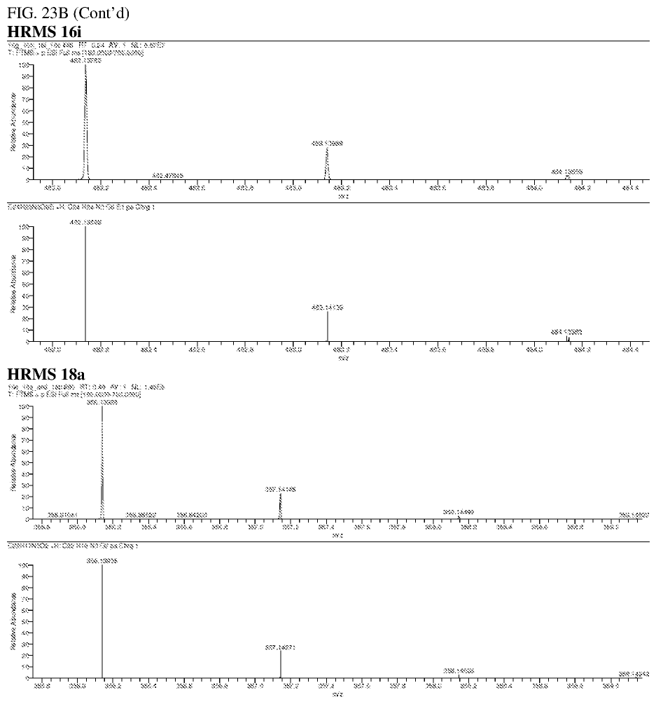

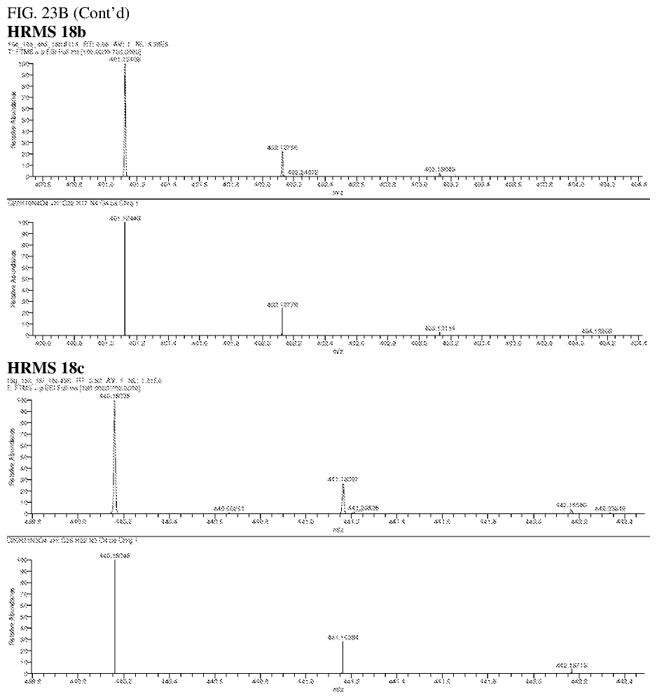

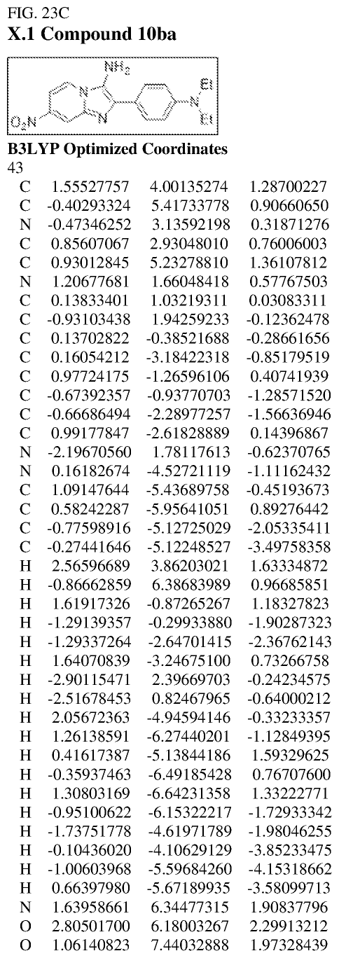

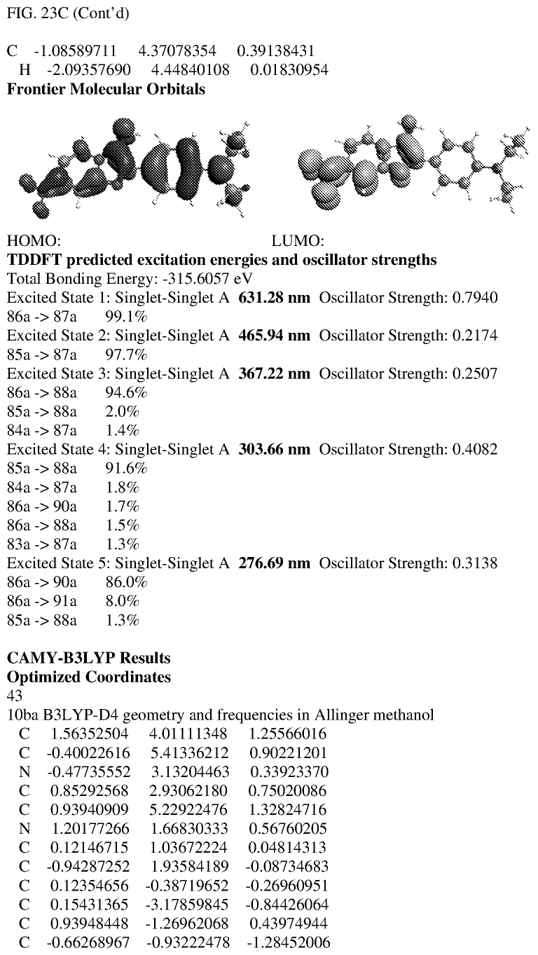

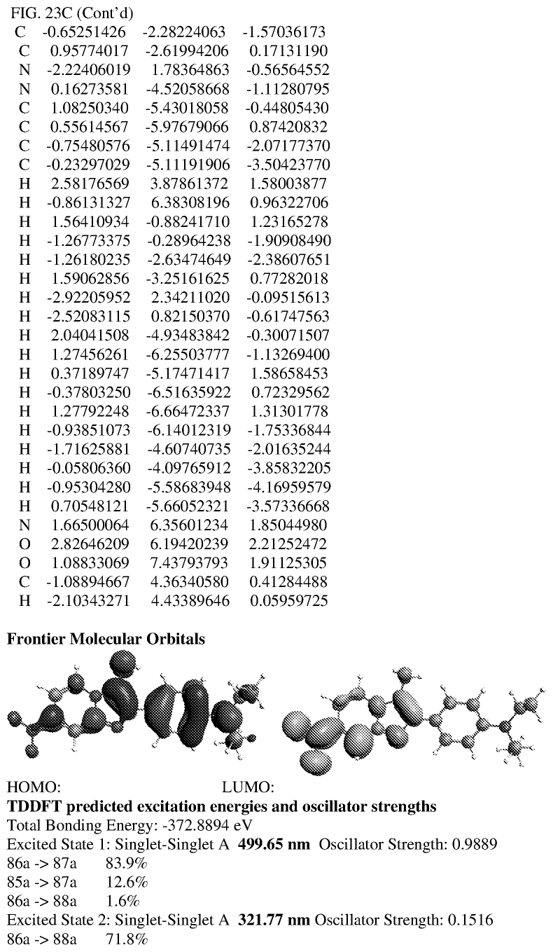

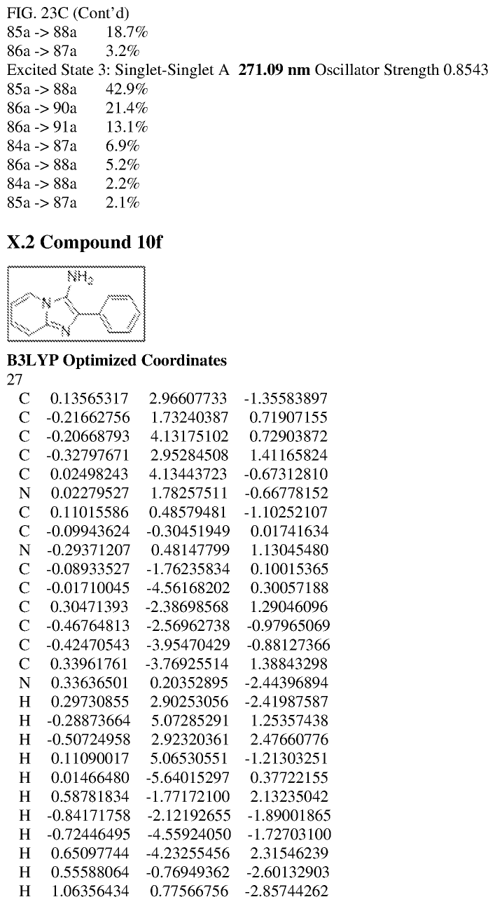

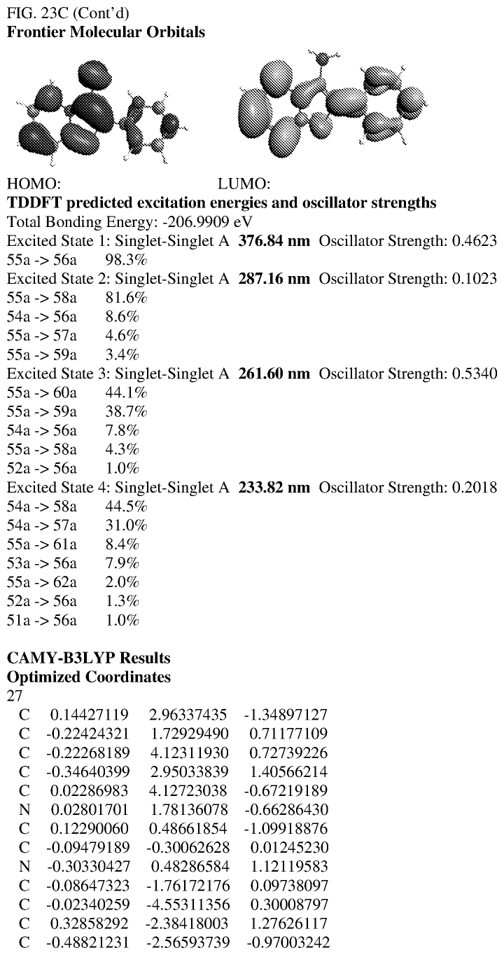

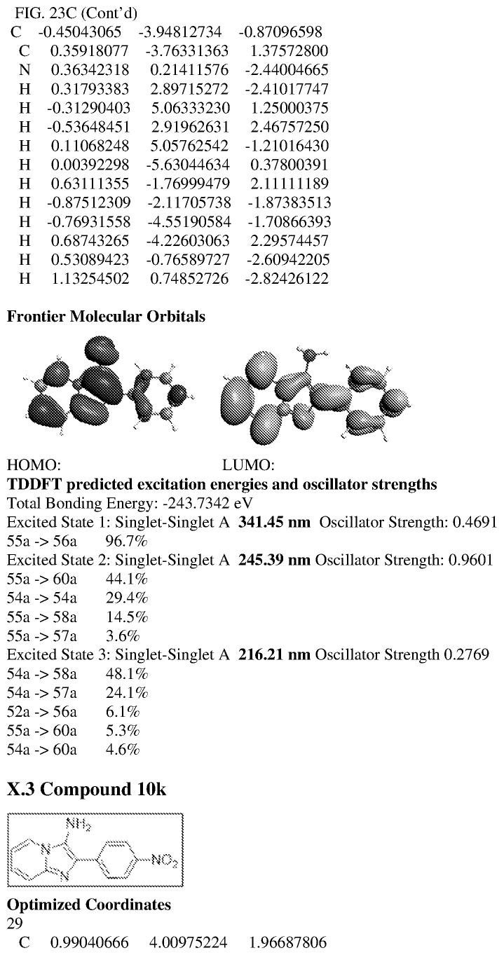

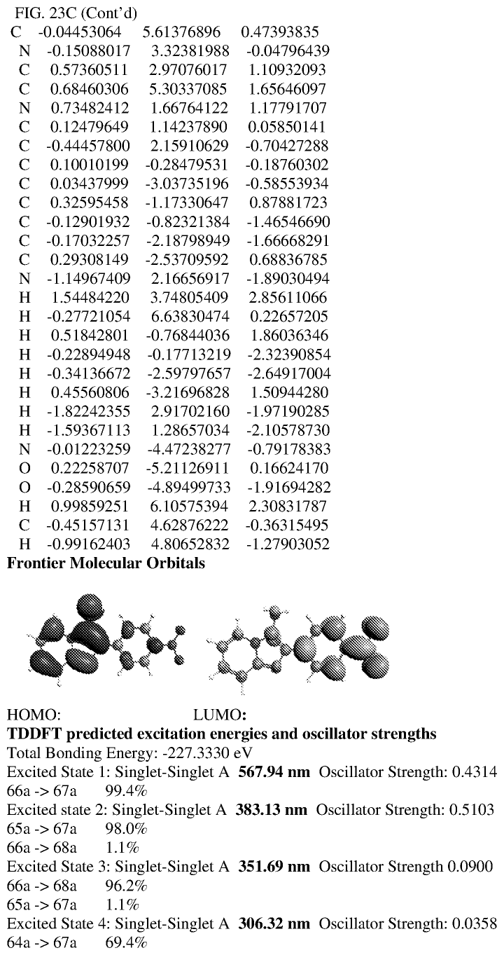

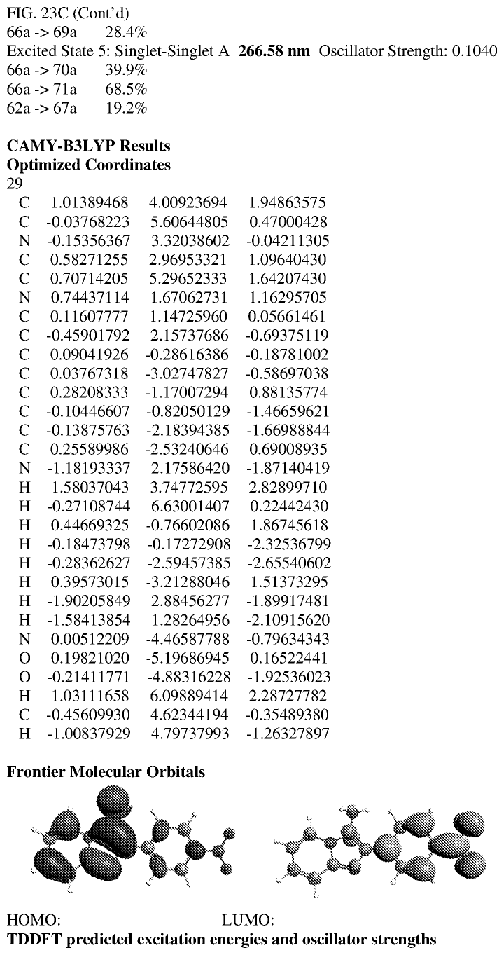

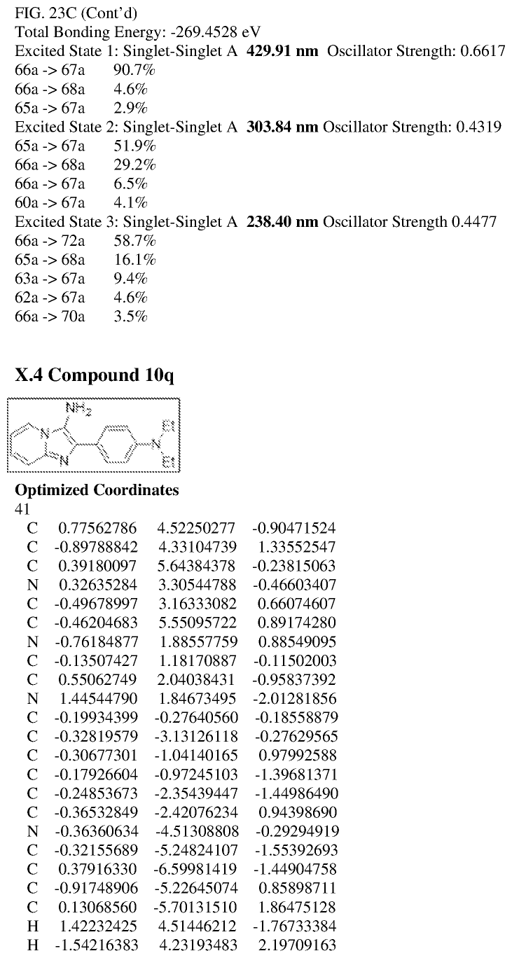

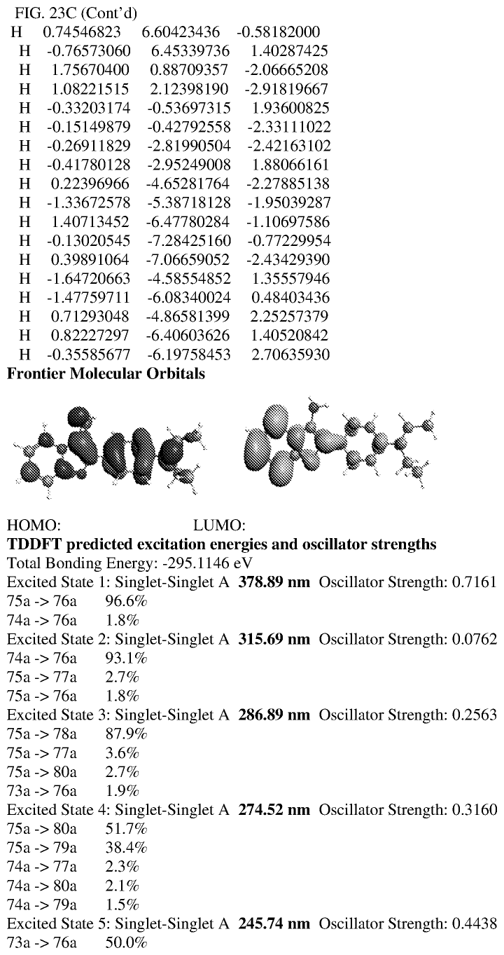

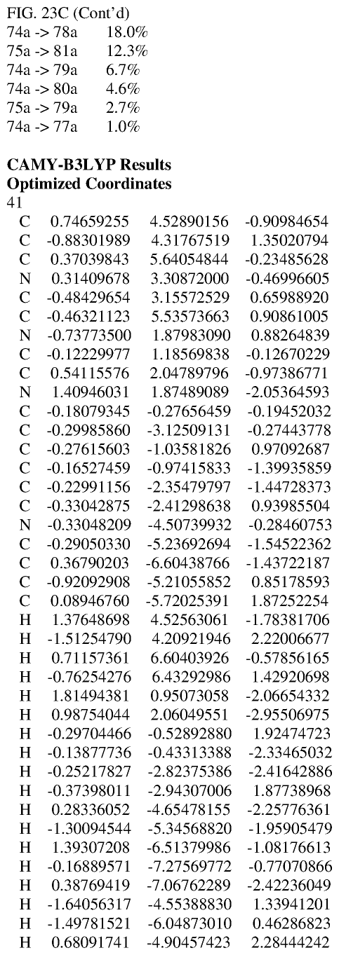

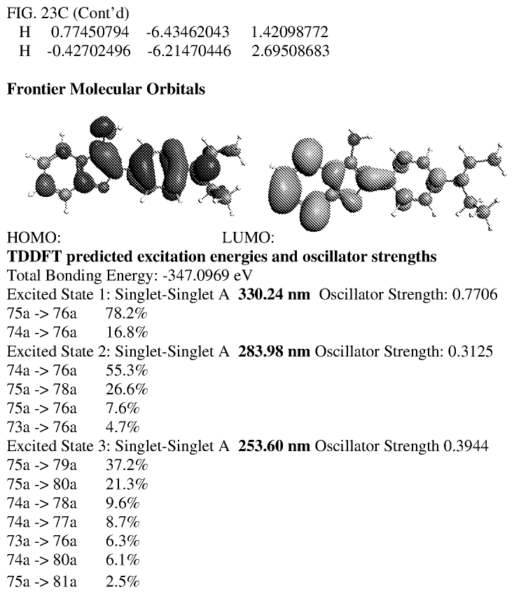

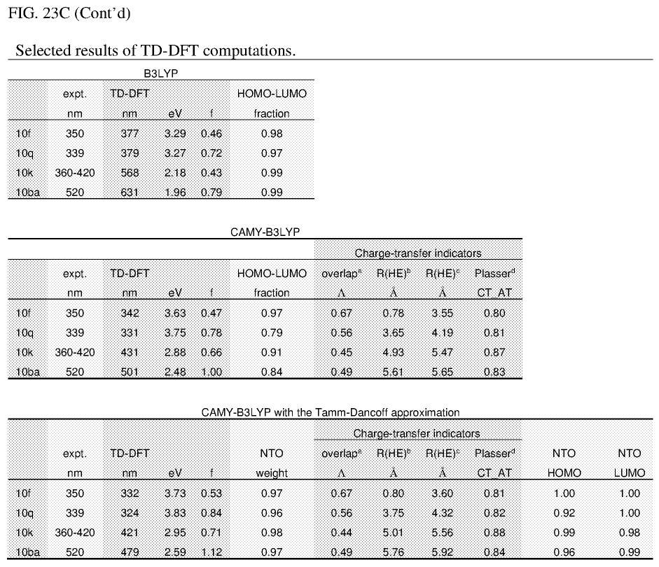

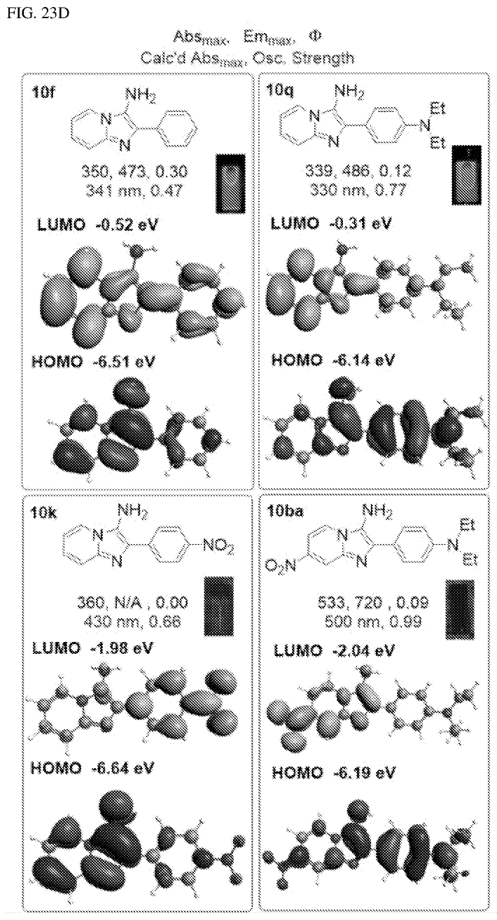

Additionally, other solvents for fluorescence were explored (FIG. 22). DCM produced the highest quantum yield which is likely due to its nonpolar nature diminishing quenching in sterically hindered molecules (Chatterjee, S.; Basu, S.; Ghosh, N.; Chakrabarty, M. Steric Effect on Fluorescence Quenching. Spectrochimica Acta—Part A: Molecular and Biomolecular Spectroscopy 2005, 61 (9), 2199-2201). DMSO garnered the highest wavelength emission which follows previously reported findings on polarity and fluorescence.

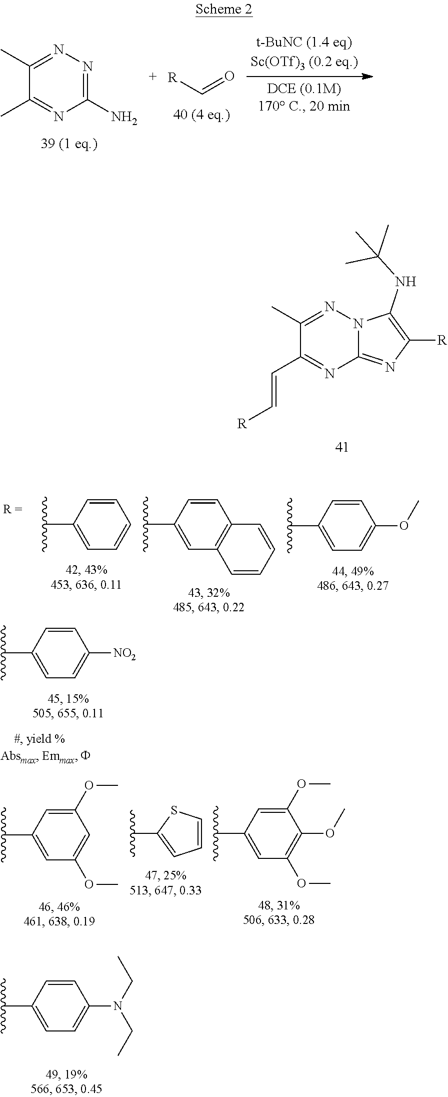

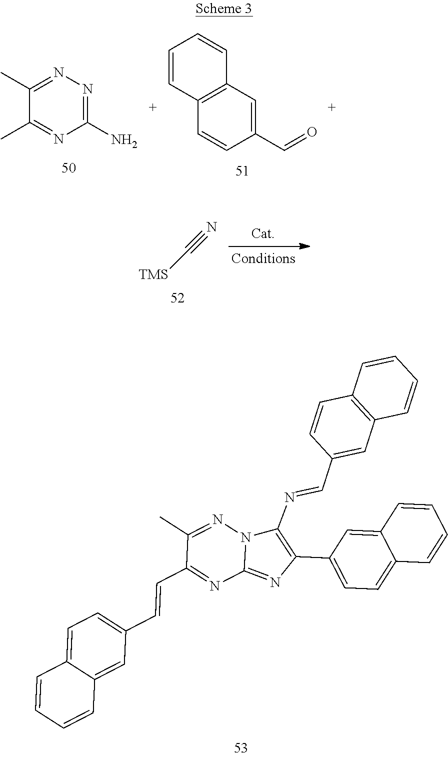

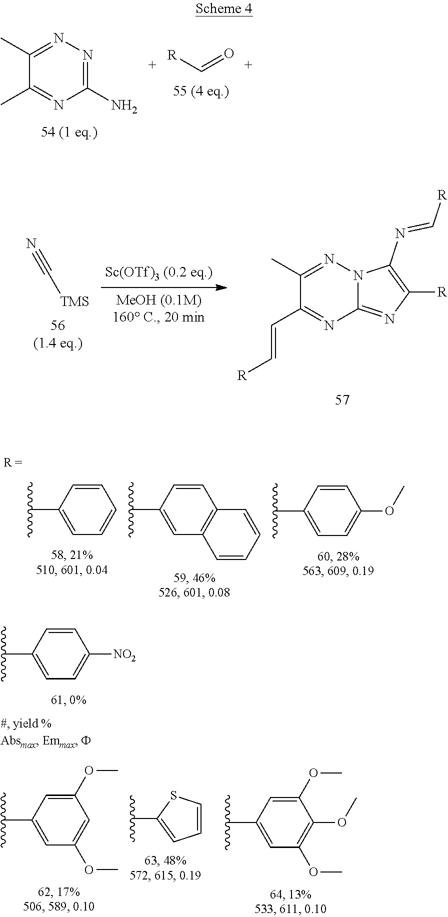

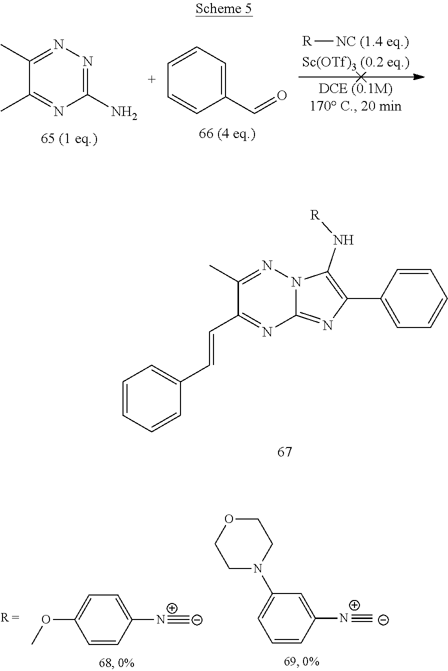

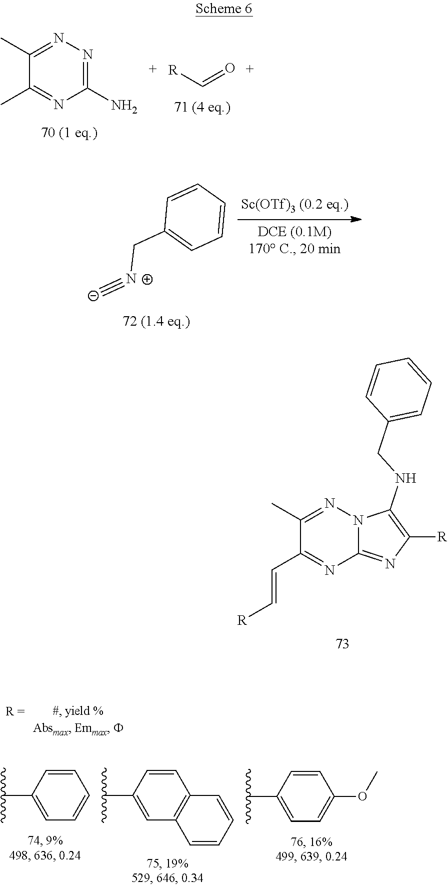

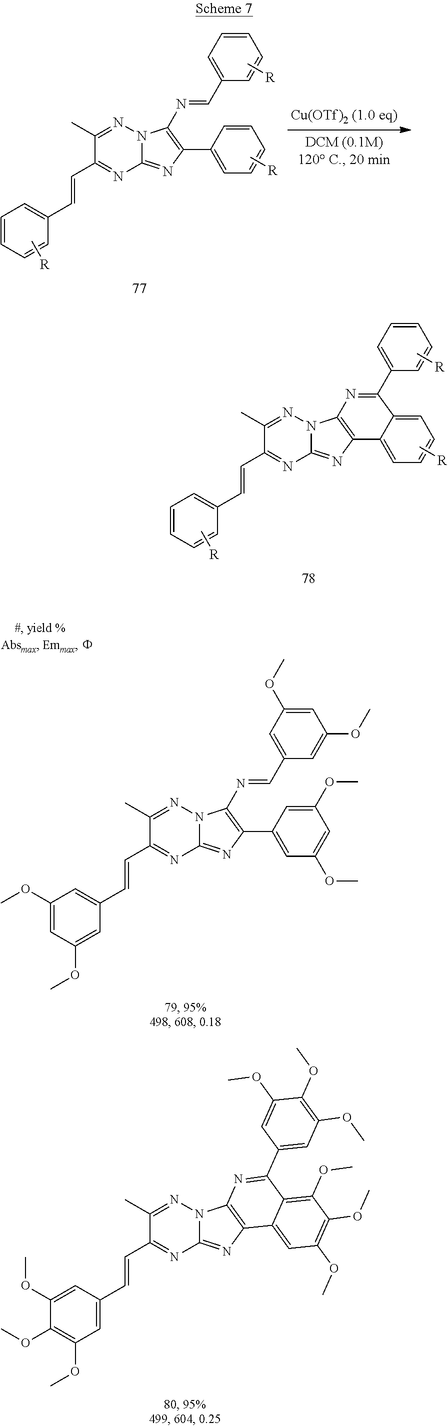

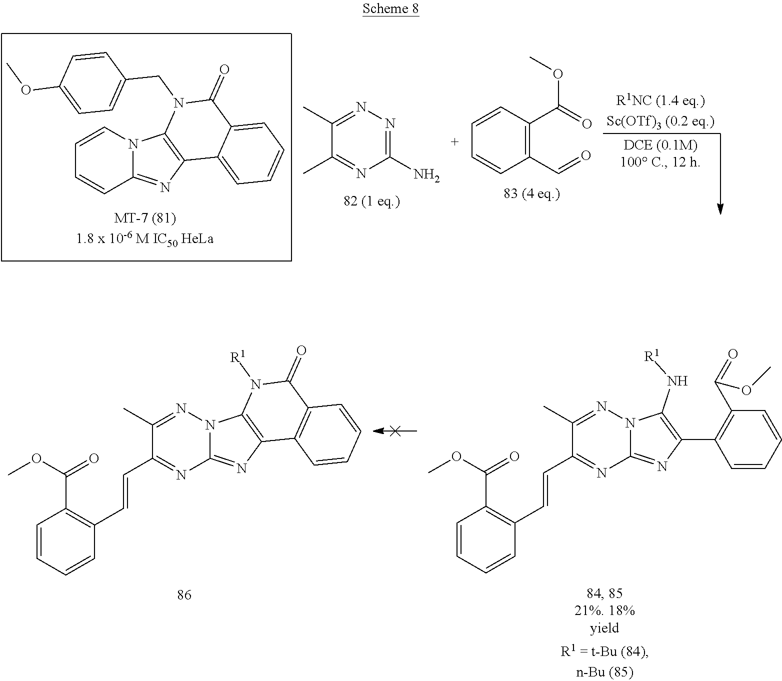

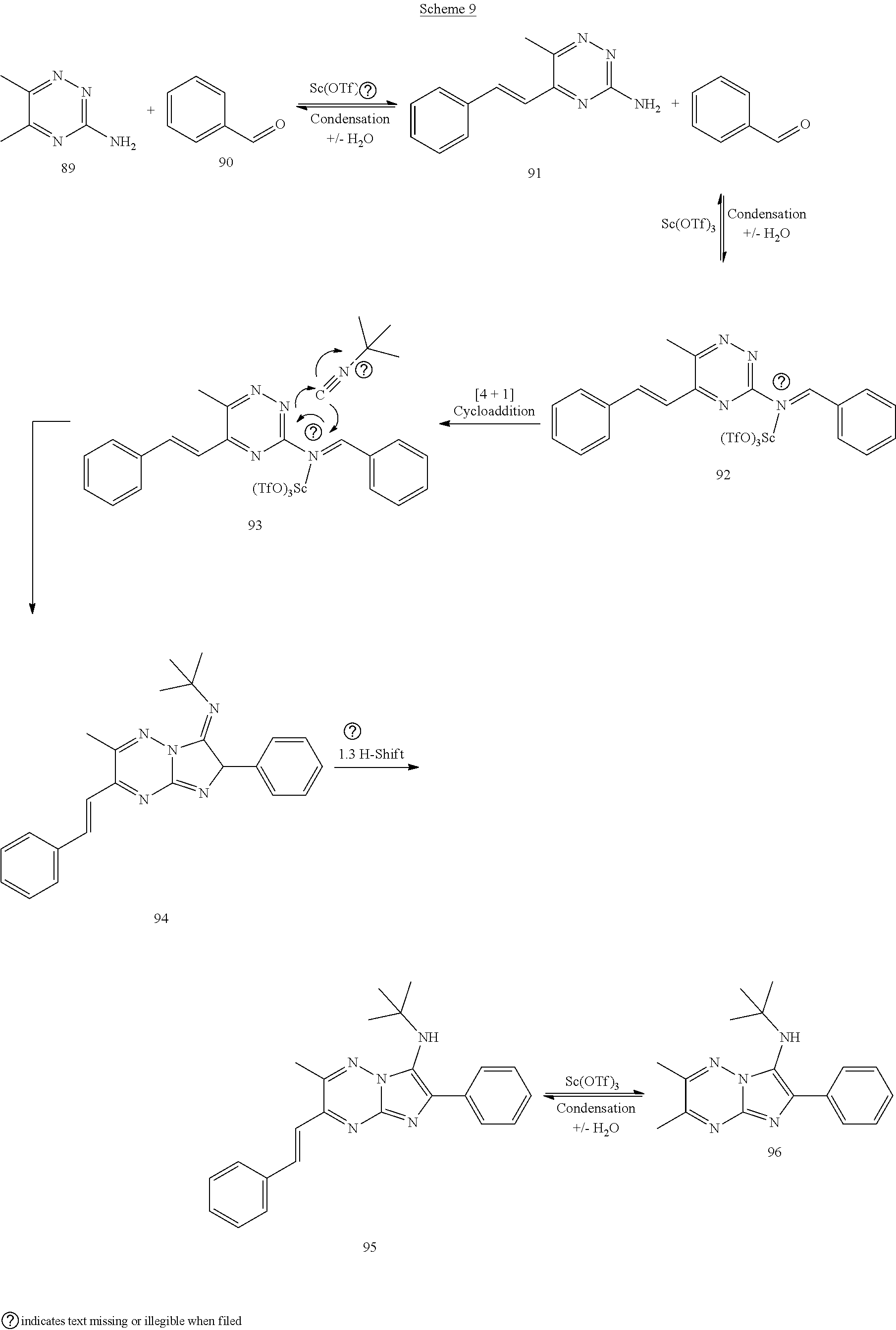

TABLE 15
Time Optimizations

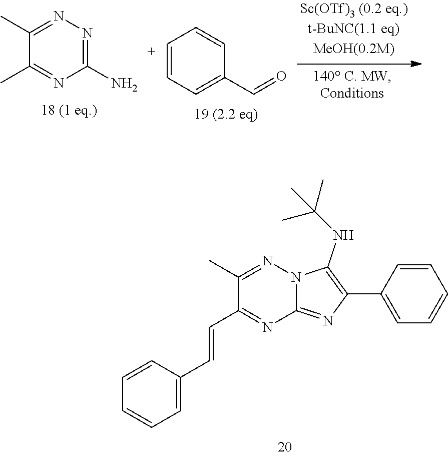

| Entry^{a} | Time (min) | Yield (%) |
|---|---|---|
| 1 | 10 | 3 |
| 2 | 20 | 10 |
| 3 | 40 | 15 |
| 4 | 80 | 21 |
| 5 | 140 | 28 |

TABLE 16
Solvent Optimizations Indicating DCE as Optimal Solvent

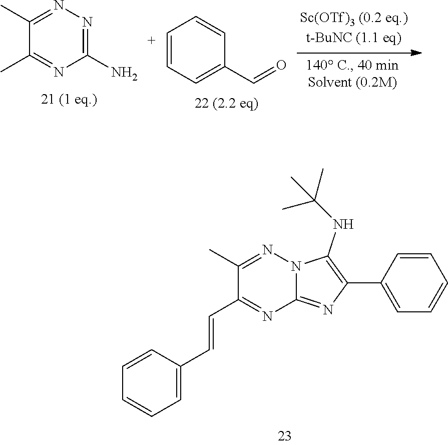

| Entry^{a} | Solvent (M) | Yield (%) |
|---|---|---|
| 3 | MeOH (0.2) | 15 |
| 6 | EtOH (0.2) | 28 |
| 7 | DCE (0.2) | 42 |
| 8 | ACN (0.2) | 33 |
| 9 | CHCl₃ (0.2) | 38 |
| 10 | DiOx (0.2) | 36 |
| 11 | THF (0.2) | 27 |
| 12 | TFE (0.2) | 9 |
| 13 | DCM (0.2) | 13 |

TABLE 17
Equivalent Optimizations

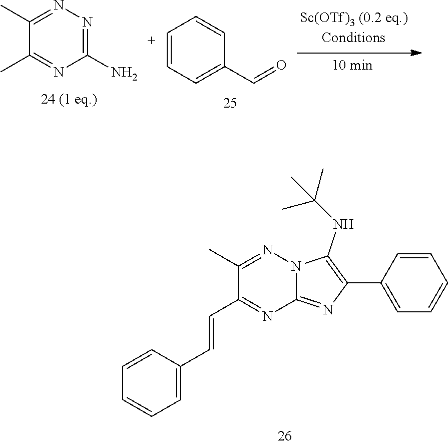

| Entry^{a} | t-BuNC (eq.) | Aldehyde (eq.) | Solvent (0.2M) | T (° C.) | Yield (%) |
|---|---|---|---|---|---|
| 1 | 1.1 | 2.2 | MeOH | 140 | 3 |
| 14 | 1.1 | 2.2 | MeOH | 160 | 15 |
| 15 | 1.1 | 2.2 | DCE | 170 | 29 |
| 16 | 2 | 2.2 | MeOH | 160 | 8 |
| 17 | 2 | 2.2 | DCE | 170 | 27 |
| 18 | 1.1 | 4 | MeOH | 160 | 21 |
| 19 | 1.1 | 4 | DCE | 170 | 33 |
| 20 | 2 | 4 | MeOH | 160 | 9 |
| 21 | 2 | 4 | DCE | 170 | 31 |

TABLE 18
Catalyst Optimization Indicating Sc(OTf)₃ as Efficient Catalyst

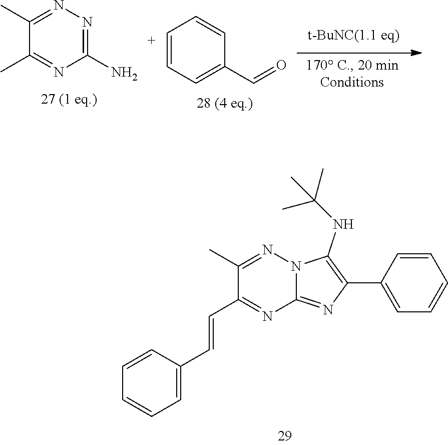

| Entry^{a} | Cat. (eq.) | Solvent (M) | Yield (%) |
|---|---|---|---|
| 22 | Piperidine(0.4) | DCE (0.2) | 23 |
| 23 | AcOH (2) | MeOH (0.2) | 25 |
| 24 | Yb(OTf)₃ (0.16) | DCE (0.2) | 22 |
| 25 | H₂SO₄ | MeOH (0.2) | 0 |
| 26 | H₂SO₄ + Sc(OTf)₃ | MeOH (0.2) | 0 |
| 27 | NH₄Cl (1) | MeOH (0.2) | 10 |
| 28 | Sc(OTf)₃ (0.2) | DCE (0.2) | 35 |

TABLE 19
Additional Equivalents Optimizations

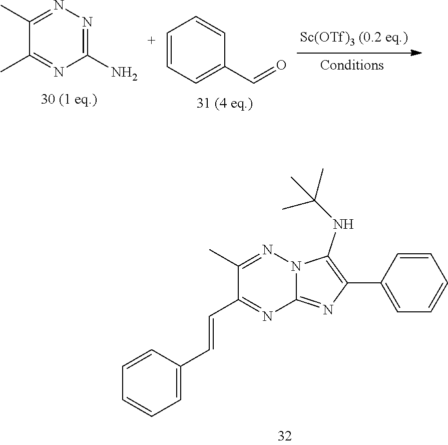

| Entry^{a} | t-BuNC (eq.) | Solvent (M) | T (° C.) | Time (min) | Yield (%) |
|---|---|---|---|---|---|
| 19 | 1.1 | DCE (0.2) | 170 | 10 | 33 |
| 29 | 1.1 | DCE (0.1) | 170 | 10 | 34 |
| 28 | 1.1 | DCE (0.2) | 170 | 20 | 35 |
| 30 | 1.1 | DCE (0.1) | 170 | 20 | 39 |
| 31 | 1.4 | DCE (0.1) | 170 | 20 | 43 |
| 32 | 1.4 | DCE (0.1) | r.t. | 24 h | 10 |
| 33 | 1.4 | DCE (0.1) | 80 | 24 h | 27 |

TABLE 20
Scandium Triflate Equivalents Optimizations

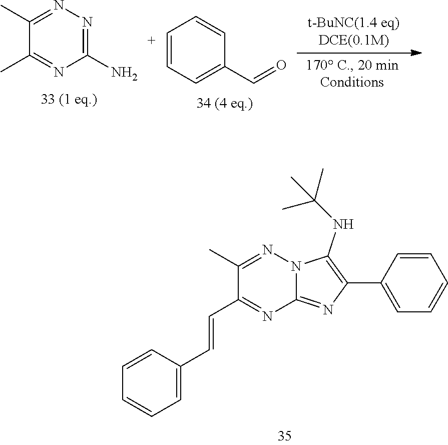

| Entry^{a} | Catalyst (eq) | Yield (%) |
|---|---|---|
| 34 | Sc(OTf)₃ (0.05) | 33 |
| 35 | Sc(Otf)₃ (0.1) | 22 |
| 31 | Sc(Otf)₃ (0.2) | 43 |
| 36 | Sc(Otf)₃ (0.5) | 25 |
| 37 | Sc(Otf)₃ (1.0) | 26 |

TABLE 21
Optimizations of Solvents

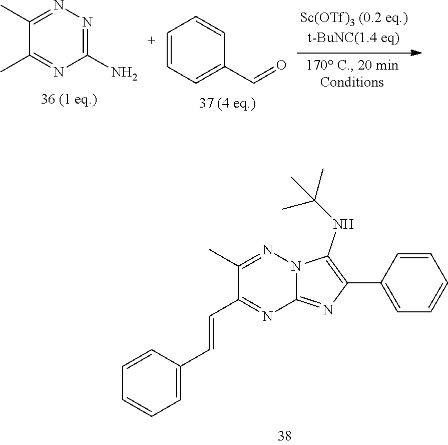

| Entry^{a} | Solvent (M) | Yield (%) |
|---|---|---|
| 38 | 1:1 DCE:MeOH (0.1) | 21 |
| 39 | 2:1 DCE:MeOH (0.1) | 24 |
| 40 | 3:1 DCE:MeOH (0.1) | 22 |
| 41 | Toluene (0.1) | 25 |
| 42 | NEAT | 9 |
| 31 | DCE (0.1) | 43 |

^{a}Scale: 0.25 mmol

TABLE 22
Isocyanide Optimization

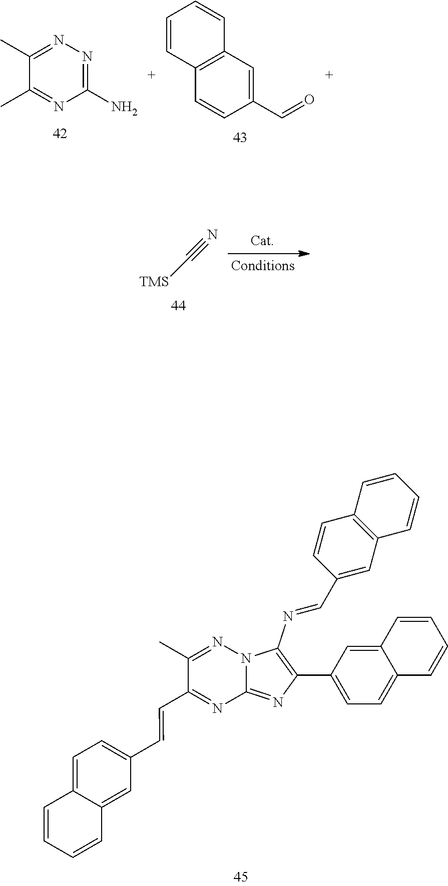

| Entry^{a} | X | Y | Cat. | Solvent (M) | T (° C.) | Time (min.) | Yield (%) |
|---|---|---|---|---|---|---|---|
| 43 | 4.0 eq. | 1.4 eq. | Sc(OTf)₃ (0.2 eq.) | DCE 0.1M | 170 | 20 | 5 |
| 44 | 3.1 eq. | 1.1 eq. | Sc(OTf)₃ (0.2%) | MeOH 0.2M | 140 | 60 | 10 |
| 45 | 3.1 eq. | 2.0 eq. | Sc(OTf)₃ (0.33%) | MeOH 0.2M | 160 | 10 | 10 |
| 46 | 4.0 eq. | 1.1 eq. | Sc(OTf)₃ (0.33%) | MeOH 0.2M | 160 | 10 | 16 |
| 47 | 5.0 eq. | 2.1 eq. | Sc(OTf)₃ (0.2%) | MeOH 0.2M | 160 | 20 | 15 |
| 48 | 3.1 eq. | 1.1 eq. | Yb(OTf)₃ (0.2%) | MeOH 0.2M | 160 | 20 | 7 |
| 49 | 3.1 eq. | 1.1 eq. | In(OTf)₃ (0.2%) | MeOH 0.2M | 160 | 20 | 17 |
| 50 | 3.1 eq. | 1.1 eq. | ZnCl₂ (0.2%) | MeOH 0.2M | 160 | 20 | 3 |
| 51 | 3.1 eq. | 1.1 eq. | ZrCl₄ (0.2%) | MeOH 0.2M | 160 | 20 | 3 |
| 52 | 4.0 eq. | 1.4 eq. | Sc(OTf)₃ (0.2 eq.) | MeOH 0.2M | 160 | 20 | 26 |
| 53 | 4.0 eq. | 1.4 eq. | Sc(OTf)₃ (0.2 eq.) | MeOH 0.1M | 160 | 20 | 46 |

^{a}Scale: 0.25 mmol

TABLE 23
Methodology to produce fully cyclyzed product

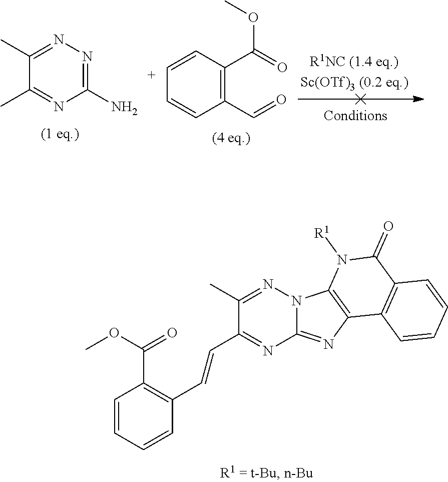

| Entry^{a} | R¹ (1.4 Eq) | Solvent (M) | T (° C.) | Time | Yield (%) |
|---|---|---|---|---|---|
| 54 | t-BuNC | DCE (0.1) | 170 | 20 min | 0 |
| 55 | n-BuNC | DCE (0.1) | 170 | 20 min | 0 |
| 56 | t-BuNC | Neat | 170 | 20 min | 0 |
| 57 | n-BuNC | Neat | 170 | 20 min | 0 |
| 58 | t-BuNC | MeOH (0.1) | 170 | 20 min | 0 |
| 59 | n-BuNC | MeOH (0.1) | 170 | 20 min | 0 |
| 60 | t-BuNC | MeOH (0.1) | 100 | 12 h | 0 |
| 61 | n-BuNC | MeOH (0.1) | 100 | 12 h | 0 |

### Example 6

### Additional Compounds

Table 33 shows a summary of exemplary fluorescent compounds of the present disclosure.

TABLE 33

| Compound | Structure |
|---|---|
| 1 | 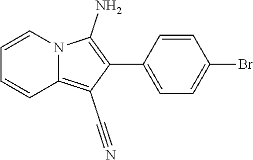 [SMILES: N#Cc1c(-c2ccc(Br)cc2)c(N)n2ccccc12] |
| 2 | 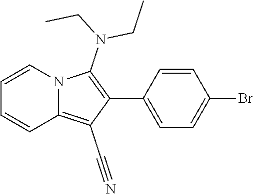 [SMILES: CCN(CC)c1c(-c2ccc(Br)cc2)c(C#N)c2ccccn12] |
| 3 | 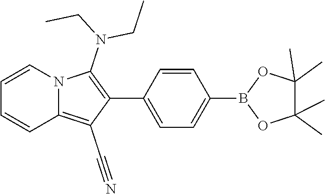 [SMILES: CCN(CC)c1c(-c2ccc(B3OC(C)(C)C(C)(C)O3)cc2)c(C#N)c2ccccn12] |
| 4 | 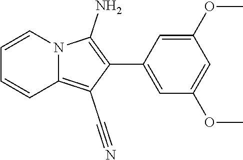 [SMILES: COc1cc(OC)cc(-c2c(C#N)c3ccccn3c2N)c1] |
| 5 | 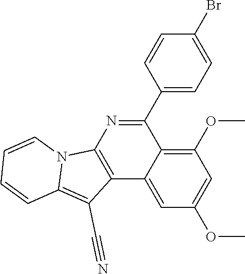 [SMILES: COc1cc(OC)c2c(-c3ccc(Br)cc3)nc3c(c(C#N)c4ccccn43)c2c1] |
| 6 | 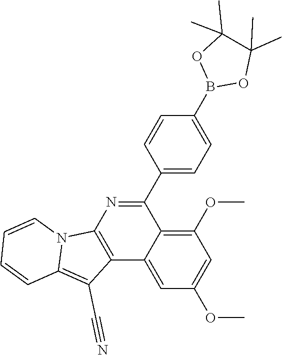 [SMILES: COc1cc(OC)c2c(-c3ccc(B4OC(C)(C)C(C)(C)O4)cc3)nc3c(c(C#N)c4ccccn43)c2c1] |
| 7 | 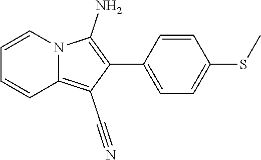 [SMILES: CSc1ccc(-c2c(C#N)c3ccccn3c2N)cc1] |
| 8 | 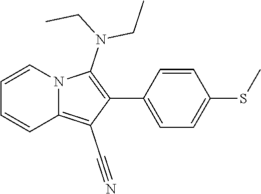 [SMILES: CCN(CC)c1c(-c2ccc(SC)cc2)c(C#N)c2ccccn12] |
| 9 | 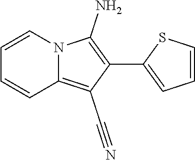 [SMILES: N#Cc1c(-c2cccs2)c(N)n2ccccc12] |
| 10 | 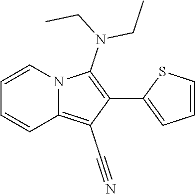 [SMILES: CCN(CC)c1c(-c2cccs2)c(C#N)c2ccccn12] |
| 11 | 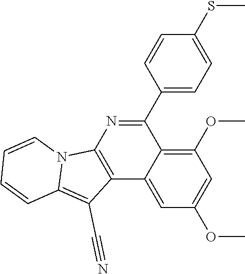 [SMILES: COc1cc(OC)c2c(-c3ccc(SC)cc3)nc3c(c(C#N)c4ccccn43)c2c1] |

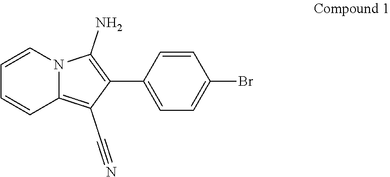 [SMILES: N#Cc1c(-c2ccc(Br)cc2)c(N)n2ccccc12]

### 3-amino-2-(4-bromophenyl)indolizine-1-carbonitrile

2-(pyridin-2-yl) acetonitrile (0.09 mL, 0.838 mmol, 1 eq) and a magnetic stir bar were added to a 5 mL microwave vial. 4-bromobenzaldehyde (0.155 g, 0.838 mmol, 1 eq) and 2,3,4,6,7,8,9,10-octahydropyrimido [1,2-a]azepine (0.026 mL, 0.168 mmol, 0.2 eq) were then added to the vial followed by 5 mL of anhydrous methanol. The microwave vial was then sealed. While stirring, trimethylsilyl cyanide (0.108 mL, 0.837 mmol, 1 eq) was added to the reaction mixture via syringe. The reaction mixture was stirred and heated via a Biotage microwave reactor up to 120° C. for 20 minutes. Upon cooling, a precipitate formed. The reaction vessel was then placed in a freezer over night to facilitate precipitation. The precipitate was then collected via vacuum filtration and washed with methanol yielding a yellowish-brown solid. The filtrate was then collected, concentrated via vacuum distillation, and purified with flash silica gel column chromatography (0-70% ethyl acetate in hexanes). The precipitated product combined with the fractions purified via column chromatography yielded 3-amino-2-(4-bromophenyl)indolizine-1-carbonitrile (177 mg, 0.567 mmol, 68%) as a yellowish-brown solid. ¹H NMR (500 MHz, CDCl3) δ 7.77 (d, J=7.0 Hz, 1H), 7.38 (d, J=8.1 Hz, 2H), 7.32 (d, J=9.0 Hz, 1H), 7.24 (s, 2H), 6.57 (t, J=6.8 Hz, 1H), 3.06 (s, 2H). LCMS (ESI) C₁₅H₁₀BrN₃ requires 312.17, found 312 [M+H].

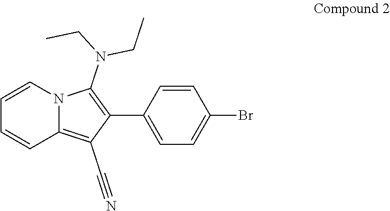 [SMILES: CCN(CC)c1c(-c2ccc(Br)cc2)c(C#N)c2ccccn12]

### 2-(4-bromophenyl)-3-(diethylamino)indolizine-1-carbonitrile

3-amino-2-(4-bromophenyl)indolizine-1-carbonitrile (1) (0.100 g, 0.320 mmol, 1 eq) was added with a stir bar to a microwave vial. 3 mL of 1,2-dichloroethane (0.1 M) was then added. While stirring, acetaldehyde (0.054 mL, 0.961 mmol, 3 eq) and acetic acid (0.055 mL, 0.961 mmol, 3 eq) were added to the reaction mixture. This was followed by the slow addition of sodium triacetoxyborohydride (0.204 g, 0.961 mmol, 3 eq). The reaction stirred at room temperature overnight and was then quenched with aqueous sodium carbonate (5%). The mixture was extracted with dichloromethane and dried over anhydrous sodium sulfate. The crude organic layer was then concentrated via vacuum distillation and purified via flash silica column chromatography (0-50% ethyl acetate in hexanes). All fractions containing the purified product were then collected and concentrated via vacuum distillation producing 2-(4-bromophenyl)-3-(diethylamino)indolizine-1-carbonitrile as a light-yellow crystalline solid (0.092 g, 0.25 mmol, 78%). ¹H NMR (500 MHz, CDCl₃) δ 8.21 (dt, J=7.0, 1.1 Hz, 1H), 7.59-7.52 (m, 3H), 7.41-7.35 (m, 2H), 7.05 (ddd, J=8.9, 6.6, 1.1 Hz, 1H), 6.76 (td, J=6.8, 1.2 Hz, 1H), 2.99 (s, 4H), 0.94 (t, J=7.2 Hz, 6H). LCMS (ESI) C₁₉H₁₈BrN₃ requires 368.28, found 370 [M+H].

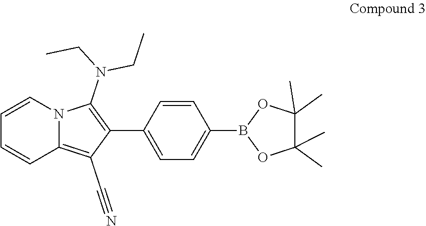 [SMILES: CCN(CC)c1c(-c2ccc(B3OC(C)(C)C(C)(C)O3)cc2)c(C#N)c2ccccn12]

### 3-(diethylamino)-2-(4-(4,4,5,5-tetramethyl-1,3,2-dioxaborolan-2-yl)phenyl)indolizine-1-carbonitrile

2-(4-bromophenyl)-3-(diethylamino)indolizine-1-carbonitrile (2) (0.200 g, 0.534 mmol, 1 eq) and a magnetic stir bar were added to a microwave vial. This was followed by the addition of bis(triphenylphosphine)palladium(II) chloride (0.01 g, 0.014 mmol, 0.026 eq), potassium acetate (0.160 g, 1.63 mmol, 3 eq), and bis(pinacolato)diboron (0.138 g, 0.543 mmol, 1 eq). Anhydrous 1,4-dioxane (1 mL, 0.5M) was then added to the vial. The vial was then sealed, degassed, and purged with argon. The reaction mixture then stirred overnight at 90° C. After cooling to room temperature, the mixture was filtered over celite with ethyl acetate. The mixture was then concentrated via vacuum distillation. A minimum amount of ethyl acetate was added to the crude concentrate and placed in the freezer overnight to promote crystallization of the product. The recrystallized product was collected via vacuum filtration yielding 3-(diethylamino)-2-(4-(4,4,5,5-tetramethyl-1,3,2-dioxaborolan-2-yl)phenyl)indolizine-1-carbonitrile (0.081 g, 0.20 mmol, 36%) as a white solid. ¹H NMR (500 MHz, CDCl₃) δ 8.20 (dt, J=7.0, 1.1 Hz, 1H), 7.90-7.82 (m, 2H), 7.57 (dt, J=8.9, 1.2 Hz, 1H), 7.53-7.48 (m, 2H), 7.03 (td, J=6.6, 1.1 Hz, 1H), 6.74 (td, J=6.8, 1.3 Hz, 1H), 2.96 (s, 4H), 1.35 (s, 12H), 0.92 (t, J=7.2 Hz, 6H). LCMS (ESI) C₂₅H₃₀BN₃O₂ requires 415.34, found 416 [M+H].

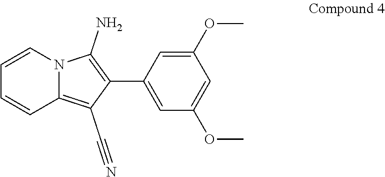 [SMILES: COc1cc(OC)cc(-c2c(C#N)c3ccccn3c2N)c1]

### 3-amino-2-(3,5-dimethoxyphenyl)indolizine-1-carbonitrile

3,5-dimethoxybenzaldehyde (0.209 g, 1.26 mmol, 1 eq) and a magnetic stir bar were added to a 5 mL microwave vial. 2-(pyridin-2-yl)acetonitrile (0.142 mL, 1.26 mmol, 1 eq) and 2,3,4,6,7,8,9,10-octahydropyrimido[1,2-a]azepine (0.038 mL, 0.251 mmol, 0.20 eq) were then added to the vial followed by 5 mL of anhydrous methanol. The microwave vial was then sealed. While stirring, trimethylsilyl cyanide (0.162 mL, 1.26 mmol, 1 eq) was added to the reaction mixture via syringe. The reaction mixture was stirred and heated via a Biotage microwave reactor up to 120° C. for 20 minutes. Upon cooling, a precipitate formed. The reaction vessel was then placed in a freezer over night to facilitate precipitation. The precipitate was then collected via vacuum filtration and washed with methanol yielding a yellowish-brown solid. The precipitated product yielded 3-amino-2-(3,5-dimethoxyphenyl)indolizine-1-carbonitrile (0.295 g, 1.01 mmol, 80%) as a yellowish-brown solid. ¹H NMR (500 MHz, CDCl₃) δ 7.95 (s, 1H), 7.57 (d, J=9.0 Hz, 1H), 6.96 (s, 1H), 6.77 (t, J=6.8 Hz, 1H), 6.73 (d, J=2.3 Hz, 2H), 6.46 (t, J=2.3 Hz, 1H), 3.83 (s, 6H). LCMS (ESI) C17H15N3O2 requires 293.33, found 294 [M+H].

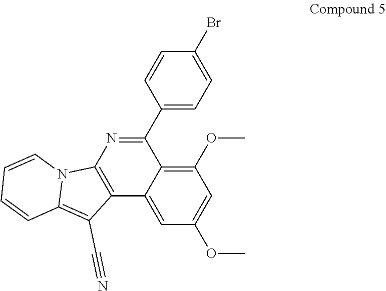 [SMILES: COc1cc(OC)c2c(-c3ccc(Br)cc3)nc3c(c(C#N)c4ccccn43)c2c1]

### 5-(4-bromophenyl)-2,4-dimethoxyindolizino[3,2-c]isoquinoline-12-carbonitrile

3-amino-2-(3,5-dimethoxyphenyl)indolizine-1-carbonitrile (4) (0.227 g, 0.712 mmol, 1 eq) and a magnetic stir bar were added to a microwave vial. This was followed by the addition of 4-bromobenzaldehyde (0.132 g, 0.712 mmol, 1 eq) and copper(II) trifluoromethanesulfonate (0.257 g, 0.712 mmol, 1 eq). After adding 3 mL of 1,2-dichloroethane (0.2M) the microwave vial was sealed. The mixture was then stirred and heated up to 120° C. via a Biotage microwave reactor for 20 minutes. The mixture was then diluted with dichloromethane, washed with saturated sodium bicarbonate, and separated via a separatory funnel. This was repeated until the aqueous layer no longer became blue following the washing of the organic layer, indicating all unreacted copper was removed. The organic layer was then dried over anhydrous sodium sulfate and concentrated via vacuum distillation. The crude mixture was purified via silica-gel column chromatography (0-70% ethyl acetate in hexanes) yielding 5-(4-bromophenyl)-2,4-dimethoxyindolizino[3,2-c]isoquinoline-12-carbonitrile (0.185 g, 0.404 mmol, 57%) as a solid yellow product. ¹H NMR (500 MHz, CDCl3) δ 8.94 (dt, J=6.9, 1.2 Hz, 1H), 7.92 (d, J=2.3 Hz, 1H), 7.81 (dt, J=9.1, 1.1 Hz, 1H), 7.57-7.52 (m, 2H), 7.39-7.31 (m, 3H), 6.90 (td, J=6.8, 1.2 Hz, 1H), 6.58 (d, J=2.3 Hz, 1H), 4.07 (s, 3H), 3.58 (s, 3H). LCMS (ESI) C24H16BrN3O2 requires 458.32, found 458 [M+H].

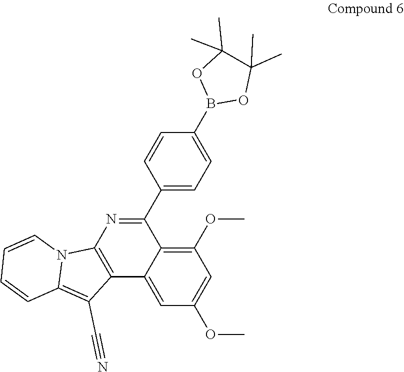 [SMILES: COc1cc(OC)c2c(-c3ccc(B4OC(C)(C)C(C)(C)O4)cc3)nc3c(c(C#N)c4ccccn43)c2c1]

### 2,4-dimethoxy-5-(4-(4,4,5,5-tetramethyl-1,3,2-dioxaborolan-2-yl)phenyl)indolizino[3,2-c]isoquinoline-12-carbonitrile

5-(4-bromophenyl)-2,4-dimethoxyindolizino[3,2-c]isoquinoline-12-carbonitrile (5) (0.205 g, 0.445 mmol, 1 eq) and a magnetic stir bar was added to a microwave vial. This was followed by the addition of [1,1′-Bis(diphenylphosphino)ferrocene]dichloropalladium(II), complex with dichloromethane (0.091 g, 0.011 mmol, 0.025 eq), potassium acetate (0.131 g, 1.34 mmol, 3 eq), and bis(pinacolato)diboron (0.113 g, 0.445 mmol, 1 eq). Anhydrous 1,4-dioxane (1 mL, 0.5M) was then added to the vial. The vial was then sealed, degassed, and purged with argon. The reaction mixture then stirred overnight at 90° C. After cooling to room temperature, the mixture was filtered over celite with ethyl acetate. The mixture was then concentrated via vacuum distillation. A minimum amount of ethyl acetate was added to the crude concentrate and placed in the freezer overnight to promote crystallization of the product. The recrystallized product was collected via vacuum filtration yielding 2,4-dimethoxy-5-(4-(4,4,5,5-tetramethyl-1,3,2-dioxaborolan-2-yl)phenyl)indolizino[3,2-c]isoquinoline-12-carbonitrile (0.080 g, 0.20 mmol, 40%) as a light yellow solid. ¹H NMR (500 MHz, CDCl3) δ 8.95 (ddt, J=7.1, 2.9, 1.4 Hz, 1H), 7.96-7.89 (m, 1H), 7.87 (d, J=7.8 Hz, 1H), 7.84-7.76 (m, 1H), 7.51-7.44 (m, 2H), 7.38-7.31 (m, 2H), 6.89 (qt, J=6.8, 1.2 Hz, 1H), 6.57 (dd, J=3.7, 2.0 Hz, 1H), 4.07 (s, 3H), 3.53 (s, 3H), 1.50 (s, 4H), 1.38 (s, 8H). LCMS (ESI) C30H28BN3O4 requires 505.38, found 506 [M+H].

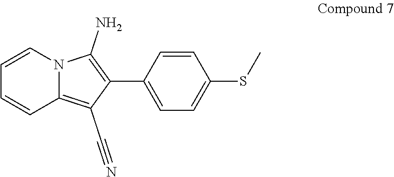 [SMILES: CSc1ccc(-c2c(C#N)c3ccccn3c2N)cc1]

### 3-amino-2-(4-(methylthio)phenyl)indolizine-1-carbonitrile

4-(methylthio)benzaldehyde (0.225 mL, 1.69 mmol, 1 eq) and a magnetic stir bar were added to a 5 mL microwave vial. 2-(pyridin-2-yl)acetonitrile (0.189 mL, 1.69 mmol, 1 eq) and 2,3,4,6,7,8,9,10-octahydropyrimido[1,2-a]azepine (0.051 mL, 0.339 mmol, 0.2 eq) were then added to the vial followed by 7 mL of anhydrous methanol (0.2 M). The microwave vial was then sealed. While stirring, trimethylsilyl cyanide (0.212 mL, 1.69 mmol, 1 eq) was added to the reaction mixture via syringe. The reaction mixture was stirred and heated in an oil bath up to 70° C. for 15 hours. The mixture was then concentrated via vacuum distillation and purified via flash silica gel column chromatography (0-70% ethyl acetate in hexanes). All fractions containing the purified product were collected and concentrated via vacuum distillation yielding 3-amino-2-(4-(methylthio)phenyl)indolizine-1-carbonitrile (0.375 mg, 1.34 mmol, 79%) as a yellowish-brown solid. ¹H NMR (500 MHz, CDCl3) δ 7.86 (s, 1H), 7.43 (d, J=9.0 Hz, 1H), 7.41-7.38 (m, 2H), 7.25-7.21 (m, 2H), 6.84 (s, 1H), 6.68-6.62 (m, 1H), 3.30 (s, 2H), 2.39 (s, 3H). LCMS (ESI) C16H13N3S requires 279.36, found 278 [M+H].

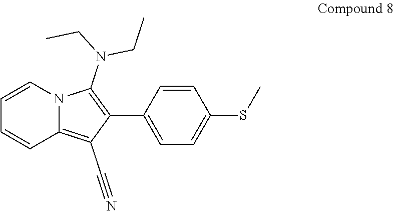 [SMILES: CCN(CC)c1c(-c2ccc(SC)cc2)c(C#N)c2ccccn12]

### 3-(diethylamino)-2-(4-(methylthio)phenyl)indolizine-1-carbonitrile

3-amino-2-(4-(methylthio)phenyl)indolizine-1-carbonitrile (7) (0.350 g, 1.25 mmol, 1 eq) was added with a stir bar to a microwave vial. 8 mL of 1,2-dichloroethane (0.1 M) was then added. While stirring, acetaldehyde (0.210 mL, 3.76 mmol, 3 eq) and acetic acid (0.215 mL, 3.76 mmol, 3 eq) were added to the reaction mixture and stirred for 20 minutes. This was followed by the slow addition of sodium triacetoxyborohydride (0.797 g, 3.76 mmol, 3 eq). The reaction stirred at room temperature under argon for 2 hours. The reaction was then quenched with aqueous sodium carbonate (5%). The mixture was extracted with dichloromethane and dried over anhydrous sodium sulfate. The crude organic layer was then concentrated via vacuum distillation and purified via flash silica-gel column chromatography (0-50% ethyl acetate in hexanes). All fractions containing the purified product were then collected and concentrated via vacuum distillation producing 3-(diethylamino)-2-(4-(methylthio)phenyl)indolizine-1-carbonitrile (0.374 g, 1.11 mmol, 89%) as a light-yellow crystalline solid. ¹H NMR (500 MHz, CDCl3) δ 8.01 (dt, J=7.0, 1.1 Hz, 1H), 7.35 (dt, J=8.9, 1.2 Hz, 1H), 7.28-7.21 (m, 2H), 7.13-7.07 (m, 2H), 6.82 (ddd, J=8.9, 6.6, 1.2 Hz, 1H), 6.54 (td, J=6.8, 1.2 Hz, 1H), 2.79 (s, 4H), 2.30 (s, 3H), 0.73 (t, J=7.2 Hz, 6H). LCMS (ESI) C20H21N3S requires 335.47, found 336 [M+H].

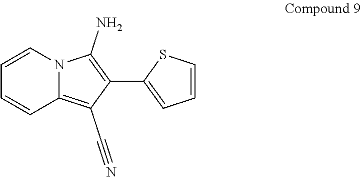 [SMILES: N#Cc1c(-c2cccs2)c(N)n2ccccc12]

### 3-amino-2-(thiophen-2-yl)indolizine-1-carbonitrile

Thiophene-2-carbaldehyde (0.200 mL, 2.12 mmol, 1 eq) and a magnetic stir bar were added to a 5 mL microwave vial. 2-(pyridin-2-yl)acetonitrile (0.236 mL, 2.12 mmol, 1 eq) and 2,3,4,6,7,8,9,10-octahydropyrimido[1,2-a]azepine (0.064 mL, 0.423 mmol, 0.2 eq) were then added to the vial followed by 8 mL of anhydrous methanol. The microwave vial was then sealed. While stirring, trimethylsilyl cyanide (0.265 mL, 2.12 mmol, 1 eq) was added to the reaction mixture via syringe. The reaction mixture was stirred and heated in an oil bath up to 70° C. for 15 hours. The mixture was then concentrated via vacuum distillation and purified via flash silica gel column chromatography (0-70% ethyl acetate in hexanes). All fractions containing the purified product were collected and concentrated via vacuum distillation yielding 3-amino-2-(thiophen-2-yl)indolizine-1-carbonitrile (0.136 g, 0.568 mmol, 27%) as a brown solid. ¹H NMR (500 MHz, DMSO) δ 8.26 (dd, J=7.1, 1.1 Hz, 1H), 7.60 (dd, J=5.1, 1.2 Hz, 1H), 7.54-7.47 (m, 2H), 7.20 (dd, J=5.1, 3.6 Hz, 1H), 7.04 (ddd, J=9.0, 6.6, 1.1 Hz, 1H), 6.90 (td, J=6.8, 1.3 Hz, 1H), 5.33 (s, 2H). LCMS (ESI) C13H9N3S requires 239.30, found 240 [M+H].

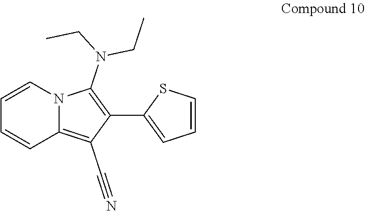 [SMILES: CCN(CC)c1c(-c2cccs2)c(C#N)c2ccccn12]

### 3-(diethylamino)-2-(thiophen-2-yl)indolizine-1-carbonitrile

3-amino-2-(thiophen-2-yl)indolizine-1-carbonitrile (9) (0.250 g, 1.04 mmol, 1 eq) was added with a stir bar to a microwave vial. 7 mL of 1,2-dichloroethane (0.1 M) was then added. While stirring, acetaldehyde (0.175 mL, 3.13 mmol, 3 eq) and acetic acid (0.179 mL, 3.13 mmol, 3 eq) were added to the reaction mixture. This was followed by the slow addition of sodium triacetoxyborohydride (0.664 g, 3.13 mmol, 3 eq). The reaction stirred at room temperature under argon for 2 hours. The reaction was then quenched with aqueous sodium carbonate (5%). The mixture was extracted with dichloromethane and dried over anhydrous sodium sulfate. The crude organic layer was then concentrated via vacuum distillation and purified via flash silica-gel column chromatography (0-50% ethyl acetate in hexanes). All fractions containing the purified product were then collected and concentrated via vacuum distillation producing 3-(diethylamino)-2-(thiophen-2-yl)indolizine-1-carbonitrile (0.291 g, 0.985 mmol, 94%) as a light-yellow crystalline solid. ¹H NMR (500 MHz, CDCl3) δ 7.97 (dd, J=7.0, 1.2 Hz, 1H), 7.59 (dd, J=3.7, 1.2 Hz, 1H), 7.46 (dt, J=8.9, 1.2 Hz, 1H), 7.24 (dd, J=5.2, 1.2 Hz, 1H), 7.01 (dd, J=5.2, 3.6 Hz, 1H), 6.94 (ddd, J=8.9, 6.6, 1.1 Hz, 1H), 6.64 (td, J=6.8, 1.3 Hz, 1H), 3.12 (p, J=6.8 Hz, 4H), 0.89 (t, J=7.2 Hz, 6H). LCMS (ESI) C₁₇H₁₇N₃S requires 295.40, found 296 [M+H].

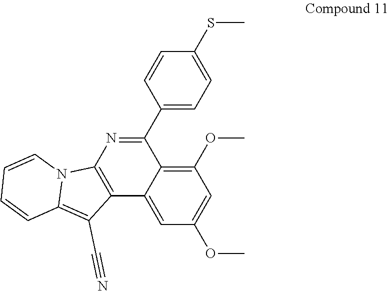 [SMILES: COc1cc(OC)c2c(-c3ccc(SC)cc3)nc3c(c(C#N)c4ccccn43)c2c1]

2,4-dimethoxy-5-(4-(methylthio)phenyl)indolizino[3,2-c]isoquinoline-12-carbonitrile 3-amino-2-(3,5-dimethoxyphenyl)indolizine-1-carbonitrile (4) (0.250 g, 0.852 mmol, 1 eq) and a magnetic stir bar were added to a microwave vial. This was followed by the addition of 4-(methylthio)benzaldehyde (0.113 mL, 0.852 mmol, 1 eq) and copper(II) trifluoromethanesulfonate (0.308 g, 0.852 mmol, 1 eq). After adding 4 mL of dichloromethane (0.2M) the microwave vial was sealed. The mixture was then stirred and heated up to 50° C. in an oil bath overnight. The mixture was then diluted with dichloromethane, washed with saturated sodium bicarbonate, and separated via a separatory funnel. This was repeated until the aqueous layer no longer became blue following the washing of the organic layer, indicating all unreacted copper was removed. The organic layer was then dried over anhydrous sodium sulfate and concentrated via vacuum distillation. The crude mixture was purified via silica-gel column chromatography (0-3% methanol in dichloromethane) yielding 2,4-dimethoxy-5-(4-(methylthio)phenyl)indolizino[3,2-c]isoquinoline-12-carbonitrile (0.235 g, 0.552 mmol, 65%) as a solid yellow product. ¹H NMR (500 MHz, CDCl3) δ 8.95 (dt, J=7.0, 1.1 Hz, 1H), 7.82 (d, J=2.3 Hz, 1H), 7.71 (dt, J=9.1, 1.2 Hz, 1H), 7.37-7.31 (m, 2H), 7.29-7.20 (m, 3H), 6.81 (td, J=6.8, 1.2 Hz, 1H), 6.49 (d, J=2.3 Hz, 1H), 3.98 (s, 3H), 3.49 (s, 3H), 2.47 (s, 3H). LCMS (ESI) C₂₅H₁₉N₃O₂S requires 425.51, found 426 [M+H].

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes.

What is claimed is:

1. A composition, comprising a molecule selected from the group consisting of 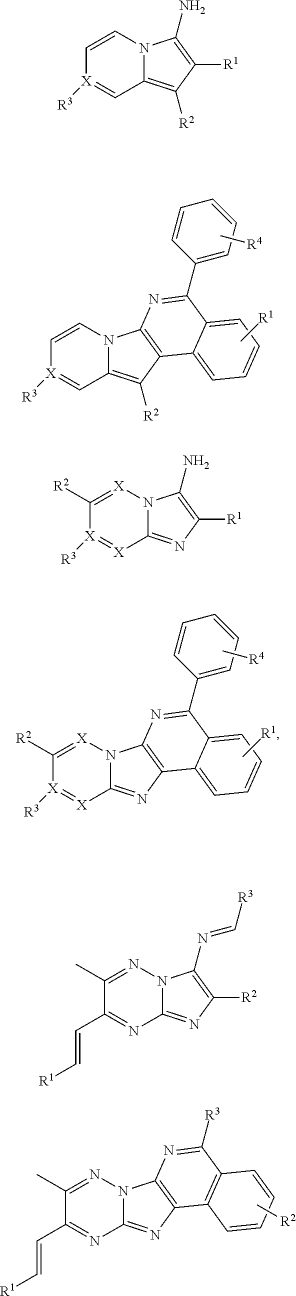 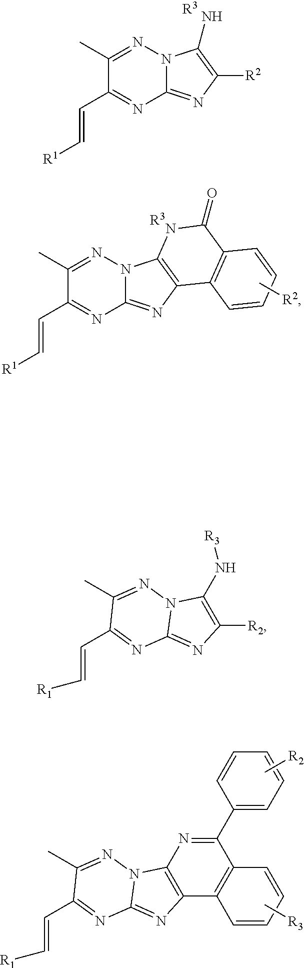 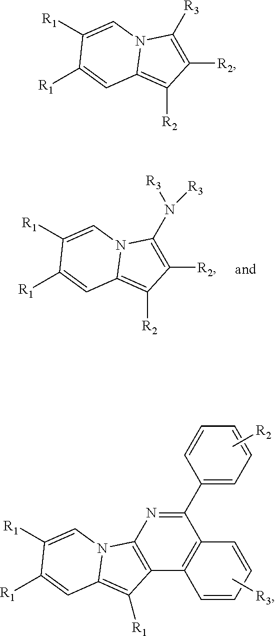
wherein X is CR, CH, or N, and R₁, R₂, R₃, and R₄ are independently selected from the group consisting of null, H, (CH₂)ₙ—O—(CH₂)ₘ, substituted or unsubstituted aryl, substituted or unsubstituted C₁-C₆ alkyl, where n and m are independently integers between 0 and 6, CN, halide, N-alkyl, O-alkyl, and N-aryl.

2. The composition of claim 1, wherein R₁ and R₄ are —OCH₃.

3. The composition of claim 1 or 2, wherein R₂ is CH₂CN.

4. The composition of any of the preceding claims, wherein R₁ and R₄ are independently ortho, meta, para, or a combination thereof.

5. The composition of any of the preceding claims, wherein R₁, R₂, R₃, and R₄ are independently selected from the group consisting of isobutyl,

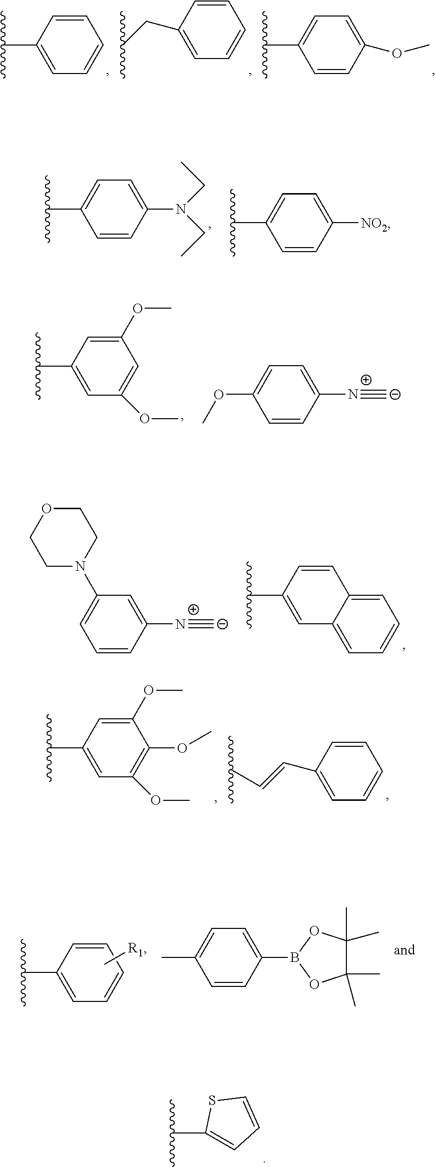

6. The composition of any of the preceding claims, wherein said compound is selected from the group consisting of

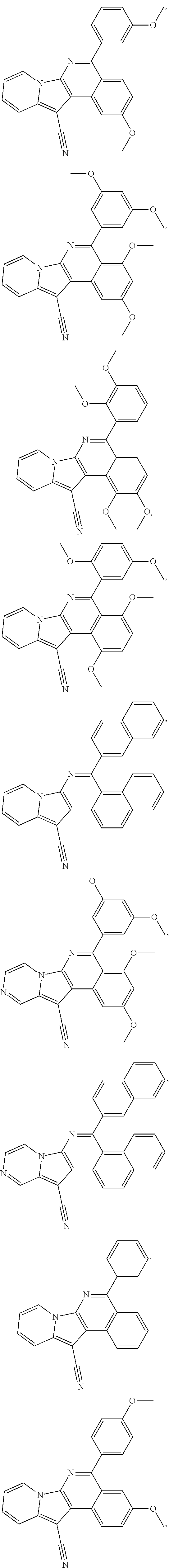

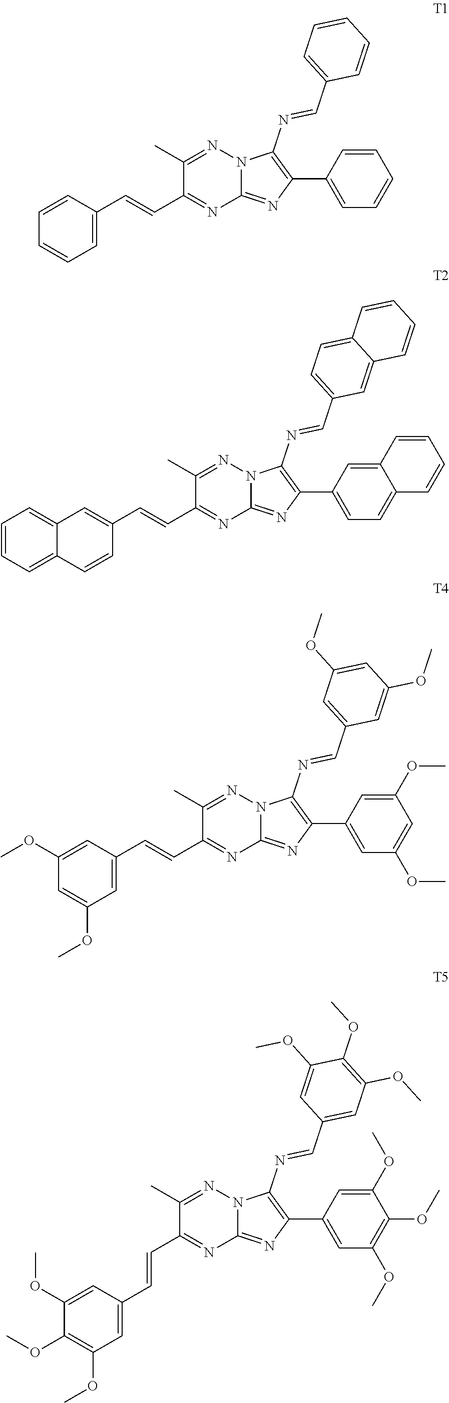

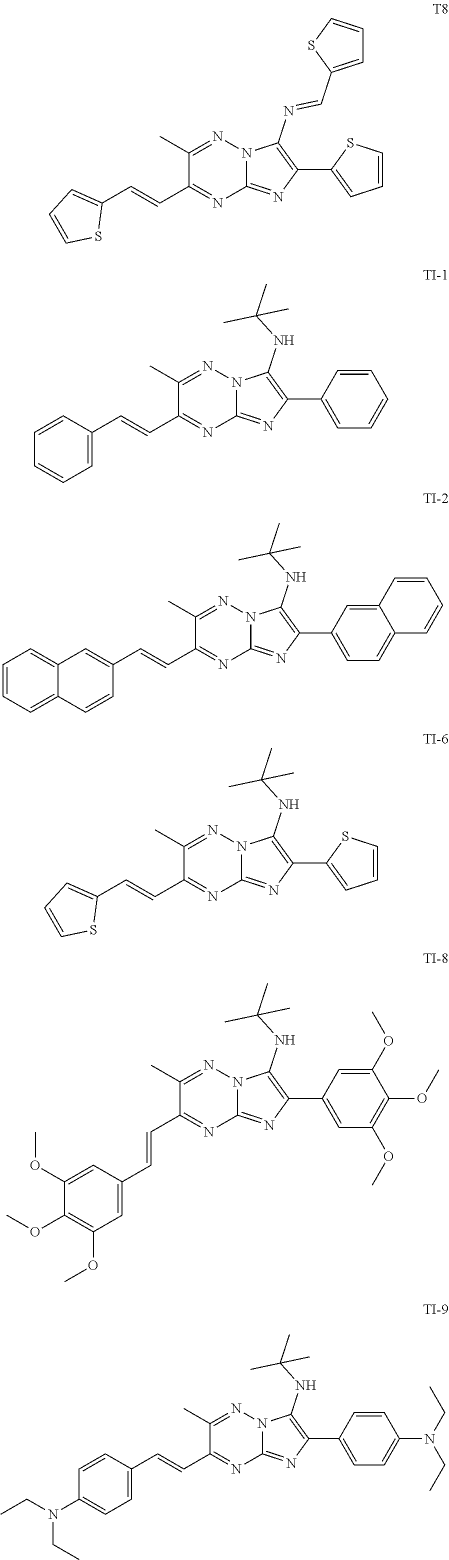

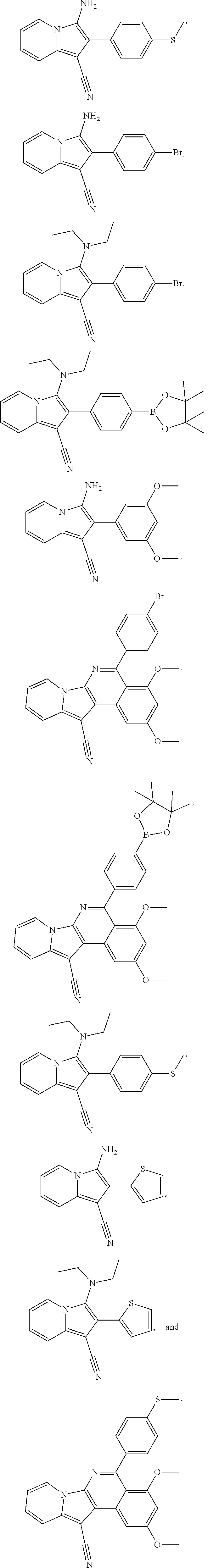

7. A composition comprising a compound selected from

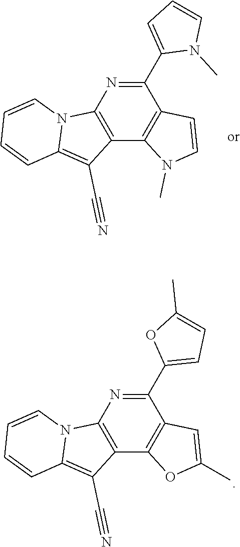 [SMILES: Cn1cccc1-c1nc2c(c(C#N)c3ccccn32)c2c1ccn2C ; Cc1ccc(-c2nc3c(c(C#N)c4ccccn43)c3oc(C)cc23)o1]

8. The composition of any of the preceding claims, wherein said compound is fluorescent.

9. The composition of any of the preceding claims, wherein said compound is a probe.

10. The composition of any of the preceding claims, wherein said compound is conjugated to a second molecule.

11. The composition of claim 10, wherein said conjugation is via a linker.

12. The composition of claim 10 or 11, wherein said conjugation is at R₃.

13. A kit or system, comprising one or more compounds of any of the preceding claims.

14. The kit or system of claim 13, wherein said one or more compounds comprises two or more compounds, wherein each of said compounds emits fluorescence of a different wavelength.

15. The kit or system of claim 13 or 14, wherein said kit or system further comprises one or more additional component selected from the group consisting of one or more buffers, one or more detection reagents, a fluorescent spectrometer, one or more control agents, and software for analysis or detection of an analyte of interest.

16. A method of detecting an analyte of interest, comprising:
a) contacting a sample comprising the analyte of interest with the kit or system of any one of claims 13 to 15; and
b) detecting fluorescence from said compound.

17. The method of claim 16, wherein said analyte is selected from the group consisting of a protein, a small molecule, and an acid.

18. The method of claim 17, wherein said method detects the pH of said sample.

19. The use of the kit or system of any one of claims 13 to 15 to detect an analyte of interest.
